(19) United States
(12) Patent Application Publication
(10) Pub. No.: US 2025/0312345 A1
(43) Pub. Date: Oct. 9, 2025

(54) NON-COVALENT INHIBITORS OF THE MAIN PROTEASE OF SARS-COV-2 AND METHODS OF USE

(71) Applicant: YALE UNIVERSITY, New Haven, CT (US)

(72) Inventor: Willliam L. Jorgensen, New Haven, CT (US)

(21) Appl. No.: 18/881,672

(22) PCT Filed: Jul. 7, 2022
(86) PCT No.: PCT/US2022/036408

§ 371 Date: Jan. 6, 2025

(51) Int. Cl. A61K 31/506; A61K 31/444; A61P 31/14; C07D 401/04; C07D 401/10; C07D 401/14; C07D 409/14; C07D 413/14; C07D 417/14

(52) U.S. Cl. CPC A61K 31/506; A61K 31/444; A61P 31/14; C07D 401/04; C07D 401/10; C07D 401/14; C07D 409/14; C07D 413/14; C07D 417/14

(57) ABSTRACT

Provided herein are compounds of formula (I) or (I-A), which act as non-covalent inhibitors of SARS-CoV2 main protease (M^{pro}). In certain embodiments, the compounds of formula (I) or (I-A) are useful in treating, ameliorating, and/or preventing COVID-19 infections, as well as reducing or ameliorating symptoms associated with COVID-19 infection.

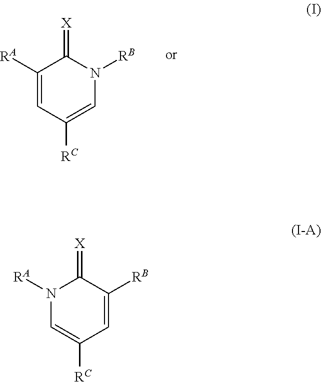

### STATEMENT REGARDING FEDERALLY SPONSORED RESEARCH

This invention was made with government support under GM032136 awarded by National Institutes of Health. The government has certain rights in the invention.

### BACKGROUND

The coronavirus SARS-CoV-2, which is the cause of the COVID-19 pandemic, encodes several enzymes that are essential to its ability to replicate. After cell entry, viral RNA is translated by host ribosomes into two polyproteins that are cleaved to produce the viral proteins that are needed for assembling new virions. As potential targets for discovery of therapeutic agents, the two cysteine proteases that are responsible for cleaving the two polyproteins have been highlighted, namely, the chymotrypsin-like or main protease, known as 3CL^{pro} or M^{pro}, and the papain-like protease, PL^{pro}.

Following the disease outbreak in 2002 from SARS-CoV, these proteins have received much attention for characterization of their structural biology and development of inhibitors. The high sequence homology between the proteins from the two coronaviruses, 83% for PL^{pro} and 96% for M^{pro}, has allowed the prior studies to provide a solid foundation for current efforts targeting the new isoforms. Thus, crystal structures of M^{pro} from SARS-CoV-2 have quickly emerged along with initial reports of inhibitors. As for the earlier virus, the designed inhibitors have largely been peptide-like with incorporation of a reactive warhead that covalently binds to the catalytic cysteine, Cys145. These features are generally not optimal for drug development owing to potential proteolytic degradation, limited antiviral activity, and toxicities from off-target covalent modification of other biomolecules.

Due to the global scope and severity of the COVID-19 pandemic, there is a pressing medical need for new drugs. Described herein are non-peptidic, non-covalent inhibitors of SARS-CoV-2 M^{pro} that are drug-like and show both high inhibitory and anti-viral activity.

### BRIEF SUMMARY

In various aspects, a compound of formula (I) or formula (I-A), or a salt, solvate, enantiomer, diastereomer, tautomer, or N-oxide thereof, has the structure:

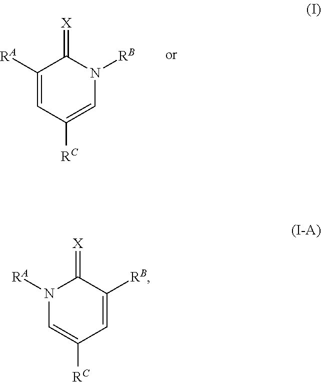

wherein:
each occurrence of R^{A} is independently a 5, 6, 7, or 8-membered heterocyclyl;
each occurrence of R^{B} is independently a 5, 6, 7, or 8-membered heterocyclyl;
each occurrence of R^{C} is independently a 5, 6, 7, or 8-membered heterocyclyl; and
each occurrence of X is independently O, S, or N—OR;
wherein each of R^{A}, R^{B}, and R^{C} is independently substituted by 1 to 5 substituents independently selected from the group consisting of hydrogen, C₆₋₁₄ aryl, C₆₋₁₄ heteroaryl, C₁₋₁₀ alkoxy, C₁₋₁₀ alkyl, C₃₋₁₀ cycloalkyl, C₃₋₁₀ cycloheteroalkyl, C₂₋₁₀ alkenyl, C₂₋₁₀ alkynyl, OR, OC(O)N(R)₂, OCH₂C(O)N(R)₂, O (oxo), F, Cl, Br, I, NO₂, CN, CF₃, OCF₃, N(R)₂, SR, SOR, SO₂R, SO₂N(R)₂, SO₃R, C(O)R, C(O)OR, OC(O)R, C(O)N(R)₂, and combinations thereof; and
wherein each occurrence of R is independently hydrogen, C₁₋₁₂ alkyl, C₂₋₁₂ alkenyl, C₂₋₁₂ alkynyl, C₅₋₁₂ cycloalkyl, C₆₋₁₀ aryl, C₅₋₁₀ heteroaryl, or combinations thereof.

In various aspects, a method of treating, ameliorating, and/or preventing COVID-19 is provided and includes administering a therapeutically effective amount of a compound of the disclosure to a subject in need thereof.

Advantageously, in various aspects, synergistic effects are observed when compounds of formula (I) or (I-A) are combined with at least one other anti-viral agent.

### BRIEF DESCRIPTION OF THE FIGURES

The drawings illustrate generally, by way of example, but not by way of limitation, various embodiments of the present application.

FIGS. 1A-1B show docked structures for perampanel with M^{pro}. FIG. 1A shows a surface rendering noting the binding pockets colored by atomic number. FIG. 1B shows residues near the binding site including the catalytic residues His41 and Cys145. Carbon atoms of the ligand are in yellow. Circled letters are features discussed in the text.

FIGS. 2A-2F show structures for complexes with M^{pro}. Hydrogen bonds between the ligand and protein are noted with dashed lines. FIG. 2A is a 1.6-Å crystal structure for 9. PDB ID: 7L10. FIG. 2B is a 1.8-Å crystal structure for 13. PDB ID: 7L11. FIG. 2C depicts a close-up of the P4 site with surface rendering of the protein and CPK rendering of 13. FIGS. 2D and 2E show modeled structures for complexes of 14 and 17 with M^{pro}. Mixed rendering with emphasis on placement of the alkoxy substituent in the P3-P4 channel. FIG. 2F is 1.8-Å crystal structure for 14. PDB ID: 7L12.

FIG. 3 is a 3D-plot showing synergistic antiviral behavior for 13 and remdesivir.

FIGS. 4A-4B show the crystal structure of compound 37 in the active site pf M^{pro}. Compound 37 is shown in magenta and residues of M^{pro} are shown in blue in (FIG. 4A) surface and (FIG. 4B) space-filling rendering. The ortho-chloro group of 37 is shown interacting with the pocket formed by Met165 and Leu167. This structure was determined to 2.2 Å.

FIGS. 5A-5B show dose-response curves for EIDD-1931 alone (FIG. 5A) and compound 61 alone (FIG. 5B).

FIG. 6 is a 3D plot illustrating synergy in the anti-viral effectiveness of a combination of EIDD-1931 and compound 61 at various concentrations.

FIG. 7 shows in vitro pharmacological profiling of compounds of formula (I) and (I-A) against targets for adverse drug reactions. Rows represent compounds tested and columns represent targets. Percentage inhibition at 10 μM concentration of the compounds is color-coded. A panel of 35 targets including CYP450 isoforms, ions channels, receptors examined for off-target effects. Compound 61 (MPro 61) showed no significant off-target effects except minimal inhibition of HERG ion channel at high concentrations.

FIG. 8 depicts a schematic of a preliminary pharmacokinetic study with compound 61 in mice. Compound 61: Biochemical assay: IC₅₀=44 nM; SARS-CoV-2 replicon=EC₅₀ of 175 nM; SARS-CoV-2 infectious virus EC₅₀=80 nM; CC₅₀>32 μM. Preliminarys PK studies in CB C57BL/6 (BJ6) after 15 mg/kg of Compounds 61 dosed oral & IV determined Compound 61 levels 7-fold higher than EC₉₀ (˜600 nM) at the 24 hr time point for IV and oral doses. Extracted lung tissues showed Compound 61 after 24 hr IV dose ˜3-fold higher than EC₉₀; oral ˜EC₉₀. Confirmed serum and lung tissue extracts exhibited inhibition of SARS-CoV-2 replication in cell culture by Compounds 61.

FIG. 9 describes validation models for antiviral Efficacy of Compound 61 Against Infectious SARS-CoV-2.

FIGS. 10A-10B show a comparison of effects of remdesivir and compound 61 (Mpro 61) in two multiplicity of cellular infection (MOI) scenarios, 2.5 MOI (FIG. 10A) and 0.25 MOI (FIG. 10B).

FIG. 11 depicts a sample experimental/instrumental protocol for determining antiviral efficacy of the compounds of formula (I) and (I-A).

FIG. 12 illustrates a sample dosing protocol of compounds of formula (I) and (I-A) by oral and IV administration routes in transgenic K18-hACE2 mice.

FIGS. 13A-13B show anti-viral effects of compound 61 dosed according to the protocol in FIG. 13A as measured by bioluminescence imaging (BLI) using SARS-CoV-2 with nanoluciferase reporter.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C═CH, —C═C(CH₃), —C═C(CH₂CH₃), —CH₂C═CH, —CH₂C═C(CH₃), and —CH₂C═C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbomyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. The term heterocyclyl includes rings where a CH₂ group in the ring is replaced by one or more C═O groups, such as found in cyclic ketones, lactones, and lactams. Examples of heterocyclyl groups containing a C═O group include, but are not limited to, β-propiolactam, γ-butyrolactam, δ-valerolactam, and ε-caprolactam, as well as the corresponding lactones. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring designated C_{x-y} can be any ring containing ‘x’ members up to ‘y’ members, including all intermediate integers between ‘x’ and ‘y’ and that contains one or more heteroatoms, as defined herein. In a ring designated C_{x-y}, all non-heteroatom members are carbon. Heterocyclyl rings designated C_{x-y} can also be polycyclic ring systems, such as bicyclic or tricyclic ring systems. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxy cyclohexyl)ethyl, 2-(2,3-epoxy cylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl or C_{a-b} hydrocarbyl, wherein a and b are integers and mean having any of a to b number of carbon atoms. For example, (C₁-C₄)hydrocarbyl or C₁₋₄ hydrocarbyl means the hydrocarbyl group can be methyl (C₁), ethyl (C₂), propyl (C₃), cyclopropyl (C₃), cyclobutyl (C₄), cyclopropylmethyl (C₄), or butyl (C₄), and (C₀-C_{b})hydrocarbyl or C_{0-b} hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate).

Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

### Compounds

Compounds of formula (I) or (I-A) or otherwise described herein can be prepared by the general schemes described herein, using the synthetic method known by those skilled in the art. The following examples illustrate non-limiting embodiments of the compound(s) described herein and their preparation.

In certain embodiments, a compound of formula (I) or formula (I-A), or a salt, solvate, enantiomer, diastereomer, tautomer, or N-oxide thereof is provided. The compounds of formula (I) and formula (I-A) have the following structure:

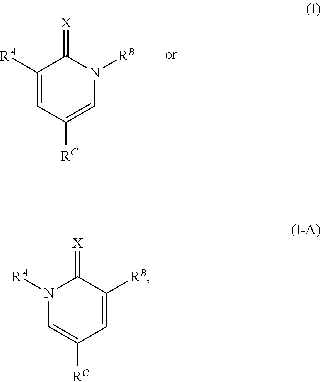

wherein:
   each occurrence of R^{A} is independently a 5, 6, 7, or 8-membered heterocyclyl;
   each occurrence of R^{B} is independently a 5, 6, 7, 9, or 10-membered heterocyclyl;
   each occurrence of R^{C} is independently a 5, 6, 7, or 8-membered heterocyclyl; and
   each occurrence of X is independently O, S, or N—OR;
wherein each occurrence of R^{A}, R^{B}, and R^{C} is independently substituted by 1 to 5 substituents selected from the group consisting of hydrogen, C₆₋₁₄ aryl, C₆₋₁₄ heteroaryl, C₁₋₁₀ alkoxy, C₁₋₁₀ alkyl, C₃₋₁₀ cycloalkyl, C₃₋₁₀ cycloheteroalkyl, C₂₋₁₀ alkenyl, C₂₋₁₀ alkynyl, OR, OC(O)N(R)₂, OCH₂C(O)N(R)₂, O (oxo), F, Cl, Br, I, NO₂, CN, CF₃, OCF₃, N(R)₂, SR, SOR, SO₂R, SO₂N(R)₂, SO₃R, C(O)R, C(O)OR, OC(O)R, C(O)N(R)₂, and combinations thereof; and
wherein each occurrence of R is independently hydrogen, C₁₋₁₂ alkyl, C₂₋₁₂ alkenyl, C₂₋₁₂ alkynyl, C₅₋₁₂ cycloalkyl, C₆₋₁₀ aryl, C₅₋₁₀ heteroaryl, or combinations thereof.

In certain embodiments, each R is independently hydrogen or C₁₋₁₀ hydrocarbyl.

By “combinations thereof” it means that the substituents can be combined to form a different substituent. For example, and without limitation, one or more CF₃ groups can be combined with a C₁₋₁₀ alkyl group and such a combination would be a single substituent.

In certain embodiments, X is O, R^{A} is a 6-membered aryl or heteroaryl, R^{B} is a 6-membered aryl or heteroaryl, and R^{C} is a 6-membered cycloheteroalkyl, aryl, or heteroaryl. In certain embodiments, R^{A} corresponds to the P2 pocket, R^{B} corresponds to the P1 pocket, and R^{C} corresponds to the P1′ pocket in SARS-CoV-2 M^{pro} as described herein.

In various embodiments, R^{A} is:

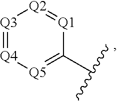

wherein Q1 is C-A¹ or N, Q2 is C-A² or N, Q3 is C-A³ or N, Q4 is C-A⁴ or N, and Q5 is C-A⁵ or N, wherein 0-3 of Q1-Q5 can be N, wherein each A¹-A⁵ is independently selected from the group consisting of hydrogen, C₆₋₁₄ aryl, C₆₋₁₄ heteroaryl, C₁₋₁₀ alkoxy, C₃₋₁₀ cycloalkyl, C₃₋₁₀ cycloheteroalkyl, C₁₋₁₀ alkyl, C₂₋₁₀ alkenyl, C₂₋₁₀ alkynyl, OR, OC(O)N(R)₂, F, Cl, Br, I, NO₂, CN, CF₃, OCF₃, N(R)₂, SR, SOR, SO₂R, SO₂N(R)₂, SO₃R, C(O)R, C(O)OR, OC(O)R, C(O)N(R)₂, and combinations thereof.

In various embodiments, R^{A} is:

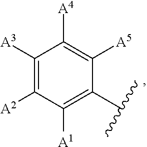

and one of the following applies to the compound of formula (I) or (I-A):
i. A¹, A³, A⁴, and A⁵ are hydrogen,
ii. A¹ and A⁵ are hydrogen, or
ii. A¹, A³, and A⁵ are hydrogen.

In various embodiments, each A¹-A⁵ is independently selected from the group consisting of hydrogen, C₆₋₁₄ aryl, C₁₋₁₀ alkoxy, C₃₋₁₀ cycloalkyl, C₃₋₁₀ cycloheteroalkyl, C₁₋₁₀ alkyl, OR, OC(O)N(R)₂, OCH₂C(O)N(R)₂, F, Cl, Br, NO₂, CN, CF₃, OCF₃, and combinations thereof. In various embodiments, each A¹-A⁵ is independently selected from the group consisting of hydrogen, F, Cl, CN, OC₁₋₆ alkyl, OC₁₋₆ alkyl substituted by 1 to 5 hydroxyl groups, OC₁₋₆ alkyl substituted by 1 to 5 CF₃ groups, O(CH₂)ₙPh, O(CH₂)ₙAr, and O(CH₂CH₂O)ₘCH₃,
wherein
   each n is independently at each occurrence an integer from 1 to 5;
   each m is independently at each occurrence an integer from 1 to 5;
   Ar is phenyl substituted with 1 to 5 substituents selected from the group consisting of C₁₋₅ hydrocarbyl, CF₃, F, Cl, Br, and combinations thereof, or
   Ar is a 5-membered heteroaryl or a 6-membered heteroaryl substituted with 1 to 5 substituents selected from the group consisting of hydrogen, C₁₋₅ hydrocarbyl, CF₃, F, Cl, Br, and combinations thereof. Suitable 5-membered heteroaryl include, but are not limited to, oxazole, isoxazole, thiazole, and/or isothiazole.

In various embodiments, R^{A} is

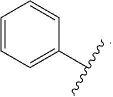 [SMILES: *c1ccccc1]

In various embodiments, R^{A} is

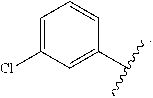 [SMILES: *c1cccc(Cl)c1]

In various embodiments, R^{A} is

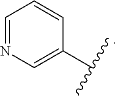 [SMILES: *c1cccnc1]

In various embodiments, R^{A} is

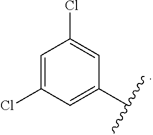 [SMILES: *c1cc(Cl)cc(Cl)c1]

In various embodiments, R^{A} is

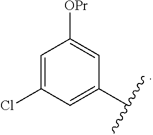

In various embodiments, R^{A} is

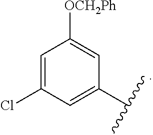

In various embodiments, R^{A} is

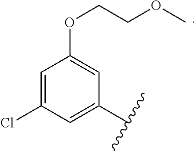 [SMILES: *c1cc(Cl)cc(OCCOC)c1]

In various embodiments, R^{A} is

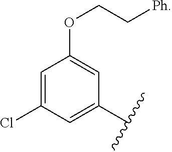 [SMILES: *c1cc(Cl)cc(OCCc2ccccc2)c1]

In various embodiments, R^{A} is

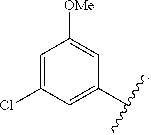 [SMILES: *c1cc(Cl)cc(OC)c1]

In various embodiments, R^{A} is

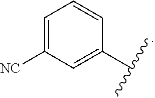 [SMILES: *c1cccc(C#N)c1]

In various embodiments, R^{A} is

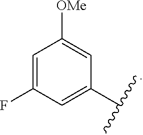 [SMILES: *c1cc(F)cc(OC)c1]

In various embodiments, R^{A} is

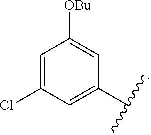

In various embodiments, R^{A} is

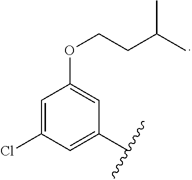 [SMILES: *c1cc(Cl)cc(OCCC(C)C)c1]

In various embodiments, R^{A} is

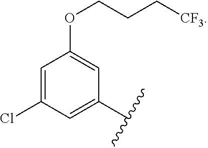 [SMILES: *c1cc(Cl)cc(OCCCC(F)(F)F)c1]

In various embodiments, R^{A} is

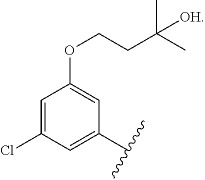 [SMILES: *c1cc(Cl)cc(OCCC(C)(C)O)c1]

In various embodiments, R^{A} is

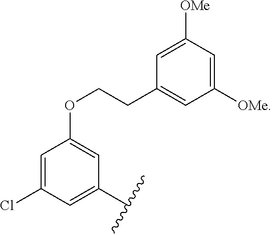 [SMILES: *c1cc(Cl)cc(OCCc2cc(OC)cc(OC)c2)c1]

In various embodiments, R^{A} is

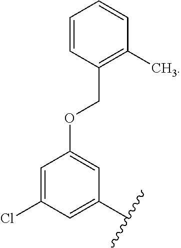 [SMILES: *c1cc(Cl)cc(OCc2ccccc2C)c1]

In various embodiments, R^{A} is

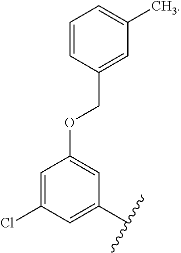 [SMILES: *c1cc(Cl)cc(OCc2cccc(C)c2)c1]

In various embodiments, R^{A} is

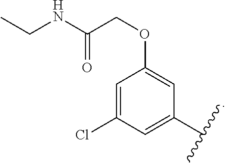 [SMILES: *c1cc(Cl)cc(OCC(=O)NCC)c1]

In various embodiments, R^{A} is

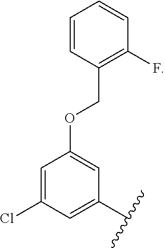 [SMILES: *c1cc(Cl)cc(OCc2ccccc2F)c1]

In various embodiments, R^{A} is

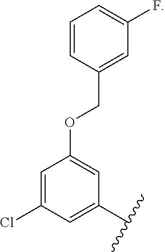 [SMILES: *c1cc(Cl)cc(OCc2cccc(F)c2)c1]

In various embodiments, R^{A} is

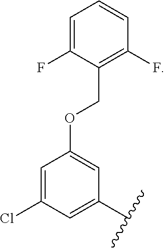 [SMILES: *c1cc(Cl)cc(OCc2c(F)cccc2F)c1]

In various embodiments, R^{A} is

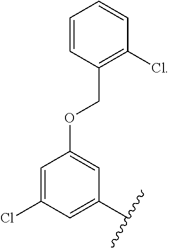 [SMILES: *c1cc(Cl)cc(OCc2ccccc2Cl)c1]

In various embodiments, R^{A} is

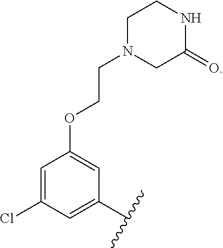 [SMILES: *c1cc(Cl)cc(OCCN2CCNC(=O)C2)c1]

In various embodiments, R^{A} is

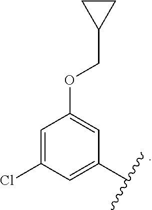 [SMILES: *c1cc(Cl)cc(OCC2CC2)c1]

In various embodiments, R^{A} is

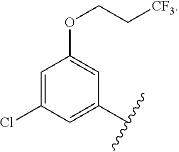 [SMILES: *c1cc(Cl)cc(OCCC(F)(F)F)c1]

In various embodiments, R^{A} is

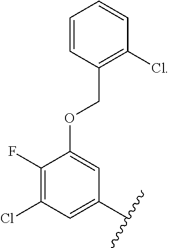 [SMILES: *c1cc(Cl)c(F)c(OCc2ccccc2Cl)c1]

In various embodiments, R^{A} is

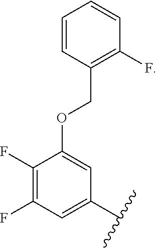 [SMILES: *c1cc(F)c(F)c(OCc2ccccc2F)c1]

In various embodiments, R^{A} is

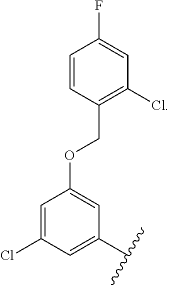 [SMILES: *c1cc(Cl)cc(OCc2ccc(F)cc2Cl)c1]

In various embodiments, R^{A} is

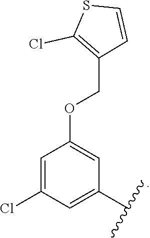 [SMILES: *c1cc(Cl)cc(OCc2ccsc2Cl)c1]

In various embodiments, R^{A} is

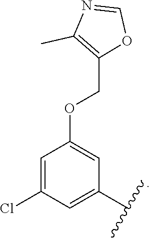 [SMILES: *c1cc(Cl)cc(OCc2ocnc2C)c1]

In various embodiments, R^{A} is

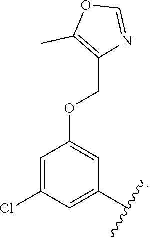 [SMILES: *c1cc(Cl)cc(OCc2ncoc2C)c1]

In various embodiments, R^{A} is

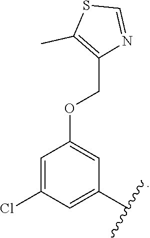 [SMILES: *c1cc(Cl)cc(OCc2ncsc2C)c1]

In various embodiments, R^{A} is

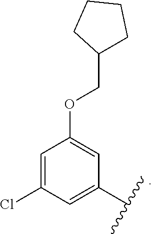 [SMILES: *c1cc(Cl)cc(OCC2CCCC2)c1]

In various embodiments, R^{A} is

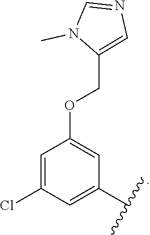 [SMILES: *c1cc(Cl)cc(OCc2cncn2C)c1]

In various embodiments, R^{A} is

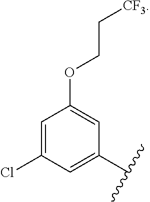 [SMILES: *c1cc(Cl)cc(OCCC(F)(F)F)c1]

In various embodiments, R^{A} is

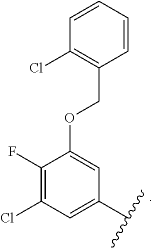 [SMILES: *c1cc(Cl)c(F)c(OCc2ccccc2Cl)c1]

In various embodiments, R^{A} is

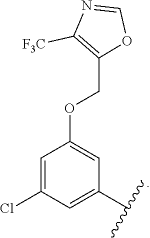 [SMILES: *c1cc(Cl)cc(OCc2ocnc2C(F)(F)F)c1]

In various embodiments, R^{A} is

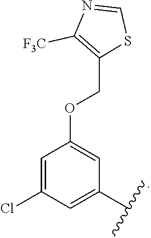 [SMILES: *c1cc(Cl)cc(OCc2scnc2C(F)(F)F)c1]

In various embodiments, R^{A} is

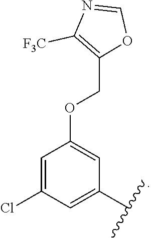 [SMILES: *c1cc(Cl)cc(OCc2ocnc2C(F)(F)F)c1]

In various embodiments, R^{A} is

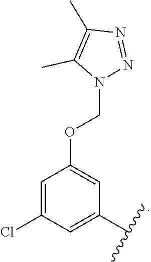 [SMILES: *c1cc(Cl)cc(OCn2nnc(C)c2C)c1]

In various embodiments, R^{A} is

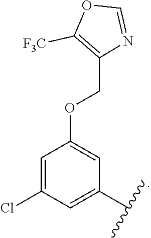 [SMILES: *c1cc(Cl)cc(OCc2ncoc2C(F)(F)F)c1]

In various embodiments, R^{A} is

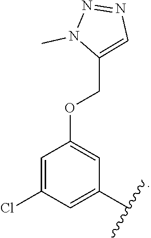 [SMILES: *c1cc(Cl)cc(OCc2cnnn2C)c1]

In various embodiments, R^{A} is

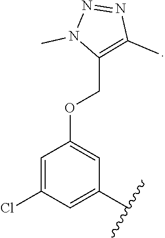 [SMILES: *c1cc(Cl)cc(OCc2c(C)nnn2C)c1]

In various embodiments, R^{A} is

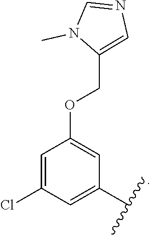 [SMILES: *c1cc(Cl)cc(OCc2cncn2C)c1]

In various embodiments, R^{A} is

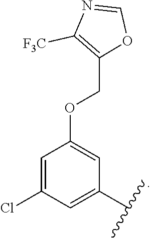 [SMILES: *c1cc(Cl)cc(OCc2ocnc2C(F)(F)F)c1]

In various embodiments, R^{B} is

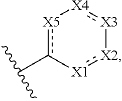

wherein
   each X1-X5 is independently C—Y, N, or NR;
   each Y is independently selected from the group consisting of hydrogen, O (oxo), F, Cl, C₁₋₄ alkyl, and OC₁₋₄ alkyl;
   [symbol] is a single or double bond; and
provided that
1 to 3 of X1-X5 is N or NR, and
if at least one of X1-X5 is NR then an adjacent position to the NR is C═O.

In various embodiments, R^{B} is

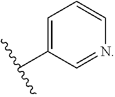 [SMILES: *c1cccnc1]

In various embodiments, R^{B} is

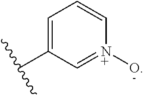

In various embodiments, R^{B} is

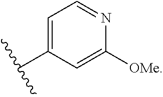 [SMILES: *c1ccnc(OC)c1]

In various embodiments, R^{B} is

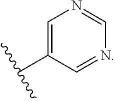 [SMILES: *c1cncnc1]

In various embodiments, R^{B} is

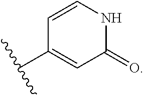 [SMILES: *c1cc[nH]c(=O)c1]

In various embodiments, R^{B} is

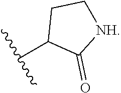 [SMILES: *C1CCNC1=O]

In various embodiments, R^{B} is

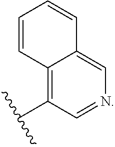 [SMILES: *c1cncc2ccccc12]

In various embodiments, R^{C} is

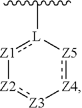

wherein
each Z1-Z5 is independently C—Y, N, or NR;
L is C or N;
each Y is independently selected from the group consisting of hydrogen, O (oxo), F, Cl, Br, I, NO₂, CN, CF₃, OCF₃, C₁₋₄ alkyl, and C₁₋₄ alkoxy;
[symbol] is a single or double bond; and
provided that
1 to 3 of Z1-Z5 is N or NR, and
if at least one of Z1-Z5 is NR then an adjacent position to the NR is C═O.

The term “adjacent position” as used herein with respect to ring R^{B} or R^{C} means the group of atoms (e.g., CH) to either side of an NR group in ring R^{B} or R^{C}. The adjacent position to an NR group does not include the atom by which ring R^{B} or R^{C} is attached to the rest of the compound of formula (I) or (I-A). If the NR group is positioned between two groups in ring R^{B} or R^{C}, then both groups can be C═O. Thus, for example, and without limitation, ring R^{B} or R^{C} can have the following structure:

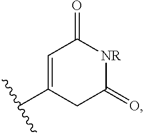

which can further tautomerize to form a hydroxy pyridone.

In various embodiments, R^{B} is

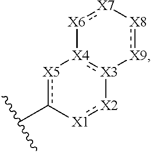

wherein
each X1, X2, X5, X6, X7, X8, and X9 is independently C—Y, N, or NR;
each X3 and X4 is independently C—Y or C;
each Y is independently selected from the group consisting of hydrogen, O (oxo), F, Cl, C₁₋₄ alkyl, and OC₁₋₄ alkyl;
[symbol] is a single or double bond; and
provided that
1 to 3 of X1, X2, X5, X6, X7, X8, and X9 is N or NR, and
if at least one of X1, X2, X5, X6, X7, X8, and X9 is NR then an adjacent position to the NR is C═O.

In various embodiments, R^{B} is

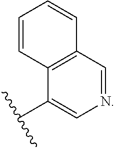 [SMILES: *c1cncc2ccccc12]

In various embodiments, R^{C} is

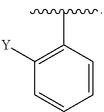

In various embodiments, R^{C} is

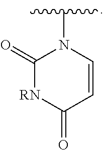

In various embodiments, R^{C} is

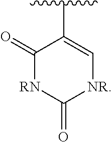

In various embodiments, R^{C} is

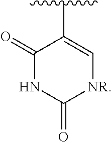

In various embodiments, R^{C} is

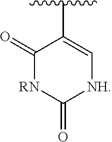

In various embodiments, R^{C} is

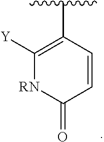

In various embodiments, R^{C} is

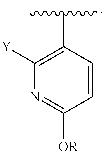

In various embodiments, Y is CN. In various embodiments, Y In various embodiments, each R bonded to an N atom in R^{C} is hydrogen. In various embodiments, each R bonded to an N atom in R^{C} is C₁₋₄ alkyl optionally substituted by at least one selected from the group consisting of F, Cl, Br, and D (deuterium). In various embodiments, each R bonded to an N atom in R^{C} is selected from the group consisting of methyl, ethyl, n-propyl, cyclopropyl, iso-propyl, n-butyl, s-butyl, and t-butyl optionally substituted by at least one selected from the group consisting of F, Cl, Br, and D (deuterium).

In various embodiments, R^{C} is

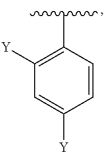

wherein each Y is independently selected from the group consisting of CN, F, Cl, Br, NR₂, CF₃, CHF₂, and CH₂F. In various embodiments, R^{C} is

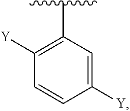

wherein each Y is independently selected from the group consisting of CN, F, Cl, Br, NR₂, CF₃, CHF₂, and CH₂F. In various embodiments, R^{C} is

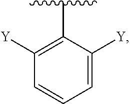

wherein each Y is independently selected from the group consisting of CN, F, Cl, Br, NR₂, CF₃, CHF₂, and CH₂F. In various embodiments, R^{C} is

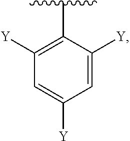

wherein each Y is independently selected from the group consisting of CN, F, Cl, Br, NR₂, CF₃, CHF₂, and CH₂F.

In various embodiments, R^{C} is

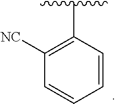 [SMILES: *c1ccccc1C#N]

In various embodiments, R^{C} is

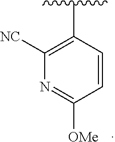 [SMILES: *c1ccc(OC)nc1C#N]

In various embodiments, R^{C} is

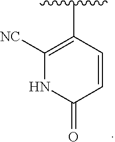 [SMILES: *c1ccc(=O)[nH]c1C#N]

In various embodiments, R^{C} is

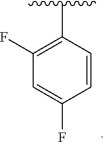 [SMILES: *c1ccc(F)cc1F]

In various embodiments, R^{C} is

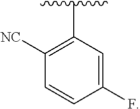 [SMILES: *c1cc(F)ccc1C#N]

In various embodiments, R^{C} is

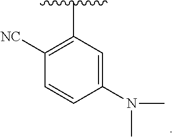 [SMILES: *c1cc(N(C)C)ccc1C#N]

In various embodiments, R^{C} is

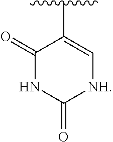 [SMILES: *c1c[nH]c(=O)[nH]c1=O]

In various embodiments, R^{C} is

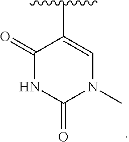 [SMILES: *c1cn(C)c(=O)[nH]c1=O]

In various embodiments, R^{C} is

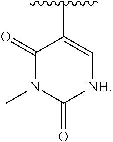 [SMILES: *c1c[nH]c(=O)n(C)c1=O]

In various embodiments, provided herein is a pharmaceutical composition that includes a compound of formula (I) or (I-A) and at least one pharmaceutically acceptable excipient.

In various embodiments, the compound of formula (I) or (I-A) is

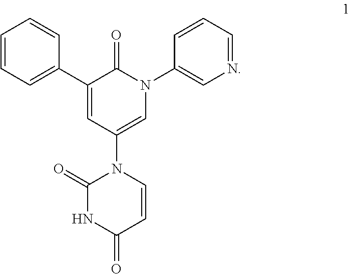 [SMILES: O=c1ccn(-c2cc(-c3ccccc3)c(=O)n(-c3cccnc3)c2)c(=O)[nH]1]

In various embodiments, the compound of formula (I) or (I-A) is

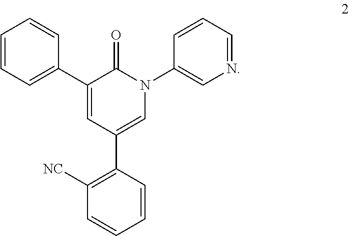 [SMILES: N#Cc1ccccc1-c1cc(-c2ccccc2)c(=O)n(-c2cccnc2)c1]

In various embodiments, the compound of formula (I) or (I-A) is

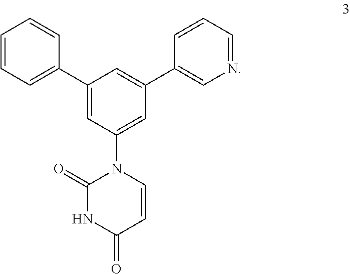 [SMILES: O=c1ccn(-c2cc(-c3ccccc3)cc(-c3cccnc3)c2)c(=O)[nH]1]

In various embodiments, the compound of formula (I) or (I-A) is

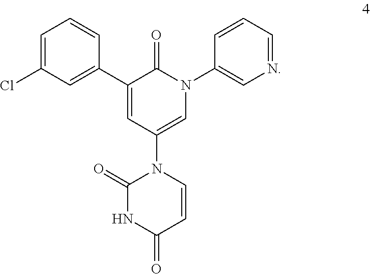 [SMILES: O=c1ccn(-c2cc(-c3cccc(Cl)c3)c(=O)n(-c3cccnc3)c2)c(=O)[nH]1]

In various embodiments, the compound of formula (I) or (I-A) is

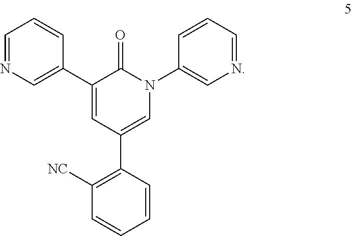 [SMILES: N#Cc1ccccc1-c1cc(-c2cccnc2)c(=O)n(-c2cccnc2)c1]

In various embodiments, the compound of formula (I) or (I-A) is

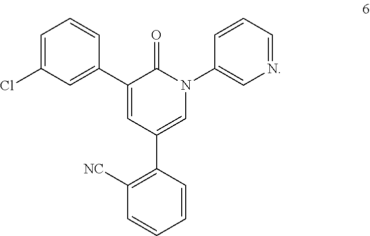 [SMILES: N#Cc1ccccc1-c1cc(-c2cccc(Cl)c2)c(=O)n(-c2cccnc2)c1]

In various embodiments, the compound of formula (I) or (I-A) is

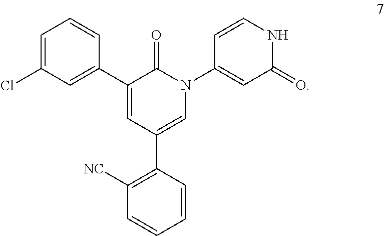 [SMILES: N#Cc1ccccc1-c1cc(-c2cccc(Cl)c2)c(=O)n(-c2cc[nH]c(=O)c2)c1]

In various embodiments, the compound of formula (I) or (I-A) is

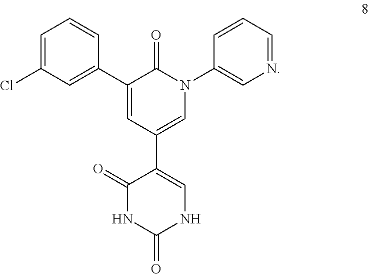 [SMILES: O=c1[nH]cc(-c2cc(-c3cccc(Cl)c3)c(=O)n(-c3cccnc3)c2)c(=O)[nH]1]

In various embodiments, the compound of formula (I) or (I-A) is

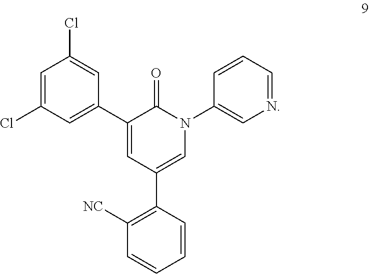 [SMILES: N#Cc1ccccc1-c1cc(-c2cc(Cl)cc(Cl)c2)c(=O)n(-c2cccnc2)c1]

In various embodiments, the compound of formula (I) or (I-A) is

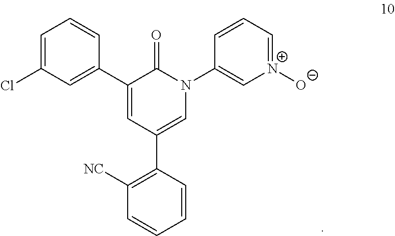 [SMILES: N#Cc1ccccc1-c1cc(-c2cccc(Cl)c2)c(=O)n(-c2ccc[n+]([O-])c2)c1]

In various embodiments, the compound of formula (I) or (I-A) is

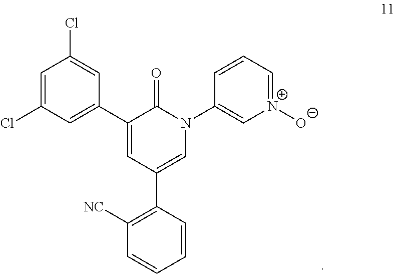 [SMILES: N#Cc1ccccc1-c1cc(-c2cc(Cl)cc(Cl)c2)c(=O)n(-c2ccc[n+]([O-])c2)c1]

In various embodiments, the compound of formula (I) or (I-A) is

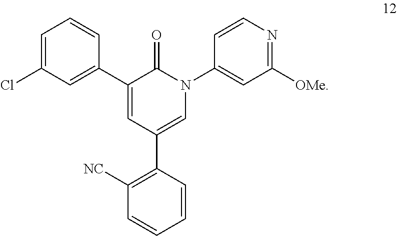 [SMILES: COc1cc(-n2cc(-c3ccccc3C#N)cc(-c3cccc(Cl)c3)c2=O)ccn1]

In various embodiments, the compound of formula (I) or (I-A) is

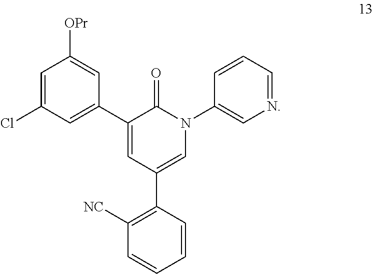

In various embodiments, the compound of formula (I) or (I-A) is

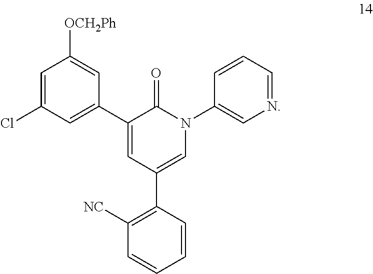

In various embodiments, the compound of formula (I) or (I-A) is

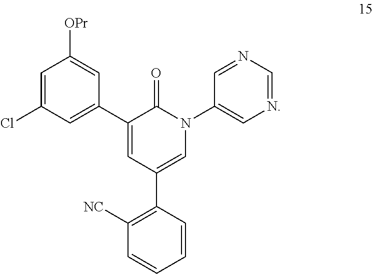

In various embodiments, the compound of formula (I) or (I-A) is

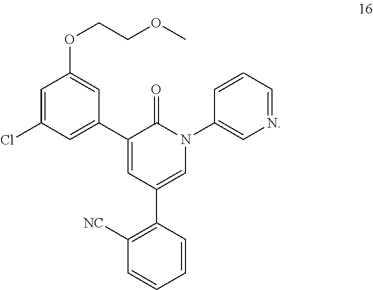 [SMILES: COCCOc1cc(Cl)cc(-c2cc(-c3ccccc3C#N)cn(-c3cccnc3)c2=O)c1]

In various embodiments, the compound of formula (I) or (I-A) is

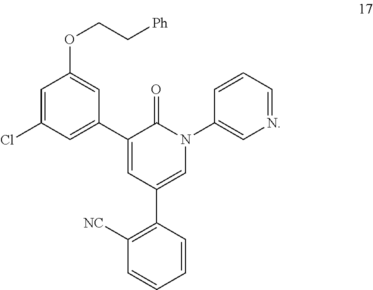 [SMILES: N#Cc1ccccc1-c1cc(-c2cc(Cl)cc(OCCc3ccccc3)c2)c(=O)n(-c2cccnc2)c1]

In various embodiments, the compound of formula (I) or (I-A) is

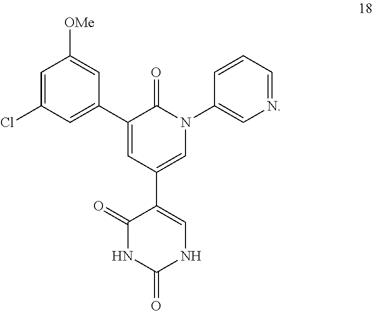 [SMILES: COc1cc(Cl)cc(-c2cc(-c3c[nH]c(=O)[nH]c3=O)cn(-c3cccnc3)c2=O)c1]

In various embodiments, the compound of formula (I) or (I-A) is

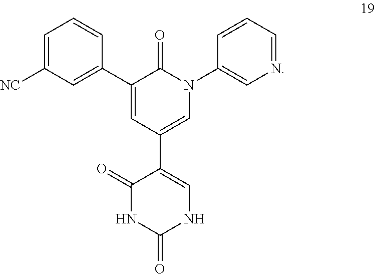 [SMILES: N#Cc1cccc(-c2cc(-c3c[nH]c(=O)[nH]c3=O)cn(-c3cccnc3)c2=O)c1]

In various embodiments, the compound of formula (I) or (I-A) is

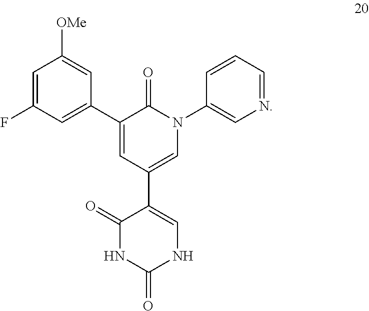 [SMILES: COc1cc(F)cc(-c2cc(-c3c[nH]c(=O)[nH]c3=O)cn(-c3cccnc3)c2=O)c1]

In various embodiments, the compound of formula (I) or (I-A) is

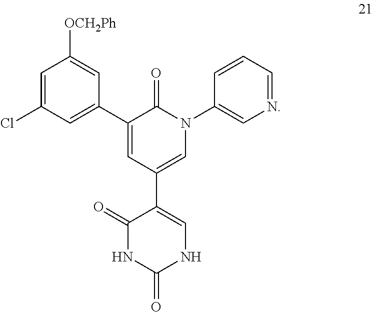

In various embodiments, the compound of formula (I) or (I-A) is

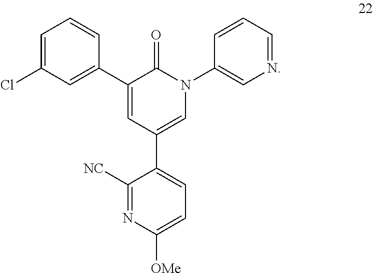 [SMILES: COc1ccc(-c2cc(-c3cccc(Cl)c3)c(=O)n(-c3cccnc3)c2)c(C#N)n1]

In various embodiments, the compound of formula (I) or (I-A) is

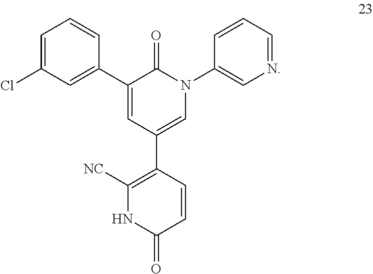 [SMILES: N#Cc1[nH]c(=O)ccc1-c1cc(-c2cccc(Cl)c2)c(=O)n(-c2cccnc2)c1]

In various embodiments, the compound of formula (I) or (I-A) is

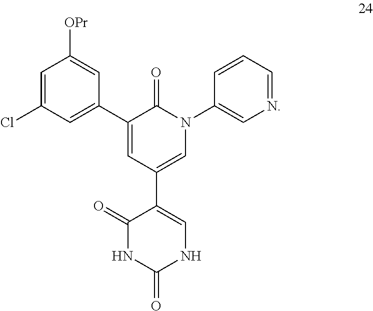

In various embodiments, the compound of formula (I) or (I-A) is

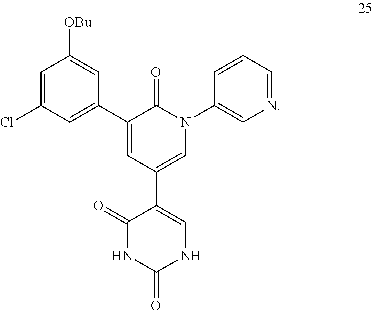

In various embodiments, the compound of formula (I) or (I-A) is

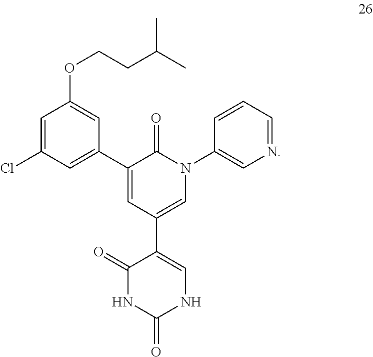 [SMILES: CC(C)CCOc1cc(Cl)cc(-c2cc(-c3c[nH]c(=O)[nH]c3=O)cn(-c3cccnc3)c2=O)c1]

In various embodiments, the compound of formula (I) or (I-A) is

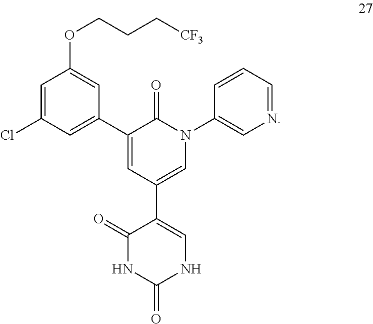 [SMILES: O=c1[nH]cc(-c2cc(-c3cc(Cl)cc(OCCCC(F)(F)F)c3)c(=O)n(-c3cccnc3)c2)c(=O)[nH]1]

In various embodiments, the compound of formula (I) or (I-A) is

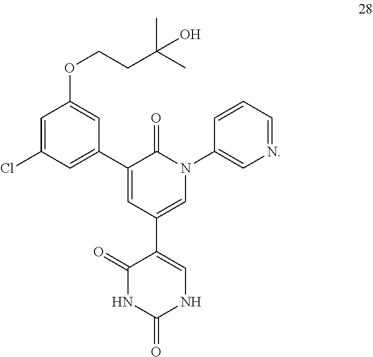 [SMILES: CC(C)(O)CCOc1cc(Cl)cc(-c2cc(-c3c[nH]c(=O)[nH]c3=O)cn(-c3cccnc3)c2=O)c1]

In various embodiments, the compound of formula (I) or (I-A) is

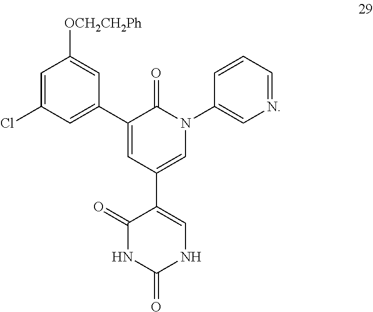

In various embodiments, the compound of formula (I) or (I-A) is

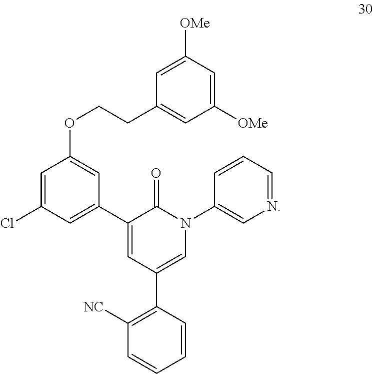 [SMILES: COc1cc(CCOc2cc(Cl)cc(-c3cc(-c4ccccc4C#N)cn(-c4cccnc4)c3=O)c2)cc(OC)c1]

In various embodiments, the compound of formula (I) or (I-A) is

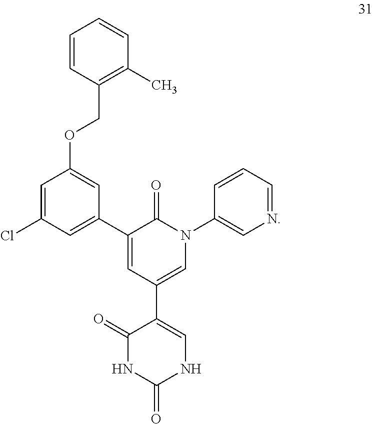 [SMILES: Cc1ccccc1COc1cc(Cl)cc(-c2cc(-c3c[nH]c(=O)[nH]c3=O)cn(-c3cccnc3)c2=O)c1]

In various embodiments, the compound of formula (I) or (I-A) is

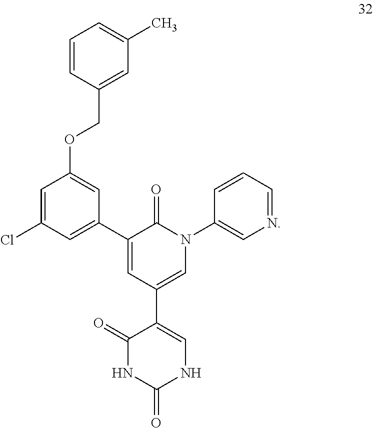 [SMILES: Cc1cccc(COc2cc(Cl)cc(-c3cc(-c4c[nH]c(=O)[nH]c4=O)cn(-c4cccnc4)c3=O)c2)c1]

In various embodiments, the compound of formula (I) or (I-A) is

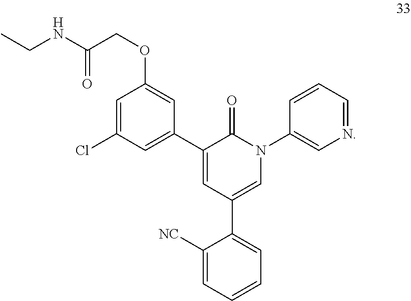 [SMILES: CCNC(=O)COc1cc(Cl)cc(-c2cc(-c3ccccc3C#N)cn(-c3cccnc3)c2=O)c1]

In various embodiments, the compound of formula (I) or (I-A) is

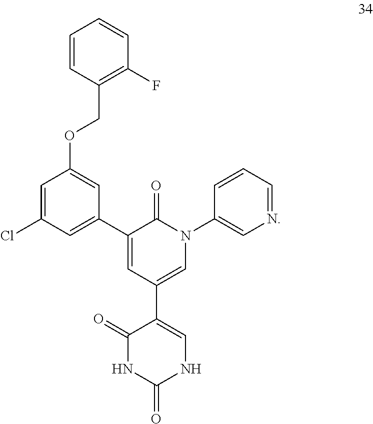 [SMILES: O=c1[nH]cc(-c2cc(-c3cc(Cl)cc(OCc4ccccc4F)c3)c(=O)n(-c3cccnc3)c2)c(=O)[nH]1]

In various embodiments, the compound of formula (I) or (I-A) is

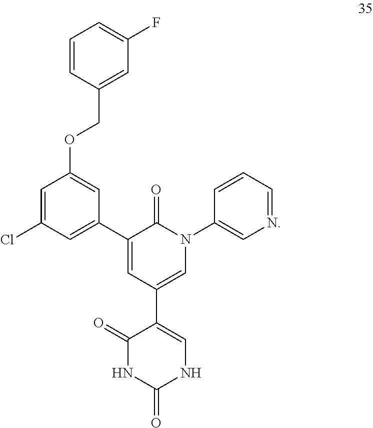 [SMILES: O=c1[nH]cc(-c2cc(-c3cc(Cl)cc(OCc4cccc(F)c4)c3)c(=O)n(-c3cccnc3)c2)c(=O)[nH]1]

In various embodiments, the compound of formula (I) or (I-A) is

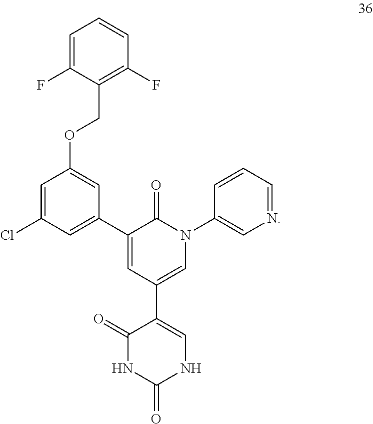 [SMILES: O=c1[nH]cc(-c2cc(-c3cc(Cl)cc(OCc4c(F)cccc4F)c3)c(=O)n(-c3cccnc3)c2)c(=O)[nH]1]

In various embodiments, the compound of formula (I) or (I-A) is

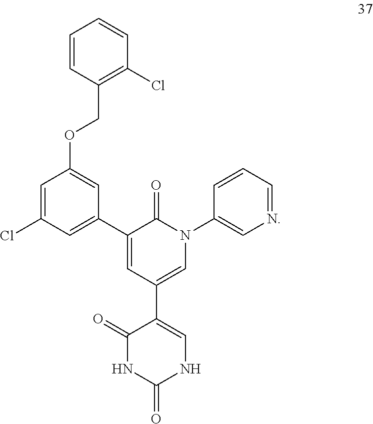 [SMILES: O=c1[nH]cc(-c2cc(-c3cc(Cl)cc(OCc4ccccc4Cl)c3)c(=O)n(-c3cccnc3)c2)c(=O)[nH]1]

In various embodiments, the compound of formula (I) or (I-A) is

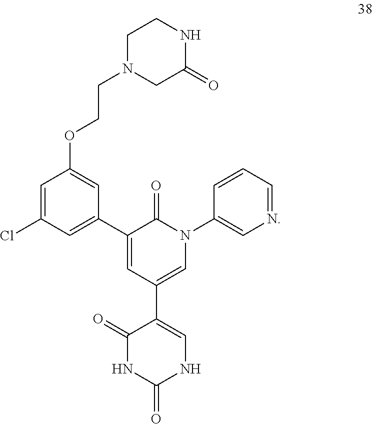 [SMILES: O=C1CN(CCOc2cc(Cl)cc(-c3cc(-c4c[nH]c(=O)[nH]c4=O)cn(-c4cccnc4)c3=O)c2)CCN1]

In various embodiments, the compound of formula (I) or (I-A) is

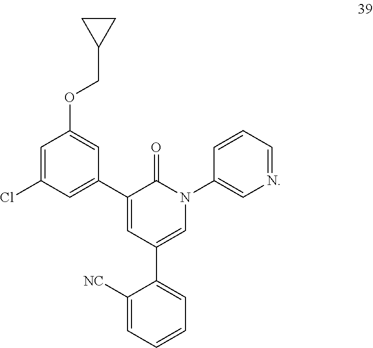 [SMILES: N#Cc1ccccc1-c1cc(-c2cc(Cl)cc(OCC3CC3)c2)c(=O)n(-c2cccnc2)c1]

In various embodiments, the compound of formula (I) or (I-A) is

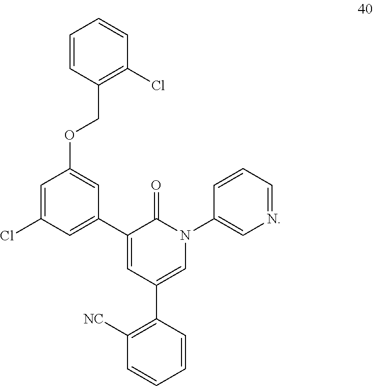 [SMILES: N#Cc1ccccc1-c1cc(-c2cc(Cl)cc(OCc3ccccc3Cl)c2)c(=O)n(-c2cccnc2)c1]

In various embodiments, the compound of formula (I) or (I-A) is

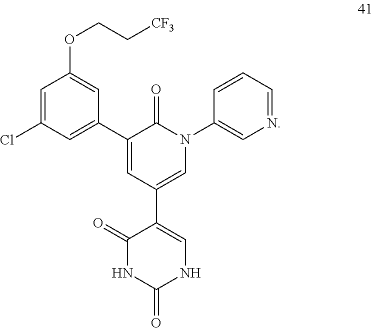 [SMILES: O=c1[nH]cc(-c2cc(-c3cc(Cl)cc(OCCC(F)(F)F)c3)c(=O)n(-c3cccnc3)c2)c(=O)[nH]1]

In various embodiments, the compound of formula (I) or (I-A) is

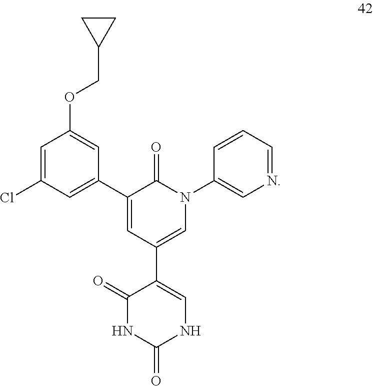 [SMILES: O=c1[nH]cc(-c2cc(-c3cc(Cl)cc(OCC4CC4)c3)c(=O)n(-c3cccnc3)c2)c(=O)[nH]1]

In various embodiments, the compound of formula (I) or (I-A) is

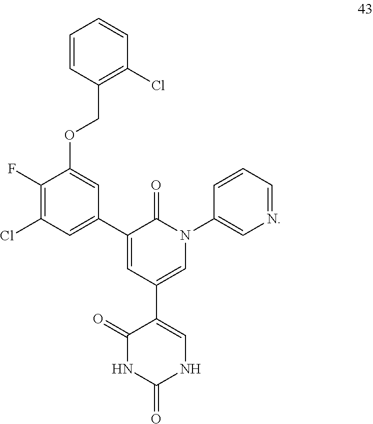 [SMILES: O=c1[nH]cc(-c2cc(-c3cc(Cl)c(F)c(OCc4ccccc4Cl)c3)c(=O)n(-c3cccnc3)c2)c(=O)[nH]1]

In various embodiments, the compound of formula (I) or (I-A) is

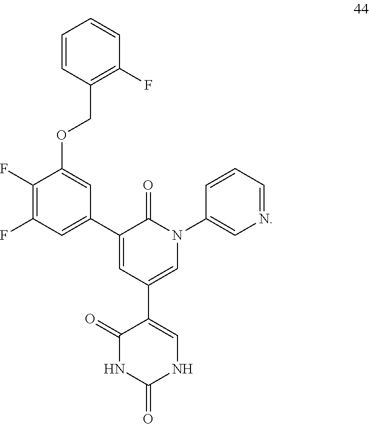 [SMILES: O=c1[nH]cc(-c2cc(-c3cc(F)c(F)c(OCc4ccccc4F)c3)c(=O)n(-c3cccnc3)c2)c(=O)[nH]1]

In various embodiments, the compound of formula (I) or (I-A) is

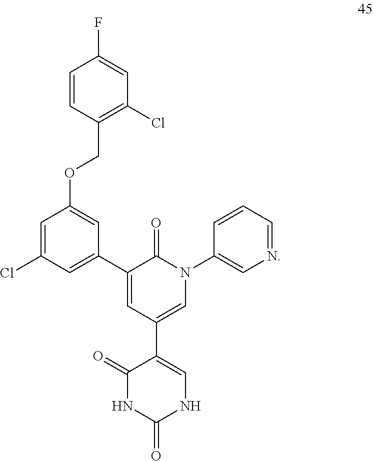 [SMILES: O=c1[nH]cc(-c2cc(-c3cc(Cl)cc(OCc4ccc(F)cc4Cl)c3)c(=O)n(-c3cccnc3)c2)c(=O)[nH]1]

In various embodiments, the compound of formula (I) or (I-A) is

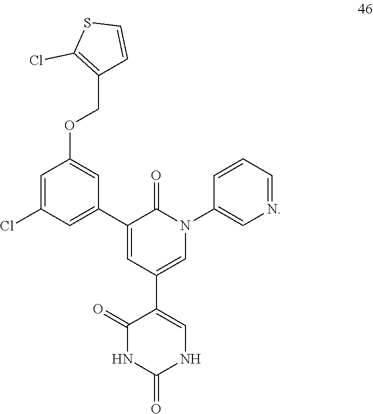 [SMILES: O=c1[nH]cc(-c2cc(-c3cc(Cl)cc(OCc4ccsc4Cl)c3)c(=O)n(-c3cccnc3)c2)c(=O)[nH]1]

In various embodiments, the compound of formula (I) or (I-A) is

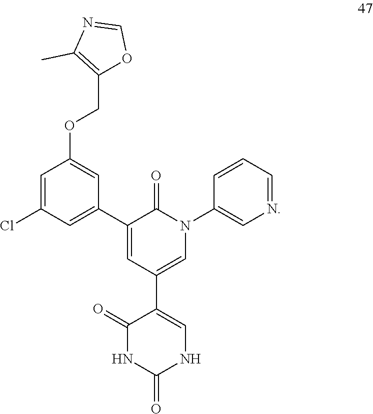 [SMILES: Cc1ncoc1COc1cc(Cl)cc(-c2cc(-c3c[nH]c(=O)[nH]c3=O)cn(-c3cccnc3)c2=O)c1]

In various embodiments, the compound of formula (I) or (I-A) is

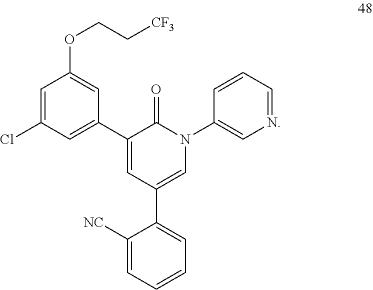 [SMILES: N#Cc1ccccc1-c1cc(-c2cc(Cl)cc(OCCC(F)(F)F)c2)c(=O)n(-c2cccnc2)c1]

In various embodiments, the compound of formula (I) or (I-A) is

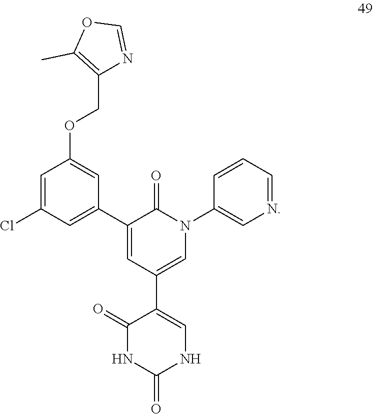 [SMILES: Cc1ocnc1COc1cc(Cl)cc(-c2cc(-c3c[nH]c(=O)[nH]c3=O)cn(-c3cccnc3)c2=O)c1]

In various embodiments, the compound of formula (I) or (I-A) is

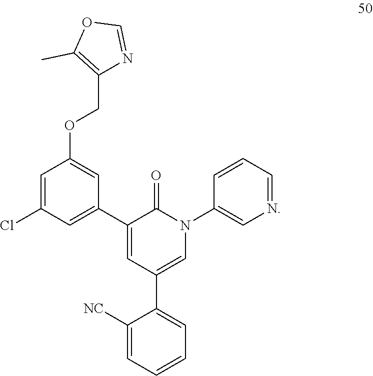 [SMILES: Cc1ocnc1COc1cc(Cl)cc(-c2cc(-c3ccccc3C#N)cn(-c3cccnc3)c2=O)c1]

In various embodiments, the compound of formula (I) or (I-A) is

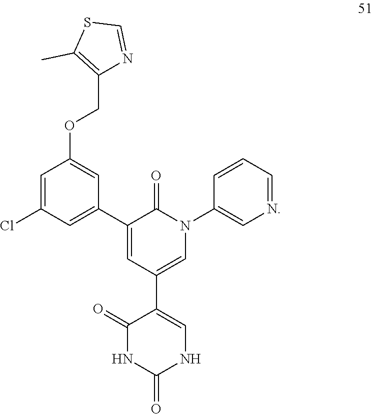 [SMILES: Cc1scnc1COc1cc(Cl)cc(-c2cc(-c3c[nH]c(=O)[nH]c3=O)cn(-c3cccnc3)c2=O)c1]

In various embodiments, the compound of formula (I) or (I-A) is

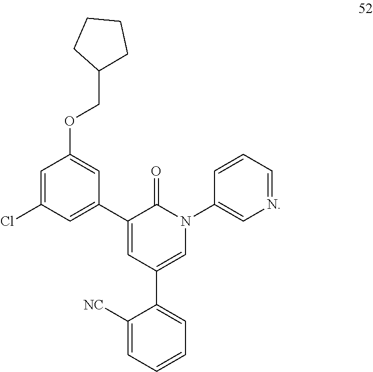 [SMILES: N#Cc1ccccc1-c1cc(-c2cc(Cl)cc(OCC3CCCC3)c2)c(=O)n(-c2cccnc2)c1]

In various embodiments, the compound of formula (I) or (I-A) is

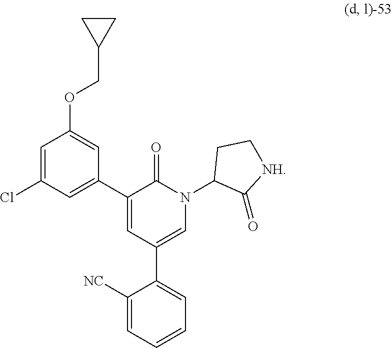 [SMILES: N#Cc1ccccc1-c1cc(-c2cc(Cl)cc(OCC3CC3)c2)c(=O)n(C2CCNC2=O)c1]

In various embodiments, the compound of formula (I) or (I-A) is

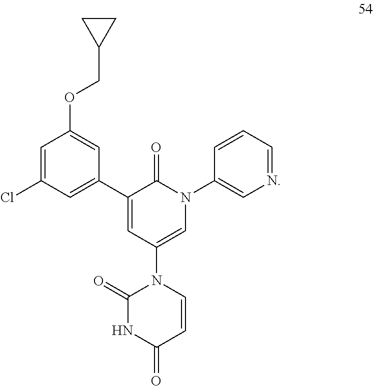 [SMILES: O=c1ccn(-c2cc(-c3cc(Cl)cc(OCC4CC4)c3)c(=O)n(-c3cccnc3)c2)c(=O)[nH]1]

In various embodiments, the compound of formula (I) or (I-A) is

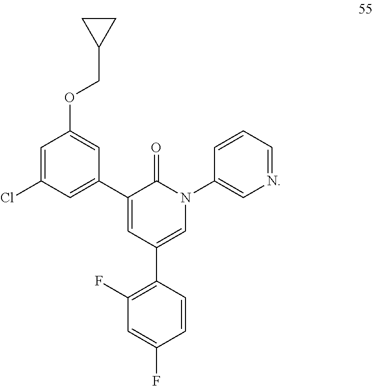 [SMILES: O=c1c(-c2cc(Cl)cc(OCC3CC3)c2)cc(-c2ccc(F)cc2F)cn1-c1cccnc1]

In various embodiments, the compound of formula (I) or (I-A) is

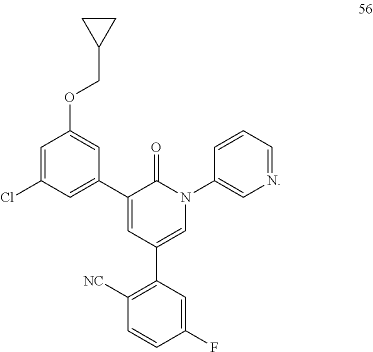 [SMILES: N#Cc1ccc(F)cc1-c1cc(-c2cc(Cl)cc(OCC3CC3)c2)c(=O)n(-c2cccnc2)c1]

In various embodiments, the compound of formula (I) or (I-A) is

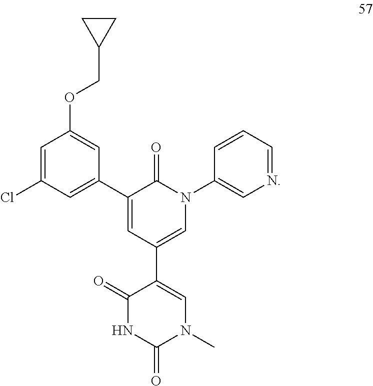 [SMILES: Cn1cc(-c2cc(-c3cc(Cl)cc(OCC4CC4)c3)c(=O)n(-c3cccnc3)c2)c(=O)[nH]c1=O]

In various embodiments, the compound of formula (I) or (I-A) is

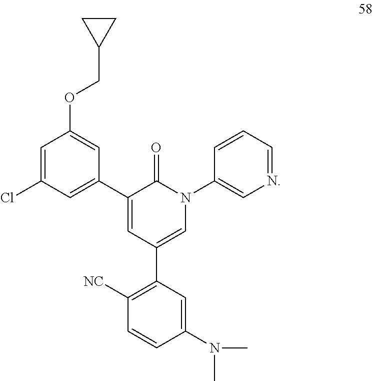 [SMILES: CN(C)c1ccc(C#N)c(-c2cc(-c3cc(Cl)cc(OCC4CC4)c3)c(=O)n(-c3cccnc3)c2)c1]

In various embodiments, the compound of formula (I) or (I-A) is

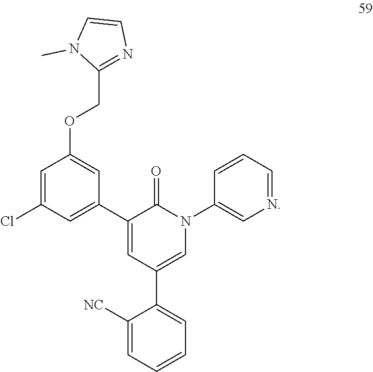 [SMILES: Cn1ccnc1COc1cc(Cl)cc(-c2cc(-c3ccccc3C#N)cn(-c3cccnc3)c2=O)c1]

In various embodiments, the compound of formula (I) or (I-A) is

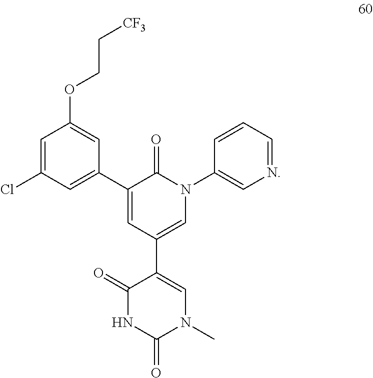 [SMILES: Cn1cc(-c2cc(-c3cc(Cl)cc(OCCC(F)(F)F)c3)c(=O)n(-c3cccnc3)c2)c(=O)[nH]c1=O]

In various embodiments, the compound of formula (I) or (I-A) is

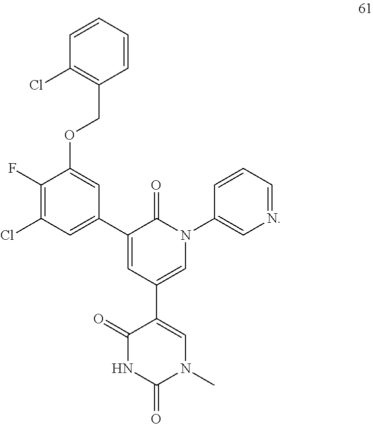 [SMILES: Cn1cc(-c2cc(-c3cc(Cl)c(F)c(OCc4ccccc4Cl)c3)c(=O)n(-c3cccnc3)c2)c(=O)[nH]c1=O]

In various embodiments, the compound of formula (I) or (I-A) is

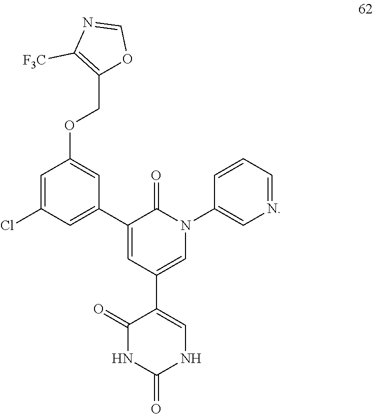 [SMILES: O=c1[nH]cc(-c2cc(-c3cc(Cl)cc(OCc4ocnc4C(F)(F)F)c3)c(=O)n(-c3cccnc3)c2)c(=O)[nH]1]

In various embodiments, the compound of formula (I) or (I-A) is

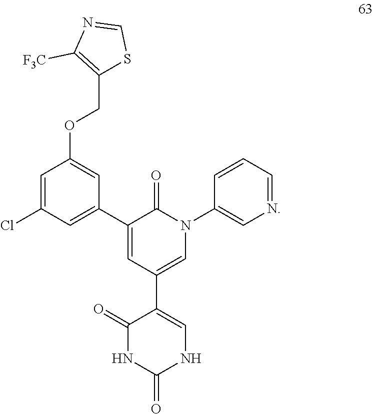 [SMILES: O=c1[nH]cc(-c2cc(-c3cc(Cl)cc(OCc4scnc4C(F)(F)F)c3)c(=O)n(-c3cccnc3)c2)c(=O)[nH]1]

In various embodiments, the compound of formula (I) or (I-A) is

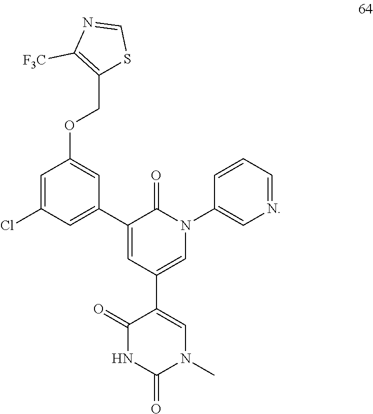 [SMILES: Cn1cc(-c2cc(-c3cc(Cl)cc(OCc4scnc4C(F)(F)F)c3)c(=O)n(-c3cccnc3)c2)c(=O)[nH]c1=O]

In various embodiments, the compound of formula (I) or (I-A) is

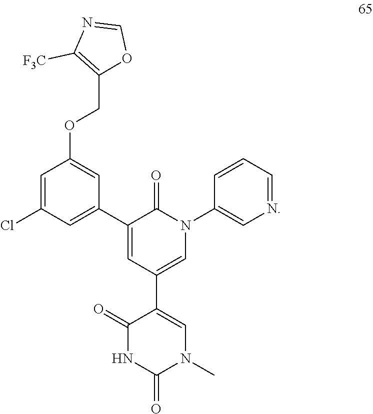 [SMILES: Cn1cc(-c2cc(-c3cc(Cl)cc(OCc4ocnc4C(F)(F)F)c3)c(=O)n(-c3cccnc3)c2)c(=O)[nH]c1=O]

In various embodiments, the compound of formula (I) or (I-A) is

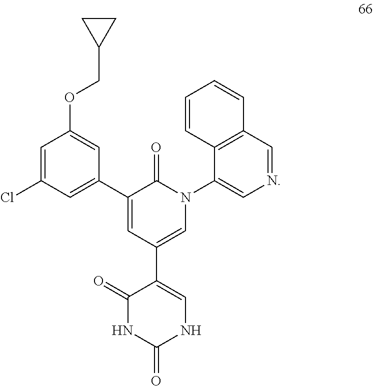 [SMILES: O=c1[nH]cc(-c2cc(-c3cc(Cl)cc(OCC4CC4)c3)c(=O)n(-c3cncc4ccccc34)c2)c(=O)[nH]1]

In various embodiments, the compound of formula (I) or (I-A) is

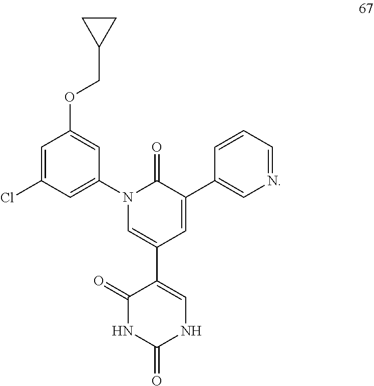 [SMILES: O=c1[nH]cc(-c2cc(-c3cccnc3)c(=O)n(-c3cc(Cl)cc(OCC4CC4)c3)c2)c(=O)[nH]1]

In various embodiments, the compound of formula (I) or (I-A) is

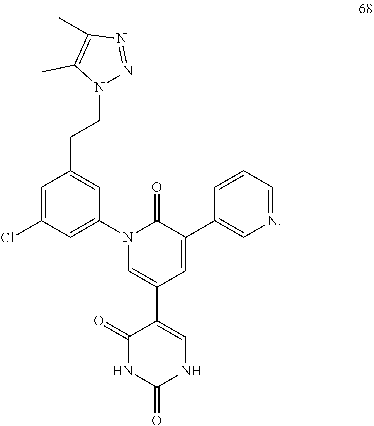 [SMILES: Cc1nnn(CCc2cc(Cl)cc(-n3cc(-c4c[nH]c(=O)[nH]c4=O)cc(-c4cccnc4)c3=O)c2)c1C]

In various embodiments, the compound of formula (I) or (I-A) is

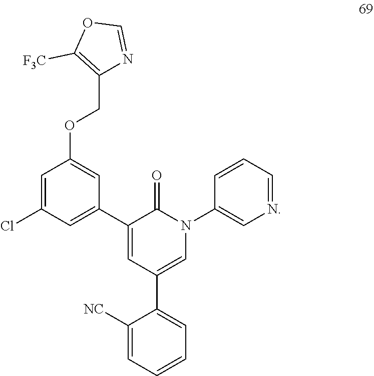 [SMILES: N#Cc1ccccc1-c1cc(-c2cc(Cl)cc(OCc3ncoc3C(F)(F)F)c2)c(=O)n(-c2cccnc2)c1]

In various embodiments, the compound of formula (I) or (I-A) is

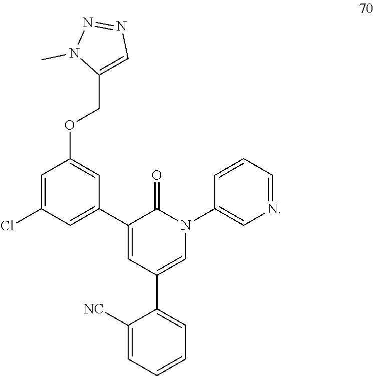 [SMILES: Cn1nncc1COc1cc(Cl)cc(-c2cc(-c3ccccc3C#N)cn(-c3cccnc3)c2=O)c1]

In various embodiments, the compound of formula (I) or (I-A) is

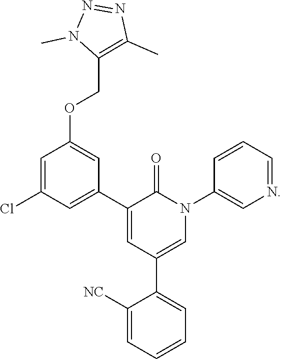 [SMILES: Cc1nnn(C)c1COc1cc(Cl)cc(-c2cc(-c3ccccc3C#N)cn(-c3cccnc3)c2=O)c1]

In various embodiments, the compound of formula (I) or (I-A) is

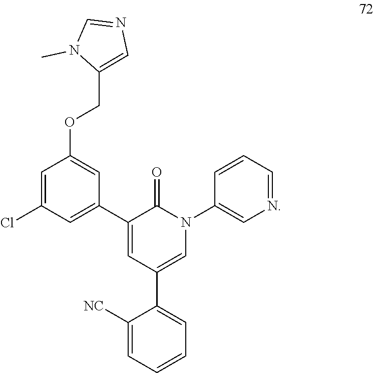 [SMILES: Cn1cncc1COc1cc(Cl)cc(-c2cc(-c3ccccc3C#N)cn(-c3cccnc3)c2=O)c1]

In various embodiments, the compound of formula (I) or (I-A) is

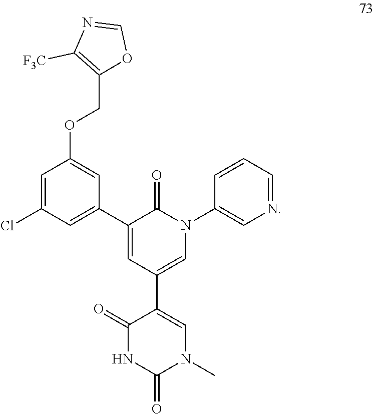 [SMILES: Cn1cc(-c2cc(-c3cc(Cl)cc(OCc4ocnc4C(F)(F)F)c3)c(=O)n(-c3cccnc3)c2)c(=O)[nH]c1=O]

In various embodiments, the compound of formula (I) or (I-A) is not

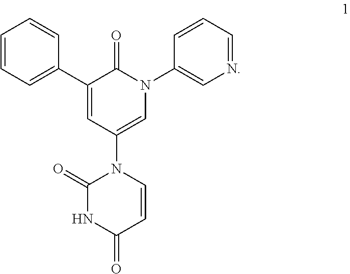 [SMILES: O=c1ccn(-c2cc(-c3ccccc3)c(=O)n(-c3cccnc3)c2)c(=O)[nH]1]

In various embodiments, the compound of formula (I) or (I-A) is not

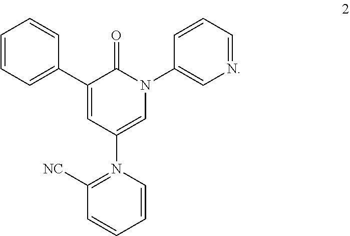

In various embodiments, the compound of formula (I) or (I-A) is not

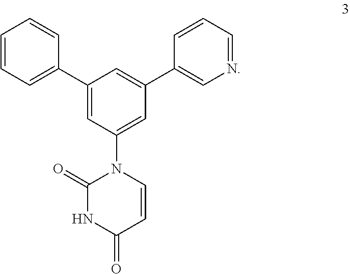 [SMILES: O=c1ccn(-c2cc(-c3ccccc3)cc(-c3cccnc3)c2)c(=O)[nH]1]

In various embodiments, the compound of formula (I) or (I-A) is not

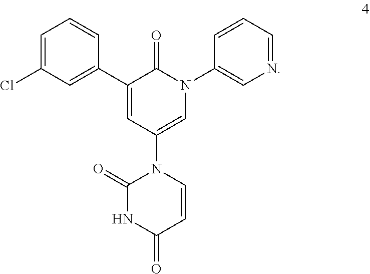 [SMILES: O=c1ccn(-c2cc(-c3cccc(Cl)c3)c(=O)n(-c3cccnc3)c2)c(=O)[nH]1]

In various embodiments, the compound of formula (I) or (I-A) is not

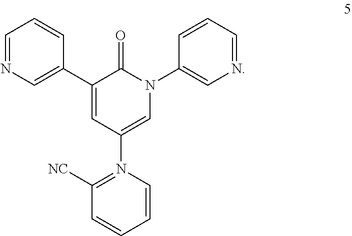

In various embodiments, the compound of formula (I) or (I-A) is not

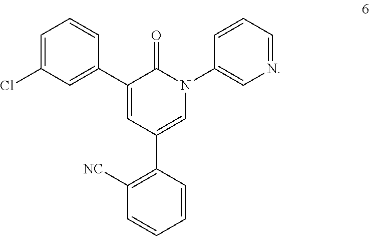 [SMILES: N#Cc1ccccc1-c1cc(-c2cccc(Cl)c2)c(=O)n(-c2cccnc2)c1]

In various embodiments, the compound of formula (I) or (I-A) is not

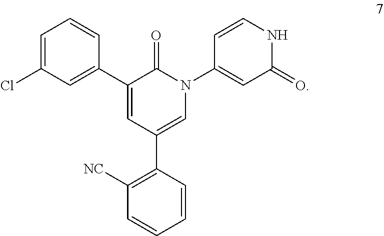 [SMILES: N#Cc1ccccc1-c1cc(-c2cccc(Cl)c2)c(=O)n(-c2cc[nH]c(=O)c2)c1]

In various embodiments, the compound of formula (I) or (I-A) is not

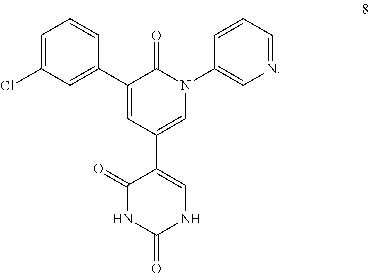 [SMILES: O=c1[nH]cc(-c2cc(-c3cccc(Cl)c3)c(=O)n(-c3cccnc3)c2)c(=O)[nH]1]

In various embodiments, the compound of formula (I) or (I-A) is not

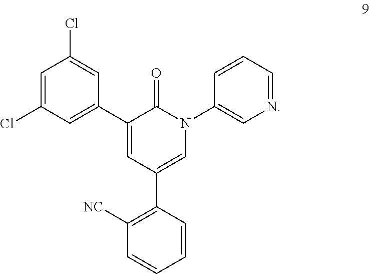 [SMILES: N#Cc1ccccc1-c1cc(-c2cc(Cl)cc(Cl)c2)c(=O)n(-c2cccnc2)c1]

In various embodiments, the compound of formula (I) or (I-A) is not

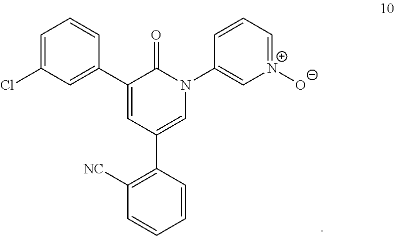 [SMILES: N#Cc1ccccc1-c1cc(-c2cccc(Cl)c2)c(=O)n(-c2ccc[n+]([O-])c2)c1]

In various embodiments, the compound of formula (I) or (I-A) is not

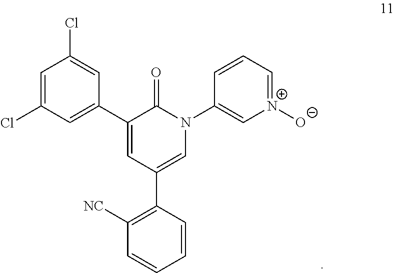 [SMILES: N#Cc1ccccc1-c1cc(-c2cc(Cl)cc(Cl)c2)c(=O)n(-c2ccc[n+]([O-])c2)c1]

In various embodiments, the compound of formula (I) or (I-A) is not

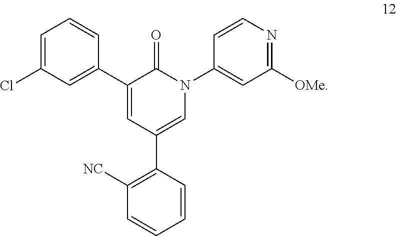 [SMILES: COc1cc(-n2cc(-c3ccccc3C#N)cc(-c3cccc(Cl)c3)c2=O)ccn1]

In various embodiments, the compound of formula (I) or (I-A) is not

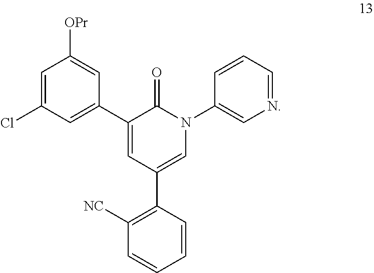

In various embodiments, the compound of formula (I) or (I-A) is not

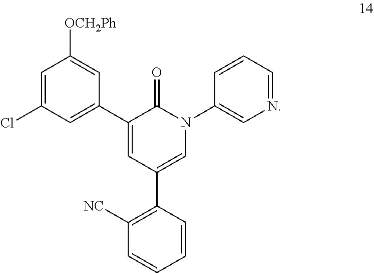

In various embodiments, the compound of formula (I) or (I-A) is not

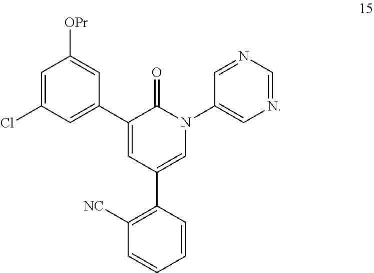

In various embodiments, the compound of formula (I) or (I-A) is not

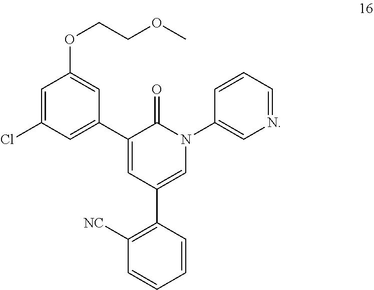 [SMILES: COCCOc1cc(Cl)cc(-c2cc(-c3ccccc3C#N)cn(-c3cccnc3)c2=O)c1]

In various embodiments, the compound of formula (I) or (I-A) is not

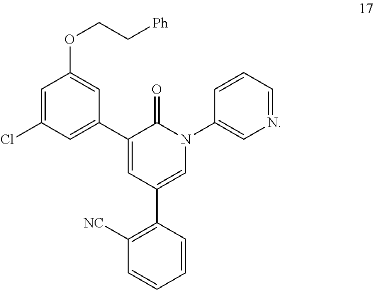 [SMILES: N#Cc1ccccc1-c1cc(-c2cc(Cl)cc(OCCc3ccccc3)c2)c(=O)n(-c2cccnc2)c1]

In various embodiments, the compound of formula (I) or (I-A) is not

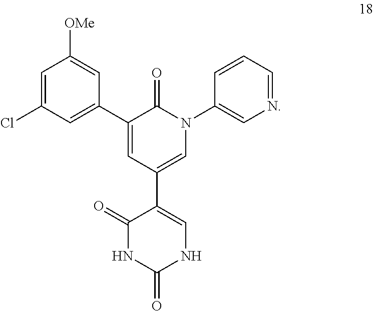 [SMILES: COc1cc(Cl)cc(-c2cc(-c3c[nH]c(=O)[nH]c3=O)cn(-c3cccnc3)c2=O)c1]

In various embodiments, the compound of formula (I) or (I-A) is not

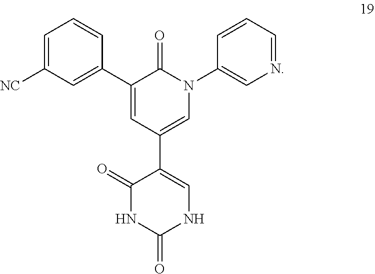 [SMILES: N#Cc1cccc(-c2cc(-c3c[nH]c(=O)[nH]c3=O)cn(-c3cccnc3)c2=O)c1]

In various embodiments, the compound of formula (I) or (I-A) is not

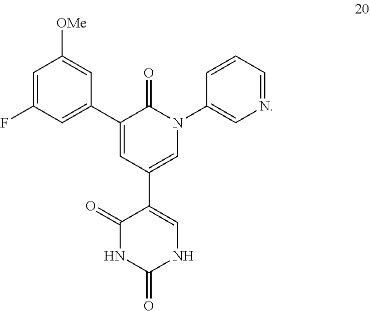 [SMILES: COc1cc(F)cc(-c2cc(-c3c[nH]c(=O)[nH]c3=O)cn(-c3cccnc3)c2=O)c1]

In various embodiments, the compound of formula (I) or (I-A) is not

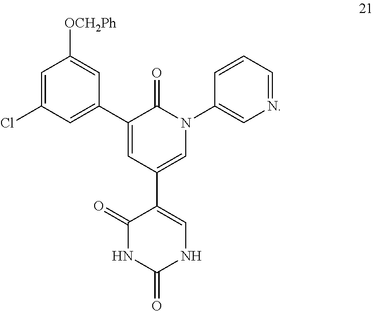

In various embodiments, the compound of formula (I) or (I-A) is not

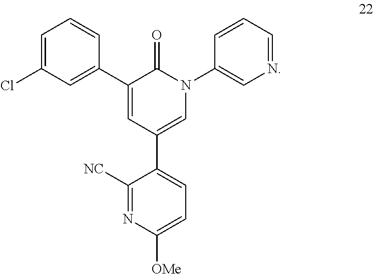 [SMILES: COc1ccc(-c2cc(-c3cccc(Cl)c3)c(=O)n(-c3cccnc3)c2)c(C#N)n1]

In various embodiments, the compound of formula (I) or (I-A) is not

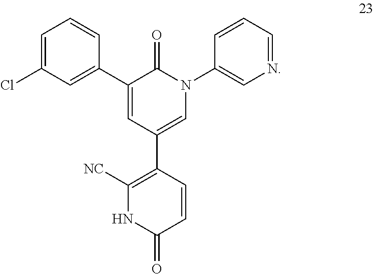 [SMILES: N#Cc1[nH]c(=O)ccc1-c1cc(-c2cccc(Cl)c2)c(=O)n(-c2cccnc2)c1]

In various embodiments, the compound of formula (I) or (I-A) is not

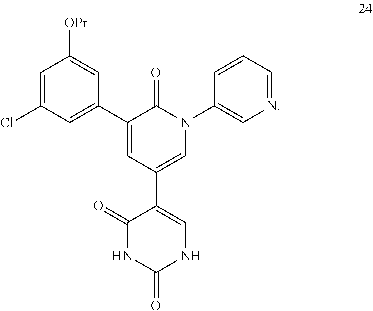

In various embodiments, the compound of formula (I) or (I-A) is not

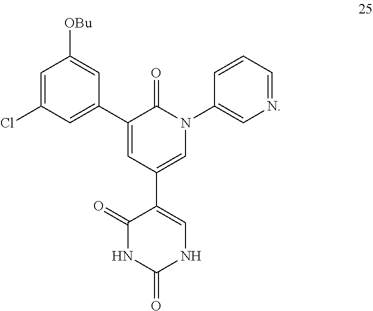

In various embodiments, the compound of formula (I) or (I-A) is not

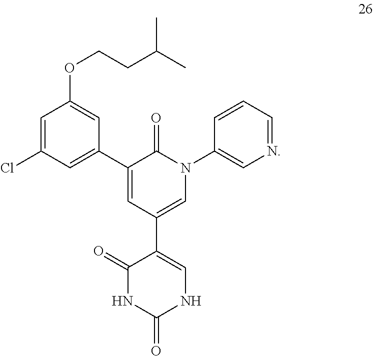 [SMILES: CC(C)CCOc1cc(Cl)cc(-c2cc(-c3c[nH]c(=O)[nH]c3=O)cn(-c3cccnc3)c2=O)c1]

In various embodiments, the compound of formula (I) or (I-A) is not

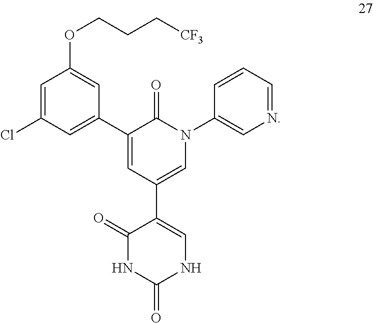 [SMILES: O=c1[nH]cc(-c2cc(-c3cc(Cl)cc(OCCCC(F)(F)F)c3)c(=O)n(-c3cccnc3)c2)c(=O)[nH]1]

In various embodiments, the compound of formula (I) or (I-A) is not

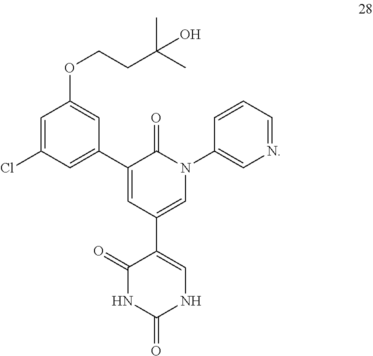 [SMILES: CC(C)(O)CCOc1cc(Cl)cc(-c2cc(-c3c[nH]c(=O)[nH]c3=O)cn(-c3cccnc3)c2=O)c1]

In various embodiments, the compound of formula (I) or (I-A) is not

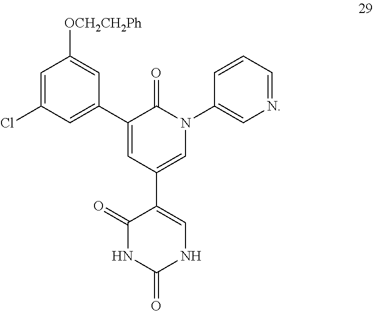

In various embodiments, the compound of formula (I) or (I-A) is not

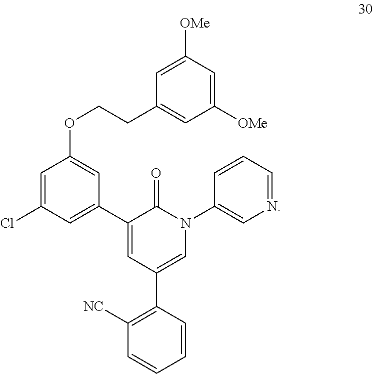 [SMILES: COc1cc(CCOc2cc(Cl)cc(-c3cc(-c4ccccc4C#N)cn(-c4cccnc4)c3=O)c2)cc(OC)c1]

In various embodiments, the compound of formula (I) or (I-A) is not

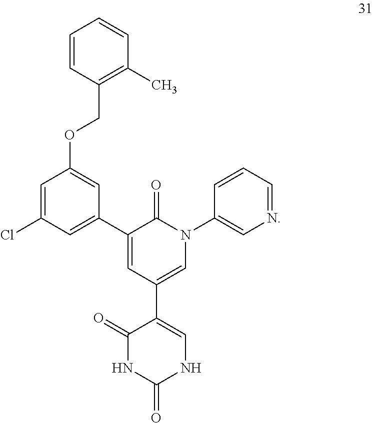 [SMILES: Cc1ccccc1COc1cc(Cl)cc(-c2cc(-c3c[nH]c(=O)[nH]c3=O)cn(-c3cccnc3)c2=O)c1]

In various embodiments, the compound of formula (I) or (I-A) is not

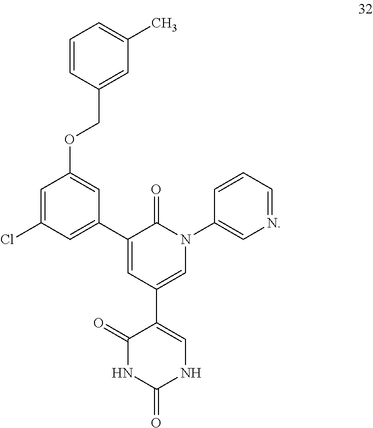 [SMILES: Cc1cccc(COc2cc(Cl)cc(-c3cc(-c4c[nH]c(=O)[nH]c4=O)cn(-c4cccnc4)c3=O)c2)c1]

In various embodiments, the compound of formula (I) or (I-A) is not

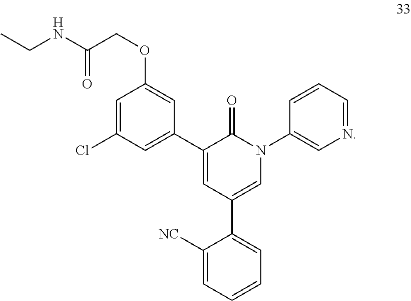 [SMILES: CCNC(=O)COc1cc(Cl)cc(-c2cc(-c3ccccc3C#N)cn(-c3cccnc3)c2=O)c1]

In various embodiments, the compound of formula (I) or (I-A) is not

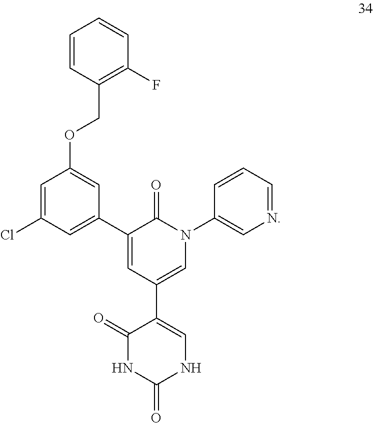 [SMILES: O=c1[nH]cc(-c2cc(-c3cc(Cl)cc(OCc4ccccc4F)c3)c(=O)n(-c3cccnc3)c2)c(=O)[nH]1]

In various embodiments, the compound of formula (I) or (I-A) is not

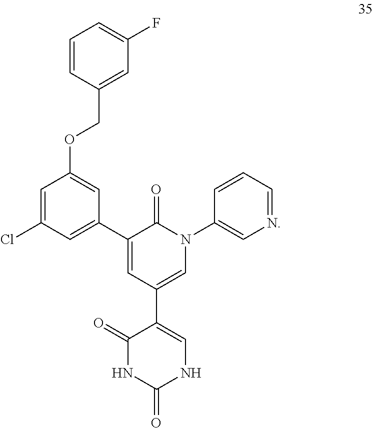 [SMILES: O=c1[nH]cc(-c2cc(-c3cc(Cl)cc(OCc4cccc(F)c4)c3)c(=O)n(-c3cccnc3)c2)c(=O)[nH]1]

In various embodiments, the compound of formula (I) or (I-A) is not

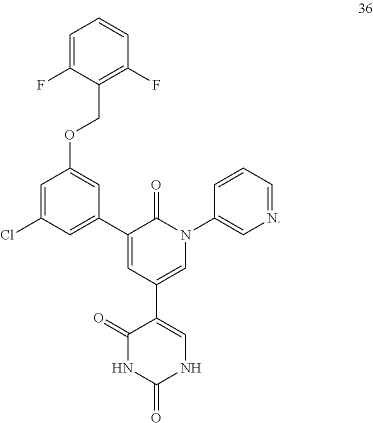 [SMILES: O=c1[nH]cc(-c2cc(-c3cc(Cl)cc(OCc4c(F)cccc4F)c3)c(=O)n(-c3cccnc3)c2)c(=O)[nH]1]

In various embodiments, the compound of formula (I) or (I-A) is not

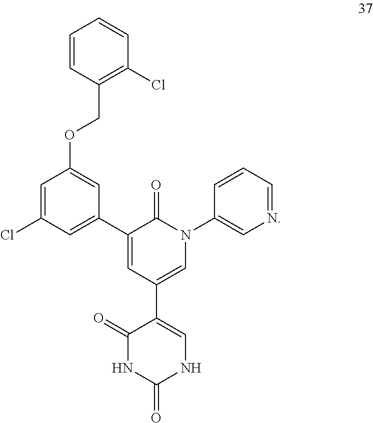 [SMILES: O=c1[nH]cc(-c2cc(-c3cc(Cl)cc(OCc4ccccc4Cl)c3)c(=O)n(-c3cccnc3)c2)c(=O)[nH]1]

In various embodiments, the compound of formula (I) or (I-A) is not

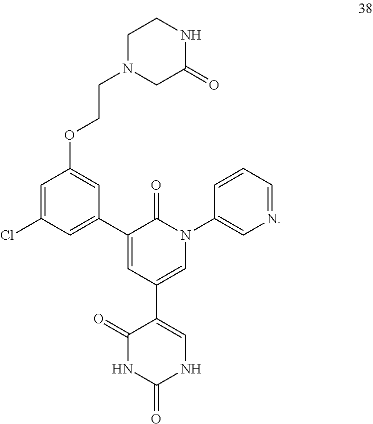 [SMILES: O=C1CN(CCOc2cc(Cl)cc(-c3cc(-c4c[nH]c(=O)[nH]c4=O)cn(-c4cccnc4)c3=O)c2)CCN1]

In various embodiments, the compound of formula (I) or (I-A) is not

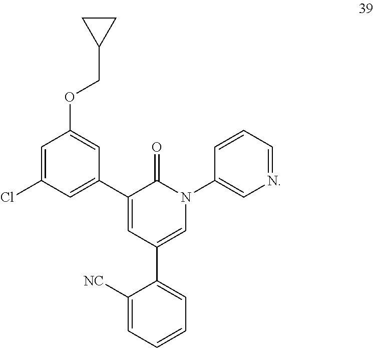 [SMILES: N#Cc1ccccc1-c1cc(-c2cc(Cl)cc(OCC3CC3)c2)c(=O)n(-c2cccnc2)c1]

In various embodiments, the compound of formula (I) or (I-A) is not

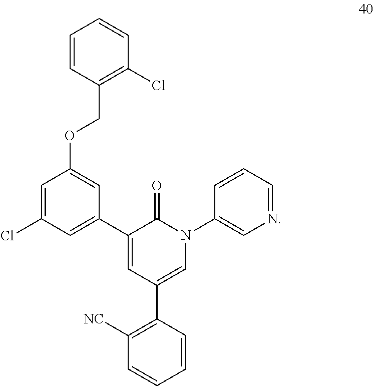 [SMILES: N#Cc1ccccc1-c1cc(-c2cc(Cl)cc(OCc3ccccc3Cl)c2)c(=O)n(-c2cccnc2)c1]

In various embodiments, the compound of formula (I) or (I-A) is not

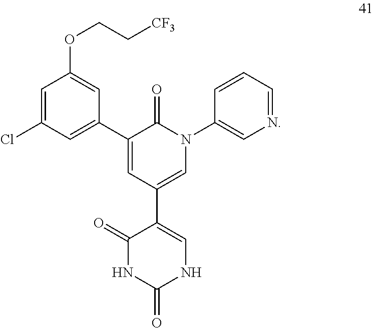 [SMILES: O=c1[nH]cc(-c2cc(-c3cc(Cl)cc(OCCC(F)(F)F)c3)c(=O)n(-c3cccnc3)c2)c(=O)[nH]1]

In various embodiments, the compound of formula (I) or (I-A) is not

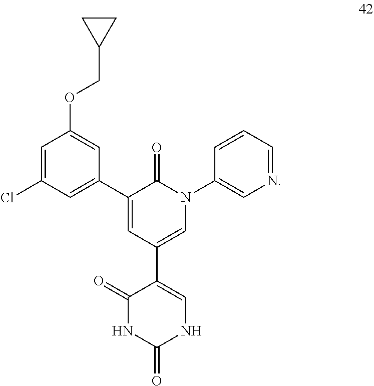 [SMILES: O=c1[nH]cc(-c2cc(-c3cc(Cl)cc(OCC4CC4)c3)c(=O)n(-c3cccnc3)c2)c(=O)[nH]1]

In various embodiments, the compound of formula (I) or (I-A) is not

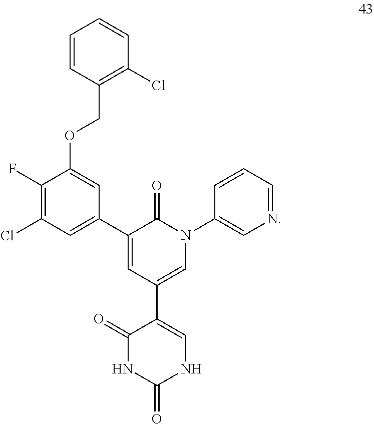 [SMILES: O=c1[nH]cc(-c2cc(-c3cc(Cl)c(F)c(OCc4ccccc4Cl)c3)c(=O)n(-c3cccnc3)c2)c(=O)[nH]1]

In various embodiments, the compound of formula (I) or (I-A) is not

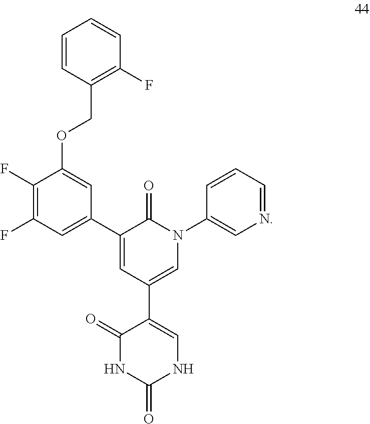 [SMILES: O=c1[nH]cc(-c2cc(-c3cc(F)c(F)c(OCc4ccccc4F)c3)c(=O)n(-c3cccnc3)c2)c(=O)[nH]1]

In various embodiments, the compound of formula (I) or (I-A) is not

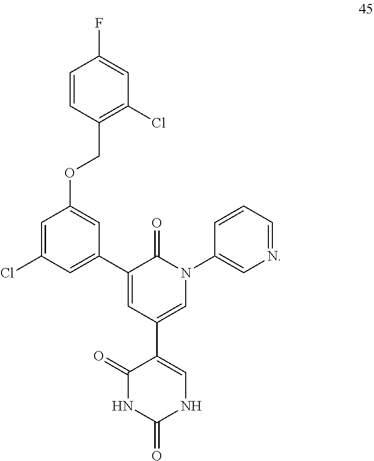 [SMILES: O=c1[nH]cc(-c2cc(-c3cc(Cl)cc(OCc4ccc(F)cc4Cl)c3)c(=O)n(-c3cccnc3)c2)c(=O)[nH]1]

In various embodiments, the compound of formula (I) or (I-A) is not

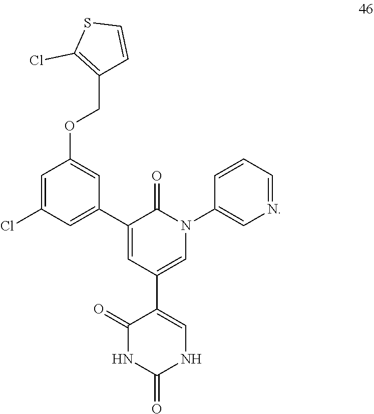 [SMILES: O=c1[nH]cc(-c2cc(-c3cc(Cl)cc(OCc4ccsc4Cl)c3)c(=O)n(-c3cccnc3)c2)c(=O)[nH]1]

In various embodiments, the compound of formula (I) or (I-A) is not

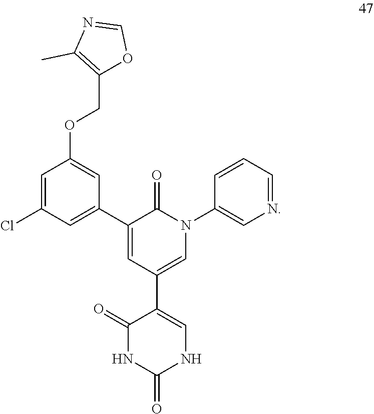 [SMILES: Cc1ncoc1COc1cc(Cl)cc(-c2cc(-c3c[nH]c(=O)[nH]c3=O)cn(-c3cccnc3)c2=O)c1]

In various embodiments, the compound of formula (I) or (I-A) is not

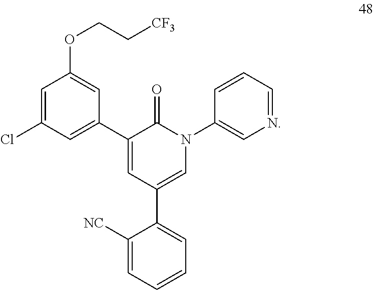 [SMILES: N#Cc1ccccc1-c1cc(-c2cc(Cl)cc(OCCC(F)(F)F)c2)c(=O)n(-c2cccnc2)c1]

In various embodiments, the compound of formula (I) or (I-A) is not

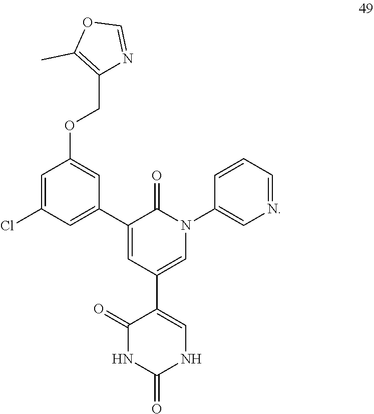 [SMILES: Cc1ocnc1COc1cc(Cl)cc(-c2cc(-c3c[nH]c(=O)[nH]c3=O)cn(-c3cccnc3)c2=O)c1]

In various embodiments, the compound of formula (I) or (I-A) is not

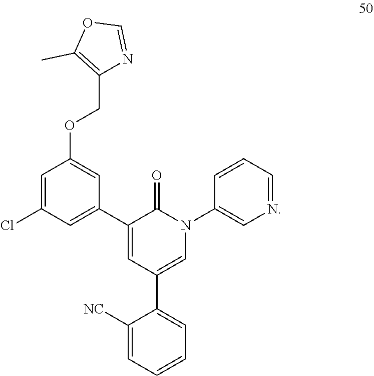 [SMILES: Cc1ocnc1COc1cc(Cl)cc(-c2cc(-c3ccccc3C#N)cn(-c3cccnc3)c2=O)c1]

In various embodiments, the compound of formula (I) or (I-A) is not

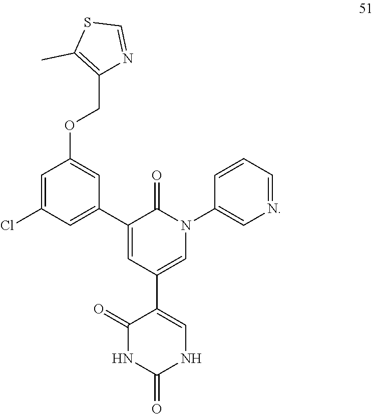 [SMILES: Cc1scnc1COc1cc(Cl)cc(-c2cc(-c3c[nH]c(=O)[nH]c3=O)cn(-c3cccnc3)c2=O)c1]

In various embodiments, the compound of formula (I) or (I-A) is not

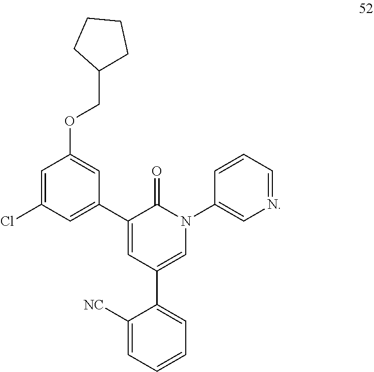 [SMILES: N#Cc1ccccc1-c1cc(-c2cc(Cl)cc(OCC3CCCC3)c2)c(=O)n(-c2cccnc2)c1]

In various embodiments, the compound of formula (I) or (I-A) is not

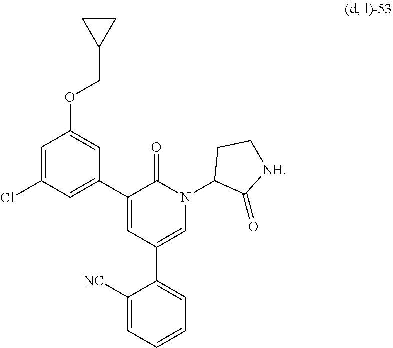 [SMILES: N#Cc1ccccc1-c1cc(-c2cc(Cl)cc(OCC3CC3)c2)c(=O)n(C2CCNC2=O)c1]

In various embodiments, the compound of formula (I) or (I-A) is not

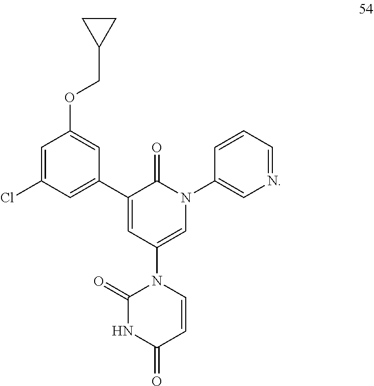 [SMILES: O=c1ccn(-c2cc(-c3cc(Cl)cc(OCC4CC4)c3)c(=O)n(-c3cccnc3)c2)c(=O)[nH]1]

In various embodiments, the compound of formula (I) or (I-A) is not

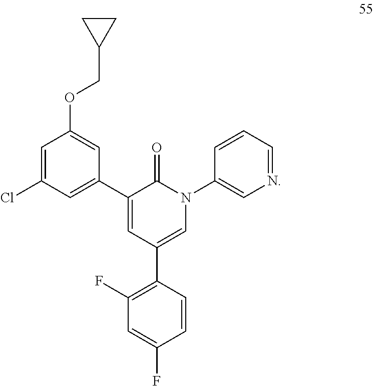 [SMILES: O=c1c(-c2cc(Cl)cc(OCC3CC3)c2)cc(-c2ccc(F)cc2F)cn1-c1cccnc1]

In various embodiments, the compound of formula (I) or (I-A) is not

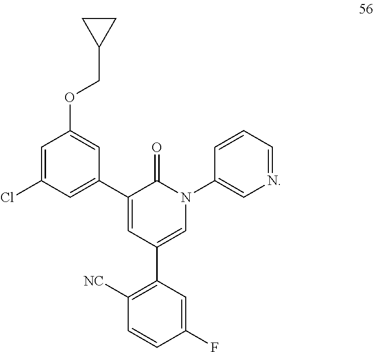 [SMILES: N#Cc1ccc(F)cc1-c1cc(-c2cc(Cl)cc(OCC3CC3)c2)c(=O)n(-c2cccnc2)c1]

In various embodiments, the compound of formula (I) or (I-A) is not

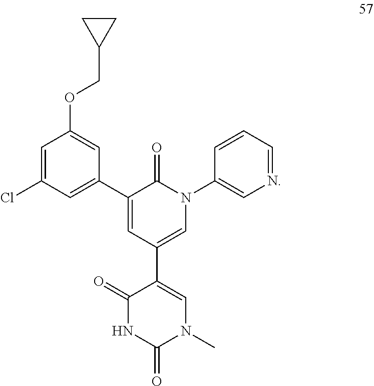 [SMILES: Cn1cc(-c2cc(-c3cc(Cl)cc(OCC4CC4)c3)c(=O)n(-c3cccnc3)c2)c(=O)[nH]c1=O]

In various embodiments, the compound of formula (I) or (I-A) is not

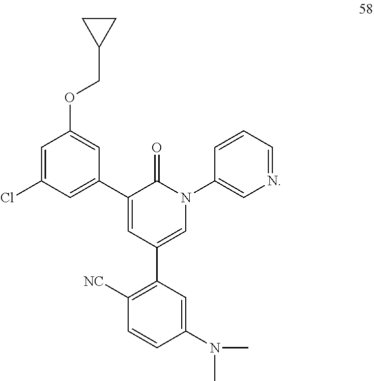 [SMILES: CN(C)c1ccc(C#N)c(-c2cc(-c3cc(Cl)cc(OCC4CC4)c3)c(=O)n(-c3cccnc3)c2)c1]

In various embodiments, the compound of formula (I) or (I-A) is not

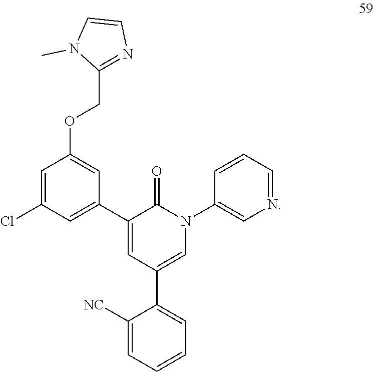 [SMILES: Cn1ccnc1COc1cc(Cl)cc(-c2cc(-c3ccccc3C#N)cn(-c3cccnc3)c2=O)c1]

In various embodiments, the compound of formula (I) or (I-A) is not

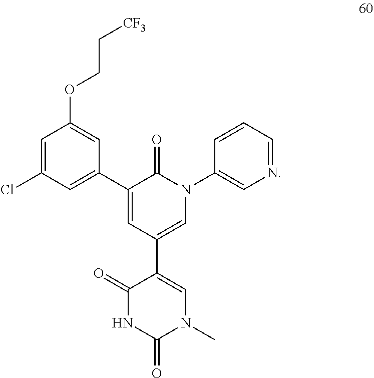 [SMILES: Cn1cc(-c2cc(-c3cc(Cl)cc(OCCC(F)(F)F)c3)c(=O)n(-c3cccnc3)c2)c(=O)[nH]c1=O]

In various embodiments, the compound of formula (I) or (I-A) is not

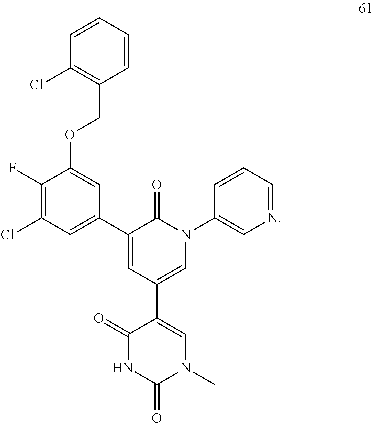 [SMILES: Cn1cc(-c2cc(-c3cc(Cl)c(F)c(OCc4ccccc4Cl)c3)c(=O)n(-c3cccnc3)c2)c(=O)[nH]c1=O]

In various embodiments, the compound of formula (I) or (I-A) is not

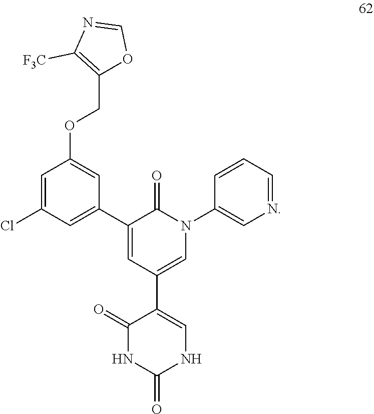 [SMILES: O=c1[nH]cc(-c2cc(-c3cc(Cl)cc(OCc4ocnc4C(F)(F)F)c3)c(=O)n(-c3cccnc3)c2)c(=O)[nH]1]

In various embodiments, the compound of formula (I) or (I-A) is not

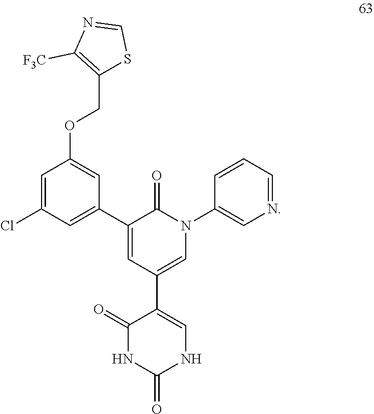 [SMILES: O=c1[nH]cc(-c2cc(-c3cc(Cl)cc(OCc4scnc4C(F)(F)F)c3)c(=O)n(-c3cccnc3)c2)c(=O)[nH]1]

In various embodiments, the compound of formula (I) or (I-A) is not

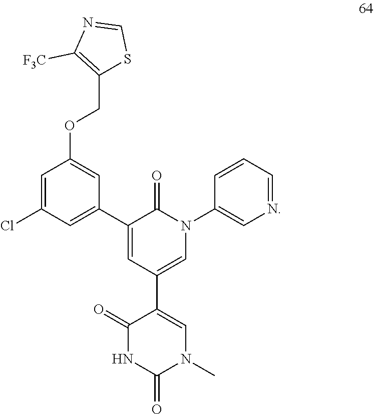 [SMILES: Cn1cc(-c2cc(-c3cc(Cl)cc(OCc4scnc4C(F)(F)F)c3)c(=O)n(-c3cccnc3)c2)c(=O)[nH]c1=O]

In various embodiments, the compound of formula (I) or (I-A) is not

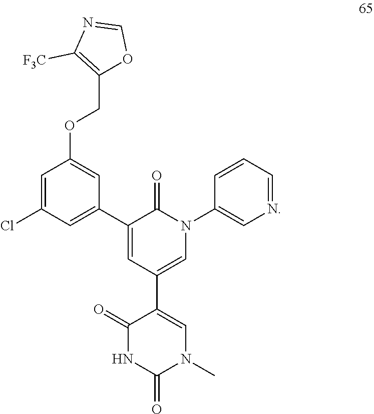 [SMILES: Cn1cc(-c2cc(-c3cc(Cl)cc(OCc4ocnc4C(F)(F)F)c3)c(=O)n(-c3cccnc3)c2)c(=O)[nH]c1=O]

In various embodiments, the compound of formula (I) or (I-A) is not

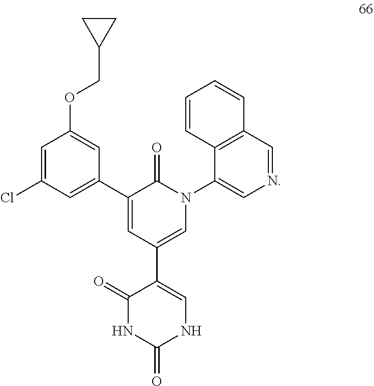 [SMILES: O=c1[nH]cc(-c2cc(-c3cc(Cl)cc(OCC4CC4)c3)c(=O)n(-c3cncc4ccccc34)c2)c(=O)[nH]1]

In various embodiments, the compound of formula (I) or (I-A) is not

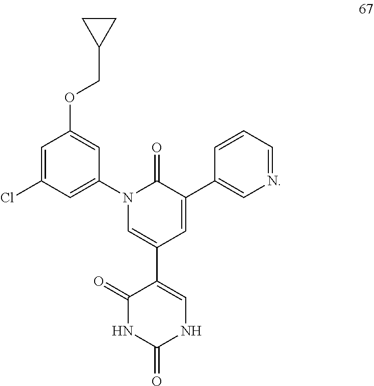 [SMILES: O=c1[nH]cc(-c2cc(-c3cccnc3)c(=O)n(-c3cc(Cl)cc(OCC4CC4)c3)c2)c(=O)[nH]1]

In various embodiments, the compound of formula (I) or (I-A) is not

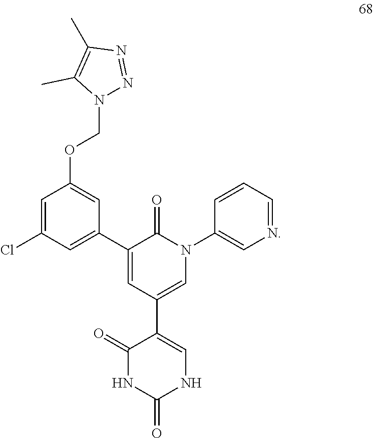 [SMILES: Cc1nnn(COc2cc(Cl)cc(-c3cc(-c4c[nH]c(=O)[nH]c4=O)cn(-c4cccnc4)c3=O)c2)c1C]

In various embodiments, the compound of formula (I) or (I-A) is not

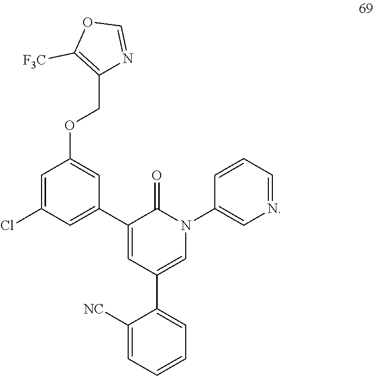 [SMILES: N#Cc1ccccc1-c1cc(-c2cc(Cl)cc(OCc3ncoc3C(F)(F)F)c2)c(=O)n(-c2cccnc2)c1]

In various embodiments, the compound of formula (I) or (I-A) is not

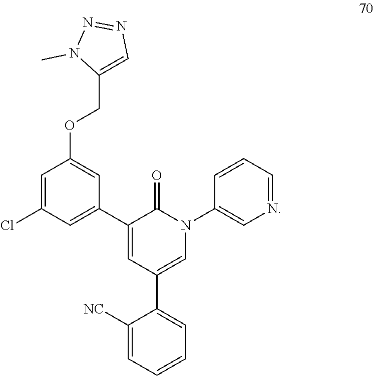 [SMILES: Cn1nncc1COc1cc(Cl)cc(-c2cc(-c3ccccc3C#N)cn(-c3cccnc3)c2=O)c1]

In various embodiments, the compound of formula (I) or (I-A) is not

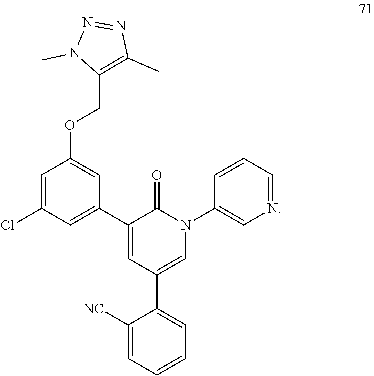 [SMILES: Cc1nnn(C)c1COc1cc(Cl)cc(-c2cc(-c3ccccc3C#N)cn(-c3cccnc3)c2=O)c1]

In various embodiments, the compound of formula (I) or (I-A) is not

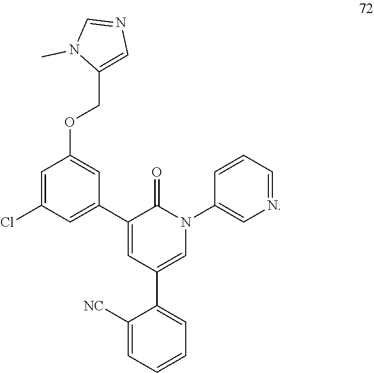 [SMILES: Cn1cncc1COc1cc(Cl)cc(-c2cc(-c3ccccc3C#N)cn(-c3cccnc3)c2=O)c1]

In various embodiments, the compound of formula (I) or (I-A) is not

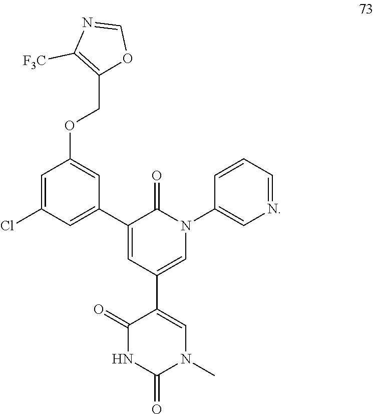 [SMILES: Cn1cc(-c2cc(-c3cc(Cl)cc(OCc4ocnc4C(F)(F)F)c3)c(=O)n(-c3cccnc3)c2)c(=O)[nH]c1=O]

In various embodiments, a compound of formula II is provided:

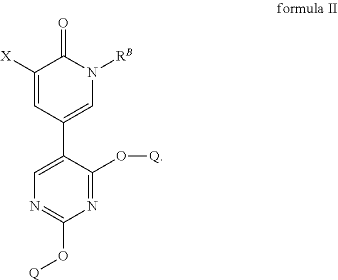

In the compound of formula II, R^{B} is as defined or described herein for the compounds of formula I or formula I-A, X is a group that can undergo, participate in, or be reactive in a cross-coupling reaction, and Q is independently at each occurrence C₁₋₄ alkyl or a protecting group. Suitable cross-coupling reactions include, but are not limited to, Suzuki cross-coupling, Negishi cross-coupling, Sonogashira cross-coupling, Suzuki-Miyaura cross-coupling, Stille cross-coupling, Heck cross-coupling, Pd-catalyzed cross coupling, Grignard reactions, and the like. In various embodiments, X is selected from the group consisting of F, Cl, Br, and I. In various embodiments, Q is methyl. In various embodiments, the compound for formula II has the structure

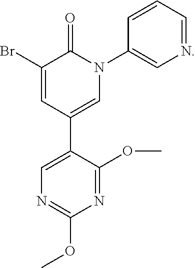

In various embodiments, the compound for formula II is not

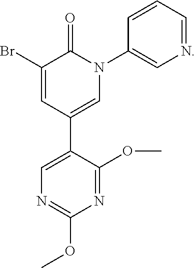

In various embodiments, a compound of formula II-A is provided:

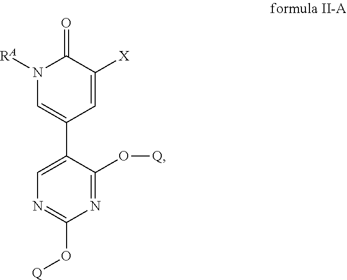

In the compound of formula II-A, R^{A} is as defined or described herein for the compounds of formula I or formula I-A, X is any group that can undergo, participate in, or be reactive in a cross-coupling reaction, and Q is independently at each occurrence C₁₋₄ alkyl or a protecting group as described herein. Suitable cross-coupling reactions include, but are not limited to, Suzuki cross-coupling, Negishi cross-coupling, Sonogashira cross-coupling, Suzuki-Miyaura cross-coupling, Stille cross-coupling, Heck cross-coupling, Pd-catalyzed cross coupling, Grignard reactions, and the like. In various embodiments, X is selected from the group consisting of F, Cl, Br, and I.

### Structural Analysis, FEP Calculations, and Initial Designs

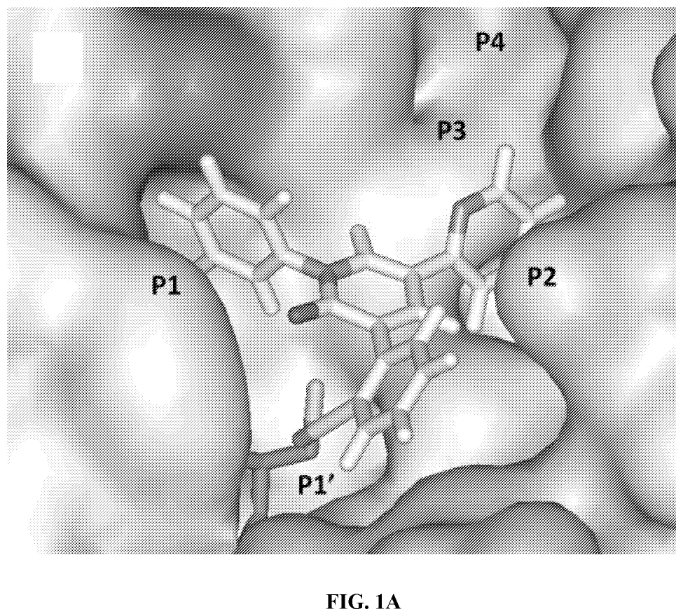

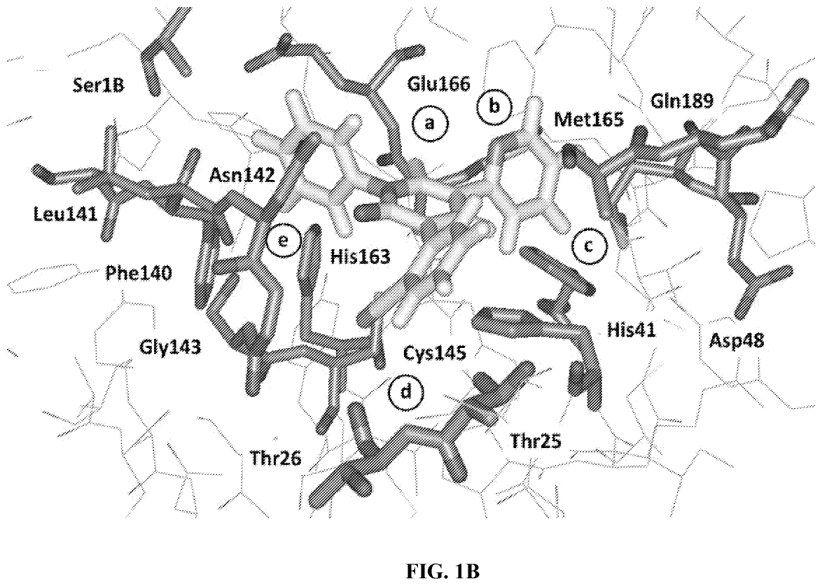

In identifying potential starting points for developing SARS-CoV-2 M^{pro} inhibitors, perampanel was deemed amenable to synthesis of analogs and its docked structure, illustrated in FIGS. 1A-1B, was unexpectedly compelling. The structure has a cloverleaf motif, with the three leaves occupying the binding pockets referred to as P1, P1′, and P2, as identified in FIG. 1A. The phenyl, cyanophenyl, and pyridinyl groups of perampanel are predicted to reside in the three pockets with the central pyridinone ring acting as the connecting hub. The catalytic residues Cys145 and His41 are located at the bottom of the site, as drawn, and other key surrounding residues are noted in FIG. 1B. However, perampanel has an approximate IC₅₀ of 100-250 μM in the SARS-CoV-2 M^{pro} inhibition assay described herein, which is generally considered as insufficiently potent for the therapeutic uses described herein.

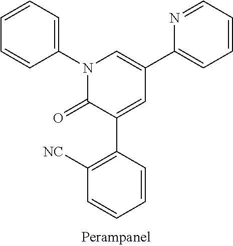 [SMILES: N#Cc1ccccc1-c1cc(-c2ccccn2)cn(-c2ccccc2)c1=O]

Close examination of the docked complex including all contacts between perampanel and M^{pro} was conducted. The locations of some notable points are highlighted in FIG. 1B points highlighted in FIG. 1B by the circled letters. (a) The backbone NH of Glu166 is directed at the pyridinone but it is not forming a hydrogen bond. (b) The pyridine nitrogen is directed towards the solvent so it is not helpful to binding. (c) The pyridine ring makes an edge-to-face aryl-aryl interaction with His41. It appears that there might be room for an additional small group in the meta position. (d) The cyano group of 1 is directed well at the NH of Cys145. In the docked structure the N . . . N distance is 3.94 Å, which shortens to 3.46 Å upon conjugate-gradient optimization of the complex using the MCPRO program with the OPLS-AA/M force field for the protein and OPLS/CM1A for the ligand. The backbone NH of Glu166 is directed at the pyridinone but does not appear to form a hydrogen bond. The ring is not rotated 180° so the pyridine nitrogen atom would be pointed towards His41 since there is no group to donate a hydrogen bond. The pyridine ring makes an edge-to-face aryl-aryl interaction with His41. Examination suggested that there might be room for an additional small group in the meta position to increase the contact.

The cyano group of perampanel is directed well at the NH of Cys145. In the docked structure the N . . . N distance is 3.94 Å, which shortens to 3.46 Å upon conjugate-gradient optimization of the complex using the MCPRO program with the OPLS-AA/M force field for the protein and OPLS/CM1A for the ligand. However, the carbonyl group of the pyridinone ring is not in a hydrogen bond and is blocked from solvation by the sidechain of Asn142. Also, the C₃-C₄ edge of the cyanophenyl ring is proximal to the opposing backbone oxygen and NH of Thr26. The phenyl ring in the P1 pocket appears mismatched with the polar environment, which includes the sidechains of Ser1B, His163, and Glu166. It is noted that a meta-CH is well directed at Ne of His163 with a C . . . N separation of 3.38 Å.

Considering these features, several modifications of perampanel were undertaken to enhance binding: switch the carbonyl group from C₂ to C₆ to form a hydrogen bond with the NH of Glu166, remove the pyridine nitrogen and add a small group at C₃ of the pyridine ring, leave the cyano group and/or introduce a hydrogen bonding edge at C₂-C₄ of the cyanophenyl ring, and replace the phenyl ring in P1 with a heterocycle that could hydrogen-bond with His163.

FEP calculations were used to explore the possible benefits of such changes. The necessary structures were built with the BOMB program and the FEP calculations were carried out using standard protocols with the MCPRO program and the above-mentioned force fields. Relative free energies of binding, AAGb, are obtained by mutating the ligand from structure A to structure B for both the protein-ligand complex in water and the unbound ligand. The configurational sampling for the systems was carried out at 25° C. with Monte Carlo simulations including the 242 protein residues nearest to the active site and 1250 and 2000 TIP4P water molecules for the ligand-bound and ligand-free calculations.

Briefly, starting from perampanel, switching the carbonyl group from C₂ to C₆ was predicted to be very favorable (ΔΔG_{b}=−4.7±0.3 kcal/mol) with formation of the hydrogen bond with Glu166; replacement of the P1 benzene ring by 2-, 3-, or 4-pyridine, 2,4-pyrimidine, 2,4,6-triazine, and 4-pyridine-N-oxide showed no benefit except for the 3-pyridine (ΔΔG_{b}=−3.6±0.2 kcal/mol), which gave a hydrogen bond with His163; and, a chlorine-scan for benzene in the P2 site predicted significant benefit for a meta-C₁ directed inward towards His41, neutral effects for a Cl at the exposed ortho and meta positions, and strong disfavoring (4-6 kcal/mol) for a Cl at the para and inward-ortho positions.

The combination led us to focus immediately on 6 as a target. Additional model building with BOMB/MCPRO for numerous heterocycles replacing the cyanophenyl group in the P1′ site also led us to 8 for which the central HNC═O of the uracil forms hydrogen bonds with the Thr26 backbone. 3,5-Dichloro analogs such as 9 were also anticipated to be viable in view of the FEP results and the expected factor-of-two benefit for binding due to the added symmetry and the predicted strong preference for the chlorine atoms in 6 and 8 to be directed inward.

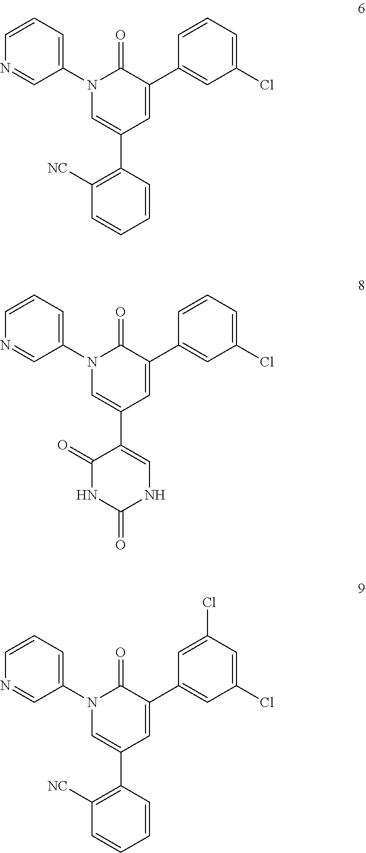 [SMILES: N#Cc1ccccc1-c1cc(-c2cccc(Cl)c2)c(=O)n(-c2cccnc2)c1 ; O=c1[nH]cc(-c2cc(-c3cccc(Cl)c3)c(=O)n(-c3cccnc3)c2)c(=O)[nH]1 ; N#Cc1ccccc1-c1cc(-c2cc(Cl)cc(Cl)c2)c(=O)n(-c2cccnc2)c1]

Initial Designs were Confirmed by Assay and Crystallography

In view of the structural studies described herein, compounds 6, 8, and 9 were synthesized. Inhibition of proteolytic activity was tested using recombinant SARS-Cov-2 M^{pro} as described herein in the Experimental section. For the kinetic assays, 100 nM M^{pro} in reaction buffer (20 mM Tris, 100 mM NaCl, 1 mM DTT, pH 7.3) was incubated with or without compound in DMSO at varying concentrations to a final DMSO concentration of 6% for 15 minutes with shaking at room temperature. The reaction was initiated by addition of substrate (Dabcyl-KTSAVLQ↓SGFRKM-E (Edans-NH2); GL Biochem) in reaction buffer, which is cleaved by M^{pro}, generating a product containing a free Edans group. Fluorescence was monitored at an excitation wavelength of 360 nm and emission wavelength of 460 nm. Baseline subtraction controlled for intrinsic fluorescence of each compound as well as intrinsic fluorescence of the un-cleaved FRET substrate. All tested compounds had purity of at least 95% based on HPLC, and all measurements were performed in triplicate and averaged.

As reflected in Table 1, the initial results were gratifying with IC₅₀ values for 6, 8, and 9 of 10.0, 6.4, and 4.2 μM showing striking improvement over the >100 μM for perampanel. Both the cyanophenyl and uracilyl alternatives are viable, with a small preference for the uracil 8. Furthermore, addition of the second chlorine atom to 6 in going to 9 did provide the expected ca. factor-of-2 enhancement in inhibitory activity.

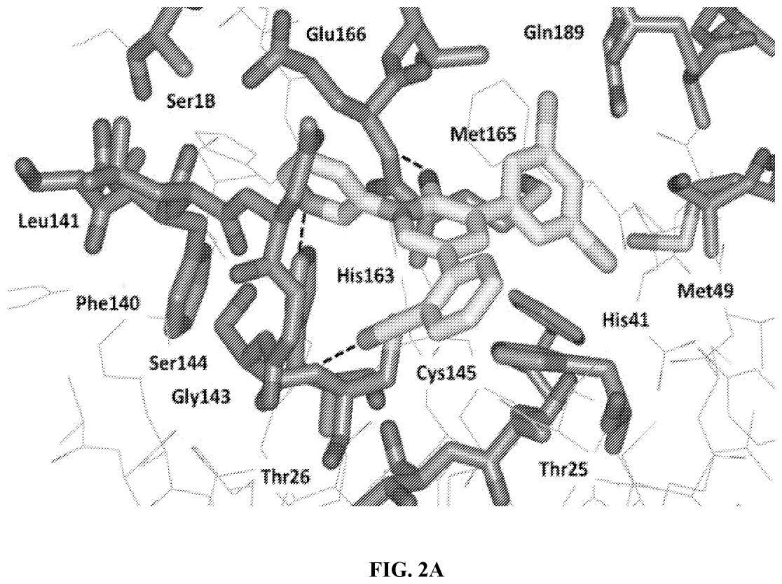

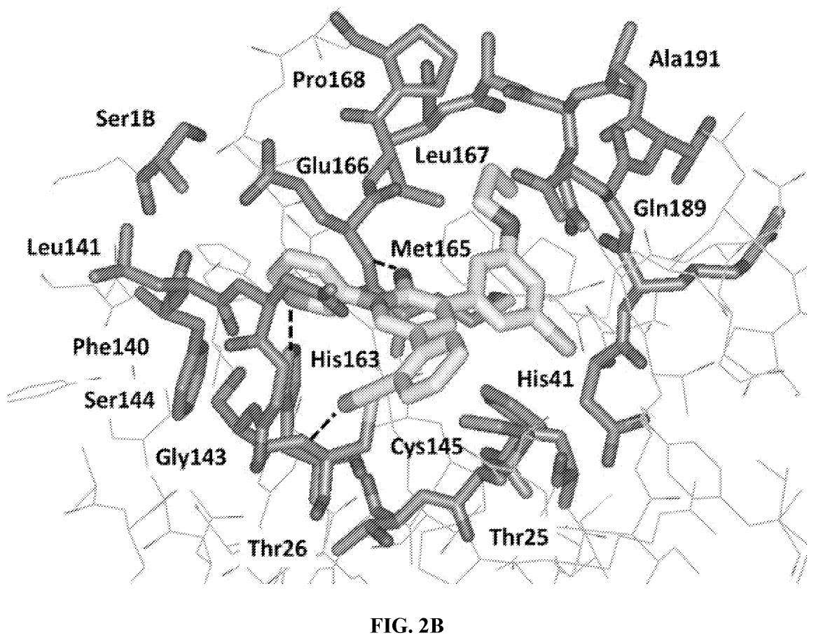

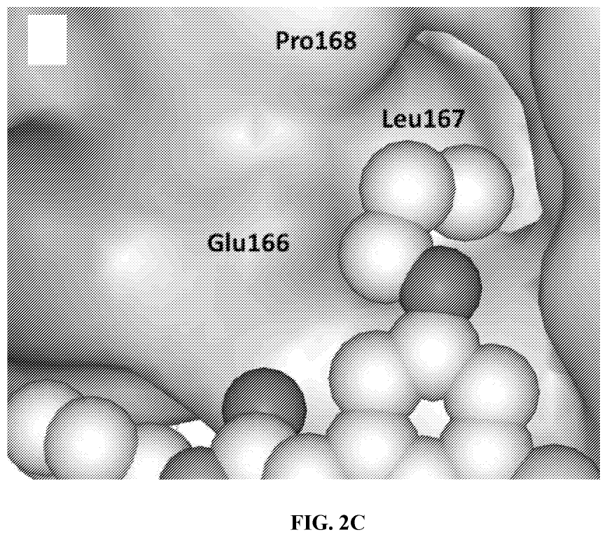

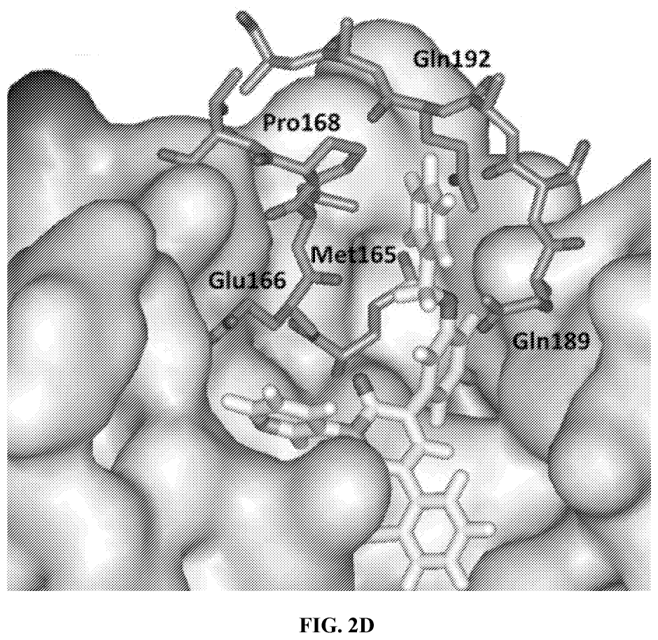

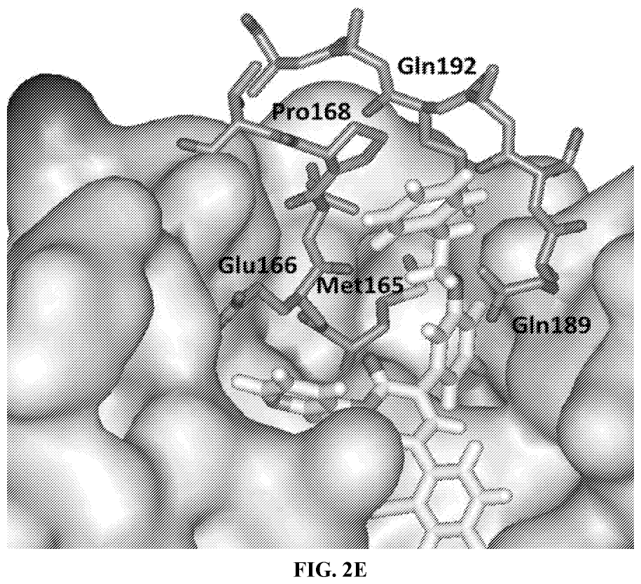

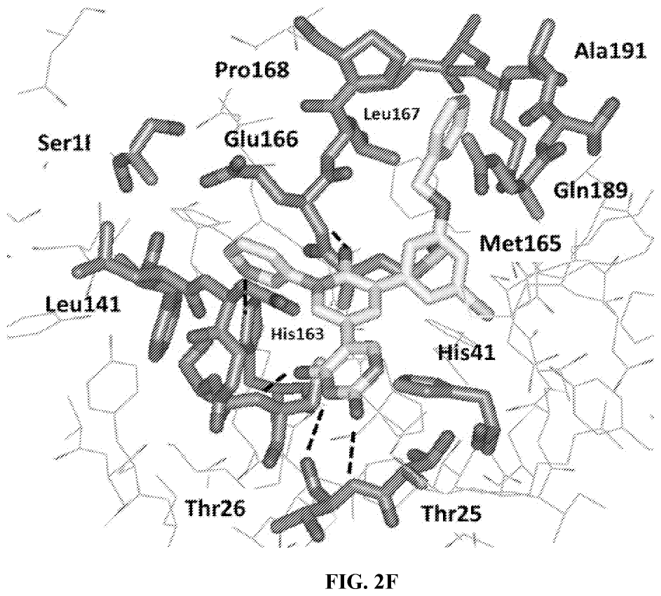

Fortunately, it was also possible to obtain a high-resolution (1.7 Å) X-ray crystal structure for the complex of 9 with SARS-CoV-2 M^{pro} (FIG. 2A). As shown in FIG. 2, the crystal structure fully confirmed the expectations from the modeling. There are now three protein-ligand hydrogen bonds between the pyridinone oxygen and Glu166 nitrogen (2.84 Å), nitrile nitrogen and nitrogen of Cys145 (3.14 Å), and pyridine nitrogen and Ne of His163 (2.92 Å). In addition, a chlorophenyl edge packs well against the imidazole ring of His41 in the P2 pocket with no indication of room for expansion. The overall structure of the protein is essentially identical to that used for the original modeling (PDB ID: 5R82) with an rms deviation of 0.62 Å between the protein Ca atoms.

TABLE 1
Measured activities for inhibition of SARS-CoV-2 M^{pro}.

| Cmpd | IC₅₀ (μM) | Cmpd | IC₅₀ (μM) | Cmpd | IC₅₀ (μM) |
|---|---|---|---|---|---|
| Perampanel | 100-250^{a} | 15 | 1-10 | 32 | 0.110 ± 0.035 |
| 6 | 9.99 ± 2.50 | 18 | 1.20 ± 0.03 | 34 | 0.024 ± 0.007 |
| 8 | 6.38 ± 1.21 | 24 | 0.120 ± 0.016 | 35 | 0.037 ± 0.007 |
| 9 | 4.02 ± 1.36 | 25 | 0.25 ± 0.09 | 36 | 0.036 ± 0.003 |
| 13 | 0.14 ± 0.02 | 26 | 0.19 ± 0.03 | 37 | 0.018 ± 0.002 |
| 16 | 0.47 ± 0.02 | 21 | 0.128 ± 0.015 | | |
| 14 | 0.28 ± 0.05 | 29 | 0.110 ± 0.013 | | |
| 17 | 0.51 ± 0.02 | 31 | 0.100 ± 0.007 | | |

^{a}Fluorescence of compound interfered with assay.

### Lead Optimization in P3-P4 Region

After this initial advance, consideration turned towards growth into the P3-P4 region (FIG. 1A) to obtain increased potency. Model building and the crystal structure for 9 made it clear that it should be possible to replace the meta-chlorine near Gln189 with a variety of alkyl or alkoxy groups. Again, FEP calculations were executed to obtain ΔΔG_{b} values for replacing the chlorine with 11 alternatives yielding ΔΔG_{b} values in kcal/mol: methyl (1.36), ethyl (−0.13), propyl (−2.88), methoxyl (0.09), ethoxyl (−3.02), propoxyl (−3.54), butoxyl (−3.44), methoxyethoxyl (−3.90), hydroxymethyl (1.58), hydroxyethyl (−0.74), and methoxymethyl (−0.15) with uncertainties of ca. ±0.4 kcal/mol. The results predict significant improvements especially with alkoxy groups containing 4 or 5 non-hydrogen atoms, which place a CH₂ or CH₃ group in the hydrophobic P4 site. Past experience has indicated that the range of the FEP ΔΔG_{b} values is larger than observed by experiment, but that improvements in activity are almost always found when ΔΔG_{b} is more favorable than 2-3 kcal/mol.

TABLE 2
Computed changes in free energy of binding (kcal/mol)
for conversion of a chlorine to X in 9.^{a}

| X | ΔΔG_{b} | X | ΔΔG_{b} |
|---|---|---|---|
| Me | 1.36 | OBu | −3.44 |
| Et | −0.13 | OCH₂CH₂OCH₃ | −3.90 |
| Pr | −2.88 | CH₂OH | 1.58 |
| OMe | 0.09 | CH₂CH₂OH | −0.74 |
| OEt | −3.02 | CH₂OCH₃ | −0.15 |
| OPr | −3.54 | | |

^{a}Statistical uncertainties in the results are ±0.3 to ±0.5 kcal/mol.

Thus, the propoxyl 13 and methoxyethoxyl 16 analogs of 9 were synthesized, and they were found to have IC₅₀ values of 0.14 and 0.47 μM, respectively (Table 1). The FEP results were again nicely predictive, and the factor of ca. 30 improvement in the potency for 13 over 9 is striking. It was also possible to obtain a crystal structure for the complex of 13 with SARS-CoV-2 M^{pro} (FIG. 2B). In this case, the asymmetric unit contains two M^{pro} monomers and two copies of 13 with nearly identical binding sites. As for 4, there is close packing of the chlorophenyl fragment and His41 along with the three protein-inhibitor hydrogen bonds. The propoxyl group with its terminal methyl group indeed extends into the hydrophobic region at the juncture of Met165, Leu167, and Pro168 in the P4 site. The electron density for 5 is very well defined and shows that the terminal OCCC dihedral angle is gauche to allow contact of the methyl group with terminal methyl groups of Met165 and Leu 167. The packing in this region is illustrated in FIG. 2C. The CCᵢₚₛₒOC anisole fragment is planar and directed towards Glu166; there is steric blockage in the opposite direction towards Gln189 (FIG. 2C).

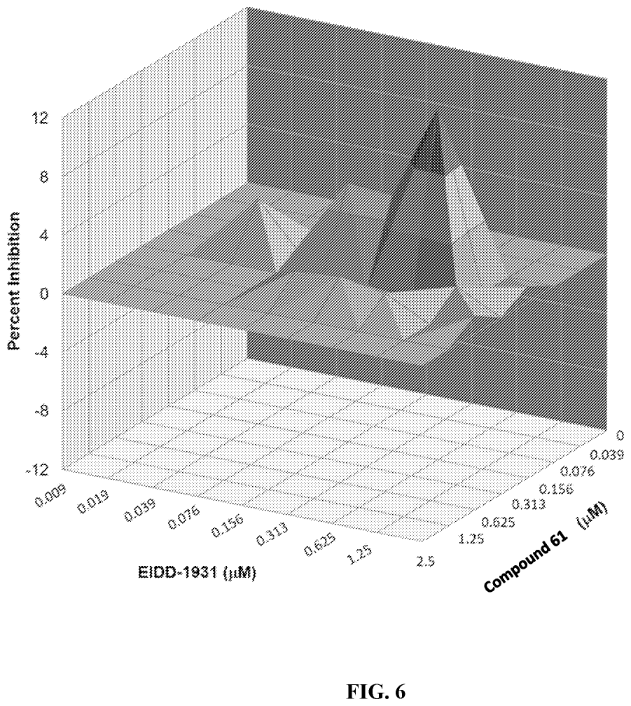

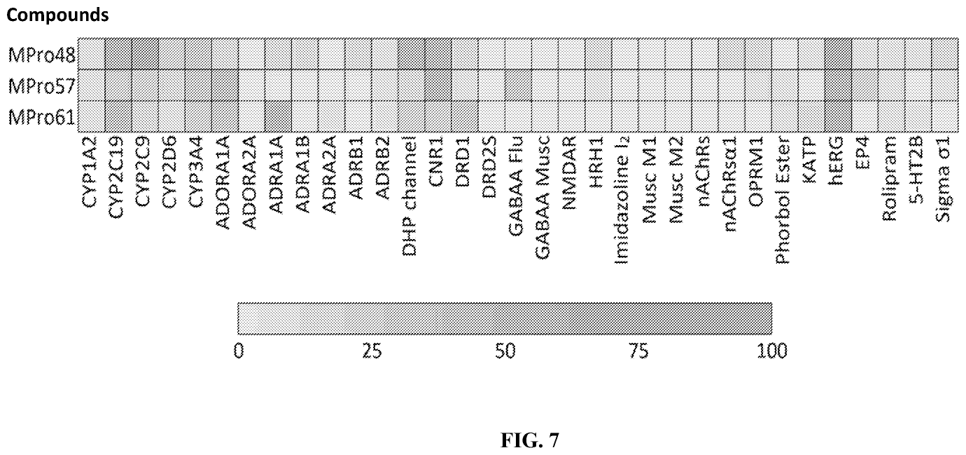

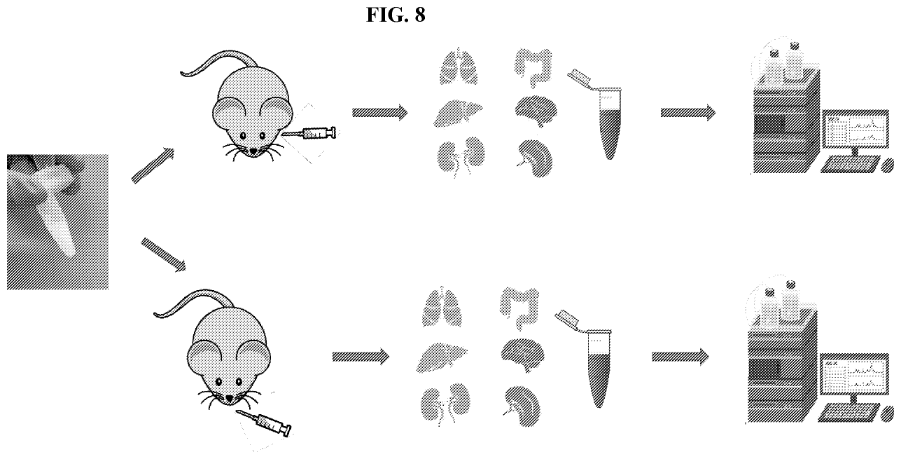

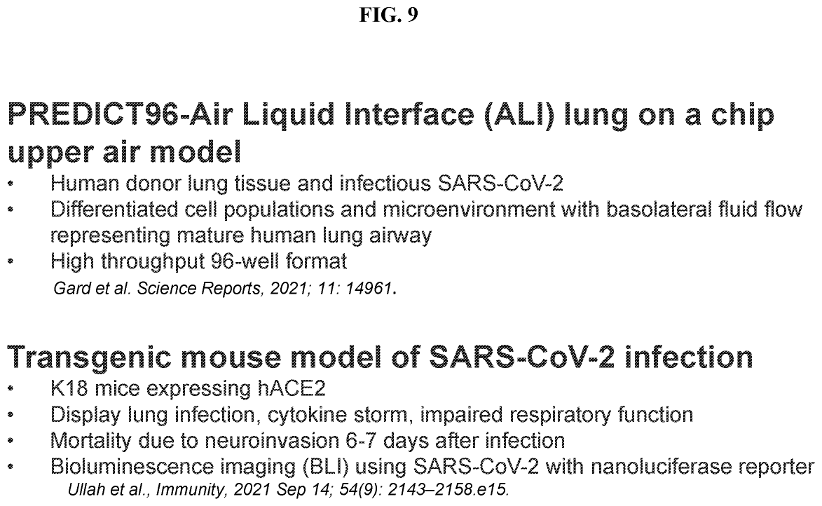

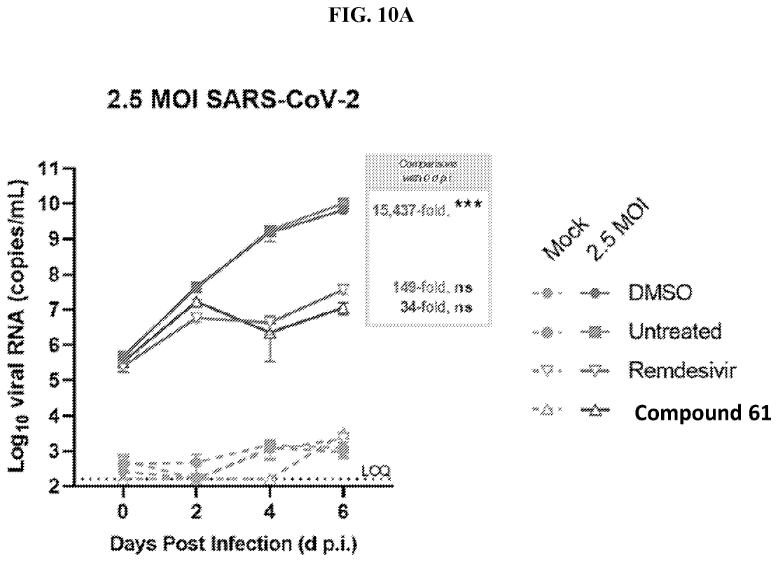

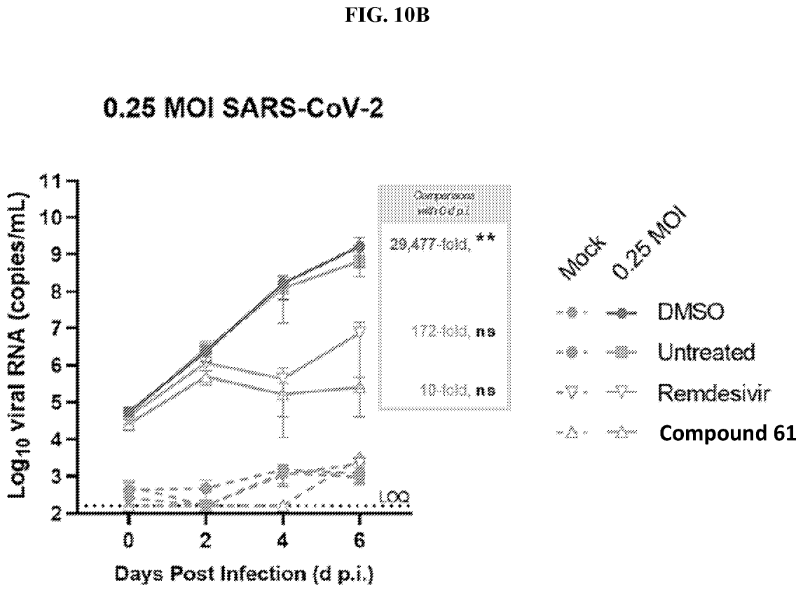

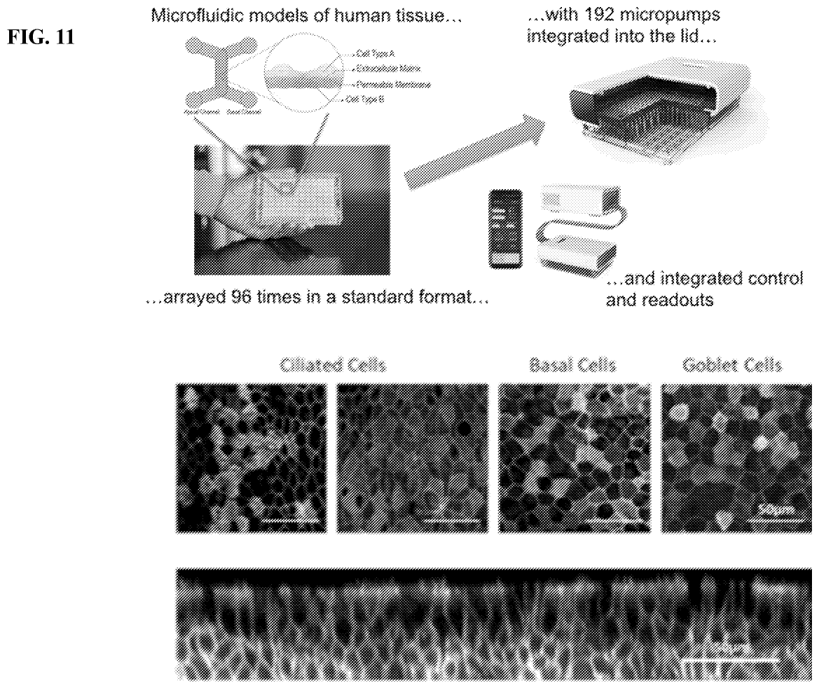

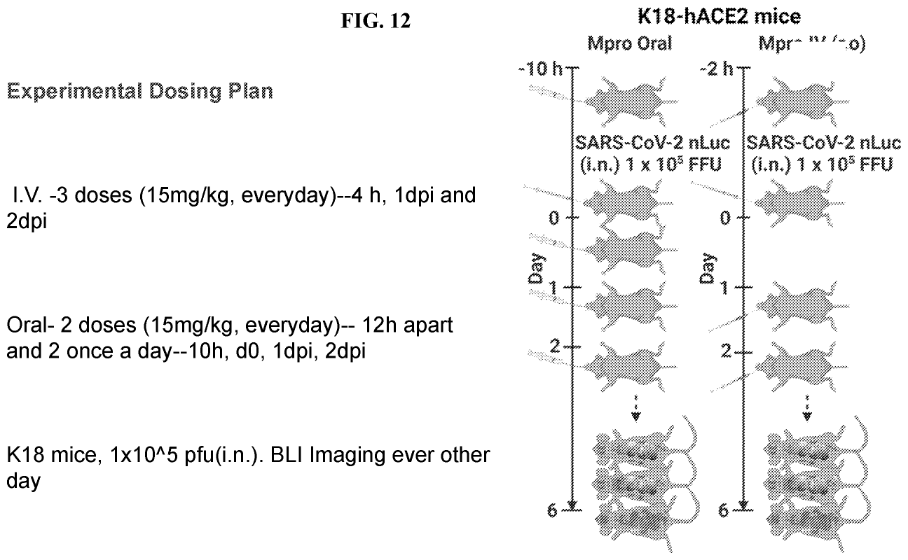

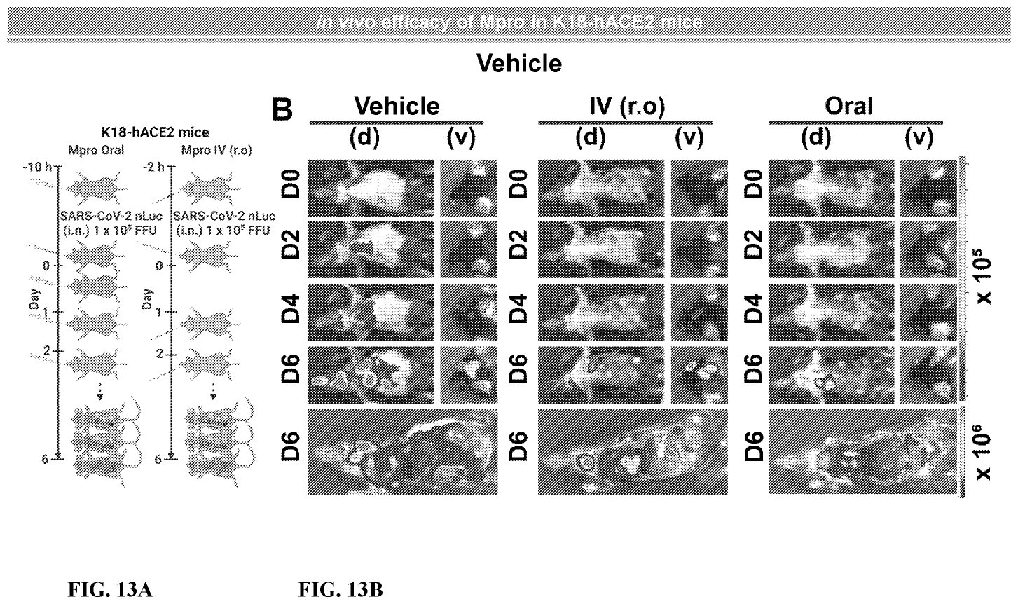

Many other possibilities for the P3-P4 appendage were modeled by building structures of the complexes with BOMB including ones that incorporated phenyl or heterocyclic rings. Both benzyloxy and phenethyloxy groups appeared promising, as illustrated in FIG. 6, and synthetic access to these and substituted analogs from the common phenolic precursor was also an attractive feature. The benzyloxy analog was predicted to place a phenyl edge in the P4 site (FIG. 2D), while the higher homolog is fully extended with face-to-face contact between Pro168 and the phenyl ring (FIG. 2E). Compounds 14 and 17 were synthesized, which yielded IC₅₀ values of 0.28 and 0.51 μM, respectively, in the enzyme inhibition assay. Thus, they are competitive in potency with 13 and 6, and modeling of substituted analogs was auspicious and is considered further below.

At this point, before turning back to the uracil series, curiosity arose for preparation of 15, the 5-pyrimidinyl analog of 9. The thoughts were that the second nitrogen atom would be solvent exposed or possibly form a hydrogen bond with the sidechain amino group of Asn142, which itself is solvent-exposed; however, there would be the factor-of-two symmetry gain for binding the pyrimidine over the pyridine. On the other hand, the added nitrogen atom would decrease the basicity and hydrogen-bond-accepting ability of the nitrogen atom that is hydrogen-bonded to His163. In the event, 15 was prepared and assayed yielding an IC₅₀ in the 1-10 μM range. So, the latter consideration appears to dominate, and it was decided to continue with 3-pyridinyl for the P1 site.

The meta-methoxy uracil analog 18 was synthesized and did give an improvement in IC₅₀ to 1.2 μM from the 6.4 μM for the unsubstituted 8. The 3,5-dichloro uracil analog corresponding to 9 was not prepared, but based on the results for 6, 8, and 9, it would be expected to have an IC₅₀ of 2-3 μM. Thus, little benefit is apparent from changing the chlorine to a methoxy group, which is consistent with the FEP prediction in Table 2. A larger alkoxy group is needed as in 13 and 16 to extend to the P4 site. Compounds 24, 25, and 26 were then prepared to explore the effects of propoxy, butoxy, and isopentoxy alternatives for the uracil series. As expected from the FEP results, the activities are not improved for expansion of the alkyl ether substituent beyond the propoxy analog 24 (0.120 μM).

The benzyloxy uracil analog 21 was prepared and also showed good activity with an IC₅₀ of 0.128 μM and there was additional gain for the phenethyloxy homolog 29 at 0.110 μM. It was expected that further progress was more likely to arise by addition of small groups at the ortho and meta positions in 21, which might better fill the P4 site as suggested in FIG. 2D. FEP calculations were carried out and predicted gains in free energy of binding of 2-3 kcal/mol for methyl, fluorine, or chlorine substituents, which is large enough to usually yield observed benefits. The mono-methyl analogs 31 and 32 were prepared and did show small improvement over 21 to 0.11 and 0.10 μM, respectively, (Table 1) in spite of the expected loss from the reduced symmetry.

At this point, a crystal was obtained for the complex of 21, which was of particular interest since it was the first structure for the uracil series (FIG. 2F). It was gratifying to see that the five anticipated hydrogen bonds between the ligand and M^{pro} were all present: pyridinone carbonyl oxygen with Glu166 N (2.79 Å), the pyridine nitrogen with His163 (2.97 Å), and the uracil O—NH—O edge with the NH of Cys145, and backbone O and NH of Thr26 (3.55, 3.45, and 3.37 Å). No water molecules are located in the crystal structure between the protein and the ligand, while there are three hydrogen-bonded water molecules bridging between the backbone oxygen of Glu166 and the pyridinone oxygen, and between the central uracilyl NH and CO and the backbone oxygen and NH of Thr26. Furthermore, the conformation of the benzyloxy side chain is extended with a phenyl edge in the P4 site as in FIG. 2D. This supported further exploration of small substituents at the ortho and meta positions, which was realized by synthesis of the fluorine and chlorine substituted analogs 34, 35, 36, and 37. The outcome was highly productive yielding inhibitors with IC₅₀ values of 0.018-0.037 μM with the ortho-chloro analog 37 being the most potent (Table 1).

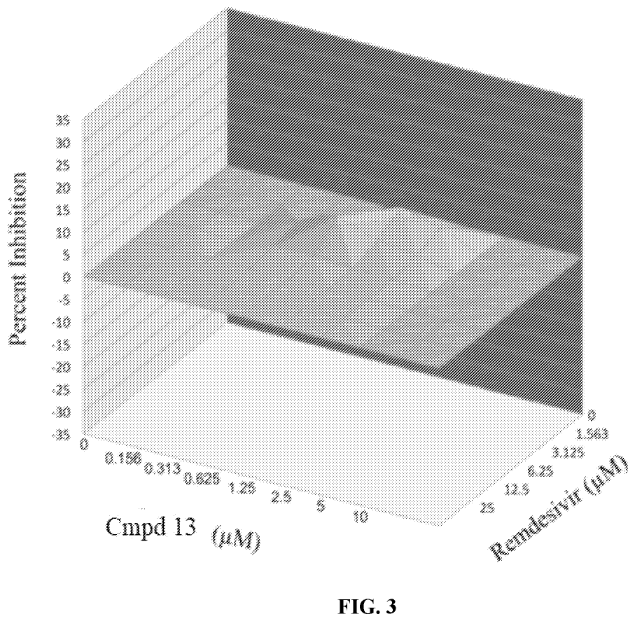

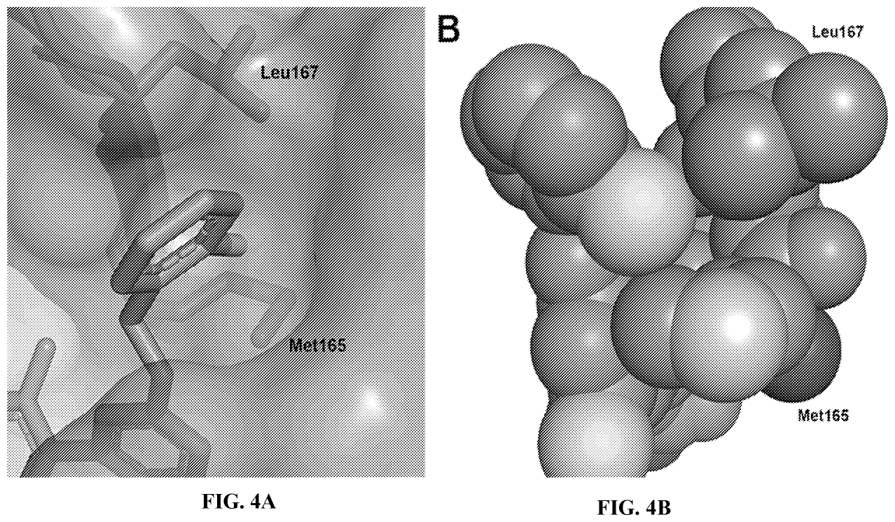

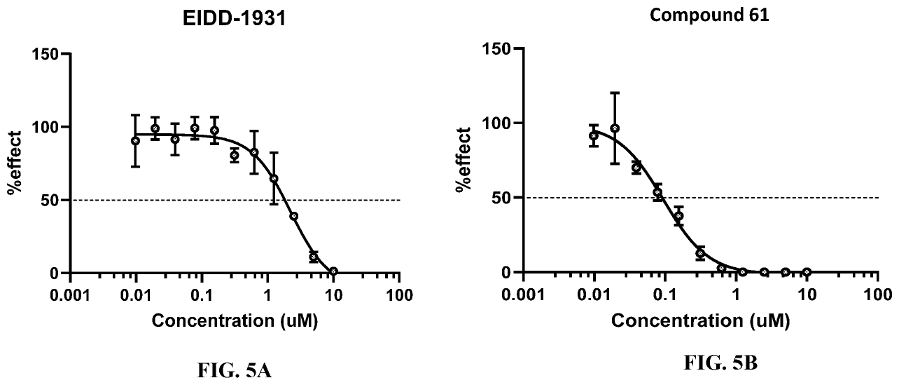

An X-ray crystal structure for the complex of 37 with M^{pro} was obtained at 2.2-Å resolution (PDB ID: 7L13), which clearly documents that the ortho-C₁ resides in the Met165/Leu167 pocket (FIG. 4). Monofluoro analogs 48 and 44 were then prepared, and they are also potent inhibitors at 0.036 and 0.020 μM.

Simultaneously, additional inhibitors in the low-nM range were obtained by replacing the propyloxyl group in the uracil 24 with cyclopropylmethoxly (40, 0.037 μM) and 3,3,3-trifluoropropoxyl (41, 0.025 μM). In various embodiments, these analogs have enhanced metabolic stability compared to 24. The corresponding analogs in the cyanophenyl series (39, 47) were also prepared, but with IC₅₀ values of 0.170 and 0.120 μM, they showed similar potency to 13 (0.140 μM) in contrast to the three-fold boost in the uracil series for 40 vs. 24. A crystal structure for the complex of 39 with M^{pro} was obtained at 1.8-Å resolution (PDB ID: 7L14); it shows the C₂-C₃ edge of the cyclopropyl ring in close contact with Leu167, but with less ideal contact with Met165 compared to the ortho-chlorine atom in 37.

### Evaluation of Antiviral Activity Against SARS-CoV-2

To explore the series' potential for therapeutic value, several compounds were tested for inhibition of infectious SARS-CoV-2 replication in Vero E6 cells. Protection against the viral cytopathic effect was tested in two assays, as detailed in the Supplementary Information. Due to the ability to multiplex in 96-well plates and concurrently evaluate compound general cytotoxicity, a methylthiazolyl-diphenyl-tetrazolium bromide (MTT) dye was used in the primary assay, while the more labor intensive, lower throughput viral plaque assay was used to confirm antiviral activity. Previous studies have shown excellent correlation between the two assays. In addition to the Vero E6 cells, compound cytotoxicity was also evaluated in normal human bronchial epithelial cells (NHBE) via MTT assays. The results are summarized in Table 3. For the MTT assays, three independent measurements were performed in triplicate to yield the indicated statistical uncertainties (±1). The viral plaque assay, which requires serial dilutions using 6-well plates, was only performed once for each compound except 13. In that case the results of three independent experiments provided an uncertainty of ±0.15 μM.

It was found that 13 has antiviral potency against infectious SARS-CoV-2 in both the MTT and viral titer plaque and MTT assays with EC₅₀ values of 2.5 and 1.5 μM, a little above a reported value for the SARS-CoV-2 polymerase inhibitor, remdesivir. For 46, antiviral activity was found in the viral plaque assay, but not in the MTT assay, perhaps due to infringing cytotoxicity or compound efflux. Likewise, 37 and 44 showed activity in the viral plaque assay, but lacked antiviral activity in the MTT assay; and, they showed the greatest cytotoxicity. The most auspicious results are for 39, which exhibits potency near 1 μM in both antiviral assays, and it shows no cytotoxicity to the highest concentration tested (100 μM). The closely related 47, which just replaces the cyclopropylmethoxyl group in 39 with trifluoropropoxyl, is more active in the MTT assay at 1.1 μM, similar to remdesivir, but it is also more cytotoxic towards the NHBE cells.

TABLE 3
Anti-SARS-CoV-2 activity and cellular toxicity (μM).

| | | EC₅₀ | | CC₅₀ | | | |
|---|---|---|---|---|---|---|---|
| compd | IC₅₀ | replicon | infectious virus | Vero E6 | | NHBE | |
| remdesivir | — | 0.59 ± 0.25 | 0.77^{a}, 2.1^{b} | 72 ± 28 | g | 41 ± 2 | g |
| 13 | 0.14 ± 0.020^{g} | 1.51 ± 0.01 | 1.5^{c, g} | 22 ± 7.2 | g | 20 ± 2 | g |
| 39 | 0.170 ± 0.02^{g} | 1.76 ± 0.31 | 0.98^{c, g} | >100 | g | >100 | g |
| 48 | 0.12 ± 0.006^{g} | 120 ± 0.20 | 1.1 ± 0.5^{g} | 22 ± 8 | g | 25 ± 5 | g |

| | | | | | |
|---|---|---|---|---|---|
| 50 | 0.083 ± 0.023 | 0.73 ± 0.26 | 1.7 ± 0.1^{h} | ND^{e} | ND^{e} |
| 51 | 0.042 ± 0.015 | >40 | ND^{e} | >100 | 22 |
| 62 | 0.105 ± 0.015 | NA^{d} | ND^{e} | >100 | >100 |
| 63 | 0.038 ± 0.015 | NA^{d} | ND^{e} | >100 | >100 |
| 57 | 0.061 ± 0.007 | 0.53 ± 0.23 | 1.2 | 82 | >100 |
| 60 | 0.059 ± 0.014 | 0.82 ± 0.28 | ND^{e} | >100 | ca. 95 |
| 43 | 0.028 ± 0.007 | 3.2 ± 1.00 | ca. 1 | >80 | >95 |
| 61 | 0.044 ± 0.009 | 0.175 ± 0.005 | 0.08^{b} | >32.5^{f} | >32.5^{f} |
| 64 | 0.061 ± 0.011 | 1.08 | ND^{e} | >100 | >100 |

^{a}From Wang, M et al., Cell Res. 2020, 30, 269-271;
^{b}alamarBlue;
^{c}Plaque Assay;
^{d}not active;
^{e}not determined;
^{f}Limited by solubility in DMSO;
^{g}Zhang et al., ACS Cent. Sci. 2021, 7, 467-475;
^{h}MTT.

In various embodiments, the numbered compounds in Table 4 have the following IC50 values for inhibiting SARS-CoV-2 M^{pro}.

TABLE 4
IC₅₀ values for compounds of formula (I) or
(I-A) for inhibition of SARS-Cov-2 M^{pro}.

| Cmpd | IC50 (μM) |
|---|---|
| 1 | 100 |
| (perampanel) | |
| 2 | ND |
| 4 | ND |
| 5 | ND |
| 6 | 9.99 ± 2.50 |
| 7 | ND |
| 8 | 6.38 ± 1.21 |
| 9 | 4.02 ± 1.36 |
| 10 | 15.6 ± 2.37 |
| 11 | 5-20* |
| 12 | NA |
| 13 | 0.14 ± 0.02 |
| 14 | 0.28 ± 0.05 |
| 15 | 1-10 |
| 16 | 0.47 ± 0.02 |
| 17 | 0.51 ± 0.015 |
| 18 | 1.2 ± 0.03 |
| 19 | ca. 10 |
| 20 | 10-100 |
| 21 | 0.128 ± 0.015 |
| 22 | 10-100 |
| 23 | ca. 10 |
| 24 | 0.12 ± 0.016 |
| 25 | 0.25 ± 0.089 |
| 26 | 0.19 ± 0.026 |
| 27 | 0.21 ± 0.023 |
| 28 | 0.25-0.50 |
| 29 | 0.11 ± 0.013 |
| 30 | >1 |
| 31 | 0.100 ± 0.0072 |
| 32 | 0.110 ± 0.035 |
| 33 | 0.5-1.0 |
| 34 | 0.024 |
| 35 | 0.037 |
| 36 | 0.036 |
| 37 | 0.018 |
| 38 | 0.2 |
| 39 | 0.170 ± 0.022 |
| 40 | ca. 290 |
| 41 | 0.025 ± 0.003 |
| 42 | 0.037 ± 0.004 |
| 43 | 0.028 ± 0.007 |
| 44 | 0.030 ± 0.004 |
| 45 | 0.036 ± 0.005 |
| 46 | 0.0265 ± 0.004 |
| 47 | 0.131 ± 0.009 |
| 48 | 0.120 ± 0.006 |
| 49 | 0.085 ± 0.005 |
| 50 | 0.083 ± 0.023 |
| 51 | 0.042 ± 0.015 |
| 52 | 0.328 ± 0.061 |
| 53 | NA |
| 54 | 1.2 ± 0.1 |
| 55 | ca. 10 |
| 56 | 0.427 ± 0.027 |
| 57 | 0.059 ± 0.014 |
| 58 | 1.3 ± 0.2 |
| 59 | ca. 0.194 |
| 60 | 0.059 ± 0.014 |
| 61 | 0.044 ± 0.009 |
| 62 | 0.105 ± 0.013 |
| 63 | 0.038 ± 0.015 |
| 64 | 0.061 ± 0.011 |
| 65 | 0.130 ± 0.018 |
| 66 | 0.040 ± 0.002 |

^{a} ND = not determined.
^{b} NA = not available.

Synergistic Effects with Compounds of Formula (I) and Formula (I-a)

A desirable feature for an antiviral drug candidate is synergistic behavior when used in combination with other antiviral agents. In various embodiments, compounds of formula (I) or (I-A) display synergistic behavior with at least one other antiviral agent. In certain embodiments, compound 13 has synergistic anti-viral properties when combined with remdesivir. A replicon assay with non-infectious SARS-CoV-2 clone with a nanoluciferase reporter was utilized to evaluate combinations of 13 and remdesivir. The inhibitory data were analyzed using MacSynergy II, a 3D model for statistical evaluation of combination assays. In this model, a simple additive effect results in a horizontal plane at 0% inhibition, whereas a synergistic or antagonistic effect will render a hill or depression above or below the plane. As shown in the 3D plot in FIG. 3, a range of combinations of 13 and remdesivir do provide values above the plane. This reflects statistically significant synergistic behavior with a ratio of 30.8/0 M²% for the mean synergy volume/antagonism volume, as detailed in Supporting Information. In various embodiments, at least one compound of formula (I) or (I-A) displays synergistic behavior with at least one additional antiviral agent such as, without limitation, amantadine, rimantadine, oseltamivir, zanamivir, peramivir, acyclovir, valacyclovir, penciclovir, famciclovir, ganciclovir, foscamet, cidofovir, fomivirsen, remdesivir, lopinavir, ritonavir, molnupiravir, nirmatrelvir (PF-07321332), and the like, and combinations thereof. The at least one additional antiviral agent can be administered according to any of the dosing amounts and/or regimens described herein.

In certain embodiments, compounds of formula (I) or (I-A) are synergistic with remdesivir.

In certain embodiments, compounds of formula (I) or (I-A) are synergistic with molnupiravir.

In certain embodiments, compounds of formula (I) or (I-A) are synergistic with nirmatrelvir, ritonavir, or a combination of nirmatrelvir and ritonavir.

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means.

Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

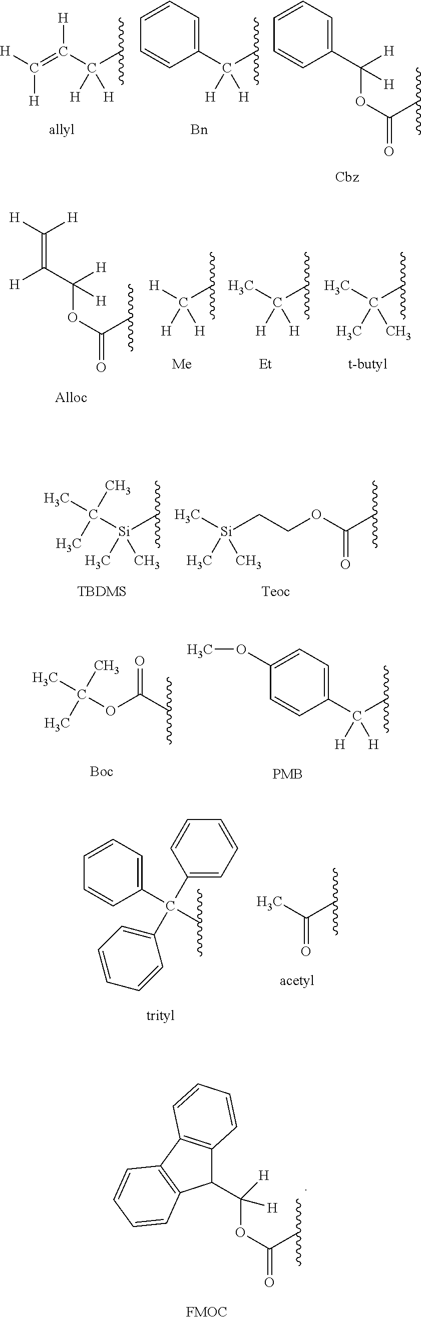

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

The disclosure includes a method of treating a COVID-19 infection using the compounds formula (I) or (I-A). Coronavirus disease 2019 (COVID-19) is caused by one or more strains of severe acute respiratory syndrome coronavirus 2 (SARS-CoV-2). In certain embodiments, the method of treating a COVID-19 infection includes administering a composition that includes a therapeutically effective amount of at least one compound of formula (I) or (I-A) to a subject in need thereof.

Also provided herein is a method of inhibiting SARS-CoV-2 main protease. The method includes contacting SARS-CoV-2 main protease (M^{pro}) with at least one compound of formula (I) or (I-A). The contacting of the main protease can be in vitro or in vivo. In various embodiments, the contacting includes administering the compound of formula (I) or (I-A) to a subject in an amount sufficient to inhibit the biological activity of SARS-CoV-2 main protease. After entering the cells of a subject, SARS-CoV-2 uses host cellular machinery to replicate. Using the host, the virus synthesizes two open reading frames (ORFs): ORF1a and ORF1b. ORF1a encodes for viral replicases and ORF1b encodes for two polyproteins that are auto-catalytically processed to produce two proteases: M^{pro} (a chymotrypsin-like serine protease) and PL^{pro} (a papain-cysteine protease). These proteases are subsequently further processed to produce additional proteins, both structural and non-structural, that are essential for viral replication. In certain embodiments, the term “inhibit the biological activity” as used herein means that a compound of formula (I) or (I-A) inhibits M^{pro} sufficiently to prevent its function or role in the replication of additional viral particles of SARS-CoV-2.

Also provided herein is a method of treating, reducing, or ameliorating one or more symptoms associated with COVID-19 infection, by administering a composition containing a therapeutically effective amount of the compound of formula (I) or (I-A) to a subject in need thereof. In various embodiments, the one or more symptoms is at least one of fever, cough, myalgia, fatigue, sputum production, headache, diarrhea, vomiting, dyspnea, lymphopenia, and hypoalbuminemia.

The composition includes, in various embodiments, at least one pharmaceutically acceptable excipient, carrier, or diluent. In various embodiments, the administering of the composition of formula (I) or (I-A) is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

In various embodiments, the method includes administering at least one additional therapeutic agent. The at least one additional therapeutic agent can administered sequentially or concurrently with the compound of formula (I) or (I-A). Suitable additional therapeutic agents include other antiviral agents, steroids, anti-malaria agents, non-steroidal anti-inflammatory agents (NSAIDS), analgesics, and the like. In various embodiments, the subject is a mammal. In various embodiments, the mammal is a human.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, any of the methods described herein can further comprise administering to the subject an additional therapeutic agent that treats COVID-19 or symptoms associated with COVID-19.

In certain embodiments, administering the compound(s) described herein to the subject allows for administering a lower dose of the additional therapeutic agent as compared to the dose of the additional therapeutic agent alone that is required to achieve similar results in treating COVID-19 or symptoms associated with COVID-19 in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

### Combination Therapies

The compounds useful within the methods described herein can be used in combination with one or more additional therapeutic agents useful for treating COVID-19 or symptoms associated with COVID-19. These additional therapeutic agents may comprise compounds that are commercially available or synthetically accessible to those skilled in the art. These additional therapeutic agents are known to treat or reduce the symptoms of a COVID-19 infection or symptoms associated with a COVID-19 infection.

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Schemer, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a COVID-19 infection or symptoms associated with COVID-19. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a COVID-19 infection or symptoms associated with COVID-19 in the patient. An effective amount of the therapeutic compound necessary to achieve a therapeutic effect may vary according to factors such as the state of the disease or disorder in the patient; the age, sex, and weight of the patient; and the ability of the therapeutic compound to treat a COVID-19 infection or symptoms associated with COVID-19 in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of COVID-19 or symptoms associated with COVID-19 in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Inhaled Administration

The compounds of formula (I) or (I-A) can be, in some embodiments, formulated for administration by inhalation. Inhalation can include the use of a nebulizer to administer, for example and without limitation, an aerosol containing the compound of formula (I) or (I-A). The nebulizer can be used to administer a composition containing the compound of formula (I) or (I-A) over a period of about 5 to about 180 minutes, or over a period of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or about 180 minutes. The amount of the compound of formula (I) or (I-A) delivered by a nebulizer can be any of the amounts described herein, and the concentration of the composition containing the compound of formula (I) or (I-A) used in the nebulizer can be suitably determined based on the desired total amount of the compound of formula (I) or (I-A) to be delivered.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%0, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10, 1%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In certain embodiments, the compound(s) described herein are administered to a patient, alone or in combination with another pharmaceutical agent, using a sustained release formulation. In certain embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a COVID-19 infection or symptoms associated with COVID-19 in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### I. General Synthesis Information

All reactions were carried out under ambient atmosphere unless otherwise noted. Room temperature (rt) is defined as 21-23° C. Acetonitrile (MeCN), dichloromethane (DCM), Tetrahydrofuran (THF), N,N-dimethylformamide (DMF), and toluene (PhMe) were dried over alumina and dispensed under argon from a Glass Contour Seca Solvent Purification System. All reagents were obtained from commercial sources and used without further purification. Deionized water was used for reactions, extraction solutions, and reverse-phase chromatography. All other solvents used for chromatography were HPLC grade.

Column chromatography was carried out on silica gel (300-400 mesh) unless otherwise specified. If specified, flash chromatography was performed using a Biotage Isolera One purification system equipped with a 10, 25, 50, or 100 g SNAP Ultra (HP Sphere, 25 μm silica) cartridge for normal-phase column chromatography, and 12, 30, 60, or 120 g SNAP-C18 columns for reverse-phase column chromatography. Analytical thin-layer chromatography (TLC) was performed using 60 Å Silica Gel F254 pre-coated plates (0.25 mm thickness). TLC plates were visualized by irradiation with a UV lamp. Lyophilization was conducted on a Labconco FreeZone 4.5 L −84C Benchtop Freeze Dryer (Kansas City, MO, USA).

All of the final compounds were purified to >95% purity, as determined by high-performance liquid chromatography (HPLC). HPLC analysis was performed using an Agilent 1260 Infinity II HPLC system with the use of a Agilent prep-C18 scalar reversed-column (4.6 mm×100 mm, 5 μm). The binary solvent system was 0.1% formic acid in water (A) and acetonitrile (B), and eluted in a gradient manner from 5% to 100% (A/B) in 15 minutes. The absorbance was detected at 254 nm, and the flow rate was 1.5 mL/min.

High-resolution mass spectrometry (HRMS) was conducted by the Chemical and Biophysical Instrumentation Center in the Chemistry Department at Yale University using a Waters Xevo Q-TOF high-resolution mass spectrometer using electrospray ionization (ESI). For routine data analysis, ultra high-performance liquid chromatography-mass spectrometry (UPLC/MS) was performed with a Waters Acquity UPLC/MS instrument equipped with a reversed-phase BEH Cis column (1.7 mm particle size, 2.1×50 mm), a dual atmospheric pressure chemical ionization (API)/electrospray ionization (ESI) mass spectrometry detector, and a photodiode array detector. An Advion Express mass spectrometer (Ithaca, NY, USA) was also used for routine mass spectral (MS or TLC/MS) analysis.

Routine ¹H NMR spectra were recorded on Agilent 400, 500 or 600 MHz spectrometers at ambient temperature unless otherwise stated. NMR solvents, chloroform-d (CDCl₃), dimethyl suloxide-d6 (DMSO-d6) and methanol-d4 (CD₃OD) were purchased from Cambridge Isotope Laboratories and used without further purification. CDCl₃ was stored at ambient temperature over 4 Å molecular sieves, and fresh DMSO-d6 and CD₃OD ampules were used immediately after opening. Spectra were processed using MestReNova 10.0.1 using the automatic phasing and S-4 polynomial baseline correction capabilities. Splitting was determined using the automatic multiplet analysis function with intervention as necessary. Spectral data are reported as follows: chemical shift (multiplicity [singlet (s), broad singlet (bs), doublet (d), triplet (t), quartet (q), pentet (p), multiplet (m), doublet of doublets (dd), doublet of doublet of doublets (ddd), doublet of triplet of doublets (dtd), doublet of doublet of doublet of doublets (dddd), doublet of triplets (dt), triplet of doublets (td), complex (comp), etc.], coupling constant (Hz), integration). Chemical shifts are reported in ppm (δ), and coupling constants are reported in Hz. ¹H Resonances are referenced to solvent residual peaks for CDCl₃ (7.26 ppm), DMSO-d6 (2.50), CD₃OD (3.31 ppm) or TMS (0 ppm). Routine ¹³C NMR spectra were recorded on Agilent 400, 500 or 600 MHz spectrometers with protons fully decoupled. ¹³C Resonances are reported in ppm relative to solvent residual peaks for CDCl₃ (77.2 ppm) DMSO-d6 (39.5), or CD₃OD (49.0 ppm). ¹⁹F-NMR spectra were recorded on Agilent 400 or 500 MHz spectrometers without proton decoupling.

### II. Synthesis of Compounds of Formula (I) or (I-a)

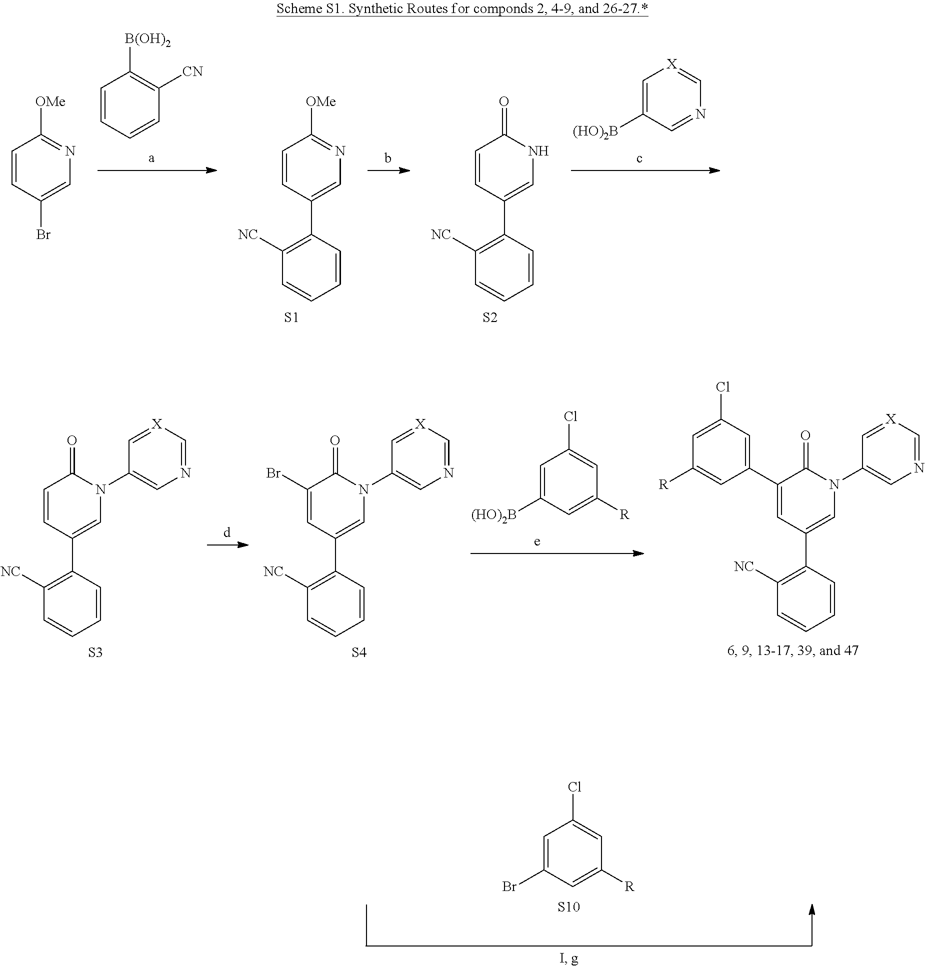

Reagents and Conditions: (a) Cs₂CO₃, Pd(PPh₃)₄, DMF, 120° C., N₂; (b) LiCl, p-TsOH, DMF, 120° C.; (c) Cu-TMEDA, DMF, H₂O, O₂; (d) NBS, DMF; (e) Cs₂CO₃, Pd(PPh₃)₄, DMF, 120° C., N₂; (f) KOAc, PdCl₂(PPh₃)₂, B₂Pin₂, DMF, 80° C., N₂; (g) MeOH, DMF, K₂CO₃, 120° C., N₂. shows a general route for the synthesis of 6, 9, 13-17, 39, and 47.

Briefly, 5-bromo-2-methoxypyridine was coupled with (2-cyanophenyl) boronic acid to afford S1, followed by deprotection to produce intermediates S2, which was subject to a Chan-Lam coupling to yield intermediates S3. Bromination of S3 yielded key intermediates S4, which were coupled with commercially available aryl boronic acids or aryl boronic acid pinacol esters (S10), which were prepared in a one-pot two-step sequence to afford the target compounds 6, 9, 13-17, 39, and 47.

Synthetic routes of other compounds of formula (I) or (I-A) are illustrated in Scheme S2. The starting material 5-bromo-2-fluoropyridine underwent nucleophilic substitution to afford S5, which was subsequently subject to a Suzuki cross-coupling reaction to produce compound S6, followed by deprotection to yield intermediate S7. The key intermediate S9 was synthesized from S8 using a classical Chan-Lam coupling and a subsequent bromination. Other key intermediates S10 were prepared through an electrophilic substitution of commercially available 3-bromo-5-chlorophenol or through a Mitsunobu reaction.

### i. Synthesis and Characterization

### 2-6-Methoxypyridin-3-yl)benzonitrile (S1)

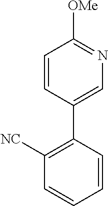 [SMILES: COc1ccc(-c2ccccc2C#N)cn1]

To a 250-mL thick walled round bottom flask 2-cyanophenyl boronic acid (2.204 g, 1.5 mmol, 15.0 equiv), 5-bromo-2-methoxypyridine (1.30 mL, 10.0 mmol, 1.0 equiv), palladium tetrakis (1.156 g, 10 mol %), Cs₂CO₃ (6.516 g, 20.0 mmol, 2.0 equiv) and DMF (100 mL) were added. The solution was sparged with N₂ then the flask was sealed with a screw cap. The reaction was allowed to stir at 120° C. for 15 hours. Once cooled to room temperature, the solvent was removed. The crude material was resuspended in EtOAc and extracted from water with EtOAc (3×) and DCM (3×). The combined organic layers were then dried over Na₂SO₄, filtered, and concentrated in vacuo. The crude material was then subjected to normal phase column chromatography (SNAP Ultra 100 g, gradient=0-10% EtOAc/Hex over 4 CV, then 10-15% EtOAc/Hex over 4 CV, then 15-100% EtOAc/Hex over 2 CV) to afford a white solid (1.497 g, 7.1 mmol, 71% yield). ¹H NMR (600 MHz, CDCl₃) δ 8.33 (d, J=2.4 Hz, 1H), 7.82 (dd, J=8.6, 2.6 Hz, 1H), 7.79-7.75 (m, 1H), 7.66 (td, J=7.7, 1.4 Hz, 1H), 7.49-7.48 (m, 1H), 7.46 (td, J=7.7, 1.2 Hz, 1H), 6.87 (dd, J=8.6, 0.8 Hz, 1H), 4.00 (s, 3H). ¹³C NMR (151 MHz, CDCl₃) δ 164.5, 146.8, 142.2, 138.9, 134.0, 133.2, 129.9, 127.9, 127.3, 118.6, 111.5, 111.0, 53.9. HRMS (ESI) m/z: [M+H]⁺ calcd for C₁₃H₁₁N₂O⁺211.0866, found 211.0880.

### 2-(6-Oxo-1,6-dihydropyridin-3-yl)benzonitrile (S2)

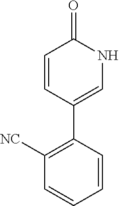 [SMILES: N#Cc1ccccc1-c1ccc(=O)[nH]c1]

A 40-mL thick walled flask equipped with a stir bar was charged with S1 (1.000 g, 4.76 mmol, 1.0 equiv), lithium chloride (1.008 g, 23.80 mmol, 5.0 equiv), p-toluenesulfonic acid (4.095 g, 23.80 mmol, 5.0 equiv), and DMF (16 mL). The flask was sealed with a screw cap and then heated to 120° C. for 2 hours. Once cooled to room temperature, the solution was quenched with water and subsequently filtered to afford a white solid (0.934 g, quant. yield). ¹H NMR (600 MHz, DMSO-d6) δ 11.98 (s, 1H), 7.96-7.87 (m, 1H), 7.74 (td, J=7.8, 1.2 Hz, 1H), 7.71-7.66 (m, 2H), 7.60 (d, J=7.9 Hz, 1H), 7.52 (t, J=7.6 Hz, 1H), 6.51-6.42 (m, 1H). ¹³C NMR (151 MHz, DMSO-d6) δ 161.6, 141.3, 140.4, 135.7, 133.8, 133.6, 129.4, 127.8, 119.7, 118.5, 115.4, 109.7. HRMS (ESI) m/z: [M+H]⁺ calcd for C₁₂H₉N₂O⁺ 197.0709, found 197.0714.

### 2-(2-Oxo-2H-[1,3′-bipyridin]-5-yl)benzonitrile (S3a)

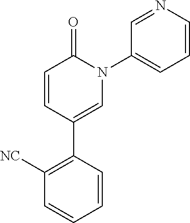 [SMILES: N#Cc1ccccc1-c1ccc(=O)n(-c2cccnc2)c1]

To a 2-dram vial equipped with a stir bar S2 (0.196 g, 1.00 mmol, 1.0 equiv), 3-pyridinylboronic acid (0.246 g, 2.00 mmol, 2.0 equiv), Cu-TMEDA (0.0464 g, 10 mol %), DMF (4.0 mL), and water (0.2 mL) were added. The reaction vessel was closed using a screw-cap with bonded septum, then affixed with an oxygen balloon and stirred under an oxygen atmosphere for 4 days. The mixture was then diluted with DCM and water. The aqueous layer was rinsed with DCM (5×). The combined organic layer was then dried with Na₂SO₄, filtered, and concentrated in vacuo. The crude material was purified using normal phase column chromatography (SNAP Ultra 50 g, gradient=0-6% DCM/MeOH over 5 CV, then 6-7% DCM/MeOH over 3 CV, then 7-10% DCM/MeOH over 2 CV) to afford 0.184 g (67% yield) of a white solid. ¹H NMR (500 MHz, DMSO-d6) δ 8.74 (d, J=2.5 Hz, 1H), 8.66 (dd, J=4.8, 1.4 Hz, 1H), 8.13 (d, J=2.6 Hz, 1H), 8.01 (ddd, J=8.2, 2.6, 1.5 Hz, 1H), 7.94 (dd, J=7.8, 1.2 Hz, 1H), 7.86 (dd, J=9.5, 2.7 Hz, 1H), 7.77 (td, J=7.7, 1.4 Hz, 1H), 7.72 (dd, J=8.0, 1.2 Hz, 1H), 7.60 (dd, J=8.2, 4.8 Hz, 1H), 7.56 (td, J=7.6, 1.3 Hz, 1H), 6.69 (d, J=9.5 Hz, 1H). ¹³C NMR (126 MHz, DMSO-d6) δ 160.3, 149.2, 147.4, 141.5, 139.8, 138.7, 136.9, 134.7, 133.8, 133.6, 129.6, 128.2, 123.8, 120.2, 118.6, 116.3, 110.0. HRMS (ESI) m/z: [M+H]⁺ calcd for C₁₇H₁₂N₃O⁺ 274.0975, found 274.0995.

### 2-(6-Oxo-1-(pyrimidin-5-yl)-1,6-dihydropyridin-3-yl)benzonitrile (S3b)

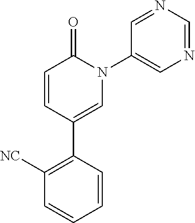 [SMILES: N#Cc1ccccc1-c1ccc(=O)n(-c2cncnc2)c1]

To a 2-dram vial equipped with a stir bar S2 (39.6 mg, 0.20 mmol, 1.0 equiv), pyrimidine 5-boronic acid (45.9 g, 0.40 mmol, 2.0 equiv), Cu-TMEDA (9.3 mg, 10 mol %), DCM (1.0 mL) and DMF (1.0 mL) were added. The reaction vessel was closed using a screw-cap with bonded septum, then affixed with an oxygen balloon and stirred under an oxygen atmosphere for 6 days. The solution was diluted with water and extracted with EtOAc/Hex (1:1; 3×), then DCM (3×). The combined organic layer was dried over Na₂SO₄, filtered, and concentrated in vacuo. The crude material was used in the following step without further purification. 2-(3-Bromo-2-oxo-2H-[1,3′-bipyridin]-5-yl)benzonitrile (S4a)

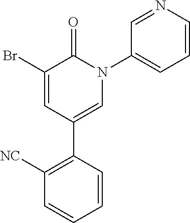 [SMILES: N#Cc1ccccc1-c1cc(Br)c(=O)n(-c2cccnc2)c1]

A 2-dram vial equipped with a stir bar was charged with crude S3a (0.273 g, 1.00 mmol, 1.0 equiv) and DMF (2 mL). While stirring at room temperature NBS (0.214 g, 1.20 mmol, 1.2 equiv) was added. After 15 hours, the reaction was quenched with ice water, and subsequently filtered to afford 0.313 g of a white solid (89% yield), which was used without further purification. Analytically pure material was obtained using normal phase column chromatography (SNAP Ultra 50 g, gradient=0-100% EtOAc/Hex over 4 CV, then 100% EtOAc/Hex over 6 CV). 1H NMR (500 MHz, CDCl₃) δ 8.77 (d, J=2.5 Hz, 1H), 8.70 (dd, J=4.8, 1.5 Hz, 1H), 8.05 (d, J=2.5 Hz, 1H), 7.90 (ddd, J=8.2, 2.6, 1.5 Hz, 1H), 7.77 (dt, J=7.4, 1.1 Hz, 1H), 7.72-7.65 (m, 2H), 7.50 (d, J=7.8 Hz, 2H), 7.49-7.46 (m, 1H). ¹³C NMR (126 MHz, CDCl₃) δ 157.7, 150.3, 147.3, 142.7, 138.9, 137.26, 136.6, 134.3, 134.1, 133.6, 129.4, 128.8, 124.0, 118.3, 118.2, 117.5, 111.1. HRMS (ESI) m/z: [M+H]⁺ calcd for C₁₇H₁₁BrN₃O⁺352.0080, found 352.0100.

### 2-(5-Bromo-6-oxo-1-(pyrimidin-5-yl)-1,6-dihydropyridin-3-yl)benzonitrile (S4b)

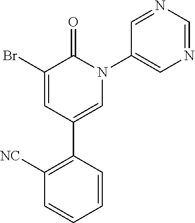 [SMILES: N#Cc1ccccc1-c1cc(Br)c(=O)n(-c2cncnc2)c1]

A 2-dram vial equipped with a stir bar was charged with crude S3b (54.9 mg, 0.20 mmol, 1.0 equiv) and DMF (1 mL). While stirring at room temperature NBS (71.2 mg, 0.40 mmol, 2.0 equiv) was added. After 15 hours, the reaction was quenched with ice water, and subsequently filtered to afford 30.9 mg of a beige solid (44% yield over 2 steps). 1H NMR (400 MHz, DMSO-d6) δ 9.29 (s, 1H), 9.10 (s, 2H), 8.45 (d, J=2.5 Hz, 1H), 8.31 (d, J=2.5 Hz, 1H), 7.99-7.92 (m, 1H), 7.79 (td, J=7.7, 1.3 Hz, 1H), 7.76-7.70 (m, 1H), 7.59 (td, J=7.6, 1.3 Hz, 1H). ¹³C NMR (101 MHz, DMSO-d6) δ 157.8, 156.9, 155.0, 143.5, 138.5, 138.3, 135.42, 133.8, 133.6, 129.9, 128.6, 118.3, 117.0, 115.1, 110.3. HRMS (ESI) m z: [M+H]⁺ calcd for C₁₆H₁₀BrN₄O 353.0032, found 353.0051.

### 2-(Benzyloxy)-5-bromopyridine (S5)

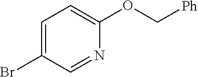 [SMILES: Brc1ccc(OCc2ccccc2)nc1]

Benzyl alcohol (6.75 g, 62.48 mmol, 1.1 eq) was dissolved in anhydrous THF (120 mL) and cooled to 0° C. Then, 60% sodium hydride (2.73 g, 68.2 mmol, 1.2 eq) was added and stirred for 30 min at 0° C. Next, 5-bromo-2-fluoropyridine (10 g, 56.8 mmol, 1.0 eq) was added and the mixture was heated at 70° C. overnight. The reaction was quenched by the dropwise addition of water, then brine and more ethyl acetate were added for extraction. The organic layer was dried over Na₂SO₄ and the solvent removed under reduced pressure, the residue was recrystallized in hexane to afford a white solid (12 g, 80% yield). ¹H NMR (400 MHz, CDCl₃) δ 8.21 (d, J=2.3 Hz, 1H), 7.65 (dd, J=8.8, 2.5 Hz, 1H), 7.44 (d, J=7.2 Hz, 2H), 7.39-7.30 (m, 3H), 6.72 (d, J=8.8 Hz, 1H), 5.34 (s, 2H). MS (ESI) m/z: [M+H]⁺ calcd for C₁₂H₁₁BrNO⁺264.00, found 264.0, 266.0.

### 5-(6-(Benzyloxy)pyridin-3-yl)-2,4-dimethoxypyrimidine (S6)

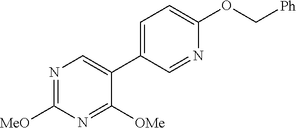 [SMILES: COc1ncc(-c2ccc(OCc3ccccc3)nc2)c(OC)n1]

To a 250 mL round bottom flask, (S5, 7 g, 26.5 mmol, 1.0 eq), (2,4-dimethoxypyrimidin-5-yl)boronic acid (6.34 g, 34.45 mmol, 1.3 eq), Cs₂CO₃ (17.3 g, 53 mmol, 2.0 eq) and bis(triphenylphosphine)-palladium(II) chloride (0.93 g, 1.3 mmol, 0.05 eq) were added and suspended in DMF (150 mL). The mixture underwent three cycles of vacuum/filling with N₂, then stirred at 80° C. for 5 h. The mixture was concentrated in vacuo and the residue was resuspended in water (80 mL) and extracted with dichloromethane (2×80 mL). The combined organic layer was concentrated in vacuo and the crude product was purified using silica gel chromatography with an ethyl acetate/hexanes gradient (0-10%) to afford a white solid (6.2 g, 72% yield). ¹H NMR (400 MHz, CDCl₃) δ 8.28 (s, 1H), 8.24 (s, 1H), 7.75 (d, J=8.6 Hz, 1H), 7.48 (d, J=7.7 Hz, 2H), 7.39 (t, J=7.3 Hz, 2H), 7.34 (d, J=6.6 Hz, 1H), 6.88 (d, J=8.6 Hz, 1H), 5.42 (s, 2H), 4.04 (s, 3H), 4.03 (s, 3H). MS (ESI) m/z: [M+H]⁺ calcd for C₁₈H₁₈N₃O₃+324.13, found 324.2.

### 5-(2,4-Dimethoxypyrimidin-5-yl)pyridin-2(1H)-one (S7)

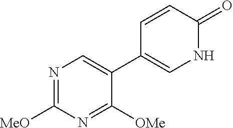 [SMILES: COc1ncc(-c2ccc(=O)[nH]c2)c(OC)n1]

To a 250 mL round bottom flask, S6 (6.2 g) and palladium on activated carbon (10%, 500 mg) were suspended in methanol (150 mL) and water (10 mL). The mixture underwent 3 cycles of vacuum/filling with H₂ and then stirred at 40° C. for 4 h. After the reaction was complete, dichloromethane was added to dissolve the solid product, then the mixture was filtered. The filtrate was concentrated in vacuo to give the desired product as a gray solid (4.4 g, 99% yield). ¹H NMR (400 MHz, CDCl₃) δ 13.21 (br.s, 1H), 8.20 (s, 1H), 7.67 (dd, J=9.4, 2.6 Hz, 1H), 7.63 (d, J=2.2 Hz, 1H), 6.67 (d, J=9.4 Hz, 1H), 4.04 (s, 3H), 4.03 (s, 3H). MS (ESI) m/z: [M+H]⁺ calcd for C₁₁H₁₂N₃O₃⁺234.09, found 234.1.

### 5-(2,4-Dimethoxypyrimidin-5-yl)-2H-[1,3′-bipyridin]-2-one (S8)

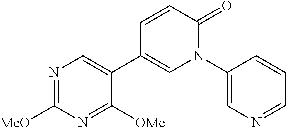 [SMILES: COc1ncc(-c2ccc(=O)n(-c3cccnc3)c2)c(OC)n1]

To a 250 mL round bottom flask, S7 (4.4 g, 18.9 mmol, 1.0 eq), 3-pyridylboronic acid (4.65 g, 37.8 mmol, 2.0 eq), cupric acetate (3.43 g, 18.9 mmol, 1.0 eq) and N,N,N′,N′-Tetramethylethylenediamine (4.4 g, 37.8 mmol, 2.0 eq) were suspended in anhydrous DMF (120 mL). Dry air was bubbled through the mixture and the solution was then stirred at room temperature for 4 days. After the reaction was complete, the mixture was concentrated in vacuo and the residue was diluted with aq. ammonium (5%, 40 mL) and the organic layer was extracted with dichloromethane (3×40 mL). The combined organic layer was concentrated in vacuo and the crude product was purified using silica gel chromatography with a methanol/dichloromethane gradient (0-5%) to yield the desired product as a white solid (4.3 g, 74% yield)¹H NMR (400 MHz, CDCl₃) δ 8.70 (s, 2H), 8.23 (s, 1H), 7.89 (d, J=8.2 Hz, 1H), 7.62 (dd, J=9.6, 2.2 Hz, 1H), 7.54 (d, J=2.5 Hz, 1H), 7.48 (dd, J=8.1, 4.8 Hz, 1H), 6.75 (d, J=9.6 Hz, 1H), 4.05 (s, 3H), 4.03 (s, 3H). MS (ESI) m/z: [M+H]⁺ calcd for C₁₆H₁₅N₄O₃⁺311.11, found 311.2.

### 3-Bromo-5-(2,4-dimethoxypyrimidin-5-yl)-2H-[1,3′-bipyridin]-2-one (S9)

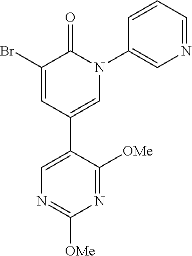 [SMILES: COc1ncc(-c2cc(Br)c(=O)n(-c3cccnc3)c2)c(OC)n1]

To a 250 mL round bottom flask, S8 (4.3 g 13.9 mmol, 1.0 eq) was dissolved in anhydrous DMF (80 mL). The mixture underwent 3 cycles of vacuum/filling with N₂, then N-bromosuccinimide (9.9 g, 55.6 mmol, 4 eq) was added and the solution was stirred at room temperature for 4 h. Then the reaction was quenched with aqueous sodium thiosulfate solution (1 M, 50 mL) at 0° C. and stirred at this temperature for 2 h. The aqueous layer was extracted with dichloromethane (3×60 mL) and then the combined organic layer was concentrated in vacuo at low temperature (20° C.). The residue was further dried using a vacuum pump, then the crude product was purified using silica gel chromatography with a methanol/dichloromethane gradient (0-5%) to afford an orange solid (3.7 g, 69% yield). ¹H NMR (400 MHz, CDCl₃) δ 8.72 (s, 2H), 8.22 (s, 1H), 8.04 (d, J=2.3 Hz, 1H), 7.95 (d, J=8.2 Hz, 1H), 7.53 (dd, J=8.0, 3.7 Hz, 2H), 4.07 (s, 3H), 4.04 (s, 3H). MS (ESI) m/z: [M+H]⁺ calcd for C₁₆H₁₄BrN₄O₃⁺389.02, found 389.1, 391.1.

### General Procedure a for the Synthesis of S10

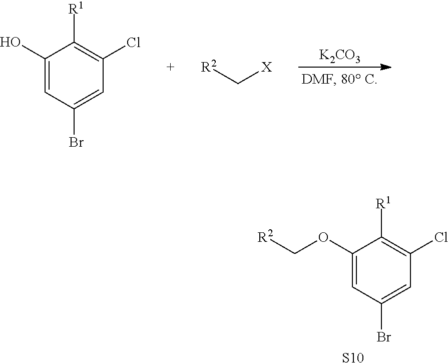

To a solution of 3-bromo-5-chlorophenol (1.04 g, 5.0 mmol, 1.0 eq) and K₂CO₃ (1.38 g, 10 mmol, 2.0 eq) in DMF (30 mL) the requisite alkyl halide (5.1 mmol, 1.02 eq) was added and the solution was stirred at 80° C. for 1 h. Once complete, the mixture was concentrated in vacuo and the residue was resuspended in water (40 mL) and extracted with dichloromethane (2×40 mL). The combined organic layer was concentrated in vacuo and the crude product was purified using silica gel chromatography (100% hexane) to yield the desired compound.

### 1-Bromo-3-chloro-5-(cyclopropylmethoxy)benzene (S10a)

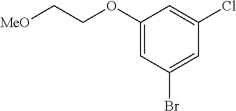 [SMILES: COCCOc1cc(Cl)cc(Br)c1]

Compound S10a was prepared according to General Procedure A described using 1-bromo-2-methoxyethane. ¹H NMR (400 MHz, CDCl₃) δ 7.07 (t, J=1.7 Hz, 1H), 6.98-6.92 (m, 1H), 6.84 (t, J=2.0 Hz, 1H), 4.10-4.01 (m, 2H), 3.70 (dd, J=5.3, 3.9 Hz, 2H), 3.41 (s, 3H).

### 1-Bromo-3-chloro-5-phenethoxybenzene (S10b)

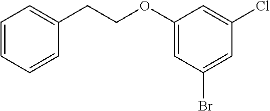 [SMILES: Clc1cc(Br)cc(OCCc2ccccc2)c1]

Compound S10b was prepared according to General Procedure A using 3-bromo-5-chlorophenol and (2-bromoethyl)benzene. ¹H NMR (400 MHz, CDCl₃) δ 7.35-7.29 (m, 2H), 7.27-7.22 (m, 3H), 7.08 (s, 1H), 6.94 (s, 1H), 6.82 (t, J=1.8 Hz, 1H), 4.13 (t, J=7.0 Hz, 2H), 3.08 (t, J=6.9 Hz, 2H).

### 1-Bromo-3-butoxy-5-chlorobenzene (S10c)

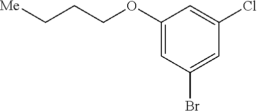 [SMILES: CCCCOc1cc(Cl)cc(Br)c1]

Compound S10c was prepared according to General Procedure A using 1-bromobutane. ¹H NMR (400 MHz, CDCl₃) δ 7.08 (t, J=1.7 Hz, 1H), 6.96-6.92 (m, 1H), 6.82 (t, J=2.0 Hz, 1H), 3.92 (t, J=6.5 Hz, 2H), 1.81-1.69 (m, 2H), 1.52-1.42 (m, 2H), 0.97 (t, J=7.4 Hz, 3H).

### 1-Bromo-3-chloro-5-(isopentyloxy)benzene (S10d)

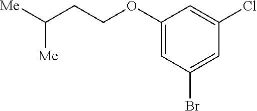 [SMILES: CC(C)CCOc1cc(Cl)cc(Br)c1]

Compound S10d was prepared according to General Procedure A using 1-bromo-3-methylbutane. ¹H NMR (400 MHz, CDCl₃) δ 7.08 (t, J=1.6 Hz, 1H), 6.94 (t, J=1.9 Hz, 1H), 6.82 (t, J=2.0 Hz, 1H), 3.94 (t, J=6.6 Hz, 2H), 1.86-1.76 (m, 1H), 1.66 (q, J=6.7 Hz, 2H), 0.96 (s, 3H), 0.95 (s, 3H).

### 1-Bromo-3-chloro-5-((2-methylbenzyl)oxy)benzene (S10e)

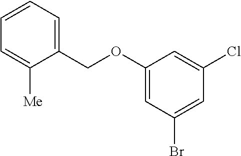 [SMILES: Cc1ccccc1COc1cc(Cl)cc(Br)c1]

Compound S10e was prepared according to General Procedure A using 1-(bromomethyl)-2-methylbenzene. ¹H NMR (400 MHz, CDCl₃) δ 7.35 (d, J=6.9 Hz, 1H), 7.30-7.19 (m, 3H), 7.12 (t, J=1.6 Hz, 1H), 7.04 (t, J=1.9 Hz, 1H), 6.92 (t, J=2.0 Hz, 1H), 4.98 (s, 2H), 2.35 (s, 3H).

### 1-Bromo-3-chloro-5-((3-methylbenzyl)oxy)benzene (S10f)

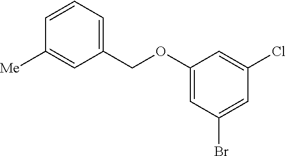 [SMILES: Cc1cccc(COc2cc(Cl)cc(Br)c2)c1]

Compound S10f was prepared according to General Procedure A using 1-(bromomethyl)-3-methylbenzene. ¹H NMR (400 MHz, CDCl₃) δ 7.30-7.24 (m, 1H), 7.21-7.15 (m, 3H), 7.11 (d, J=1.5 Hz, 1H), 7.05-7.00 (m, 1H), 6.93-6.89 (m, 1H), 4.97 (s, 2H), 2.37 (s, 3H).

### 1-Bromo-3-chloro-5-((2-fluorobenzyl)oxy)benzene (S10g)

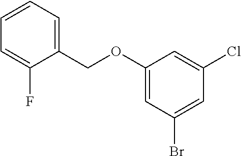 [SMILES: Fc1ccccc1COc1cc(Cl)cc(Br)c1]

Compound S10g was prepared according to General Procedure A using 1-(bromomethyl)-2-fluorobenzene. ¹H NMR (400 MHz, CDCl₃) δ 7.45 (t, J=7.2 Hz, 1H), 7.37-7.32 (m, 1H), 7.21-7.07 (m, 3H), 7.05 (s, 1H), 6.93 (t, J=1.7 Hz, 1H), 5.09 (s, 2H).

### 1-Bromo-3-chloro-5-((3-fluorobenzyl)oxy)benzene (S10h)

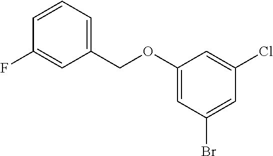 [SMILES: Fc1cccc(COc2cc(Cl)cc(Br)c2)c1]

Compound S10h was prepared according to General Procedure A using 1-(bromomethyl)-3-fluorobenzene. ¹H NMR (400 MHz, CDCl₃) δ 7.36 (td, J=7.9, 5.9 Hz, 1H), 7.19-7.09 (m, 3H), 7.07-7.00 (m, 2H), 6.91 (t, J=2.0 Hz, 1H), 5.02 (s, 2H).

### 2-((3-Bromo-5-chlorophenoxy)methyl)-1,3-difluorobenzene (S10i)

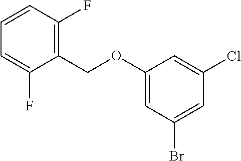 [SMILES: Fc1cccc(F)c1COc1cc(Cl)cc(Br)c1]

Compound S10i was prepared according to General Procedure A using 2-(bromomethyl)-1,3-difluorobenzene. ¹H NMR (400 MHz, CDCl₃) δ 7.41-7.31 (m, 1H), 7.16-7.12 (m, 1H), 7.06 (d, J=1.7 Hz, 1H), 7.00-6.91 (m, 3H), 5.09 (s, 2H).

### 1-Bromo-3-chloro-5-((2-chlorobenzyl)oxy)benzene (S10j)

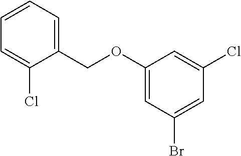 [SMILES: Clc1cc(Br)cc(OCc2ccccc2Cl)c1]

Compound S10j was prepared according to General Procedure A using 1-chloro-2-(chloromethyl)benzene. ¹H NMR (400 MHz, CDCl₃) δ 7.51-7.49 (m, 1H), 7.44-7.40 (m, 1H), 7.33-7.28 (m, 2H), 7.14 (t, J=1.7 Hz, 1H), 7.07-7.04 (m, 1H), 6.94 (t, J=2.0 Hz, 1H), 5.13 (s, 2H).

### 1-((3-Bromo-5-chlorophenoxy)methyl)-2-chloro-4-fluorobenzene (S10k)

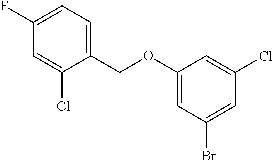 [SMILES: Fc1ccc(COc2cc(Cl)cc(Br)c2)c(Cl)c1]

Compound S10k was prepared according to General Procedure A using 1-(bromomethyl)-2-chloro-4-fluorobenzene. ¹H NMR (400 MHz, CDCl₃) δ 7.48 (dd, J=8.5, 6.1 Hz, 1H), 7.18 (dd, J=8.4, 2.5 Hz, 1H), 7.15 (s, 1H), 7.07-6.99 (m, 2H), 6.92 (t, J=1.8 Hz, 1H), 5.07 (s, 2H).

### 5-Bromo-1-chloro-3-((2-chlorobenzyl)oxy)-2-fluorobenzene (S10l)

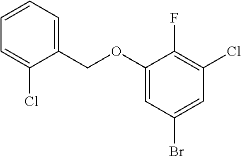 [SMILES: Fc1c(Cl)cc(Br)cc1OCc1ccccc1Cl]

Compound S10m was prepared according to General Procedure A using 5-bromo-3-chloro-2-fluorophenol and 1-chloro-2-(chloromethyl)benzene. ¹H NMR (400 MHz, CDCl₃) δ 7.55 (dd, J=6.8, 2.5 Hz, 1H), 7.44-7.39 (m, 1H), 7.33-7.29 (m, 2H), 7.17 (dd, J=5.6, 2.2 Hz, 1H), 7.07 (dd, J=6.7, 2.2 Hz, 1H), 5.20 (s, 2H).

### 1-Bromo-3-chloro-5-(cyclopropylmethoxy)benzene (S10m)

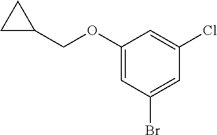 [SMILES: Clc1cc(Br)cc(OCC2CC2)c1]

Compound S10l was prepared according to General Procedure A using (chloromethyl)cyclopropane. ¹H NMR (400 MHz, CDCl₃) δ 7.06 (s, 1H), 6.92 (s, 1H), 6.81 (s, 1H), 3.75 (d, J=6.9 Hz, 2H), 1.28-1.18 (m, 1H), 0.64 (q, J=5.2, 4.5 Hz, 2H), 0.32 (d, J=4.8 Hz, 2H).

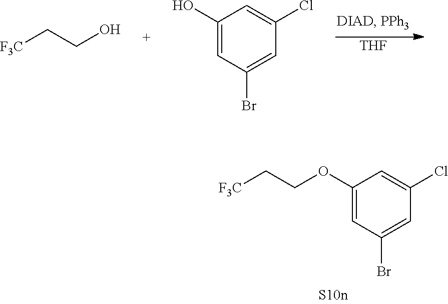 [SMILES: OCCC(F)(F)F ; Oc1cc(Cl)cc(Br)c1 ; FC(F)(F)CCOc1cc(Cl)cc(Br)c1 ; "THF"]

### 1-Bromo-3-chloro-5-(3,3,3-trifluoropropoxy)benzene (S10n)

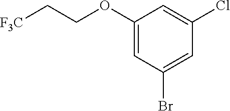 [SMILES: FC(F)(F)CCOc1cc(Cl)cc(Br)c1]

3-Bromo-5-chlorophenol (1.04 g, 5.0 mmol, 1.0 eq), 3,3,3-trifluoropropan-1-ol (0.63 g, 5.5 mmol, 1.1 eq) and PPh₃ (1.97 g, 7.5 mmol, 1.5 eq) were dissolved in dry THF (60 mL). The mixture was cooled to 0° C. and underwent 3 cycles of vacuum/filling with N₂. Diisopropyl azodicarboxylate (1.52 g, 7.5 mmol, 1.5 eq) was added dropwise and the mixture was warmed to room temperature slowly while stirring for 30 min. Then, the mixture was heated to 80° C. and stirred overnight. After the reaction was complete, the mixture was concentrated in vacuo and the residue was redissolved in dichloromethane, and washed with saturated aqueous NH₄Cl, water, then brine. The combined organic layer was dried over MgSO₄, and the crude product was purified using silica gel chromatography (100% hexanes) to afford S10n (0.68 g, 45% yield). ¹H NMR (400 MHz, CDCl₃) δ 7.14 (t, J=1.6 Hz, 1H), 6.97-6.93 (m, 1H), 6.84 (t, J=2.0 Hz, 1H), 4.16 (t, J=6.5 Hz, 2H), 2.62 (qt, J=10.4, 6.5 Hz, 2H).

### General Procedure B for Suzuki-Miyaura Cross-Coupling

Aryl bromide (1.0 equiv), boronic acid (2.0 equiv), palladium tetrakis (10 mol %), cesium carbonate (2.5 equiv) and DMF (0.07 M) were added to a 2-dram vial equipped with a stir bar. The solution was sparged with N₂, then the vial was sealed with a Teflon cap and heated to 120° C. for 15-20 hours. Once cooled to room temperature, the DMF was removed, and the crude material was purified via column chromatography to afford the desired product.

### General Procedure C for Suzuki-Miyaura Cross-Coupling

Aryl bromide (1.0 equiv), boronic acid (1.5 equiv), PdCl₂(PPh₃)₂ (10 mol %), potassium carbonate (2.0 equiv) were suspended in DMF (10 mL). The mixture underwent three cycles of vacuum/filling with N₂, then stirred at 120° C. for 1 h. After the reaction was complete, the mixture was concentrated in vacuo, then the residue was extracted with DCM (2×40 mL) and water (40 mL). The combined organic layer was concentrated, and the crude product was purified using silica gel chromatography with a dichloromethane/ethyl acetate/methanol (92%/5%/3%) gradient to afford the desired product as a white solid.

### General Procedure D for Suzuki-Miyaura Cross-Coupling

Aryl bromide (1.1 equiv), bis(pinacolato)diboron (1.15 equiv), KOAc (2.2 equiv), and (PPh₃)₂PdCl₂ (20 mol %) and DMF (0.04 M) were added to a 2-dram vial equipped with a stir bar. The solution was sparged with N₂, then the vial was sealed with a Teflon cap and heated to 80° C. for 1-3 hours. Once cooled to room temperature, methanol (0.4 mL) was added to scavenge excess of pinacolborane. Pyridone bromide (1.0 equiv) and K₂CO₃ (2.0 equiv) were added to the solution, which was then sparged with N₂, sealed with a Teflon cap, and heated to 120° C. for 1-15 hours. Once cooled to room temperature, the DMF was removed, water was added, and the solution was extracted with DCM (3×). The combined organic layer was dried with Na₂SO₄, filtered, and then concentrated in vacuo. Uracil compounds were purified using purified using silica gel chromatography with a dichloromethane/ethyl acetate/methanol (92%/5%/3%) gradient to afford the target compound as a white solid. The purification of the cyanophenyl compounds is specified for each compound below.

### General Procedure E for Final Uracil Demethylation

Substituted 2,4-dimethyoxypyrimidine (1.0 equiv) was dissolved in DMF (10 mL) along with LiCl (10 equiv) and p-toluenesulfonic acid (10 equiv). The solution was then stirred at 80° C. for 30 min. After completion, the mixture was concentrated in vacuo. The residue was suspended in saturated aqueous NaHCO₃ (15 mL), then the mixture was filtered. The solid was washed with saturated aqueous NaHCO₃, water, and then hexanes. The material was then dried with a lyophilizer to yield the desired compound as a pale yellow solid.

### 3-(3-Chloro-5-propoxyphenyl)-5-(2,4-dimethoxypyrimidin-5-yl)-2H-[1,3′-bipyridin]-2-one (S11a)

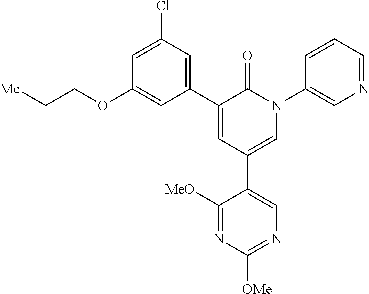 [SMILES: CCCOc1cc(Cl)cc(-c2cc(-c3cnc(OC)nc3OC)cn(-c3cccnc3)c2=O)c1]

General Procedure C was employed using S9 and (3-chloro-5-propoxyphenyl)boronic acid to afford the title compound as a white solid (130.6 mg, 68% yield). 1H NMR (400 MHz, CDCl₃) δ 8.75 (d, J=15.5 Hz, 2H), 8.28 (s, 1H), 7.99 (d, J=8.2 Hz, 1H), 7.76 (d, J=2.3 Hz, 1H), 7.62-7.50 (m, 2H), 7.27 (s, 1H), 7.26-7.19 (m, 1H), 6.92 (t, J=1.9 Hz, 1H), 4.07 (s, 3H), 4.05 (s, 3H), 3.94 (t, J=6.5 Hz, 2H), 1.81-1.75 (m, 2H), 1.02 (t, J=7.4 Hz, 3H). MS (ESI) m/z: [M+H]⁺ calcd for C₂₅H₂₄ClN₄O₄⁺479.15, found 479.2.

### 3-(3-Butoxy-5-chlorophenyl)-5-(2,4-dimethoxypyrimidin-5-yl)-2H-[1,3′-bipyridin]-2-one (S11b)

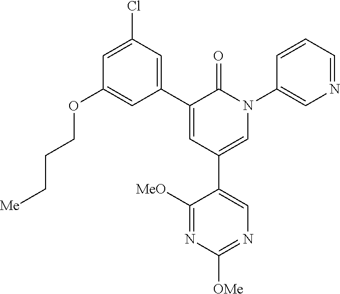 [SMILES: CCCCOc1cc(Cl)cc(-c2cc(-c3cnc(OC)nc3OC)cn(-c3cccnc3)c2=O)c1]

General Procedure D was employed using S9 and 1-bromo-3-butoxy-5-chlorobenzene (S10c) to afford the title compound (66 mg, 67% yield). 1H NMR (400 MHz, CDCl₃) δ 8.79 (s, 1H), 8.73 (d, J=4.5 Hz, 1H), 8.29 (s, 1H), 8.06 (d, J=7.9 Hz, 1H), 7.76 (d, J=2.3 Hz, 1H), 7.60 (d, J=7.5 Hz, 2H), 7.27 (s, 1H), 7.22 (s, 1H), 6.92 (s, 1H), 4.07 (s, 3H), 4.05 (s, 3H), 3.98 (t, J=6.4 Hz, 2H), 1.75 (dt, J=14.4, 6.5 Hz, 2H), 1.55-1.41 (m, 2H), 0.96 (t, J=7.4 Hz, 3H). MS (ESI) m/z: [M+H]⁺ calcd for C₂₆H₂₆ClN₄O₄⁺493.16, found 493.3.

### 3-(3-Chloro-5-(isopentyloxy)phenyl)-5-(2,4-dimethoxypyrimidin-5-yl)-2H-[1,3′-bipyridin]-2-one (S11c)

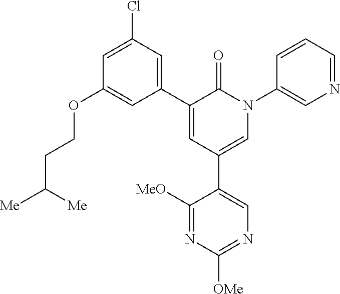 [SMILES: COc1ncc(-c2cc(-c3cc(Cl)cc(OCCC(C)C)c3)c(=O)n(-c3cccnc3)c2)c(OC)n1]

General Procedure D was employed using S9 and 1-bromo-3-chloro-5-(isopentyloxy)benzene (S10d) to afford the title compound as a white solid (73.9 mg, 73% yield). 1H NMR (400 MHz, CDCl₃) δ 8.73 (d, J=11.8 Hz, 2H), 8.27 (s, 1H), 7.95 (d, J=8.2 Hz, 1H), 7.76 (d, J=2.5 Hz, 1H), 7.71-7.63 (m, 1H), 7.57 (d, J=2.5 Hz, 1H), 7.28 (t, J=1.6 Hz, 1H), 7.25-7.21 (m, 1H), 6.91 (t, J=2.1 Hz, 1H), 4.06 (s, 3H), 4.04 (s, 3H), 4.00 (t, J=6.6 Hz, 2H), 1.82 (dt, J=13.4, 6.7 Hz, 1H), 1.66 (q, J=6.6 Hz, 2H), 0.96 (s, 3H), 0.94 (s, 3H). MS (ESI) m/z: [M+H]⁺ calcd for C₂₇H₂₈ClN₄O₄+507.18, found 507.3.

### 3-(3-Chloro-5-phenethoxyphenyl)-5-(2,4-dimethoxypyrimidin-5-yl)-2H-[1,3′-bipyridin]-2-one (S11d)

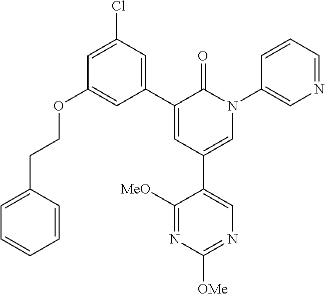 [SMILES: COc1ncc(-c2cc(-c3cc(Cl)cc(OCCc4ccccc4)c3)c(=O)n(-c3cccnc3)c2)c(OC)n1]

General Procedure D was employed using S9 and 1-bromo-3-chloro-5-phenethoxybenzene (S10b) to afford the title compound as a white solid (65.9 mg, 61% yield). 1H NMR (400 MHz, CDCl₃) δ 8.72 (d, J=13.5 Hz, 2H), 8.25 (s, 1H), 7.97 (d, J=8.0 Hz, 1H), 7.72 (d, J=1.9 Hz, 1H), 7.70-7.61 (m, 1H), 7.59-7.49 (m, 2H), 7.45 (td, J=7.4, 2.9 Hz, 1H), 7.33-7.17 (m, 5H), 6.89 (t, J=2.0 Hz, 1H), 4.17 (t, J=7.0 Hz, 2H), 4.04 (s, 3H), 4.03 (s, 3H), 3.07 (t, J=6.9 Hz, 2H). MS (ESI) m/z: [M+H]⁺ calcd for C₃₀H₂₆ClN₄O₄⁺541.16, found 541.3.

### 3-(3-Chloro-5-((2-methylbenzyl)oxy)phenyl)-5-(2,4-dimethoxypyrimidin-5-yl)-2H-[1,3′-bipyridin]-2-one (S11e)

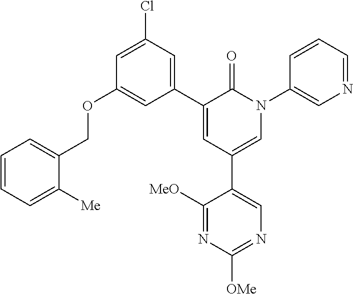 [SMILES: COc1ncc(-c2cc(-c3cc(Cl)cc(OCc4ccccc4C)c3)c(=O)n(-c3cccnc3)c2)c(OC)n1]

General Procedure D was employed using S9 and 1-bromo-3-chloro-5-((2-methylbenzyl)oxy) benzene (S10e) to afford the title compound as a white solid (83.2 mg, 77% yield). ¹H NMR (400 MHz, CDCl₃) δ 8.77 (s, 1H), 8.73 (d, J=4.6 Hz, 1H), 8.28 (s, 1H), 8.00 (d, J=8.1 Hz, 1H), 7.76 (s, 1H), 7.56 (dd, J=11.7, 7.3 Hz, 3H), 7.39 (d, J=7.2 Hz, 1H), 7.33 (s, 2H), 7.22 (d, J=6.9 Hz, 2H), 7.01 (s, 1H), 5.05 (s, 2H), 4.07 (s, 3H), 4.05 (s, 3H), 2.37 (s, 3H). MS (ESI) m/z: [M+H]⁺ calcd for C₃₀H₂₆ClN₄O₄⁺541.16, found 541.3.

### 3-(3-Chloro-5-((3-methylbenzyl)oxy)phenyl)-5-(2,4-dimethoxypyrimidin-5-yl)-2H-[1,3′-bipyridin]-2-one (S11f)

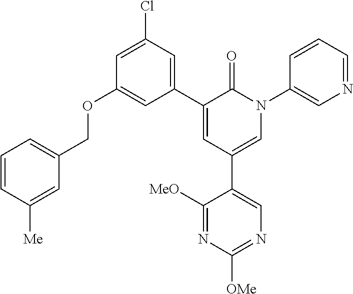 [SMILES: COc1ncc(-c2cc(-c3cc(Cl)cc(OCc4cccc(C)c4)c3)c(=O)n(-c3cccnc3)c2)c(OC)n1]

General Procedure D was employed using S9 and 1-bromo-3-chloro-5-((3-methylbenzyl)oxy) benzene (S10f) to afford the title compound as a white solid (76.7 mg, 71% yield). 1H NMR (400 MHz, CDCl₃) δ 8.76 (s, 1H), 8.72 (d, J=4.5 Hz, 1H), 8.27 (s, 1H), 7.98 (d, J=8.1 Hz, 1H), 7.75 (s, 1H), 7.58 (s, 1H), 7.56-7.49 (m, 1H), 7.33 (s, 2H), 7.31-7.18 (m, 4H), 7.15 (d, J=7.3 Hz, 1H), 7.03-6.98 (m, 1H), 5.04 (s, 2H), 4.06 (s, 3H), 4.05 (s, 3H), 2.37 (s, 3H). MS (ESI) m/z: [M+H]⁺ calcd for C₃₀H₂₆ClN₄O₄+541.16, found 541.3.

### 3-(3-Chloro-5-((2-fluorobenzyl)oxy)phenyl)-5-(2,4-dimethoxypyrimidin-5-yl)-2H-[1,3′-bipyridin]-2-one (S11g)

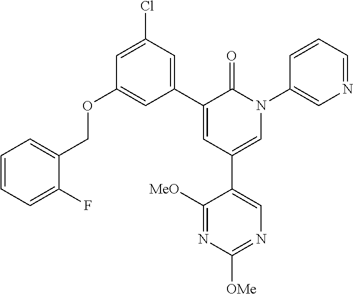 [SMILES: COc1ncc(-c2cc(-c3cc(Cl)cc(OCc4ccccc4F)c3)c(=O)n(-c3cccnc3)c2)c(OC)n1]

General Procedure D was employed using S9 and 1-bromo-3-chloro-5-((2-fluorobenzyl)oxy) benzene (S10g) to afford the title compound as a white solid (62 mg, 57% yield). ¹H NMR (400 MHz, CDCl₃) δ 8.81 (s, 1H), 8.74 (d, J=4.4 Hz, 1H), 8.30 (s, 1H), 8.09 (d, J=8.4 Hz, 1H), 7.76 (d, J=2.1 Hz, 1H), 7.60 (s, 2H), 7.49 (t, J=7.5 Hz, 1H), 7.33 (d, J=8.3 Hz, 3H), 7.17 (t, J=7.5 Hz, 1H), 7.09 (t, J=9.1 Hz, 1H), 7.02 (t, J=2.0 Hz, 1H), 5.15 (s, 2H), 4.08 (s, 3H), 4.06 (s, 3H). MS (ESI) m/z: [M+H]⁺ calcd for C₂₉H₂₃ClFN₄O₄+545.14, found 545.2.

### 3-(3-Chloro-5-((3-fluorobenzyl)oxy)phenyl)-5-(2,4-dimethoxypyrimidin-5-yl)-2H-[1,3′-bipyridin]-2-one (S11h)

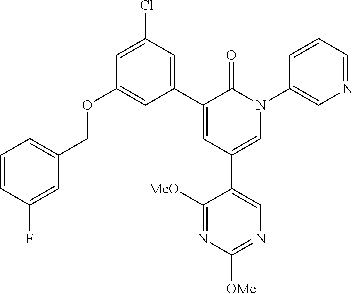 [SMILES: COc1ncc(-c2cc(-c3cc(Cl)cc(OCc4cccc(F)c4)c3)c(=O)n(-c3cccnc3)c2)c(OC)n1]

General Procedure D was employed using S9 and 1-bromo-3-chloro-5-((3-fluorobenzyl)oxy) benzene (S10h) to afford the title compound as a white solid (71.8 mg, 66% yield). 1H NMR (400 MHz, CDCl₃) δ 8.83 (s, 1H), 8.75 (d, J=4.7 Hz, 1H), 8.31 (s, 1H), 8.12 (d, J=8.1 Hz, 1H), 7.76 (s, 1H), 7.66-7.59 (m, 2H), 7.38-7.29 (m, 3H), 7.16 (dd, J=14.7, 8.7 Hz, 2H), 7.03 (d, J=8.1 Hz, 1H), 7.00 (s, 1H), 5.08 (s, 2H), 4.08 (s, 3H), 4.06 (s, 3H). MS (ESI) m/z: [M+H]⁺ calcd for C₂₉H₂₃ClFN₄O₄⁺ 545.14, found 545.2.

### 3-(3-Chloro-5-((2,6-difluorobenzyl)oxy)phenyl)-5-(2,4-dimethoxypyrimidin-5-yl)-2H-[1,3′-bipyridin]-2-one (S11i)

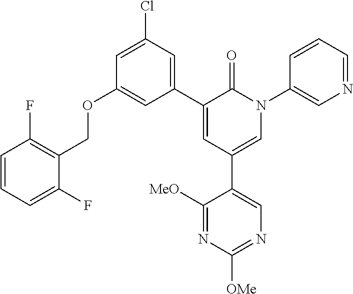 [SMILES: COc1ncc(-c2cc(-c3cc(Cl)cc(OCc4c(F)cccc4F)c3)c(=O)n(-c3cccnc3)c2)c(OC)n1]

General Procedure D was employed using S9 and 2-((3-bromo-5-chlorophenoxy)methyl)-1,3-difluorobenzene (S10i) to afford the title compound as a white solid (60.7 mg, 54% yield). ¹H NMR (400 MHz, CDCl₃) δ 8.76 (d, J=2.1 Hz, 1H), 8.72 (d, J=3.9 Hz, 1H), 8.27 (s, 1H), 7.97 (d, J=8.7 Hz, 1H), 7.76 (d, J=2.5 Hz, 1H), 7.58 (d, J=2.5 Hz, 1H), 7.52 (dd, J=8.1, 4.9 Hz, 1H), 7.39-7.35 (m, 1H), 7.33 (dd, J=3.7, 1.6 Hz, 2H), 7.03 (t, J=2.0 Hz, 1H), 6.94 (t, J=7.8 Hz, 2H), 5.14 (s, 2H), 4.07 (s, 3H), 4.05 (s, 3H). MS (ESI) m/z: [M+H]⁺ calcd for C₂₉H₂₂ClF₂N₄O₄⁺ 563.13, found 563.2.

### 3-(3-Chloro-5-((2-chlorobenzyl)oxy)phenyl)-5-(2,4-dimethoxypyrimidin-5-yl)-2H-[1,3′-bipyridin]-2-one (S11j)

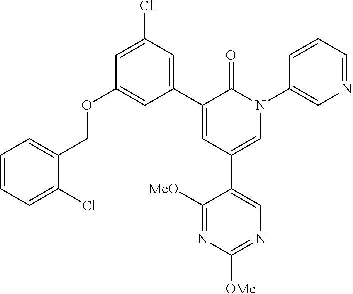 [SMILES: COc1ncc(-c2cc(-c3cc(Cl)cc(OCc4ccccc4Cl)c3)c(=O)n(-c3cccnc3)c2)c(OC)n1]

General Procedure D was employed using S9 and 1-bromo-3-chloro-5-((2-chlorobenzyl)oxy)benzene (S10j) to afford the title compound as a white solid (124.2 mg, 55% yield). ¹H NMR (400 MHz, CDCl₃) δ 8.78 (s, 1H), 8.73 (d, J=4.4 Hz, 1H), 8.28 (s, 1H), 8.03 (d, J=8.0 Hz, 1H), 7.75 (d, J=2.3 Hz, 1H), 7.59 (d, J=2.4 Hz, 1H), 7.55 (dd, J=10.3, 5.1 Hz, 2H), 7.41 (dd, J=7.0, 2.1 Hz, 1H), 7.38-7.27 (m, 4H), 7.02 (t, J=2.0 Hz, 1H), 5.18 (s, 2H), 4.07 (s, 3H), 4.05 (s, 3H). MS (ESI) m/z: [M+H]⁺ calcd for C₂₉H₂₃Cl₂N₄O₄⁺ 561.11, found 561.2.

### 3-(3-Chloro-5-((2-chloro-4-fluorobenzyl)oxy)phenyl)-5-(2,4-dimethoxypyrimidin-5-yl)-2H-[1,3′-bipyridin]-2-one (S11k)

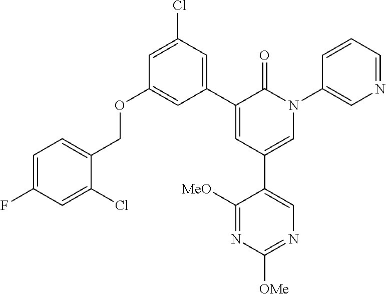 [SMILES: COc1ncc(-c2cc(-c3cc(Cl)cc(OCc4ccc(F)cc4Cl)c3)c(=O)n(-c3cccnc3)c2)c(OC)n1]

General Procedure D was employed using S9 and 1-((3-bromo-5-chlorophenoxy)methyl)-2-chloro-4-fluorobenzene (S10k) to afford the title compound as a white solid (58.9 mg, 51% yield). ¹H NMR (400 MHz, CDCl₃) δ 8.76 (s, 1H), 8.73 (d, J=4.7 Hz, 1H), 8.28 (s, 1H), 7.99 (d, J=8.2 Hz, 1H), 7.76 (d, J=1.8 Hz, 1H), 7.59 (d, J=1.9 Hz, 1H), 7.53 (td, J=9.0, 8.5, 5.7 Hz, 2H), 7.34 (s, 2H), 7.17 (dd, J=8.4, 2.0 Hz, 1H), 7.03 (dd, J=8.3, 2.1 Hz, 1H), 7.00 (s, 1H), 5.12 (s, 2H), 4.07 (s, 3H), 4.05 (s, 3H). MS (ESI) m/z: [M+H]⁺ calcd for C₂₉H₂₂Cl₂FN₄O₄⁺ 579.10, found 579.2.

### 3-(3-Chloro-5-((2-chlorobenzyl)oxy)-4-fluorophenyl)-5-(2,4-dimethoxypyrimidin-5-yl)-2H-[1,3′-bipyridin]-2-one (S111)

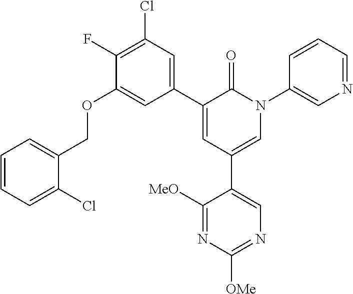 [SMILES: COc1ncc(-c2cc(-c3cc(Cl)c(F)c(OCc4ccccc4Cl)c3)c(=O)n(-c3cccnc3)c2)c(OC)n1]

General Procedure D was employed using S9 and 5-bromo-1-chloro-3-((2-chlorobenzyl)oxy)-2-fluorobenzene (S10l) to afford the title compound as a white solid (77.4 mg, 67% yield). ¹H NMR (400 MHz, CDCl₃) δ 8.81 (s, 1H), 8.75 (d, J=4.2 Hz, 1H), 8.30 (s, 1H), 8.08 (d, J=8.2 Hz, 1H), 7.70 (d, J=2.1 Hz, 1H), 7.62-7.58 (m, 2H), 7.54 (dd, J=7.4, 1.5 Hz, 1H), 7.43-7.37 (m, 3H), 7.30 (td, J=6.9, 1.8 Hz, 2H), 5.26 (s, 2H), 4.08 (s, 3H), 4.06 (s, 3H). MS (ESI) m/z: [M+H]⁺ calcd for C₂₉H₂₂Cl₂FN₄O₄⁺ 579.10, found 579.2.

### 3-(3-Chloro-5-(cyclopropylmethoxy)phenyl)-5-(2,4-dimethoxypyrimidin-5-yl)-2H-[1,3′-bipyridin]-2-one (Slim)

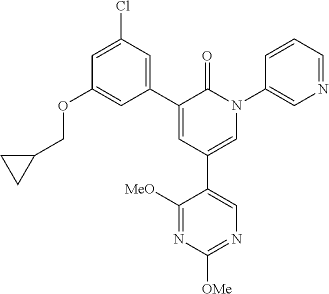 [SMILES: COc1ncc(-c2cc(-c3cc(Cl)cc(OCC4CC4)c3)c(=O)n(-c3cccnc3)c2)c(OC)n1]

General Procedure D was employed using S9 and 1-bromo-3-chloro-5-(cyclopropylmethoxy)benzene (S10m) to afford the title compound as a white solid (73.5 mg, 75% yield). 1H NMR (400 MHz, CDCl₃) δ 8.82 (s, 1H), 8.74 (s, 1H), 8.30 (s, 1H), 8.13 (d, J=7.3 Hz, 1H), 7.76 (s, 1H), 7.64 (s, 1H), 7.60 (s, 1H), 7.27 (s, 1H), 7.22 (s, 1H), 6.93 (s, 1H), 4.08 (s, 3H), 4.06 (s, 3H), 3.82 (d, J=6.8 Hz, 2H), 1.28 (s, 1H), 0.64 (d, J=7.8 Hz, 2H), 0.34 (d, J=4.0 Hz, 2H). MS (ESI) m/z: [M+H]⁺ calcd for C₂₆H₂₄ClN₄O₄⁺ 491.15, found 491.3.

### 3-(3-Chloro-5-(3,3,3-trifluoropropoxy)phenyl)-5-(2,4-dimethoxypyrimidin-5-yl)-2H-[1,3′-bipyridin]-2-one (S11n)

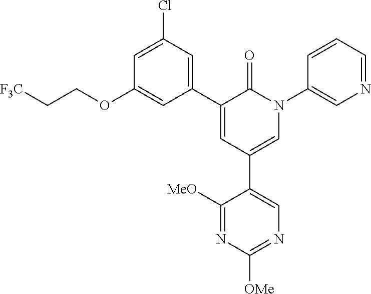 [SMILES: COc1ncc(-c2cc(-c3cc(Cl)cc(OCCC(F)(F)F)c3)c(=O)n(-c3cccnc3)c2)c(OC)n1]

General Procedure D was employed using S9 and 1-bromo-3-chloro-5-(3,3,3-trifluoropropoxy) benzene (S10n) to afford the title compound as a white solid (66 mg, 62% yield). ¹H NMR (400 MHz, CDCl₃) δ 8.80 (s, 1H), 8.74 (d, J=4.5 Hz, 1H), 8.30 (s, 1H), 8.07 (d, J=8.2 Hz, 1H), 7.77 (s, 1H), 7.64-7.57 (m, 2H), 7.31 (s, 1H), 7.27 (s, 1H), 6.93 (s, 1H), 4.22 (t, J=6.5 Hz, 2H), 4.08 (s, 3H), 4.06 (s, 3H), 2.71-2.54 (m, 2H). MS (ESI) m/z: [M+H]⁺ calcd for C₂₅H₂₁ClF₃N₄O₄⁺533.12, found 533.2.

### 2-(3-(3-Chlorophenyl)-2-oxo-2H-[1,3′-bipyridin]-5-yl)benzonitrile (6)

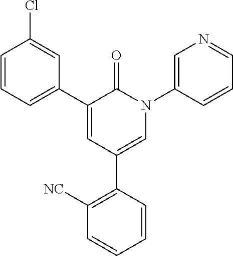 [SMILES: N#Cc1ccccc1-c1cc(-c2cccc(Cl)c2)c(=O)n(-c2cccnc2)c1]

General Procedure B was employed using S4a (12.3 mg, 0.035 mmol, 1.0 equiv) and (3-chlorophenyl)boronic acid (10.9 mg, 0.070 mmol, 2.0 equiv). Purification was accomplished using normal phase column chromatography (SNAP Ultra 25 g, gradient=0-80% EtOAc/Hex over 9 CV, then 80% EtOAc/Hex over 2 CV, then 80-100% EtOAc/Hex over 2 CV) to afford 13.0 mg (97% yield) of a white solid. ¹H NMR (600 MHz, CDCl₃) δ 8.82-8.81 (m, 1H), 8.72 (d, J=4.3 Hz, 1H), 7.96 (ddd, J=8.2, 2.5, 1.5 Hz, 1H), 7.83 (d, J=2.7 Hz, 1H), 7.80 (ddd, J=5.1, 2.7, 1.4 Hz, 2H), 7.72-7.66 (m, 3H), 7.55 (d, J=7.9 Hz, 1H), 7.52-7.48 (m, 2H), 7.39-7.35 (m, 2H). ¹³C NMR (151 MHz, CDCl₃) δ 160.3, 150.1, 147.5, 140.1, 139.2, 137.5, 137.5, 136.6, 134.7, 134.4, 134.1, 133.6, 131.6, 129.7, 129.3, 129.0, 128.7, 128.5, 127.1, 123.9, 118.6, 117.6, 111.1. HRMS (ESI) m/z: [M+H]⁺ calcd for C₂₃H₁₅ClN₃O⁺ 384.0898, found 384.0922.

### 5-(3-(3-Chlorophenyl)-2-oxo-2H-[1,3′-bipyridin]-5-yl)pyrimidine-2,4(1H,3H)-dione (8)

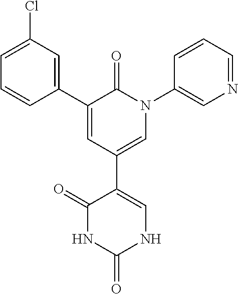 [SMILES: O=c1[nH]cc(-c2cc(-c3cccc(Cl)c3)c(=O)n(-c3cccnc3)c2)c(=O)[nH]1]

General Procedures C and E were employed using S9 and (3-chlorophenyl)boronic acid to afford 8 (81.5 mg, 52% yield). ¹H NMR (400 MHz, DMSO-d6) δ 11.34 (s, 1H), 11.26 (d, J=4.4 Hz, 1H), 8.72 (d, J=2.2 Hz, 1H), 8.63 (d, J=3.8 Hz, 1H), 8.04 (d, J=2.5 Hz, 1H), 8.02-7.99 (m, 1H), 7.98 (d, J=2.6 Hz, 1H), 7.88 (d, J=5.5 Hz, 1H), 7.82 (s, 1H), 7.67 (d, J=7.4 Hz, 1H), 7.57 (dd, J=8.1, 4.8 Hz, 1H), 7.48-7.34 (m, 2H). ¹³C NMR (101 MHz, DMSO-d6) δ 163.7, 159.8, 151.3, 149.6, 148.1, 140.1, 139.7, 138.9, 138.1, 136.8, 135.3, 133.1, 130.3, 128.7, 128.3, 127.9, 127.6, 124.3, 112.7, 107.9. HRMS (ESI) m/z: [M+H]⁺ calcd for C₂₀H₁₄ClN₄O₃+393.0749, found 393.0752.

### 2-(3-(3,5-Dichlorophenyl)-2-oxo-2H-[1,3′-bipyridin]-5-yl)benzonitrile (9)

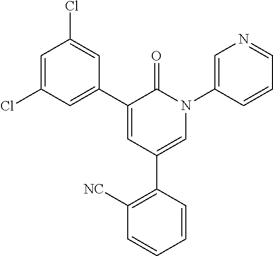 [SMILES: N#Cc1ccccc1-c1cc(-c2cc(Cl)cc(Cl)c2)c(=O)n(-c2cccnc2)c1]

General Procedure B was employed using S4a (26.0 mg, 0.074 mmol, 1.0 equiv) and (3,5-dichlorophenyl)boronic acid (28.2 mg, 0.15 mmol, 2.0 equiv). Purification was accomplished using normal phase column chromatography (SNAP Ultra 25 g, gradient=0-100% EtOAc/Hex over 10 CV, then 100% EtOAc/Hex over 3 CV) followed by reverse phase column chromatography (SNAP Ultra C18 30 g, gradient=30-68% MeCN/H₂O over 5 CV, then 68% MeCN/H₂O over 2 CV, then 68-100% MeCN/H₂O over 5 CV) to afford 9.3 mg (30% yield) of a pale pink solid. 1H NMR (600 MHz, CDCl₃) δ 8.80 (d, J=2.5 Hz, 1H), 8.72 (d, J=4.9 Hz, 1H), 7.95 (dt, J=8.2, 1.9 Hz, 1H), 7.83 (d, J=2.6 Hz, 1H), 7.80 (dd, J=7.8, 1.3 Hz, 1H), 7.75-7.65 (m, 4H), 7.55 (d, J=7.8 Hz, 1H), 7.51 (ddd, J=8.6, 6.0, 3.1 Hz, 2H), 7.37 (t, J=1.9 Hz, 1H). ¹³C NMR (151 MHz, CDCl₃) δ 160.0, 150.2, 147.4, 139.8, 139.6, 138.5, 137.3, 137.2, 135.0, 134.6, 134.1, 133.6, 130.3, 129.3, 128.7, 128.6, 127.3, 124.0, 118.5, 117.5, 111.2. HRMS (ESI) m/z: [M+H]⁺ calcd for C₂₃H₁₄Cl₂N₃O⁺418.0508, found 418.0527.

### 2-(3-(3-Chloro-5-propoxyphenyl)-2-oxo-2H-[1,3′-bipyridin]-5-yl)benzonitrile (13)

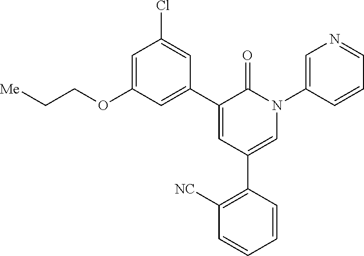 [SMILES: CCCOc1cc(Cl)cc(-c2cc(-c3ccccc3C#N)cn(-c3cccnc3)c2=O)c1]

General Procedure B was employed using S4a (26.4 mg, 0.075 mmol, 1.0 equiv) and (3-chloro-5-propoxy-phenyl)boronic acid (32.2 mg, 0.15 mmol, 2.0 equiv). Purification was accomplished using normal phase column chromatography (SNAP Ultra 25 g, gradient=0-90% EtOAc/Hex over 9 CV, then 90% EtOAc/Hex over 2 CV, then 90-100% EtOAc/Hex over 1 CV) followed by reverse phase column chromatography (SNAP Ultra C18 30 g, gradient=0-85% MeOH/H₂O over 7 CV, then 85% MeOH/H₂O over 2 CV, then 85-88% MeOH/H₂O over 2 CV, then 88-100% MeOH/H₂O over 2 CV) to afford 10.4 mg (31% yield) of a white solid. 1H NMR (600 MHz, MeOD-d₄) δ 8.81 (s, 1H), 8.68 (s, 1H), 8.10 (dt, J=8.3, 1.7 Hz, 1H), 8.06 (q, J=2.7 Hz, 2H), 7.87 (d, J=7.7 Hz, 1H), 7.78-7.71 (m, 2H), 7.66 (dd, J=8.2, 4.8 Hz, 1H), 7.56 (td, J=7.4, 1.7 Hz, 1H), 7.36 (t, J=1.6 Hz, 1H), 7.27 (t, J=1.8 Hz, 1H), 6.95 (t, J=2.1 Hz, 1H), 3.98 (t, J=6.4 Hz, 2H), 1.80 (h, J=7.1 Hz, 2H), 1.05 (t, J=7.4 Hz, 3H). ¹³C NMR (101 MHz, MeOD-d₄) δ 162.0, 161.2, 150.4, 148.6, 141.7, 141.3, 139.9, 139.2, 136.9, 135.6, 135.0, 134.8, 131.6, 130.8, 129.6, 125.7, 122.1, 119.7, 119.6, 115.8, 114.7, 112.1, 71.1, 23.6, 10.8. HRMS (ESI) m/z: [M+H]⁺ calcd for C₂₆H₂₁ClN₃O₂⁺442.1317, found 442.1320.

### 2-(3-(3-Chloro-5-(2-methoxyethoxy)phenyl)-2-oxo-2H-[1,3′-bipyridin]-5-yl)benzonitrile (16)

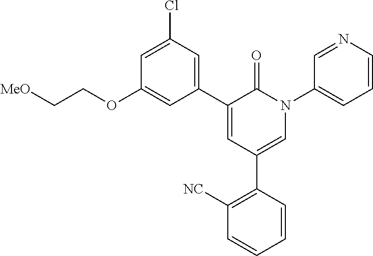 [SMILES: COCCOc1cc(Cl)cc(-c2cc(-c3ccccc3C#N)cn(-c3cccnc3)c2=O)c1]

General Procedure D was employed using S10a (39.8 mg, 0.15 mmol, 1.1 equiv) and S4a (50.0 mg, 0.14 mmol, 1.0 equiv). Purification was accomplished using reverse phase column chromatography (SNAP Ultra C18 60 g, gradient=0-60% MeCN/H₂O over 7 CV, then 60% MeCN/H₂O over 4 CV, then 60-100% MeCN/H₂O over 3 CV) to afford 18.1 mg (28% yield over 2 steps) of a white solid. ¹H NMR (500 MHz, CDCl₃) δ 8.82 (s, 1H), 8.72 (s, 1H), 7.95 (d, J=8.1 Hz, 1H), 7.82 (d, J=2.6 Hz, 1H), 7.79 (dd, J=7.8, 1.3 Hz, 1H), 7.72-7.67 (m, 2H), 7.55 (d, J=7.5 Hz, 1H), 7.49 (td, J=7.7, 1.2 Hz, 2H), 7.38 (t, J=1.6 Hz, 1H), 7.31 (dd, J=2.4, 1.5 Hz, 1H), 6.96 (t, J=2.1 Hz, 1H), 4.16-4.12 (m, 2H), 3.76-3.72 (m, 2H), 3.44 (s, 3H). ¹³C NMR (126 MHz, CDCl₃) δ 160.2, 159.4, 150.0, 147.5, 140.1, 139.3, 138.1, 137.6, 136.7, 134.9, 134.7, 134.1, 133.6, 131.5, 129.3, 128.5, 124.0, 121.6, 118.6, 117.5, 115.6, 113.5, 111.1, 71.0, 67.9, 59.4. HRMS (ESI) m/z: [M+H]⁺ calcd for C₂₆H₂₁ClN₃O₃⁺458.1266, found 458.1255.

### 2-(3-(3-(Benzyloxy)-5-chlorophenyl)-2-oxo-2H-[1,3′-bipyridin]-5-yl)benzonitrile (14)

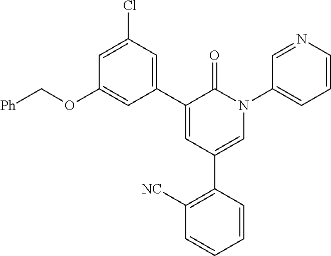 [SMILES: N#Cc1ccccc1-c1cc(-c2cc(Cl)cc(OCc3ccccc3)c2)c(=O)n(-c2cccnc2)c1]

General Procedure B was employed using S4a (26.4 mg, 0.075 mmol, 1.0 equiv) and (3-(benzyloxy)-5-chlorophenyl)boronic acid (39.4 mg, 0.15 mmol, 2.0 equiv). Purification was accomplished using reverse phase column chromatography (SNAP Ultra C18 30 g, gradient=0-80% MeOH/H₂O over 6 CV, then 80-90% MeOH/H₂O over 5 CV, then 90-100% MeOH/H₂O over 1 CV, then 100% MeOH/H₂O over 3 CV), then normal phase column chromatography (SNAP Ultra 25 g, gradient=0-90% EtOAc/Hex over 5 CV, then 90% EtOAc/Hex over 3 CV, then 90-100% EtOAc/Hex over 1 CV) to afford 18.1 mg (49% yield) of a peach-colored solid. ¹H NMR (400 MHz, CDCl₃) δ 8.82 (s, 1H), 8.72 (s, 1H), 7.95 (ddd, J=8.2, 2.5, 1.3 Hz, 1H), 7.86-7.77 (m, 2H), 7.74-7.64 (m, 2H), 7.54 (dd, J=8.1, 1.1 Hz, 1H), 7.49 (td, J=7.6, 1.2 Hz, 2H), 7.46-7.39 (m, 2H), 7.40-7.35 (m, 4H), 7.35-7.28 (m, 1H), 7.00 (t, J=2.1 Hz, 1H), 5.08 (s, 2H). ¹³C NMR (101 MHz, CDCl₃) δ 160.2, 159.4, 150.1, 147.5, 140.0, 139.3, 138.2, 136.7, 136.5, 134.9, 134.6, 134.1, 133.6, 131.5, 129.3, 128.7, 128.5, 128.2, 127.7, 124.0, 121.6, 118.6, 117.5, 115.7, 113.8, 111.1, 70.5. HRMS (ESI) m/z: [M+H]⁺ calcd for C₃₀H₂₁ClN₃O₂⁺490.1317, found 490.1329.

### 2-(3-(3-Chloro-5-phenethoxyphenyl)-2-oxo-2H-[1,3′-bi-pyridin]-5-yl)benzonitrile (17)

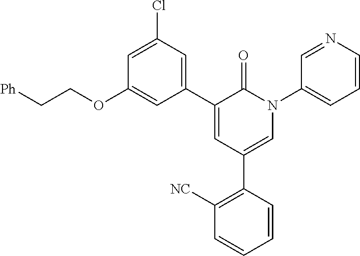 [SMILES: N#Cc1ccccc1-c1cc(-c2cc(Cl)cc(OCCc3ccccc3)c2)c(=O)n(-c2cccnc2)c1]

General Procedure D was employed using S10b (48.7 mg, 0.15 mmol, 1.1 equiv) and S4a (50.0 mg, 0.14 mmol, 1.0 equiv). Purification was accomplished using reverse phase column chromatography (SNAP Ultra C18 60 g, gradient=0-100% MeOH/H₂O over 8 CV, then 100% MeOH/H₂O over 3 CV), then normal phase column chromatography (SNAP Ultra 25 g, gradient=0-80% EtOAc/Hex over 7 CV, then 80% EtOAc/Hex over 3 CV, then 80-100% EtOAc/Hex over 2 CV) to afford 31.8 mg (44% yield over 2 steps) of a white solid. ¹H NMR (400 MHz, CDCl₃) δ 8.80 (d, J=2.6 Hz, 1H), 8.71 (d, J=4.8 Hz, 1H), 7.94 (ddd, J=8.2, 2.6, 1.5 Hz, 1H), 7.81-7.77 (m, 2H), 7.72-7.67 (m, 2H), 7.57-7.52 (m, 1H), 7.52-7.46 (m, 2H), 7.33 (q, J=1.3 Hz, 1H), 7.31 (t, J=1.2 Hz, 1H), 7.29-7.27 (m, 4H), 7.25-7.19 (m, 1H), 6.92 (t, J=2.1 Hz, 1H), 4.20 (t, J=7.0 Hz, 2H), 3.09 (t, J=7.0 Hz, 2H). ¹³C NMR (126 MHz, CDCl₃) δ 160.2, 15958, 150.0, 147.5, 140.1, 139.3, 138.1, 136.7, 134.9, 134.6, 134.1, 133.6, 132.2, 132.1, 131.5, 129.3, 129.1, 128.8, 128.6, 128.5, 126.7, 121.3, 118.6, 117.5, 115.5, 113.5, 111.1, 69.12, 35.8. HRMS (ESI) m/z: [M+H]⁺ calcd for C₃₁H₂₃ClN₃O₂⁺504.1473, found 504.1498.

### 2-(5-(3-Chloro-5-propoxyphenyl)-6-oxo-1-(pyrimidin-5-yl)-1,6-dihydropyridin-3-yl)benzo-nitrile (15)

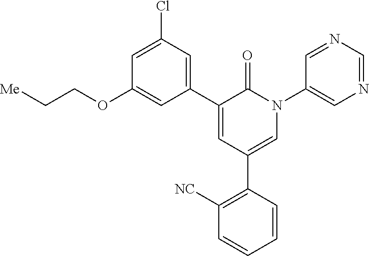 [SMILES: CCCOc1cc(Cl)cc(-c2cc(-c3ccccc3C#N)cn(-c3cncnc3)c2=O)c1]

General Procedure B was employed using S4b (15.0 mg, 0.04 mmol, 1.00 equiv) and (3-chloro-5-propoxyphenyl)boronic (18.2 mg, 0.08 mmol, 2.00 equiv). Purification was accomplished using reverse phase column chromatography (SNAP Ultra C18 30 g, gradient=0-87% MeOH/H₂O over 8 CV, then 87 MeOH/H₂O over 2 CV, then 87-100% MeOH/H₂O over 1 CV, then 100% MeOH/H₂O over 3 CV), then normal phase column chromatography (SNAP Ultra 25 g, gradient=0-8% DCM/MeOH over 8 CV) to afford 8.0 mg (43% yield) of a white solid. ¹H NMR (600 MHz, CDCl₃) δ 9.30 (s, 1H), 9.03 (s, 2H), 7.82 (d, J=2.9 Hz, 1H), 7.81 (d, J=8.9 Hz, 1H), 7.71 (t, J=7.7 Hz, 1H), 7.66 (d, J=2.6 Hz, 1H), 7.55 (d, J=7.9 Hz, 1H), 7.52 (t, J=7.7 Hz, 1H), 7.30 (s, 1H), 7.24 (s, 1H), 3.94 (t, J=6.5 Hz, 2H), 1.80 (h, J=7.1 Hz, 2H), 1.03 (t, J=7.4 Hz, 3H). ¹³C NMR (126 MHz, CDCl₃) δ 159.9, 159.9, 158.3, 154.7, 139.7, 139.7, 137.7, 136.1, 135.6, 135.0, 134.2, 133.7, 132.0, 129.3, 128.8, 121.0, 118.5, 118.2, 115.5, 113.7, 111.2, 70.1, 22.6, 10.6. HRMS (ESI) m z: [M+H]⁺ calcd for C₂₅H₂₀ClN₄O₂⁺443.1269, found 443.1278.

### 5-(3-(3-Chloro-5-methoxyphenyl)-2-oxo-2H-[1,3′-bipyridin]-5-yl)pyrimidine-2,4(1H,3H)-dione (18)

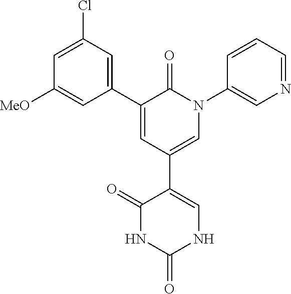 [SMILES: COc1cc(Cl)cc(-c2cc(-c3c[nH]c(=O)[nH]c3=O)cn(-c3cccnc3)c2=O)c1]

General Procedures C and E were employed using S9 and (3-chloro-5-methoxyphenyl) boronic acid to afford 18 (54 mg, 64% yield). ¹H NMR (400 MHz, DMSO-d6) δ 11.23 (br.s, 2H), 8.71 (d, J=1.9 Hz, 1H), 8.63 (d, J=4.0 Hz, 1H), 8.04 (d, J=2.1 Hz, 1H), 8.02-7.94 (m, 2H), 7.87 (s, 1H), 7.57 (dd, J=8.0, 4.8 Hz, 1H), 7.39 (s, 1H), 7.27 (s, 1H), 6.98 (s, 1H), 3.77 (s, 3H). ¹³C NMR (101 MHz, DMSO-d₆) δ 163.75, 160.24, 159.67, 151.82, 149.59, 148.05, 140.63, 140.18, 139.53, 138.08, 136.66, 135.28, 133.67, 128.17, 124.32, 121.06, 113.83, 113.49, 112.85, 107.54, 56.07. HRMS (ESI) m/z: [M+H]⁺ calcd for C₂₁H₁₆ClN₄O₄⁺423.0855, found 423.0857.

### 5-(3-(3-Chloro-5-propoxyphenyl)-2-oxo-2H-[1,3′-bipyridin]-5-yl)pyrimidine-2,4(1H,3H)-dione (24)

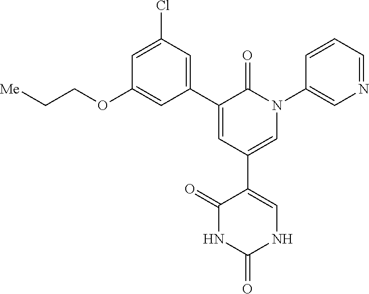 [SMILES: CCCOc1cc(Cl)cc(-c2cc(-c3c[nH]c(=O)[nH]c3=O)cn(-c3cccnc3)c2=O)c1]

General Procedure E was employed using S11a to afford 24 as a pale yellow solid (105 mg, 93% yield). 1H NMR (600 MHz, DMSO-d₆) δ 11.32 (s, 1H), 11.23 (s, 1H), 8.72 (d, J=2.1 Hz, 1H), 8.67-8.60 (m, 1H), 8.04 (d, J=2.3 Hz, 1H), 8.02-7.96 (m, 2H), 7.88 (s, 1H), 7.57 (dd, J=8.0, 4.8 Hz, 1H), 7.38 (s, 1H), 7.28 (s, 1H), 6.97 (s, 1H), 3.95 (t, J=6.5 Hz, 2H), 1.70 (q, J=6.9 Hz, 2H), 0.95 (t, J=7.4 Hz, 3H). ¹³C NMR (151 MHz, DMSO-d₆) δ 163.7, 159.7, 159.7, 151.3, 149.6, 148.1, 140.2, 139.7, 139.5, 138.1, 136.8, 135.3, 133.7, 128.2, 124.3, 120.9, 114.3, 114.0, 112.6, 107.9, 69.9, 22.4, 10.8. HRMS (ESI) m/z: [M+H]⁺ calcd for C₂₃H₂₀ClN₄O₄⁺ 451.1168, found 451.1179.

### 5-(3-(3-Butoxy-5-chlorophenyl)-2-oxo-2H-[1,3′-bipyridin]-5-yl)pyrimidine-2,4(1H,3H)-dione (25)

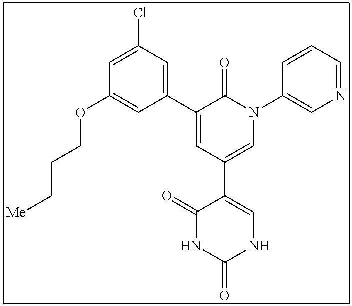 [SMILES: CCCCOc1cc(Cl)cc(-c2cc(-c3c[nH]c(=O)[nH]c3=O)cn(-c3cccnc3)c2=O)c1]

General Procedure E was employed using S11b to afford 25 (56.5 mg, 91% yield). 1H NMR (600 MHz, DMSO-d₆) δ 11.27 (br.s, 2H), 8.72 (d, J=1.9 Hz, 1H), 8.68-8.60 (m, 1H), 8.04 (d, J=2.2 Hz, 1H), 8.02-7.95 (m, 2H), 7.88 (s, 1H), 7.57 (dd, J=7.9, 4.9 Hz, 1H), 7.38 (s, 1H), 7.27 (s, 1H), 6.97 (s, 1H), 3.99 (t, J=6.4 Hz, 2H), 1.67 (p, J=6.6 Hz, 2H), 1.41 (h, J=7.4 Hz, 2H), 0.90 (t, J=7.4 Hz, 3H). ¹³C NMR (151 MHz, DMSO-d₆) δ 163.7, 159.7, 159.7, 151.5, 149.6, 148.1, 140.2, 140.0, 139.5, 138.1, 136.8, 135.3, 133.6, 128.2, 124.3, 120.9, 114.3, 113.9, 112.7, 107.8, 68.1, 31.1, 19.1, 14.1. HRMS (ESI) m/z: [M+H]⁺ calcd for C₂₄H₂₂ClN₄O₄⁺ 465.1324, found 465.1326.

### 5-(3-(3-Chloro-5-(isopentyloxy)phenyl)-2-oxo-2H-[1,3′-bipyridin]-5-yl)pyrimidine-2,4(1H, 3H)-dione (26)

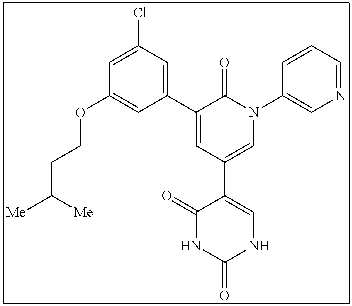 [SMILES: CC(C)CCOc1cc(Cl)cc(-c2cc(-c3c[nH]c(=O)[nH]c3=O)cn(-c3cccnc3)c2=O)c1]

General Procedure E was employed using S11c to afford 26 (62.1 mg, 89% yield). 1H NMR (600 MHz, DMSO-d₆) δ 11.27 (br.s, 2H), 8.68 (d, J=2.1 Hz, 1H), 8.60 (d, J=4.7 Hz, 1H), 8.00 (d, J=2.1 Hz, 1H), 7.95 (d, J=8.3 Hz, 1H), 7.94 (d, J=2.3 Hz, 1H), 7.84 (s, 1H), 7.53 (dd, J=8.0, 4.9 Hz, 1H), 7.34 (s, 1H), 7.23 (s, 1H), 6.95 (s, 1H), 3.97 (t, J=6.5 Hz, 2H), 1.75-1.68 (m, 1H), 1.55 (q, J=6.6 Hz, 2H), 0.87 (s, 3H), 0.86 (s, 3H). ¹³C NMR (151 MHz, DMSO-d₆) δ 163.25, 159.25, 159.22, 150.97, 149.17, 147.63, 139.74, 139.42, 139.03, 137.64, 136.36, 134.84, 133.21, 127.73, 123.88, 120.49, 113.95, 113.44, 112.19, 107.39, 66.43, 37.33, 24.54, 22.42. HRMS (ESI) m/z: [M+H]⁺ calcd for C₂₅H₂₄ClN₄O₄⁺ 479.1481, found 479.1483.

### 5-(3-(3-(Benzyloxy)-5-chlorophenyl)-2-oxo-2H-[1,3′-bipyridin]-5-yl)pyrimidine-2,4(1H,3H)-dione (46)

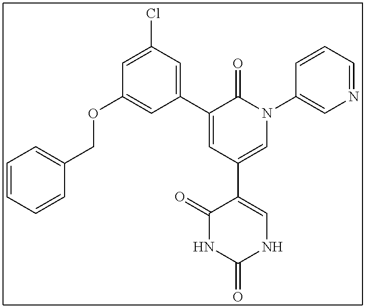 [SMILES: O=c1[nH]cc(-c2cc(-c3cc(Cl)cc(OCc4ccccc4)c3)c(=O)n(-c3cccnc3)c2)c(=O)[nH]1]

General Procedures C and E were employed using S9 and (3-(benzyloxy)-5-chlorophenyl) boronic acid to afford 46 (51.8 mg, 52% yield). ¹H NMR (600 MHz, DMSO-d₆) δ 11.32 (br.s, 2H), 8.72 (s, 1H), 8.64 (d, J=4.5 Hz, 1H), 8.04 (d, J=1.7 Hz, 1H), 8.00 (dt, J=7.8, 1.2 Hz, 1H), 7.98 (d, J=1.8 Hz, 1H), 7.88 (s, 1H), 7.58 (dd, J=8.1, 4.8 Hz, 1H), 7.47-7.40 (m, 3H), 7.40-7.34 (m, 3H), 7.31 (t, J=6.9 Hz, 1H), 7.08 (s, 1H), 5.14 (s, 2H). ¹³C NMR (151 MHz, DMSO-d₆) δ 163.7, 159.7, 159.3, 151.4, 149.6, 148.1, 140.2, 139.8, 139.5, 138.1, 137.0, 136.9, 135.3, 133.6, 128.9, 128.4, 128.3, 128.1, 124.3, 121.3, 114.7, 114.3, 112.6, 107.9, 70.1. HRMS (ESI) m/z: [M+H]⁺ calcd for C₂₇H₂₀ClN₄O₄⁺ 499.1168, found 499.1172.

### 5-(3-(3-Chloro-5-phenethoxyphenyl)-2-oxo-2H-[1,3′-bipyridin]-5-yl)pyrimidine-2,4(1H, 3H)-dione (29)

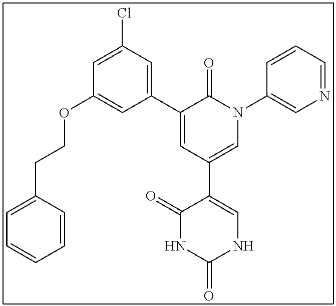 [SMILES: O=c1[nH]cc(-c2cc(-c3cc(Cl)cc(OCCc4ccccc4)c3)c(=O)n(-c3cccnc3)c2)c(=O)[nH]1]

a General Procedure E was employed using S11d to afford 29 (54.3 mg, 87% yield). 1H NMR (600 MHz, DMSO-d₆) δ 11.28 (s, 1H), 11.20 (s, 1H), 8.68 (s, 1H), 8.59 (s, 1H), 7.99 (s, 1H), 7.95 (d, J=13.0 Hz, 2H), 7.83 (s, 1H), 7.59-7.51 (m, 1H), 7.35 (s, 1H), 7.25 (d, J=9.4 Hz, 5H), 7.16 (d, J=5.2 Hz, 1H), 6.99-6.91 (m, 1H), 4.18 (t, J=5.6 Hz, 2H), 2.97 (d, J=6.4 Hz, 2H). ¹³C NMR (151 MHz, DMSO-d₆) δ 163.2, 159.3, 159.0, 150.9, 149.2, 147.6, 139.8, 139.3, 139.1, 138.2, 137.6, 136.4, 134.9, 133.2, 129.0, 128.3, 127.7, 126.3, 123.9, 120.7, 113.8, 113.7, 112.2, 107.4, 68.6, 34.8. HRMS (ESI) m/z: [M+H]⁺ calcd for C₂₈H₂₂ClN₄O₄⁺ 513.1324, found 513.1326.

### 5-(3-(3-Chloro-5-((2-methylbenzyl)oxy)phenyl)-2-oxo-2H-[1,3′-bipyridin]-5-yl)pyrimidine-2,4(1H,3H)-dione (31)

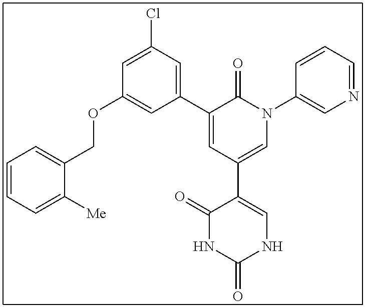 [SMILES: Cc1ccccc1COc1cc(Cl)cc(-c2cc(-c3c[nH]c(=O)[nH]c3=O)cn(-c3cccnc3)c2=O)c1]

General Procedure E was employed using S11e to afford 31 (72.5 mg, 92% yield). 1H NMR (600 MHz, DMSO-d₆) δ 11.29 (s, 1H), 11.20 (s, 1H), 8.68 (s, 1H), 8.62-8.53 (m, 1H), 8.00 (d, J=2.3 Hz, 1H), 7.98-7.88 (m, 2H), 7.86-7.78 (m, 1H), 7.55-753 (m, 1H), 7.39 (s, 1H), 7.36 (d, J=7.5 Hz, 1H), 7.34 (s, 1H), 7.19-7.13 (m, 3H), 7.10-7.05 (m, 1H), 5.08 (s, 2H), 2.27 (s, 3H). ¹³C NMR (151 MHz, DMSO-d₆) δ 163.2, 159.3, 159.0, 150.9, 149.2, 147.6, 139.8, 139.3, 139.1, 137.6, 136.8, 136.5, 134.9, 134.5, 133.2, 130.2, 128.8, 128.3, 127.7, 125.8, 123.9, 120.9, 114.3, 113.8, 112.2, 107.5, 68.5, 18.5. HRMS (ESI) m/z: [M+H]⁺ calcd for C₂₈H₂₂ClN₄O₄⁺ 513.1324, found 513.1327.

### 5-(3-(3-Chloro-5-((3-methylbenzyl)oxy)phenyl)-2-oxo-2H-[1,3′-bipyridin]-5-yl)pyrimidine-2,4(1H,3H)-dione (32)

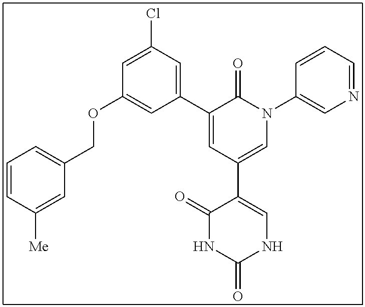 [SMILES: Cc1cccc(COc2cc(Cl)cc(-c3cc(-c4c[nH]c(=O)[nH]c4=O)cn(-c4cccnc4)c3=O)c2)c1]

General Procedure E was employed using S11f to afford 32 (62.6 mg, 86% yield). 1H NMR (600 MHz, DMSO-d₆) δ 11.29 (s, 1H), 11.21 (s, 1H), 8.68 (s, 1H), 8.64-8.56 (m, 1H), 8.00 (s, 1H), 7.96 (d, J=8.1 Hz, 1H), 7.94 (s, 1H), 7.83 (d, J=1.8 Hz, 1H), 7.58-7.51 (m, 1H), 7.38 (s, 1H), 7.33 (s, 1H), 7.25-7.13 (m, 3H), 7.08 (d, J=6.8 Hz, 1H), 7.05-6.99 (m, 1H), 5.05 (s, 2H), 2.25 (s, 3H). ³C NMR (151 MHz, DMSO-d₆) δ 163.2, 159.3, 158.9, 150.9, 149.2, 147.6, 139.8, 139.3, 139.1, 137.7, 136.5, 136.5, 134.9, 133.2, 128.6, 128.4, 127.7, 124.9, 123.9, 120.8, 114.3, 113.8, 112.2, 107.4, 69.7, 21.0. HRMS (ESI) m/z: [M+H]⁺ calcd for C₂₈H₂₂ClN₄O₄⁺ 513.1324, found 513.1329.

### 5-(3-(3-Chloro-5-((2-fluorobenzyl)oxy)phenyl)-2-oxo-2H-[1,3′-bipyridin]-5-yl)pyrimidine-2,4(1H,3H)-dione (34)

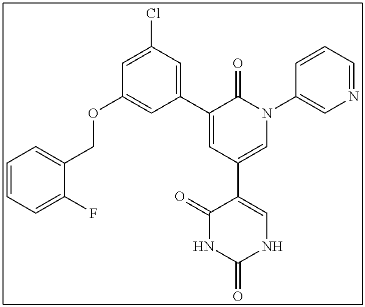 [SMILES: O=c1[nH]cc(-c2cc(-c3cc(Cl)cc(OCc4ccccc4F)c3)c(=O)n(-c3cccnc3)c2)c(=O)[nH]1]

General Procedure E was employed using S11g to afford 34 (54.1 mg, 92% yield). 1H NMR (600 MHz, DMSO-d₆) δ 11.32 (br.s, 2H), 8.72 (s, 1H), 8.63 (s, 1H), 8.05 (s, 1H), 7.99 (s, 2H), 7.88 (s, 1H), 7.64-7.50 (m, 2H), 7.45 (s, 1H), 7.44-7.40 (m, 1H), 7.38 (s, 1H), 7.31-7.18 (m, 2H), 7.12 (s, 1H), 5.17 (s, 2H). ¹³C NMR (151 MHz, DMSO-d₆) δ 163.2, δ 160.5 (d, J=246.5 Hz), 159.2, 158.7, 151.0, 149.2, 147.6, 139.8, 139.4, 139.2, 137.6, 136.5, 134.8, 133.3, 130.9 (d, J=3.7 Hz), 130.6 (d, J=8.2 Hz), 127.6, 124.6 (d, J=3.2 Hz), 123.9, 123.37 (d, J=14.6 Hz), 121.09, 115.45 (d, J=20.9 Hz), 114.16, 113.82, 112.20, 107.40, 64.10. ¹⁹F NMR (376 MHz, DMSO) δ −118.11-−118.25 (m). HRMS (ESI) m/z: [M+H]⁺ calcd for C₂₇H₁₉ClFN₄O₄⁺517.1073, 517.1077.

### 5-(3-(3-Chloro-5-((3-fluorobenzyl)oxy)phenyl)-2-oxo-2H-[1,3′-bipyridin]-5-yl)pyrimidine-2,4(1H,3H)-dione (35)

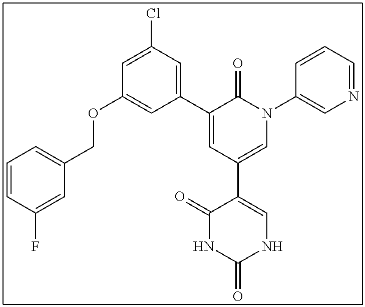 [SMILES: O=c1[nH]cc(-c2cc(-c3cc(Cl)cc(OCc4cccc(F)c4)c3)c(=O)n(-c3cccnc3)c2)c(=O)[nH]1]

General Procedure E was employed using S11h to afford 35 (60.6 mg, 89% yield). 1H NMR (600 MHz, DMSO-d₆) δ 11.23 (br.s, 2H), 8.68 (s, 1H), 8.60 (d, J=4.4 Hz, 1H), 8.01 (s, 1H), 7.98-7.91 (m, 2H), 7.86-7.80 (m, 1H), 7.57-7.50 (m, 1H), 7.38 (dd, J=14.1, 6.2 Hz, 2H), 7.34 (s, 1H), 7.27-7.18 (m, 2H), 7.10 (t, J=8.4 Hz, 1H), 7.07-6.99 (m, 1H), 5.12 (s, 2H). ¹³C NMR (151 MHz, DMSO-d₆) δ 163.3, 162.2 (d, J=243.6 Hz), 159.2, 158.7, 151.2, 149.2, 147.6, 139.8, 139.6 (d, J=7.4 Hz), 139.2, 137.6, 136.4, 134.8, 133.2, 130.5 (d, J=8.3 Hz), 127.6, 123.9, 123.6 (d, J=2.3 Hz), 121.0, 114.8, 114.7, 114.4, 114.3 (d, J=8.2 Hz), 113.9, 112.3, 107.3, 68.8. ^{19F} NMR (376 MHz, DMSO) δ −113.10 (q, J=9.4 Hz). HRMS (ESI) m/z: [M+H]⁺ calcd for C₂₇H₁₉ClFN₄O₄⁺517.1073, found 517.1079.

### 5-(3-(3-Chloro-5-((2,6-difluorobenzyl)oxy)phenyl)-2-oxo-2H-[1,3′-bipyridin]-5-yl)pyr-imidine-2,4(1H,3H)-dione (36)

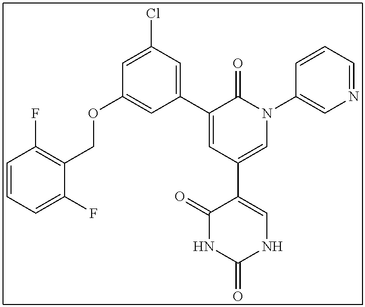 [SMILES: O=c1[nH]cc(-c2cc(-c3cc(Cl)cc(OCc4c(F)cccc4F)c3)c(=O)n(-c3cccnc3)c2)c(=O)[nH]1]

General Procedure E was employed using S11i to afford 36 (51.9 mg, 90% yield). 1H NMR (600 MHz, DMSO-d₆) δ 11.29 (s, 1H), 11.20 (s, 1H), 8.68 (s, 1H), 8.60 (s, 1H), 8.02 (s, 1H), 7.96 (s, 2H), 7.85 (s, 1H), 7.57-7.51 (m, 1H), 7.51-7.45 (m, 1H), 7.43 (s, 1H), 7.34 (s, 1H), 7.12 (dd, J=14.8, 8.6 Hz, 3H), 5.12 (s, 2H). ¹³C NMR (151 MHz, DMSO-d₆) δ 163.2, 161.2 (dd, J=249.4, 7.6 Hz), 159.2, 158.6, 150.9, 149.2, 147.6, 139.8, 139.3, 139.2, 137.6, 136.5, 134.8, 133.3, 132.0 (t, J=10.3 Hz), 127.6, 123.9, 121.3, 114.1, 113.8, 112.2, 112.0, 111.9, 111.8, 111.8, 111.8, 107.4, 58.1. ¹⁹F NMR (376 MHz, DMSO) δ −114.94-−115.06 (m). HRMS (ESI) m/z: [M+H]⁺ calcd for C₂₇H₁₈ClF₂N₄O₄⁺535.0979, found 535.0983.

### 5-(3-(3-Chloro-5-((2-chlorobenzyl)oxy)phenyl)-2-oxo-2H-[1,3′-bipyridin]-5-yl)pyrimidine-2,4(1H,3H)-dione (37)

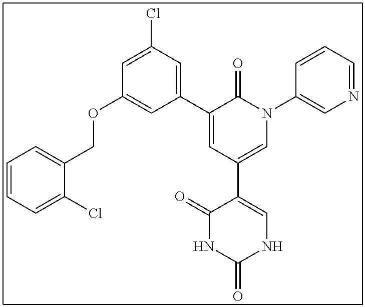 [SMILES: O=c1[nH]cc(-c2cc(-c3cc(Cl)cc(OCc4ccccc4Cl)c3)c(=O)n(-c3cccnc3)c2)c(=O)[nH]1]

General Procedure E was employed using S11j to afford 37 as a pale yellow solid (103.7 mg, 91% yield). 1H NMR (600 MHz, DMSO-d₆) δ 11.26 (br.s, 2H), 8.69 (s, 1H), 8.60 (s, 1H), 8.01 (s, 1H), 7.96 (s, 2H), 7.84 (s, 1H), 7.57-7.53 (m, 2H), 7.46 (d, J=6.0 Hz, 1H), 7.41 (s, 1H), 7.39-7.30 (m, 3H), 7.08 (s, 1H), 5.15 (s, 2H). ³C NMR (151 MHz, DMSO-d₆) δ 163.3, 159.2, 158.8, 151.0, 149.2, 147.6, 139.8, 139.5, 139.2, 137.6, 136.5, 134.8, 133.9, 133.3, 132.9, 130.5, 130.1, 129.5, 127.6, 127.4, 123.9, 121.2, 114.2, 113.8, 112.2, 107.4, 67.4. HRMS (ESI) m/z: [M+H]⁺ calcd for C₂₇H₁₉Cl₂N₄O₄⁺533.0778, found 533.0784.

### 5-(3-(3-Chloro-5-((2-chloro-4-fluorobenzyl)oxy)phenyl)-2-oxo-2H-[1,3′-bipyridin]-5-yl)pyrimidine-2,4(1H,3H)-dione (48)

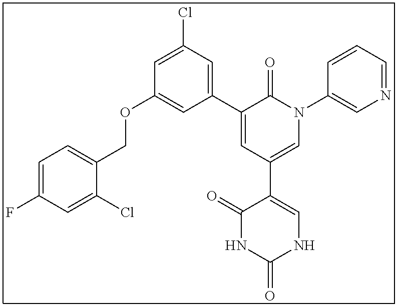 [SMILES: O=c1[nH]cc(-c2cc(-c3cc(Cl)cc(OCc4ccc(F)cc4Cl)c3)c(=O)n(-c3cccnc3)c2)c(=O)[nH]1]

General Procedure E was employed using S11k to afford 48 (52.2 mg, 93% yield). ¹H NMR (600 MHz, DMSO-d₆) δ 11.26 (br.s, 2H), 8.68 (s, 1H), 8.59 (s, 1H), 8.01 (s, 1H), 7.95 (s, 2H), 7.88-7.78 (m, 1H), 7.63 (s, 1H), 7.58-7.50 (m, 1H), 7.46 (d, J=8.0 Hz, 1H), 7.42 (s, 1H), 7.35 (s, 1H), 7.22 (t, J=8.3 Hz, 1H), 7.08 (s, 1H), 5.12 (s, 2H). ¹³C NMR (151 MHz, DMSO-d₆) δ 163.3, 161.9 (d, J=248.3 Hz), 159.2, 158.7, 151.0, 149.2, 147.6, 139.8, 139.5, 139.2, 137.6, 136.4, 134.8, 134.1 (d, J=10.8 Hz), 133.3, 132.3 (d, J=9.1 Hz), 130.4 (d, J=3.0 Hz), 127.6, 123.9, 121.2, 116.9 (d, J=25.2 Hz), 114.6 (d, J=21.0 Hz), 114.2, 113.8, 112.2, 107.4, 66.9. ¹⁹F NMR (376 MHz, DMSO) δ −111.31 (q, J=8.3 Hz). HRMS (ESI) m/z: [M+H]⁺ calcd for C₂₇H₁₈Cl₂FN₄O₄⁺ 551.0684, found 551.0689.

### 5-(3-(3-Chloro-5-((2-chlorobenzyl)oxy)-4-fluorophenyl)-2-oxo-2H-[1,3′-bipyridin]-5-yl)pyrimidine-2,4(1H,3H)-dione (44)

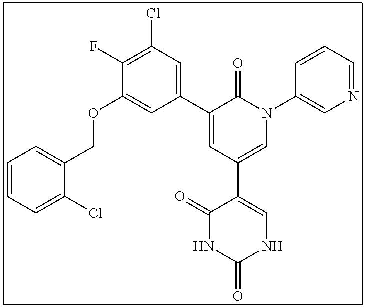 [SMILES: O=c1[nH]cc(-c2cc(-c3cc(Cl)c(F)c(OCc4ccccc4Cl)c3)c(=O)n(-c3cccnc3)c2)c(=O)[nH]1]

General Procedure E was employed using S111 to afford 44 (64.8 mg, 88% yield). ¹H NMR (600 MHz, DMSO-d₆) δ 11.30 (s, 1H), 11.23 (s, 1H), 8.69 (s, 1H), 8.60 (s, 1H), 8.02 (s, 1H), 7.97 (d, J=10.9 Hz, 2H), 7.84 (d, J=2.2 Hz, 1H), 7.64 (d, J=6.9 Hz, 1H), 7.58 (d, J=6.4 Hz, 1H), 7.56-7.52 (m, 2H), 7.50-7.46 (m, 1H), 7.39-7.32 (m, 2H), 5.23 (s, 2H). ¹³C NMR (151 MHz, DMSO-d₆) δ 163.0, 159.0, 150.7, 148.9, 147.3, 146.9 (d, J=247.6 Hz), 139.6, 139.1, 137.3, 136.2, 134.6, 133.1, 132.8, 131.2 (d, J=9.8 Hz), 130.6, 130.1, 129.3, 128.5 (d, J=11.7 Hz), 127.2, 126.8, 123.6, 121.9, 119.5 (d, J=14.8 Hz), 114.5, 111.9, 107.2, 68.4. ¹⁹F NMR (376 MHz, DMSO) δ −137.43 (t, J=6.7 Hz). HRMS (ESI) m/z: [M+H]⁺ calcd for C₂₇H₁₈Cl₂FN₄O₄⁺ 551.0684, found 551.0688.

### 5-(3-(3-Chloro-5-(cyclopropylmethoxy)phenyl)-2-oxo-2H-[1,3′-bipyridin]-5-yl)pyrimidine-2,4(1H,3H)-dione (40)

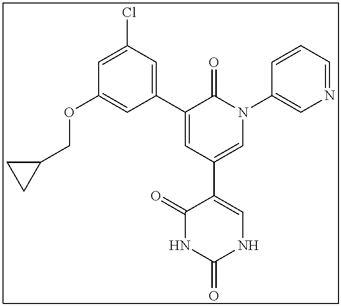 [SMILES: O=c1[nH]cc(-c2cc(-c3cc(Cl)cc(OCC4CC4)c3)c(=O)n(-c3cccnc3)c2)c(=O)[nH]1]

General Procedure E was employed using S11m to afford 40 (64.4 mg, 93% yield). 1H NMR (600 MHz, DMSO-d₆) δ 11.27 (br.s, 2H), 8.68 (s, 1H), 8.60 (s, 1H), 8.00 (s, 1H), 7.95 (d, J=13.0 Hz, 2H), 7.85 (s, 1H), 7.59-7.49 (m, 1H), 7.34 (s, 1H), 7.23 (s, 1H), 6.92 (s, 1H), 3.80 (d, J=4.3 Hz, 2H), 1.16 (s, 1H), 0.50 (d, J=6.0 Hz, 2H), 0.26 (s, 2H). ¹³C NMR (151 MHz, DMSO-d₆) δ 163.2, 159.3, 159.2, 151.0, 149.2, 147.6, 139.7, 139.4, 139.0, 137.6, 136.36, 134.8, 133.2, 127.8, 123.9, 120.5, 113.9, 113.6, 112.2, 107.4, 72.6, 10.0, 3.1. HRMS (ESI) m/z: [M+H]⁺ calcd for C₂₄H₂₀ClN₄O₄⁺ 463.1168, found 463.1172.

### 5-(3-(3-Chloro-5-(3,3,3-trifluoropropoxy)phenyl)-2-oxo-2H-[1,3′-bipyridin]-5-yl)pyrimidine-2,4(1H,3H)-dione (41)

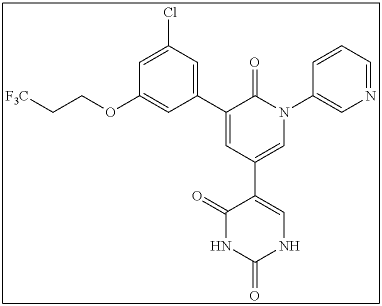 [SMILES: O=c1[nH]cc(-c2cc(-c3cc(Cl)cc(OCCC(F)(F)F)c3)c(=O)n(-c3cccnc3)c2)c(=O)[nH]1]

General Procedure E was employed using S11n to afford 41 (54.4 mg, 87% yield). 1H NMR (600 MHz, DMSO-d₆) δ 11.26 (br.s, 2H), 8.68 (s, 1H), 8.60 (d, J=3.9 Hz, 1H), 8.04-7.98 (m, 1H), 7.96 (s, 2H), 7.84 (s, 1H), 7.54 (dd, J=7.9, 4.9 Hz, 1H), 7.40 (s, 1H), 7.27 (s, 1H), 7.00 (s, 1H), 4.21 (t, J=5.7 Hz, 2H), 2.77-2.69 (m, 2H). ¹³C NMR (151 MHz, DMSO-d₆) δ 163.3, 159.2, 158.4, 151.0, 149.2, 147.6, 139.9, 139.5, 139.2, 137.6, 136.4, 134.9, 133.3, 127.5, 123.9, 121.1, 113.9, 113.6, 112.2, 107.4, 61.4, 32.7 (d, J=27.5 Hz). ¹⁹F NMR (376 MHz, DMSO) δ −63.02 (td, J=11.2, 2.7 Hz). HRMS (ESI) m/z: [M+H]⁺ calcd for C₂₃H₁₇ClF₃N₄O₄⁺505.0885, found 505.0890.

### 2-(3-(3-Chloro-5-(cyclopropylmethoxy)phenyl)-2-oxo-2H-[1,3′-bipyridin]-5-yl)benzonitrile (39)

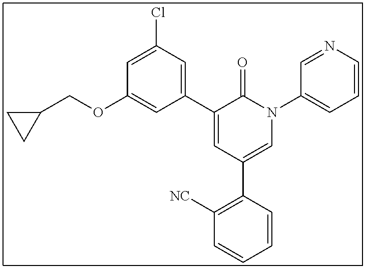 [SMILES: N#Cc1ccccc1-c1cc(-c2cc(Cl)cc(OCC3CC3)c2)c(=O)n(-c2cccnc2)c1]

General Procedure D was employed using S10m (40.3 mg, 0.15 mmol, 1.1 equiv) and S4 (50.0 mg, 0.14 mmol, 1.0 equiv). Purification was accomplished using reverse phase column chromatography (SNAP Ultra C18 60g, gradient=0-70% MeCN/H₂O over 5 CV, then 70-73% MeCN/H₂O over 4 CV) to afford 27.4 mg (43% yield over 2 steps) of a white solid. ¹H NMR (500 MHz, DMSO-d₆) δ 8.80 (d, J=2.4 Hz, 1H), 8.68 (dd, J=4.8, 1.4 Hz, 1H), 8.19 (dd, J=21.5, 2.6 Hz, 1H), 8.08 (dt, J=8.3, 1.9 Hz, 1H), 7.96 (d, J=7.6 Hz, 1H), 7.84 (d, J=7.7 Hz, 1H), 7.78 (td, J=7.7, 1.3 Hz, 1H), 7.63 (dd, J=8.2, 4.8 Hz, 1H), 7.57 (td, J=7.6, 1.2 Hz, 1H), 7.48 (d, J=1.6 Hz, 1H), 7.35 (t, J=1.9 Hz, 1H), 7.01 (t, J=2.1 Hz, 1H), 3.88 (d, J=7.0 Hz, 2H), 1.23 (tt, J=7.5, 4.9 Hz, 1H), 0.63-0.47 (m, 2H), 0.35-0.27 (m, 2H). ¹³C NMR (126 MHz, CDCl₃) δ 160.2, 159.7, 140.0, 139.3, 138.1, 136.6, 134.8, 134.1, 133.6, 132.4, 132.2, 132.2, 131.7, 129.3, 128.8, 128.7, 128.6, 121.2, 118.6, 117.6, 115.6, 113.5, 111.1, 73.3, 10.3, 3.3. HRMS (ESI) m/z: [M+H]⁺ calcd for C₂₇H₂₁ClN₃O₂+454.1317, found 454.1340.

### 2-(3-(3-Chloro-5-(3,3,3-trifluoropropoxy)phenyl)-2-oxo-2H-[1,3′-bipyridin]-5-yl)benzonitrile (47)

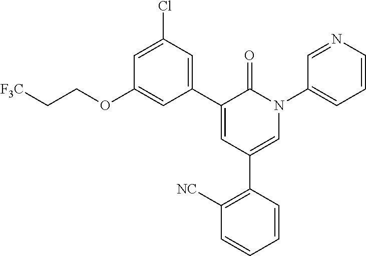 [SMILES: N#Cc1ccccc1-c1cc(-c2cc(Cl)cc(OCCC(F)(F)F)c2)c(=O)n(-c2cccnc2)c1]

General Procedure D was employed using S10n (45.5 mg, 0.15 mmol, 1.1 equiv) and S4 (50.0 mg, 0.14 mmol, 1.0 equiv). Purification was accomplished using normal phase column chromatography (SNAP Ultra 50 g, gradient=0-90% EtOAc/Hex over 8 CV, then 90% EtOAc/Hex over 2 CV, then 90-100% EtOAc/Hex over 2 CV) followed by reverse phase column chromatography (SNAP Ultra C18 60g, gradient=0-70% MeCN/H₂O over 8 CV, then 70% MeCN/H₂O over 3 CV) to afford 43.1 mg (62% yield over 2 steps) of a white solid. ¹H NMR (400 MHz, CDCl₃) δ 8.81 (s, 1H), 8.71 (s, 1H), 7.95 (ddd, J=8.2, 2.5, 1.4 Hz, 1H), 7.84 (d, J=2.6 Hz, 1H), 7.80 (dd, J=7.8, 1.3 Hz, 1H), 7.72-7.66 (m, 2H), 7.55 (d, J=7.8 Hz, 1H), 7.52-7.47 (m, 2H), 7.38 (t, J=1.6 Hz, 1H), 7.31 (t, J=1.8 Hz, 1H), 4.22 (t, J=6.5 Hz, 2H), 2.61 (qt, J=10.5, 6.5 Hz, 2H). ¹³C NMR (151 MHz, CDCl₃) δ 160.1, 158.7, 150.0, 147.4, 140.0, 139.4, 138.3, 137.5, 136.8, 135.0, 134.6, 134.1, 133.6, 131.2, 129.3, 128.6, 125.9 (q, J=276.6 Hz), 124.0, 122.0, 118.6, 117.5, 115.5, 113.4, 111.1, 61.44 (q, J=3.2 Hz), 34.09 (q, J=29.1 Hz). ¹⁹F NMR (376 MHz, CDCl₃) δ −64.73. HRMS (ESI) m/z: [M+H]⁺ calcd for C₂₆H₁₈ClF₃N_{302+496.1034}, found 496.1034.

### II. Assessment of Antiviral Activity Against SARS-CoV-2 and Cellular Cytotoxicity

i. Cells

Vero-E6 were cultured in Dulbecco's Modified Eagle Medium (DMEM) with 10% heat-inactivated fetal bovine serum (FBS), and 1% Penicillin/Streptomycin unless otherwise indicated. For Vero-E6, 5 μg/mL of puromycin (GIBCO) and 5 μg/mL blasticidin (GIBCO) were added as appropriate. Normal human bronchial epithelial cells (16HBE140−) were a kind gift of Dr. Marie Egan, Yale University School of Medicine. Cells were grown in T75 flasks coated with fibronectin, BSA, and Collagen ECM at 37° C. in a humidified incubator with 5% CO₂. Cells are grown in minimum essential media containing 10% FBS and 1% penicillin/streptomycin (Gibco).

ii. Viral stocks

To generate viral stocks, Vero-E6 cells were inoculated with the SARS-CoV-2 isolate USA-WA1/2020 (BEI Resources #NR-52281) at an MOI of 0.01 for three days to generate a P1 stock. The P1 stock was used to inoculate Vero-E6 cells for three days at approximately 50% cytopathic effects. Virus titer was determined by plaque assay using Vero-E6 cells.

iii. Viral Titer Plaque Assay

Vero-E6 cells were seeded at 4×10′ cells/well in 12-well plates and infected for 1 hour with the SARS-CoV-2 isolate USA-WA1/2020 at an MOI of 0.01. The cells were washed twice to remove residual unattached virus. Serial dilutions of each compound (0.1% DMSO in 2% FBS in DMEM media) were added to the cells and incubated at 37° C. (2 dpi). After 2 dpi, the supernatant containing virus was cleared from cell debris at 1000 rpm for 10 min and frozen until analysis via plaque assay.

For the plaque assay, Vero-E6 cells were seeded at 7.5×10′ cells/well in 6-well plates. The following day, the media was removed and replaced with 100 μL of 10-fold serial dilutions of previously frozen viral supernatant. Plates were incubated at 37° C. for 1 hour with gentle rocking. Subsequently, overlay media (DMEM, 2% FBS, 0.6% Avicel R^{C}-581) was added to each well. At 2 dpi for SARS-CoV-2 plates were fixed with 10% formaldehyde for 30 min, stained with crystal violet solution (0.5% crystal violet in 20% ethanol) for 30 min, and then rinsed with deionized water to visualize plaques.

iv. Antiviral Assay and Cellular Cytotoxicity Using MTT

The antiviral activity of compounds was examined by evaluating the cytopathic effect in Vero-E6 cells grown at 37° C. in a 5% CO₂ atmosphere for 72 h using 96 multi-well plates (50,000 cells/well) using 3-(4,5-dimethyl-2-thiazolyl)-2,5-diphenyl-2H-tetrazolium bromide (MTT; Sigma-Aldrich) method according to the manufacturer's instructions. Cells were challenged with SARS-CoV-2 at a multiplicity of infection (MOI) of 0.01. The virus was added together with the compound(s) under investigation and incubated in DMEM supplemented with 2% FBS and using 0.1% DMSO with no inhibitor as a control. To assess in vitro antiviral activity, serial dilutions of compounds in (0.1% DMSO in 2% FBS in DMEM media) were made in a concentration range of 0.1 μM to 25 μM. Optical densities were measured at 560/620 nm with a Spectramax Plate Reader. Three independent experiments with triplicate measurements were performed. Data were analyzed by a four-parameter curve-fitting from a dose-response curve using GraphPad Prism (version 7.00) to calculate the EC₅₀ (concentration of the compound that inhibited 50% of the infection) based on the MTT method. Concurrently in this experiment, general cellular cytotoxicity in the absence of virus was determined. The MTT assay was also used to assess compound cytotoxicity in human normal bronchial epithelial cells.

v. Quantification and Statistical Analysis

Statistical significance was determined as p<0.05 using GraphPad Prism 7 unless otherwise indicated. Experiments were analyzed by unpaired two-tailed t tests, Mann-Whitney test, or ANOVA, as indicated.

vi. Replicon Assay to Examine Drug Combination Synergy

A SARS-CoV-2 replicon was generated by replacing the Spike gene with a Nano luciferase (Nluc) gene within a full-length infectious cDNA clone of the viral genome stably maintained within the yeast artificial chromosome (YAC) pCC1BAC-HIS3/SARS-CoV-2 (PMID: 32365353). Briefly, the Nluc gene was amplified to add flanking SARS-CoV-2 sequences and remove an internal EagI site in two steps. First, the 5′ end of the Nluc gene was amplified by using Q5 DNA polymerase (New England Biolabs) with primers YO-3778 (5′-GAG TTG TTA TTT CTA GTG ATG TTC TTG TTA ACA ACT AAA CGA ACA ATG GTC TTC ACA CTC GAA GAT TT-3′, SEQ ID NO: 1) and YO-4096 (5′-GCC TTC ATA GGG GCG TCC GAA ATA GTC GAT-3′, SEQ ID NO: 2); the 3′ end of the Nluc gene was amplified by using Q5 DNA polymerase with primers YO-4101 (5′-CGA CTA TTT CGG ACG CCC CTA TGA AGG CAT CGC CGT GTT-3′, SEQ ID NO: 3) and YO-3779 (5′-CAG TTC CAA TTG TGA AGA TTC TCA TAA ACA AAT CCA TAA GTT CGT TTA CGC CAG AAT GCG TTC GCA CA-3′, SEQ ID NO: 4). The full-length Nluc gene was then amplified by using Q5 DNA polymerase with primers YO-3778 and YO-3779, and inserted into BamHI-linearized pCC1BAC-HIS3/SARS-CoV-2 by contransfection into yeast VL6-48N (PMID: 9207100) and selection of homologous recombinants on histidine-deficient media. YACs were recovered from liquid cultures by treatment with zymolase (Zymo Research) and ZymoPURE plasmid midiprep kits (Zymo Research), then transformed into Epi300 bacterial cells (Lucigen). Chloramphenicol-resistant colonies were picked and grown in liquid media with CopyControl induction solution, then used to prepare amplified YAC by using the ZymoPURE plasmid midiprep kit.

The replicon-bearing YAC was sequence verified by whole plasmid sequencing (Massachusetts General Hospital Genome DNA Core) and linearized by overnight digestion with EagI. Linearized transcription templates were purified by treatment at 55° C. with 0.5% (W/V) SDS and 3 units proteinase K followed by two rounds of phenol/chloroform extraction and ethanol precipitation. Replicon RNAs were transcribed from purified transcription templates with the T7 Ribomax kit (Promega) and anti-reverse cap analog (New England Biolabs). RNAs were purified by treatment with RQ1 DNase (Promega) and RNA Clean & Concentrator-25 kit (Zymo), eluted into 2 mM sodium citrate (pH 6.4), aliquoted in 1 μg portions, and stored frozen at −80° C. RNAs were transfected into BHK cells engineered to express a human codon-optimized SARS-CoV-2 nucleoprotein gene by electroporation (PMID: 9371625). Cells were then seeded in multiple replicates on 96-well plates containing serial dilutions of 5 and/or remdesivir. The following day, Nluc activity was measured by using NanoGlo reagents (Promega) with a CentroXS3 LB 960 microplate luminometer (Berthold).

vii. Data Analysis of Drug Combination Synergy with Remdesivir

The combination inhibitory effects of compound 5 and remdesivir were tested in a 2-drug combination using replicon assay. Remdesavir has the following structure:

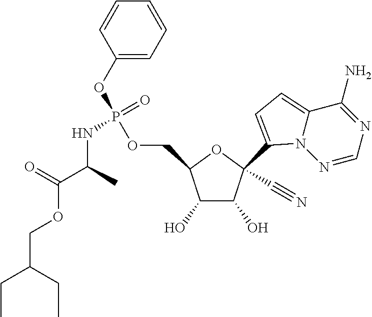 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

The combination inhibitory data were analyzed using MacSynergy II 3D plots. The resulting surface plots of the data reflect the difference between the experimental dose-response surface and the predicted additive surface. On a 3D model, a simple additive effect will result in a horizontal plane at 0% inhibition, whereas a synergistic or antagonistic effect will render a peak or depression above or below the horizontal plane. The volumes of the peaks/depressions were then calculated to quantify the effect of the drug combination on antiviral activity [synergy/antagonism volumes (μM²%)]. For these studies, synergy volumes are divided into the following categories: minor but significant synergy: 25-50 μM²%; moderate synergy: 50-100 μM²%; strong synergy: over 100 μM²%; minor antagonistic: −50 and −100 μM²%; and strong antagonistic: less than −100 μM²%.

viii. Data Analysis of Drug Combination Synergy with Molnupiravir/EIDD-1931

The combination inhibitory effects of compound 61 and molnupiravir were tested in a 2-drug combination using replicon assay. Molnupiravir is metabolized in vivo into ribonucleoside analog EIDD-1931. The structures of molnupiravir and EIDD-1931 are shown below:

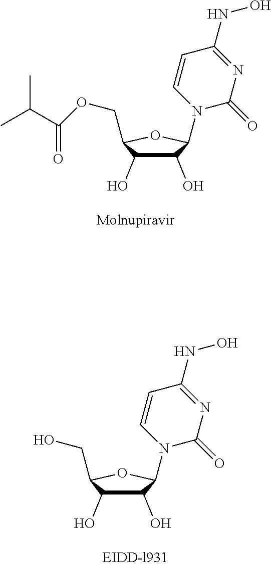

The combination inhibitory data were analyzed using MacSynergy II 3D plots. The resulting surface plots of the data reflect the difference between the experimental dose-response surface and the predicted additive surface. On a 3D model, a simple additive effect will result in a horizontal plane at 0% inhibition, whereas a synergistic or antagonistic effect will render a peak or depression above or below the horizontal plane. The volumes of the peaks/depressions were then calculated to quantify the effect of the drug combination on antiviral activity [synergy/antagonism volumes (μM²%)]. For these studies, synergy volumes are divided into the following categories: minor but significant synergy: 25-50 μM²%; moderate synergy: 50-100 μM²%; strong synergy: over 100 μM²%; minor antagonistic: −50 and −100 μM²%; and strong antagonistic: less than −100 μM²%. As shown in FIGS. 6A-6B, EIDD-1931 has a calculated IC₅₀ of 2.229 μM, and compound 9 has an IC₅₀ of 0.0938 μM. The combination of EIDD-1931 and compound 61 unexpectedly shows a synergistic effect in anti-viral efficacy. As shown in FIG. 6, a range of combinations of compound 61 and EIDD-1931 exhibit values significantly above the plane in the 3D plot.

vi. Crystallographic Data
I. Purification and Crystallization of SARS-CoV-2 M^{pro} Recombinant SARS-CoV-2 M^{pro} with native N- and C-termini was expressed and purified as previously described. Pure protein was buffer exchanged (20 mM Tris, 150 mM NaCl, 1 mM EDTA, 1 mM DTT, pH 7.8) and stored at −80° C. at 20 mg/mL. Samples were thawed on ice and subsequently incubated at 37° C. with 2 mM compound in DMSO for 30 minutes prior to centrifugation at 10,000×g. The supernatant was used to set up crystallization screens with the commercially available PEGRx1 and PEGRx2 screens (Hampton Research). Crystal screens were set up manually with 2 μL drops with a 1:1 protein/reservoir solution ratio to equilibrate with 80 μL reservoir solution using the sitting-drop vapor-diffusion method at 18° C. Plate-like or small, three-dimensional crystals appeared overnight with most compounds in several conditions. Crystals were harvested, cryo-protected with 54 glycerol and flash frozen in liquid nitrogen.
ii. Structure Determination of SARS-CoV-2 M^{pro} Bound to Ligand

Diffraction data was collected at the 24-ID-E and 24-ID-C beamlines at the Advanced Photon Source and AMX beamline at the National Synchrotron Light Source II. Datasets were indexed using the XDS software. Molecular replacement was performed using PHASER with previously solved structures from the PDB. 6Y2F was used as the search model for the structure of Mpro:Mpro4 (7L10), 6Y2E was used as the search model for the structure of Mpro:Mpro-14 (7L12), and 6Y2G was used as the search model for Mpro:Mpro5, 21, and 26 (7L11, 7L13, and 7L14, respectively). Models were built using COOT and successive rounds of refinement performed with Phenix Refine. Diffraction data processing and refinement statistics are found in Table 5. Crystallography software was compiled by SBGrid.

TABLE 5
Diffraction data and refinement statistics.

| Complex | Mpro:4 | | Mpro:5 | | Mpro:14 | | Mpro:21 | | Mpro:26 | |
|---|---|---|---|---|---|---|---|---|---|---|
| PDB Code | 7L10 | | 7L11 | | 7L12 | | 7L13 | | 7L14 | |
| Data Collection | | | | | | | | | | |
| X-Ray Source | NSLS-II | | APS 24ID-E | | APS 24ID-E | | NSLS-II | | NSLS-II | |
| | 17-ID-1 | | | | | | 17-ID-1 | | 17-ID-1 | |
| Wavelength, Å | 0.920097 | | 0.97918 | | 0.97918 | | 0.920106 | | 0.920106 | |
| Space Group | C2 | | P2₁2₁2₁ | | C2 | | P2₁2₁2₁ | | P2₁2₁2₁ | |
| Cell | | | | | | | | | | |
| Dimensions | | | | | | | | | | |
| a, b, c (Å) | 97.53, 80.98, | | 67.91, 101.23, | | 115.70, 54.02, | | 67.81, 101.22, | | 68.11, 100.60, | |
| | 54.53 | | 103.86 | | 44.52 | | 104.04 | | 104.02 | |
| α, β, γ (°) | 90.00, 116.58, | | 90.00, 90.00, | | 90.00, 101.26, | | 90.00, 90.00, | | 90.00, 90.00, | |
| | 90.00 | | 90.00 | | 90.00 | | 90.00 | | 90.00 | |
| Resolution (Å) | 29.63-1.63 | | 103.82-1.80 | | 56.84-1.80 | | 29.53-2.17 | | 29.58-1.66 | |
| | (1.73-1.63) | | (1.90-1.80) | | (1.91-1.80) | | (2.30-2.17) | | (1.75-1.66) | |
| R_{merge} | 0.087 | (0.592) | 0.093 | (0.50) | 0.107 | (0.654) | 0.111 | (0.724) | 0.121 | (0.595) |
| I/σI | 7.16 | (1.25) | 15.32 | (3.67) | 8.27 | (1.92) | 16.59 | (3.85) | 14.94 | (2.80) |
| Completeness (%) | 96.6 | (91.3) | 99.1 | (97.0) | 95.2 | (91.7) | 99.6 | (98.3) | 95.0 | (74.6) |
| Redundancy | 3.0 | (2.6) | 6.90 | (6.75) | 2.6 | (2.5) | 13.6 | (13.7) | 11.80 | (6.00) |

| | | | | | |
|---|---|---|---|---|---|
| Refinement | | | | | |
| Resolution (Å) | 29.63-1.63 | 72.46-1.80 | 56.84-1.80 | 29.53-2.17 | 29.54-1.80 |
| No. reflections | 45,256 | 66,725 | 47,023 | 38,761 | 66,679 |
| R_{work}/R_{free} | 0.2243, 0.2507 | 0.2051, 0.2316 | 0.1911, 0.2491 | 0.1967, 0.2476 | 0.1757, 0.2044 |
| No. atoms | | | | | |
| Protein | 2346 | 4731 | 2358 | 4687 | 4731 |
| Inhibitor | 29 | 64 | 36 | 37 | 66 |
| Water | 239 | 403 | 329 | 308 | 591 |
| B-factors | | | | | |
| Protein | 29.27 | 22.85 | 25.98 | 38.53 | 17.83 |
| Inhibitor | 29.02 | 22.98 | 28.38 | 36.67 | 17.41 |
| Water | 37.19 | 32.68 | 35.86 | 42.4 | 27.71 |
| R.m.s deviations | | | | | |
| Bond lengths (Å) | 0.007 | 0.006 | 0.007 | 0.006 | 0.006 |
| Bond angles (°) | 0.885 | 0.86 | 0.918 | 0.86 | 0.82 |
| Ramachandran | | | | | |
| Favored (%) | 99 | 97.86 | 98.02 | 96.18 | 97.86 |
| Allowed (%) | 1 | 1.81 | 1.65 | 3.82 | 2.14 |
| Outliers (%) | 0 | 0.33 | 0.33 | 0 | 0 |

Preliminary PK studies in CB C₅₇BL/6 (BJ6) mice after 15 mg/kg of Compound 61 dosed oral & IV determined Compound 61 levels 7-fold higher than EC₉₀ (−600 nM) at 24 hr time point for IV and oral doses. Extracted lung tissues showed Compound 61 levels after 24 hr IV dose of about 3-fold higher than EC₉₀. Confirmed serum and lung tissue extracts inhibition of SARS-CoV-2 replication in cell culture.

### Solubility Assay

The thermodynamic solubilities of compounds of formula I or formula I-A and piroxicam as control were measured following the procedure described before. The original method was adapted to a 96-well plate setting to facilitate fast absorbance readouts through a microplate reader.

### Materials and Methods

### 1. Samples and Chemicals:

All the tested compounds were synthesized in-house; piroxicam (>98% (TLC)) was purchased from Sigma Aldrich.

### 2. Buffer Solutions Used for the Solubility Experiments:

Britton-Robinson (BR) buffer pH 6.5 was used for all the tested compounds and BR buffer pH 3.7 for the control compound. BR buffers comprise acetic, phosphoric, and boric acid, each at 0.04 M. The buffers were prepared using a 0.2 M NaOH solution to adjust the mixture to the desired pH.

### 3. Apparatus:

The pH of the buffer solutions was measured using Orion Star A 111 pH meter and Orion 8102BNUWP Ross Ultra, Glass body, combination pH electrode. A Corning PC 620 magnetic stirrer was used to mix the two phases. Samples were filtered using an Agilent Syringe filter (0.45 μm pore size, 25 mm). All the samples were measured in Greiner Bio-One UV-Star™ 96-Well UV Spectroscopy Microplates. The absorbance of the samples was measured using TECAN Spark microplate reader.

### 4. Experimental Details and Setup:

All the solubility experiments were conducted at 21-22° C. The equilibrium solubility of the un-ionized form of the samples was determined by the shake-flask method.

The experiment was comprised of two parts: a) calibration and b) measurement. The calibration and measurement (after 48 h of stirring) were performed on the same day. A control sample was measured for every batch of tested compounds.

### A. Calibration.

For the quantitative determination of the aqueous solubility, a calibration curve had to be constructed for each compound: a stock solution of 1 mg/mL in DMSO was prepared and was then mixed with BR buffer (1:1 DMSO:BR buffer) in order to obtain 10 serial dilutions from 500 μg/mL to 0.98 μg/mL. 2 independent series of serial dilutions were prepared, and each concentration was measured in duplicate (2× quadruplicate measurements) for each series in a 96-well plate (200 μL per well). A mixture of BR buffer and DMSO (1:1 ratio) was used as blank, BR buffer pH 6.5 was used for the tested compounds and BR buffer pH 3.7 for the control.

Once the plate was prepared, an absorbance scan was performed, covering the full range of wavelengths from 200-800 nm. The results were processed via excel and Prism 8.0.0, to allow averaging, blank subtraction and manual baseline correction were performed (λ=800 nm was considered as the baseline). The wavelength of maximum absorption for each compound, λₘₐₓ, was then determined from its absorption spectrum. Subsequently, the data of the absorbance at different concentrations, at λₘₐₓ, were fit in a model using linear regression. Outliers were eliminated to allow for the best fit, resulting in the equation to describe the relation between absorbance (A) and concentration (C), according to Lambert-Beer law, A=ebC.

### B. Measurement.

1-2 mg of each tested compound were suspended in BR buffer pH 6.5 (1 mg/mL) in a 1-dram glass vial, equipped with a stir bar. The mixture containing solid excess of the tested compound was stirred for 48 h before separating saturated solution and precipitate by filtration.

After filtration, the saturated solution was divided in different vials, and each one was mixed with DMSO (1:1 ratio). The samples were used to fill the wells of a 96-well plate, with 200 μL per well, such that two or three measurements (in triplicate or quadruplicate) could be carried out in parallel. Again, a mixture of BR buffer and DMSO (1:1 ratio) was used as blank (2× quadruplicate measurements). The concentration of the measured samples was determined using their absorption at λₘₐₓ, and the equation that was determined for each compound previously (as described in part A). The measured values were corrected for a dilution factor of 2.

### PAMPA Assay

PAMPA experiments were performed in a 96-well 0.45 mm filter plate containing hydrophobic PVDF membranes (Millipore). The artificial membrane was formed by adding 5 mL of a solution containing 4% lecithin (Sigma P5638) in dodecane to each filter. 200 mL of donor solutions containing saturated or up to 500 mM concentrations of compound in PBS pH 7.4 and 5% DMSO were added to the top donor wells. 300 ml of PBS pH7.4 buffer were added to the receiving wells of the acceptor plate. After the stacked donor and acceptor plates were incubated at room temperature for 18 hours, the concentrations of compound in the acceptor solutions were measured by a multi-wavelength UV plate reader. Experiments were performed in duplicate for each compound. An equilibrium standard was made for each compound containing 40% concentration of the donor solution to represent the concentration of the acceptor solution if full permeability was achieved. The apparent permeability of each compound was calculated by the following equation:

$$PPAMPA = ( ( {V}_{D} \times {V}_{A} ) / ( ( {V}_{D} + {V}_{A} ) \times S \times t ) ) \times ( - ln ( 1 - ( {OD}_{A} / {OD}_{E} ) ) ) cm / s$$

Where V_{D} and V_{A} are the volumes of the donor and acceptor solutions, S is the membrane surface area, t is the time of incubation, and OD_{A} and OD_{E} are the absorbances of the acceptor solution and equilibrium standards, respectively.

TABLE 5
Measured PAMPA Permeabilities (in 10⁻⁶ cm/s)and
Aqueous Solubilities (in μg/mL)

| compd | PAMPA | aq. sol. |
|---|---|---|
| 39 | 4.27 ± 0.07 | 3 ± 2 |
| 48 | 4.0 ± 1.9 | 25.0 ± 0.1 |
| 50 | 3.04 ± 0.09 | 13.1 ± 0.3 |
| 49 | ND | 7.6 ± 0.7 |
| 47 | 0.24 ± 0.02 | 167.7 ± 1.1 |
| 62 | 0.57 ± 0.09 | ND |
| 57 | 3.25 ± 0.46 | 14.2 ± 0.7 |
| 60 | 2.26 ± 0.14 | 57.0 ± 0.4 |
| 43 | 0.73 ± 0.05 | 21.8 ± 0.9 |
| 61 | 1.50 ± 0.59 | 116.5 ± 8.8 |
| 65 | 1.03 ± 0.18 | 34.5 ± 0.3 |
| 64 | 1.16 ± 0.32 | 24.8 ± 1.4 |

As shown in Table 5, the permeabilities of the three cyanophenyl-containing compounds (39, 48, and 50) are good (>3×10⁻⁶ cm/s), while they are low (<0.8×10-6 cm/s) for the three unmethylated uracil-containing compounds (47, 62, and 43). N-Methylation significantly improved the permeabilities, such that those of 57, 60, 61, 65, and 65 are all above 1×10⁻⁶ cm/s.

The results for 47, 62, and 65 are interesting: the permeability of 47 doubles upon replacement of the methyl group by a trifluoromethyl group on the oxazole ring and then doubles again upon N-methylation of the uracil ring to a value of 1.03×10⁻⁶ cm/s for 65. Similarly, the permeability of 43 doubles to 1.50×10⁻⁶ cm/s for N-methylated 61. As discussed herein, a PAMPA result above ca. 0.7×10⁻⁶ cm/s appears to be necessary for a compound to show activity in the Vero E6 cell assays.

Aqueous solubilities were also measured for 11 compounds, as reported in Table 5, using a standard shake-flask procedure in Britton-Robinson buffer at pH 6.5. With the exception of 39, the results are all in the range observed for oral drugs. Compound 47 is the most soluble compound but has low permeability, while the final compounds 57, 60, and 61, 64, and 65 show both acceptable solubility and permeability. The curiously much greater solubility of 47 versus its isomer 49 was fully reproducible and consistent with observations from handling the compounds; it may arise from significantly different arrangements in the crystalline state with intermolecular hydrogen bonding retarded by the methyl group adjacent to the azole nitrogen atom in 47.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a compound of formula (I) or formula (I-A), or a salt, solvate, enantiomer, diastereomer, tautomer, or N-oxide thereof:

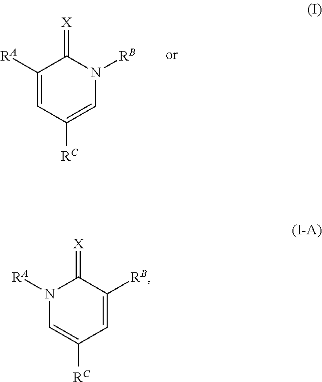

wherein:
each occurrence of R^{A} is independently a 5, 6, 7, or 8-membered heterocyclyl;
each occurrence of R^{B} is independently a 5, 6, 7, 8, 9 or 10-membered heterocyclyl;
each occurrence of R^{C} is independently a 5, 6, 7, or 8-membered heterocyclyl; and
each occurrence of X is independently O, S, or N—OR;
wherein each of R^{A}, R^{B}, and R^{C} is independently substituted by 1 to 5 substituents independently selected from the group consisting of hydrogen, C₆₋₁₄ aryl, C₆₋₁₄ heteroaryl, C₁-alkoxy, C₁₋₁₀ alkyl, C₃₋₁₀ cycloalkyl, C₃₋₁₀ cycloheteroalkyl, C₂₋₁₀ alkenyl, C₂₋₁₀ alkynyl, OR, OC(O)N(R)₂, OCH₂C(O)N(R)₂, O (oxo), F, Cl, Br, I, NO₂, CN, CF₃, OCF₃, N(R)₂, SR, SOR, SO₂R, SO₂N(R)₂, SO₃R, C(O)R, C(O)OR, OC(O)R, C(O)N(R)₂, and combinations thereof; and
wherein each occurrence of R is independently hydrogen, C₁₋₁₂ alkyl, C₂₋₁₂ alkenyl, C₂₋₁₂ alkynyl, C₅₋₁₂ cycloalkyl, C₆₋₁₀ aryl, C₅₋₁₀ heteroaryl, or combinations thereof.

Embodiment 2 provides the compound of embodiment 1, wherein:
X is O;
R^{A} is a 6-membered aryl or heteroaryl;
R^{B} is a 6-membered aryl, 6-membered heteroaryl, 10-membered aryl, or 10-membered heteroaryl; and
R^{C} is a 6-membered cycloheteroalkyl, aryl, or heteroaryl.

Embodiment 3 provides the compound of any one of embodiments 1-2, wherein R^{A} is:

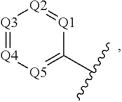

wherein Q1 is C-A¹ or N, Q2 is C-A² or N, Q3 is C-A³ or N, Q4 is C-A⁴ or N, Q5 is C-A⁵ or N, wherein 0-3 of Q1-Q5 can be N,
wherein each A¹-A⁵ is independently selected from the group consisting of hydrogen, C₆₋₁₄ aryl, C₆₋₁₄ heteroaryl, C₁₋₁₀ alkoxy, C₃₋₁₀ cycloalkyl, C₃₋₁₀ cycloheteroalkyl, C₁₋₁₀ alkyl, C₂₋₁₀ alkenyl, C₂₋₁₀ alkynyl, OR, OC(O)N(R)₂, F, Cl, Br, I, NO₂, CN, CF₃, OCF₃, N(R)₂, SR, SOR, SO₂R, SO₂N(R)₂, SO₃R, C(O)R, C(O)OR, OC(O)R, C(O)N(R)₂, and combinations thereof.

Embodiment 4 provides the compound of any one of embodiments 1-3, wherein R^{A} is:

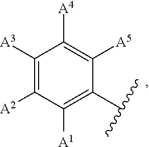

and one of the following applies:
i. A¹, A³, A⁴, and A⁵ are hydrogen,
ii. A¹ and A⁵ are hydrogen, or
iii. A¹, A³, and A⁵ are hydrogen.

Embodiment 5 provides the compound of any one of embodiments 1-4, wherein each A¹-A⁵ is independently selected from the group consisting of hydrogen, C₆₋₁₄ aryl, C₁₋₁₀ alkoxy, C₃₋₁₀ cycloalkyl, C₃₋₁₀ cycloheteroalkyl, C₁₋₁₀ alkyl, OR, OC(O)N(R)₂, OCH₂C(O)N(R)₂, F, Cl, Br, NO₂, CN, CF₃, OCF₃, and combinations thereof.

Embodiment 6 provides the compound of any one of embodiments 1-5, wherein each A¹-A⁵ is independently selected from the group consisting of hydrogen, F, Cl, CN, OC₁₋₆ alkyl, OC₁₋₆ alkyl substituted by 1 to 5 hydroxyl groups, OC₁₋₆ alkyl substituted by 1 to 5 CF₃ groups, O(CH₂)ₙPh, O(CH₂)ₙAr, and O(CH₂CH₂O)ₘCH₃, wherein
each n is independently at each occurrence an integer from 1 to 5;
each m is independently at each occurrence an integer from 1 to 5;
Ar is phenyl substituted with 1 to 5 substituents selected from the group consisting of C₁₋₅ hydrocarbyl, CF₃, F, Cl, Br, and combinations thereof, or
Ar is a 5-membered heteroaryl or a 6-membered heteroaryl substituted with 1 to 5 substituents selected from the group consisting of hydrogen, C₁₋₅ hydrocarbyl, CF₃, F, Cl, Br, and combinations thereof.

Embodiment 7 provides the compound of any one of embodiments 1-6, wherein R^{A} is independently selected from the group consisting of:

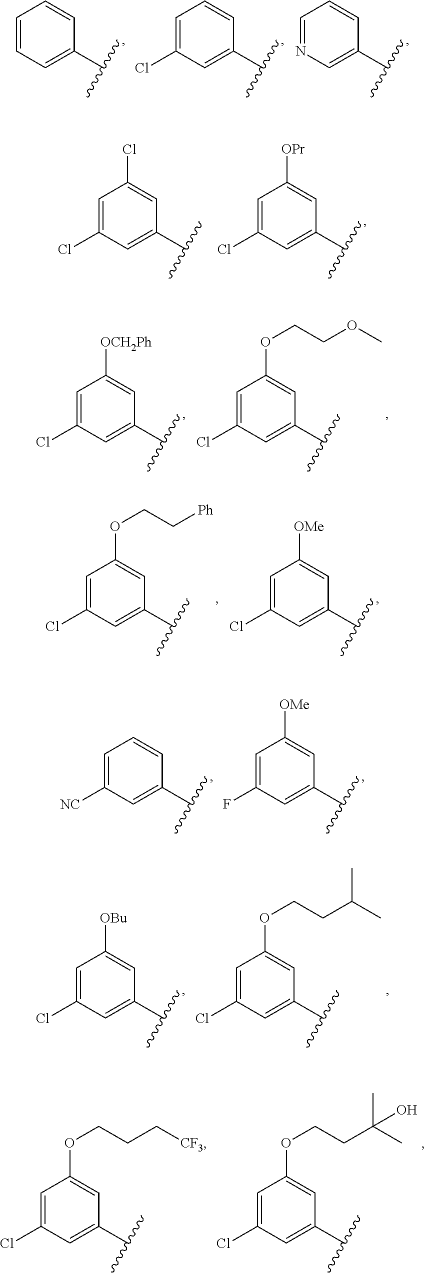

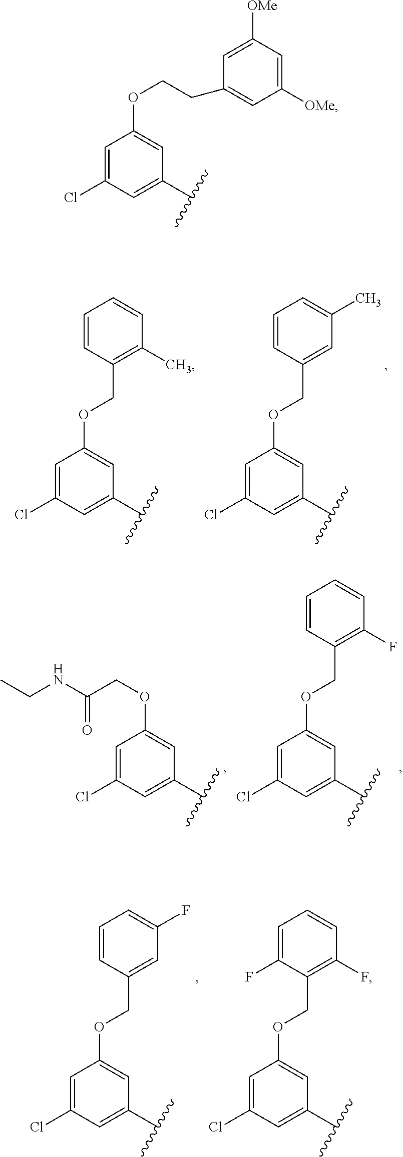

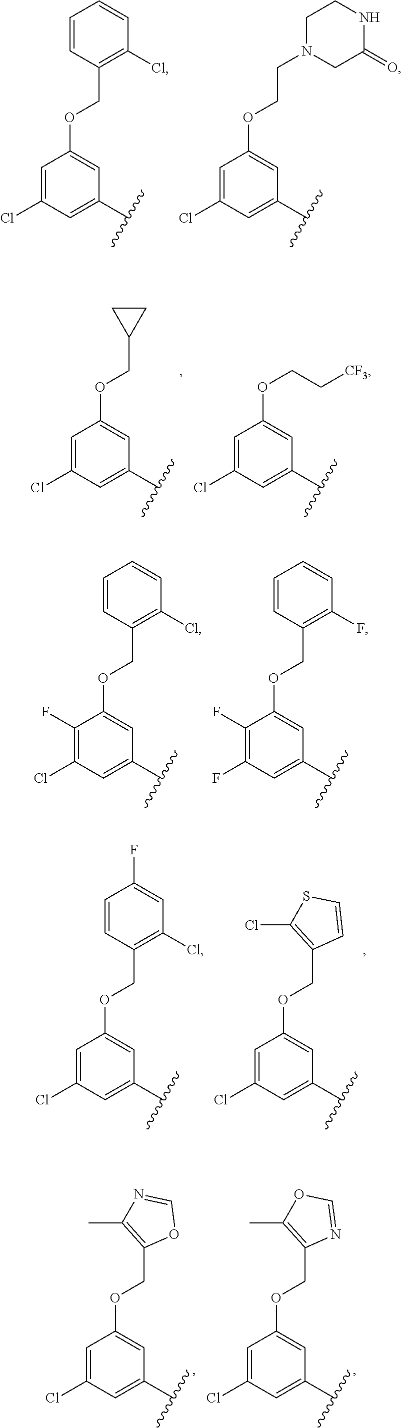

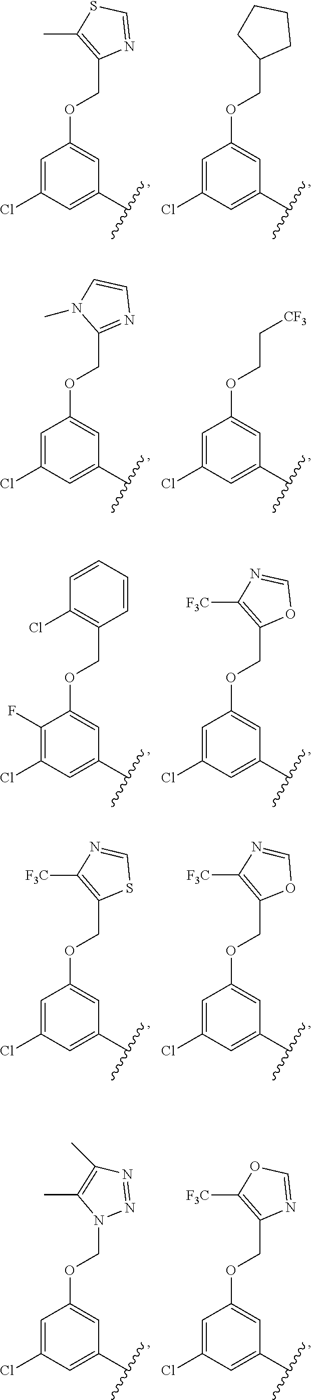

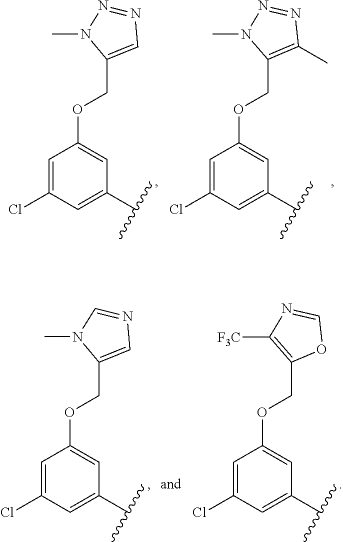

Embodiment 8 provides the compound of any one of embodiments 1-7, wherein R^{B} is

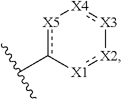

wherein
each X1-X5 is independently C—Y, N, or NR;
each Y is independently selected from the group consisting of hydrogen, O (oxo), F, Cl, C₁₋₄ alkyl, and OC₁₋₄ alkyl;
[symbol] is a single or double bond; and provided that
1 to 3 of X1-X5 is N or NR, and
if at least one of X1-X5 is NR then an adjacent position to the NR is C═O.

Embodiment 9 provides the compound of any one of embodiments 1-8, wherein R^{B} is is

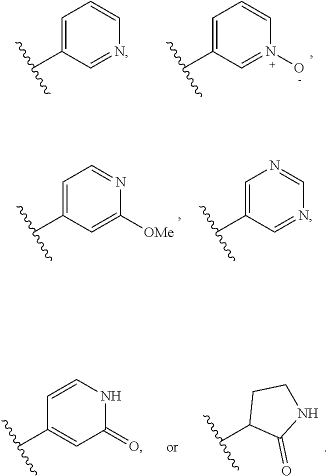

Embodiment 10 provides the compound of any one of embodiments 1-9, wherein R^{B} is

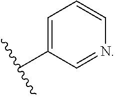 [SMILES: *c1cccnc1]

Embodiment 11 provides the compound of any one of embodiments 1-10, wherein R^{B} is

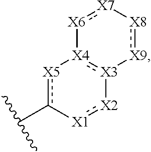

wherein
each X1, X2, X5, X6, X7, X8, and X9 is independently C—Y, N, or NR;
each X3 and X4 is independently C—Y or C;
each Y is independently selected from the group consisting of hydrogen, O (oxo), F, Cl, C₁₋₄ alkyl, and OC₁₋₄ alkyl;
[symbol] is a single or double bond; and
provided that
1 to 3 of X1, X2, X5, X6, X7, X8, and X9 is N or NR, and
if at least one of X1, X2, X5, X6, X7, X8, and X9 is NR then an adjacent position to the NR is C═O.

Embodiment 12 provides the compound of any one of embodiments 1-11, wherein R^{B} is

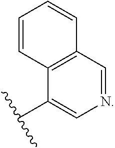 [SMILES: *c1cncc2ccccc12]

Embodiment 13 provides the compound of any one of embodiments 1-12, wherein R^{C} is

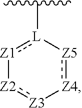

wherein
each Z1-Z5 is independently C—Y, N, or NR;
L is C or N;
each Y is independently selected from the group consisting of hydrogen, O (oxo), F, Cl, Br, I, NO₂, NR₂, CN, CF₃, OCF₃, C₁₋₄ alkyl, and OC₁₋₄ alkyl;
[symbol] is a single or double bond; and
provided that
1 to 3 of Z1-Z5 is N or NR, and
if at least one of Z1-Z5 is NR then an adjacent position to the NR is C═O.

Embodiment 14 provides the compound of any one of embodiments 1-13, wherein R^{C} is selected from the group consisting of

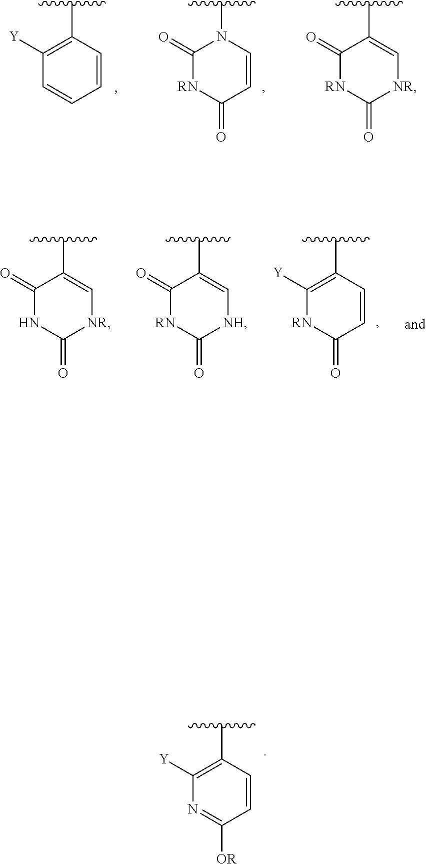

Embodiment 15 provides the compound of any one of embodiments 1-14, wherein Y is CN.

Embodiment 16 provides the compound of any one of embodiments 1-15, wherein R^{C} is selected from the group consisting of

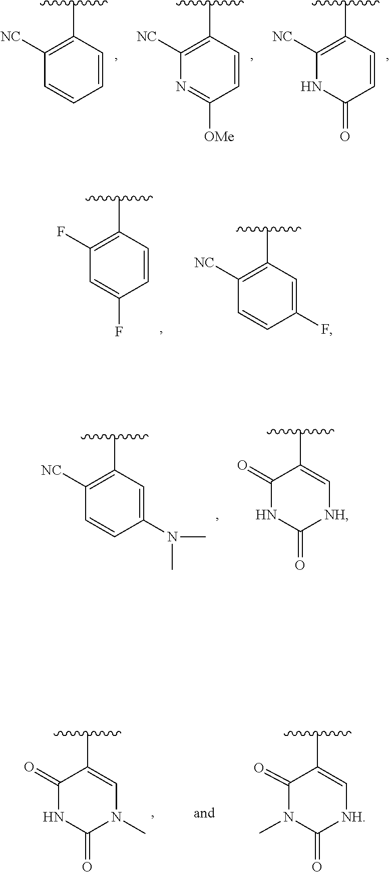

Embodiment 17 provides the compound of any one of embodiments 1-16, which is selected from the group consisting of.

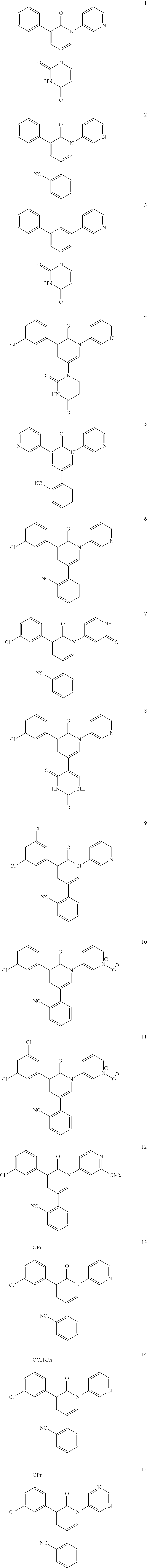

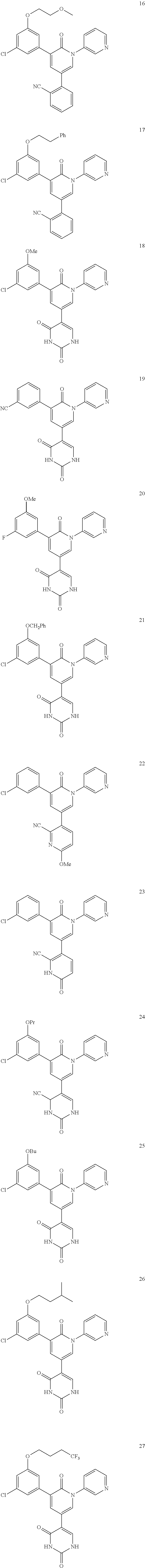

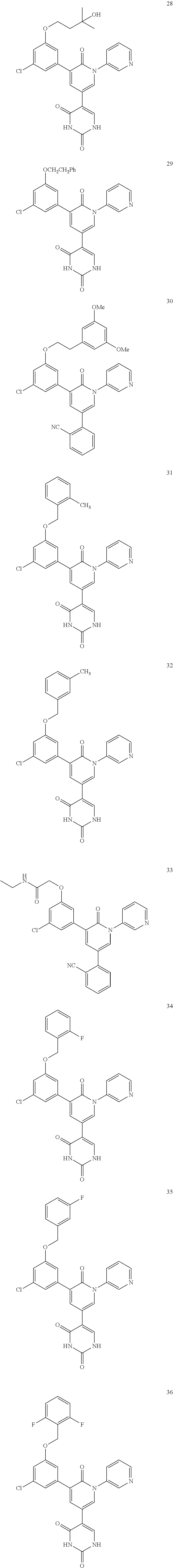

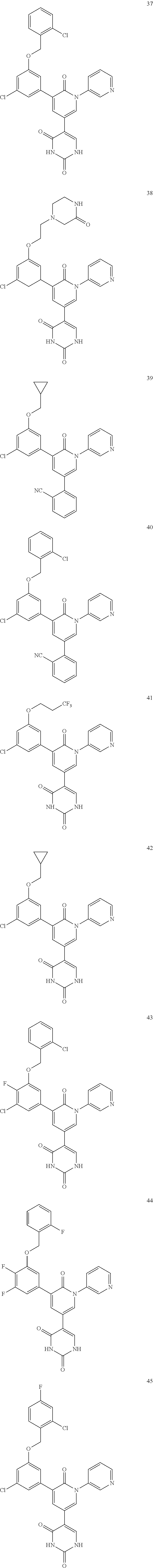

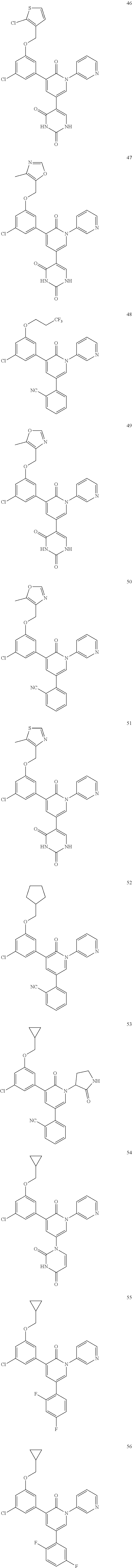

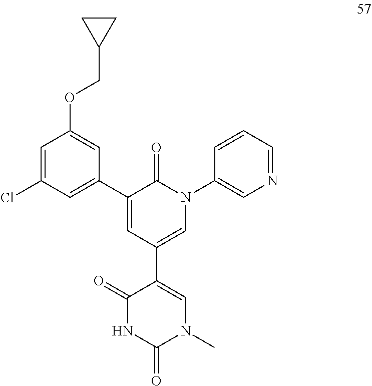 [SMILES: Cn1cc(-c2cc(-c3cc(Cl)cc(OCC4CC4)c3)c(=O)n(-c3cccnc3)c2)c(=O)[nH]c1=O]

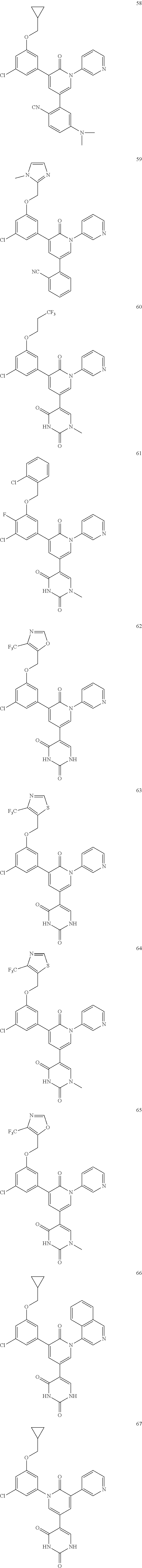

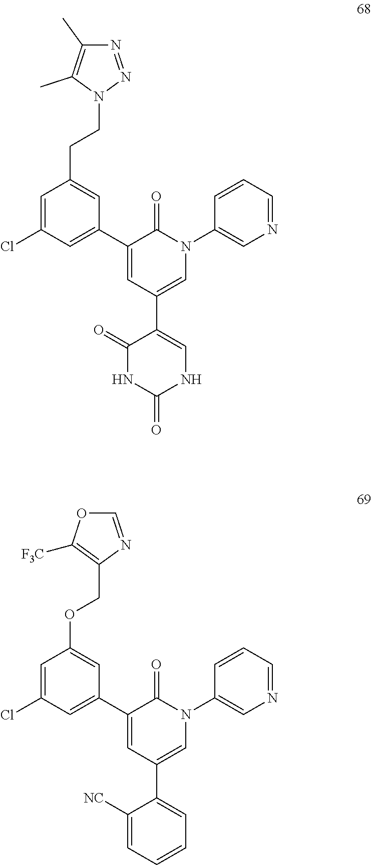 [SMILES: Cc1nnn(CCc2cc(Cl)cc(-c3cc(-c4c[nH]c(=O)[nH]c4=O)cn(-c4cccnc4)c3=O)c2)c1C ; N#Cc1ccccc1-c1cc(-c2cc(Cl)cc(OCc3ncoc3C(F)(F)F)c2)c(=O)n(-c2cccnc2)c1]

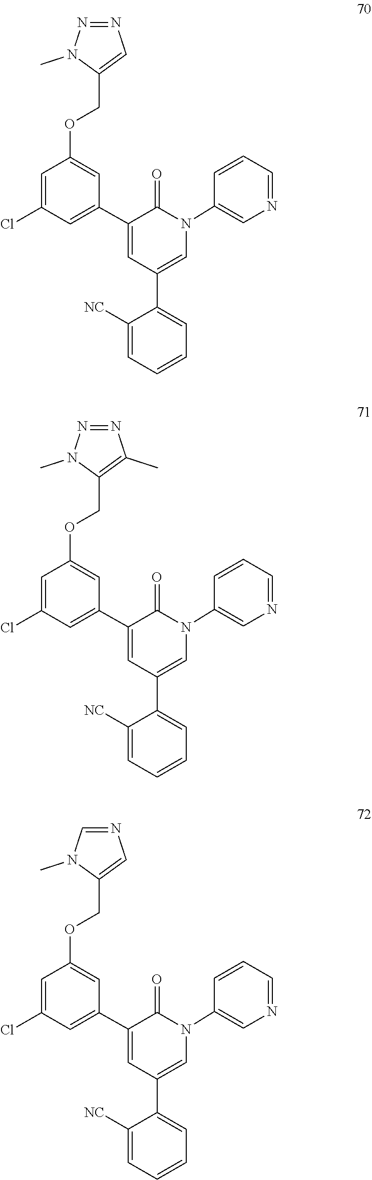 [SMILES: Cn1nncc1COc1cc(Cl)cc(-c2cc(-c3ccccc3C#N)cn(-c3cccnc3)c2=O)c1 ; Cc1nnn(C)c1COc1cc(Cl)cc(-c2cc(-c3ccccc3C#N)cn(-c3cccnc3)c2=O)c1 ; Cn1cncc1COc1cc(Cl)cc(-c2cc(-c3ccccc3C#N)cn(-c3cccnc3)c2=O)c1]

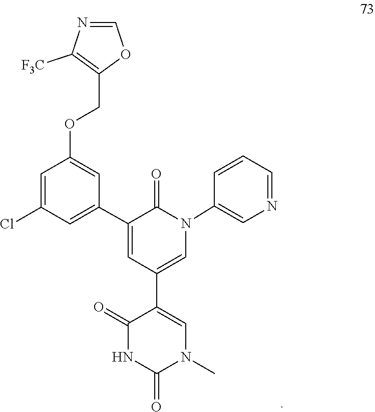 [SMILES: Cn1cc(-c2cc(-c3cc(Cl)cc(OCc4ocnc4C(F)(F)F)c3)c(=O)n(-c3cccnc3)c2)c(=O)[nH]c1=O]

Embodiment 18 provides a pharmaceutical composition comprising the compound of any one of embodiments 1-17 and at least one pharmaceutically acceptable excipient.

Embodiment 19 provides a method of treating, ameliorating, and/or preventing COVID-19, the method comprising administering a therapeutically effective amount of the compound of claim 1 to a subject in need thereof.

Embodiment 20 provides the method of embodiment 19, wherein the compound is formulated as a pharmaceutical composition comprising at least one pharmaceutically acceptable excipient.

Embodiment 21 provides the method of any one of embodiments 19-20, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

Embodiment 22 provides the method of any one of embodiments 19-21, further comprising administering at least one additional therapeutic agent.

Embodiment 23 provides the method of any one of embodiments 19-22, wherein the at least one additional therapeutic agent is administered sequentially or concurrently with the compound.

Embodiment 24 provides the method of any one of embodiments 19-23, wherein the subject is human.

Embodiment 25 provides a method of inhibiting Severe Respiratory Syndrome-Coronavirus-2 (SARS-CoV-2) main protease, the method comprising contacting SARS-CoV-2 main protease with the compound of claim 1.

Embodiment 26 provides the method of embodiment 25, wherein the contacting comprises administering the compound to a subject in an amount sufficient to inhibit the biological activity of SARS-CoV-2 main protease.

Embodiment 27 provides the method of any one of embodiments 25-26, wherein the subject is a human.

Embodiment 28 provides the method of any one of embodiments 25-27, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

Embodiment 29 provides the method of any one of embodiments 25-28, further comprising administering at least one additional therapeutic agent.

Embodiment 30 provides the method of any one of embodiments 25-29, wherein the at least one additional therapeutic agent is administered sequentially or concurrently with the compound.

Embodiment 31 provides a method of treating, preventing, reducing, and/or ameliorating one or more symptoms associated with COVID-19 infection, the method comprising administering a therapeutically effective amount of the compound of claim 1 to a subject in need thereof.

Embodiment 32 provides the method of embodiment 31, wherein the compound is formulated as a pharmaceutical composition comprising at least one pharmaceutically acceptable excipient.

Embodiment 33 provides the method of any one of embodiments 31-32, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

Embodiment 34 provides the method of any one of embodiments 31-33, further comprising administering at least one additional therapeutic agent.

Embodiment 35 provides the method of any one of embodiments 31-34, wherein the at least one additional therapeutic agent is administered sequentially or concurrently with the compound.

Embodiment 36 provides the method of any one of embodiments 31-35, wherein the subject is human.

Embodiment 37 provides the method of any one of embodiments 31-36, wherein the one or more symptoms is at least one of fever, cough, myalgia, fatigue, sputum production, headache, diarrhea, vomiting, dyspnea, lymphopenia, hypoalbuminemia, and combinations thereof.

Embodiment 38 provides a compound having the structure of formula II:

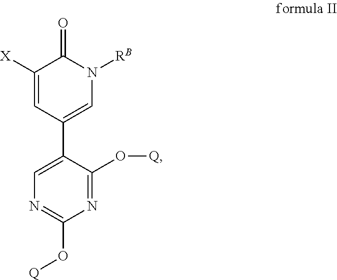

wherein:
X is selected from the group consisting of F, Cl, Br, and I; R^{B} is

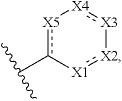

wherein: each X1-X5 is independently C—Y, N, or NR; each Y is independently selected from the group consisting of hydrogen, O (oxo), F, Cl, C₁₋₄ alkyl, and OC₁₋₄ alkyl; [symbol] is a single or double bond; and provided that 1 to 3 of X1-X5 is N or NR, and if at least one of X1-X5 is NR then an adjacent position to the NR is C═O; and wherein Q is C₁₋₄ alkyl.

Embodiment 39 provides the compound of embodiment 38, having the structure:

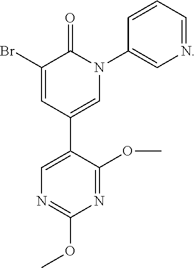

1. A compound of formula (I) or formula (I-A), or a salt, solvate, enantiomer, diastereomer, tautomer, or N-oxide thereof: 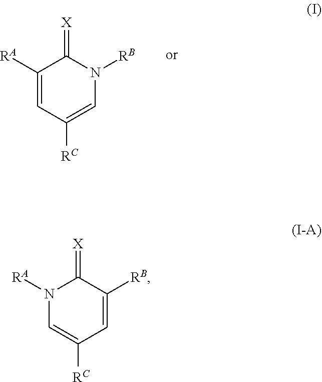
wherein:
each occurrence of R^{A} is independently a 5, 6, 7, or 8-membered heterocyclyl;
each occurrence of R^{B} is independently a 5, 6, 7, 8, 9 or 10-membered heterocyclyl;
each occurrence of R^{C} is independently a 5, 6, 7, or 8-membered heterocyclyl; and
each occurrence of X is independently O, S, or N—OR;
wherein each of R^{A}, R^{B}, and R^{C} is independently substituted by 1 to 5 substituents independently selected from the group consisting of hydrogen, C₆₋₁₄ aryl, C₆₋₁₄ heteroaryl, C₁₋₁₀ alkoxy, C₁₋₁₀ alkyl, C₃₋₁₀ cycloalkyl, C₃₋₁₀ cycloheteroalkyl, C₂₋₁₀ alkenyl, C₂₋₁₀ alkynyl, OR, OC(O)N(R)₂, OCH₂C(O)N(R)₂, O (oxo), F, Cl, Br, I, NO₂, CN, CF₃, OCF₃, N(R)₂, SR, SOR, SO₂R, SO₂N(R)₂, SO₃R, C(O)R, C(O)OR, OC(O)R, C(O)N(R)₂, and combinations thereof; and
wherein each occurrence of R is independently hydrogen, C₁₋₁₂ alkyl, C₂₋₁₂ alkenyl, C₂₋₁₂ alkynyl, C₅₋₁₂ cycloalkyl, C₆₋₁₀ aryl, C₅₋₁₀ heteroaryl, or combinations thereof.

2. The compound of claim 1, wherein:
X is O;
R^{A} is a 6-membered aryl or heteroaryl;
R^{B} is a 6-membered aryl, 6-membered heteroaryl, 10-membered aryl, or 10-membered heteroaryl; and
R^{C} is a 6-membered cycloheteroalkyl, aryl, or heteroaryl.

3. The compound of claim 2, wherein R^{A} is: 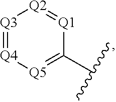
wherein Q1 is C-A¹ or N, Q2 is C-A² or N, Q3 is C-A³ or N, Q4 is C-A⁴ or N, Q5 is C-A⁵ or N, wherein 0-3 of Q1-Q5 can be N,
wherein each A¹-A⁵ is independently selected from the group consisting of hydrogen, C₆₋₁₄ aryl, C₆₋₁₄ heteroaryl, C₁₋₁₀ alkoxy, C₃₋₁₀ cycloalkyl, C₃₋₁₀ cycloheteroalkyl, C₁₋₁₀ alkyl, C₂₋₁₀ alkenyl, C₂₋₁₀ alkynyl, OR, OC(O)N(R)₂, F, Cl, Br, I, NO₂, CN, CF₃, OCF₃, N(R)₂, SR, SOR, SO₂R, SO₂N(R)₂, SO₃R, C(O)R, C(O)OR, OC(O)R, C(O)N(R)₂, and combinations thereof.

4. The compound of claim 3, wherein R^{A} is: 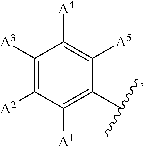
and one of the following applies:
i. A¹, A³, A⁴, and A⁵ are hydrogen,
ii. A¹ and A⁵ are hydrogen, or
iii. A¹, A³, and A⁵ are hydrogen.

5. The compound of claim 4, wherein each A¹-A⁵ is independently selected from the group consisting of hydrogen, C₆₋₁₄ aryl, C₁₋₁₀ alkoxy, C₃₋₁₀ cycloalkyl, C₃₋₁₀ cycloheteroalkyl, C₁₋₁₀ alkyl, OR, OC(O)N(R)₂, OCH₂C(O)N(R)₂, F, Cl, Br, NO₂, CN, CF₃, OCF₃, and combinations thereof.

6. The compound of claim 5, wherein each A¹-A⁵ is independently selected from the group consisting of hydrogen, F, Cl, CN, OC₁₋₆ alkyl, OC₁₋₆ alkyl substituted by 1 to 5 hydroxyl groups, OC₁₋₆ alkyl substituted by 1 to 5 CF₃ groups, O(CH₂)ₙPh, O(CH₂)ₙAr, and O(CH₂CH₂O)ₘCH₃, wherein
each n is independently at each occurrence an integer from 1 to 5;
each m is independently at each occurrence an integer from 1 to 5;
Ar is phenyl substituted with 1 to 5 substituents selected from the group consisting of C₁₋₅ hydrocarbyl, CF₃, F, Cl, Br, and combinations thereof, or
Ar is a 5-membered heteroaryl or a 6-membered heteroaryl substituted with 1 to 5 substituents selected from the group consisting of hydrogen, C₁₋₅ hydrocarbyl, CF₃, F, Cl, Br, and combinations thereof.

7. The compound of claim 1, wherein R^{A} is independently selected from the group consisting of:

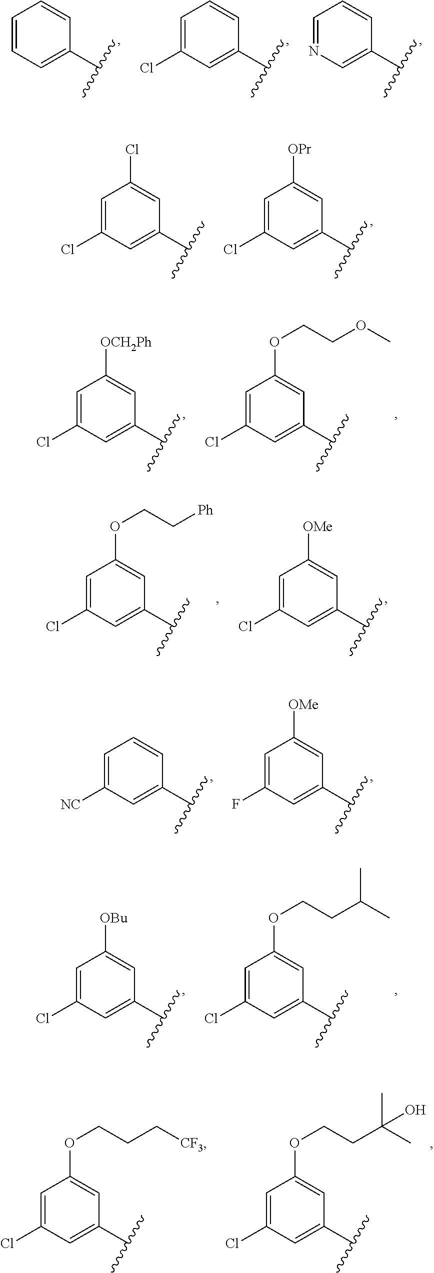

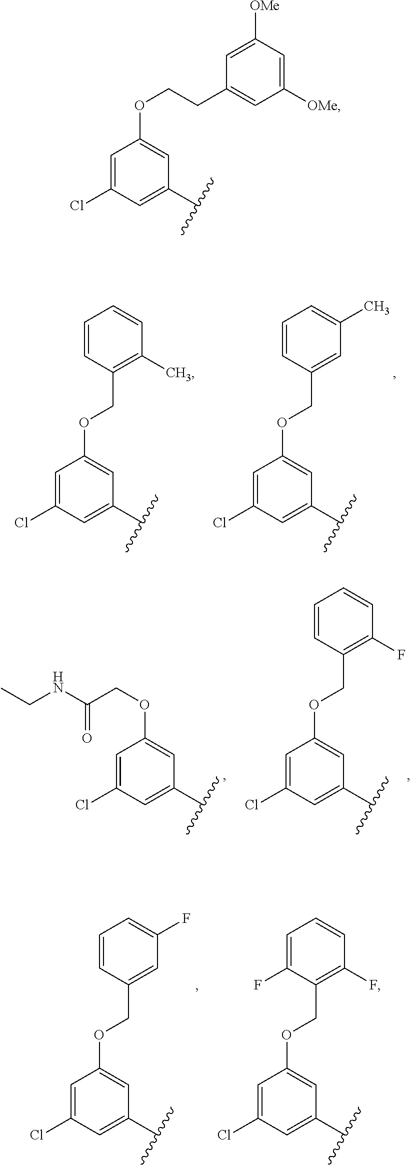

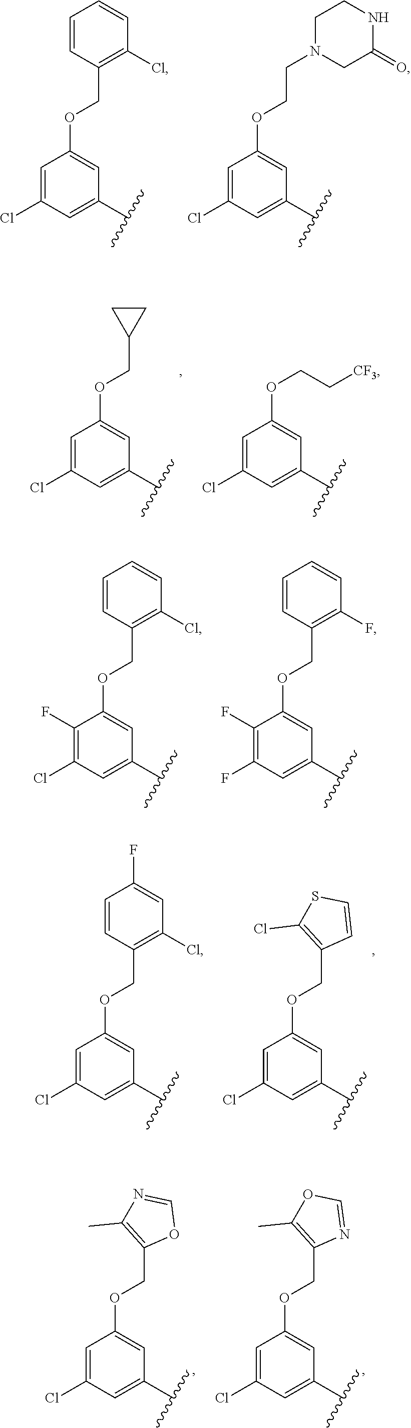

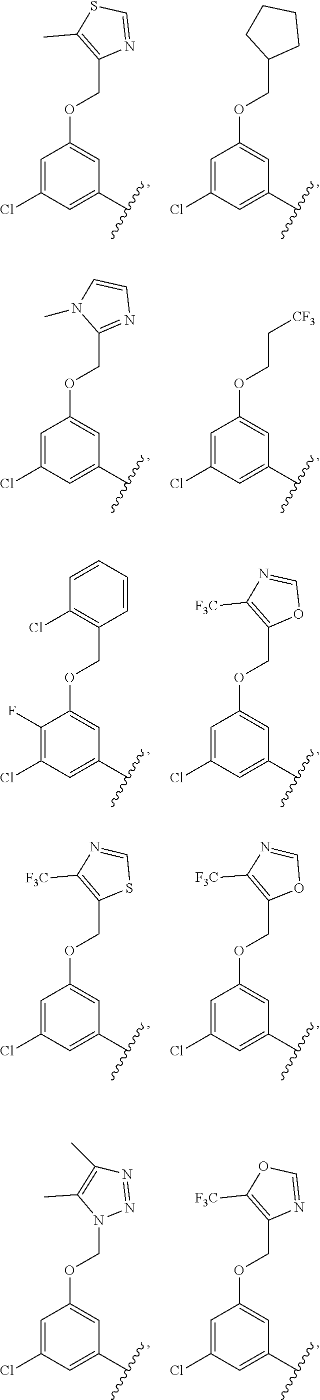

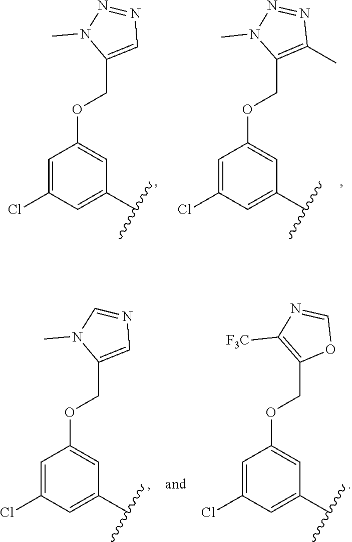 [SMILES: *c1cc(Cl)cc(OCc2cnnn2C)c1 ; *c1cc(Cl)cc(OCc2c(C)nnn2C)c1 ; *c1cc(Cl)cc(OCc2cncn2C)c1 ; *c1cc(Cl)cc(OCc2ocnc2C(F)(F)F)c1]

8. The compound of claim 2, wherein R^{B} is 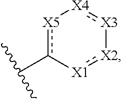
wherein
each X1-X5 is independently C—Y, N, or NR;
each Y is independently selected from the group consisting of hydrogen, O (oxo), F, Cl, C₁₋₄ alkyl, and OC₁₋₄ alkyl; [symbol]
is a single or double bond; and
provided that
1 to 3 of X1-X5 is N or NR, and
if at least one of X1-X5 is NR then an adjacent position to the NR is C═O.

9. The compound of claim 2, wherein R^{B} is

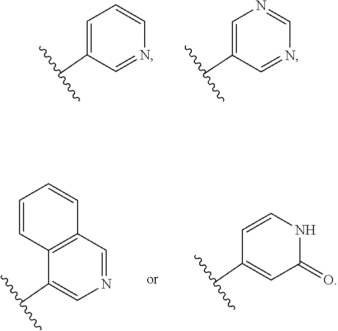 [SMILES: *c1cccnc1 ; *c1cncnc1 ; *c1cncc2ccccc12 ; *c1cc[nH]c(=O)c1]

10. (canceled)

11. The compound of claim 2, wherein R^{B} is 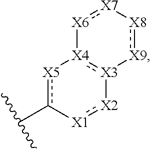
wherein
each X1, X2, X5, X6, X7, X8, and X9 is independently C—Y, N, or NR;
each X3 and X4 is independently C—Y or C;
each Y is independently selected from the group consisting of hydrogen, O (oxo), F, Cl, C₁₋₄ alkyl, and OC₁₋₄ alkyl; [symbol]
is a single or double bond; and
provided that
1 to 3 of X1, X2, X5, X6, X7, X8, and X9 is N or NR, and
if at least one of X1, X2, X5, X6, X7, X8, and X9 is NR then an adjacent position to the NR is C═O.

12. (canceled)

13. The compound of claim 2, wherein R^{C} is 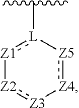
wherein
each Z1-Z5 is independently C—Y, N, or NR;
L is C or N;
each Y is independently selected from the group consisting of hydrogen, O (oxo), F, Cl, Br, I, NO₂, NR₂, CN, CF₃, OCF₃, C₁₋₄ alkyl, and OC₁₋₄ alkyl; [symbol]
is a single or double bond; and
provided that
1 to 3 of Z1-Z5 is N or NR, and
if at least one of Z1-Z5 is NR then an adjacent position to the NR is C═O.

14. The compound of claim 13, wherein R^{C} is selected from the group consisting of

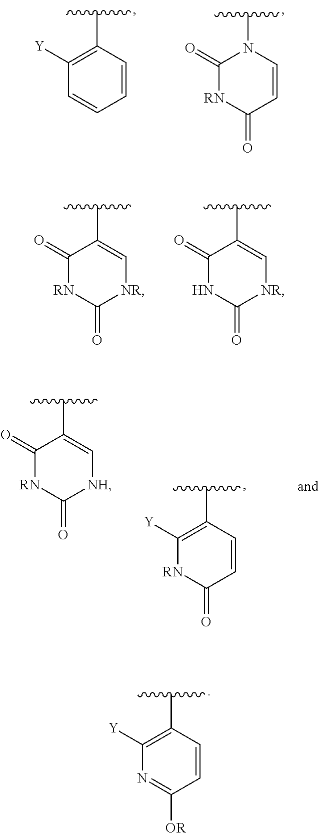

15. The compound of claim 14, wherein Y is CN.

16. The compound of claim 14, wherein R^{C} is selected from the group consisting of

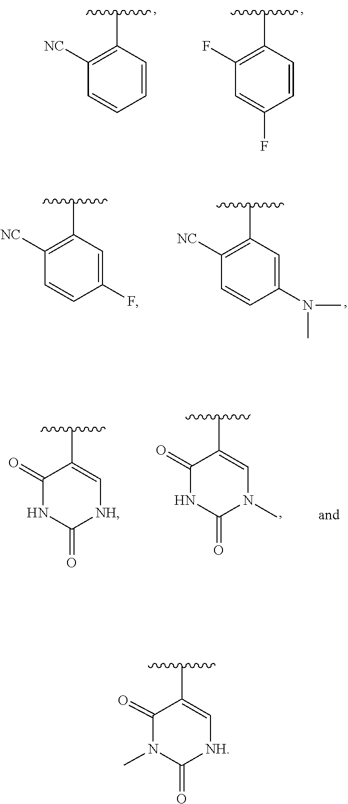

17. The compound of claim 1, which is selected from the group consisting of:

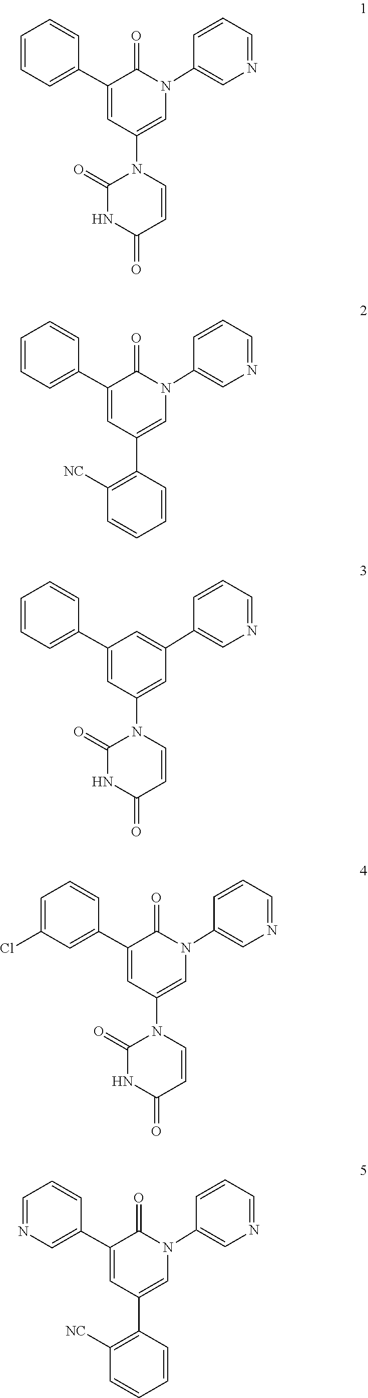 [SMILES: O=c1ccn(-c2cc(-c3ccccc3)c(=O)n(-c3cccnc3)c2)c(=O)[nH]1 ; N#Cc1ccccc1-c1cc(-c2ccccc2)c(=O)n(-c2cccnc2)c1 ; O=c1ccn(-c2cc(-c3ccccc3)cc(-c3cccnc3)c2)c(=O)[nH]1 ; O=c1ccn(-c2cc(-c3cccc(Cl)c3)c(=O)n(-c3cccnc3)c2)c(=O)[nH]1 ; N#Cc1ccccc1-c1cc(-c2cccnc2)c(=O)n(-c2cccnc2)c1]

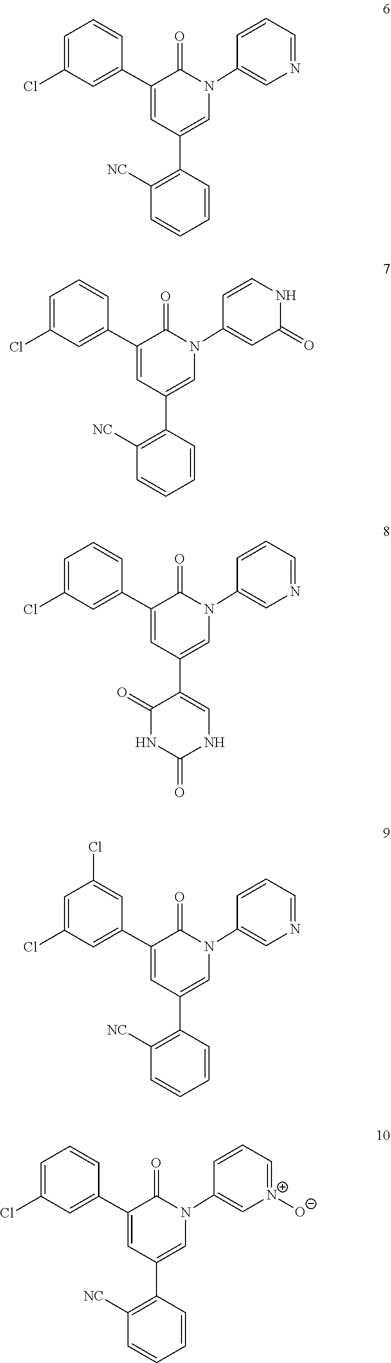 [SMILES: N#Cc1ccccc1-c1cc(-c2cccc(Cl)c2)c(=O)n(-c2cccnc2)c1 ; N#Cc1ccccc1-c1cc(-c2cccc(Cl)c2)c(=O)n(-c2cc[nH]c(=O)c2)c1 ; O=c1[nH]cc(-c2cc(-c3cccc(Cl)c3)c(=O)n(-c3cccnc3)c2)c(=O)[nH]1 ; N#Cc1ccccc1-c1cc(-c2cc(Cl)cc(Cl)c2)c(=O)n(-c2cccnc2)c1 ; N#Cc1ccccc1-c1cc(-c2cccc(Cl)c2)c(=O)n(-c2ccc[n+]([O-])c2)c1]

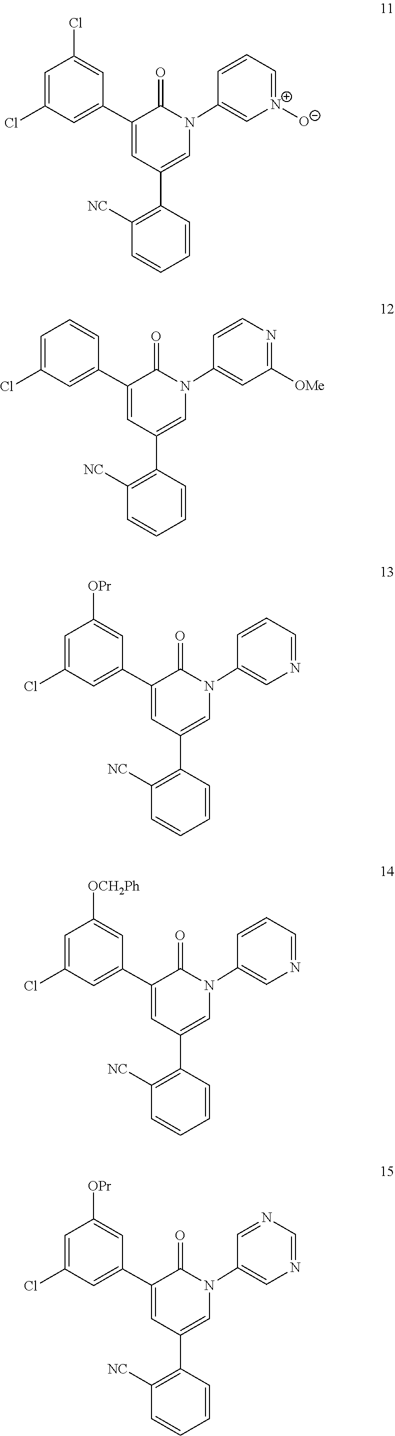

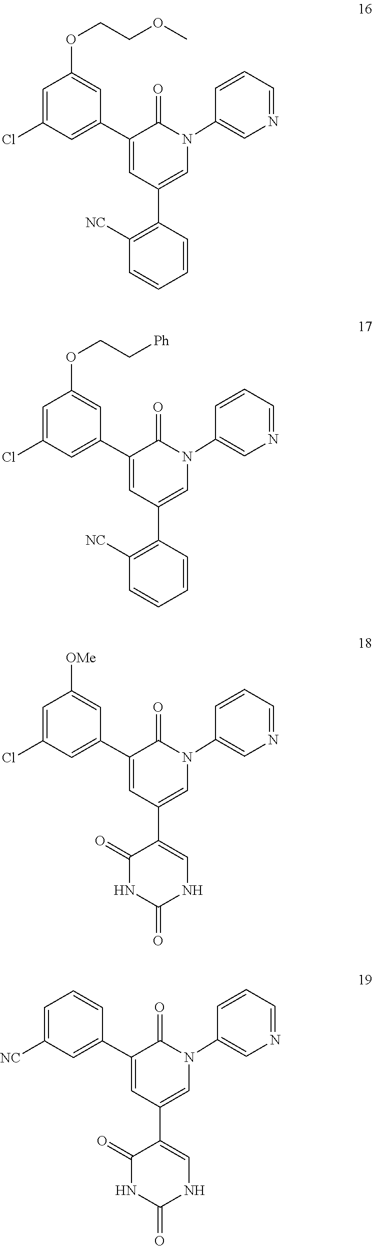 [SMILES: COCCOc1cc(Cl)cc(-c2cc(-c3ccccc3C#N)cn(-c3cccnc3)c2=O)c1 ; N#Cc1ccccc1-c1cc(-c2cc(Cl)cc(OCCc3ccccc3)c2)c(=O)n(-c2cccnc2)c1 ; COc1cc(Cl)cc(-c2cc(-c3c[nH]c(=O)[nH]c3=O)cn(-c3cccnc3)c2=O)c1 ; N#Cc1cccc(-c2cc(-c3c[nH]c(=O)[nH]c3=O)cn(-c3cccnc3)c2=O)c1]

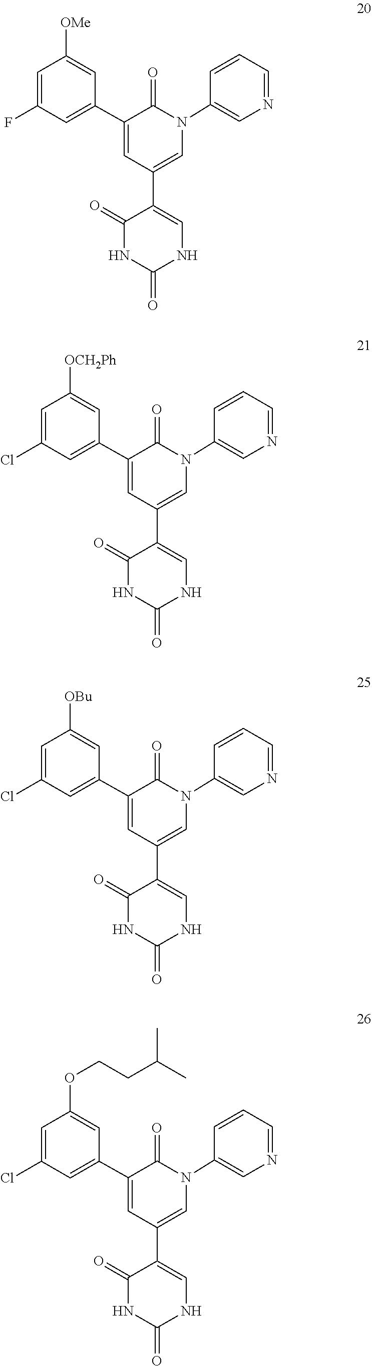

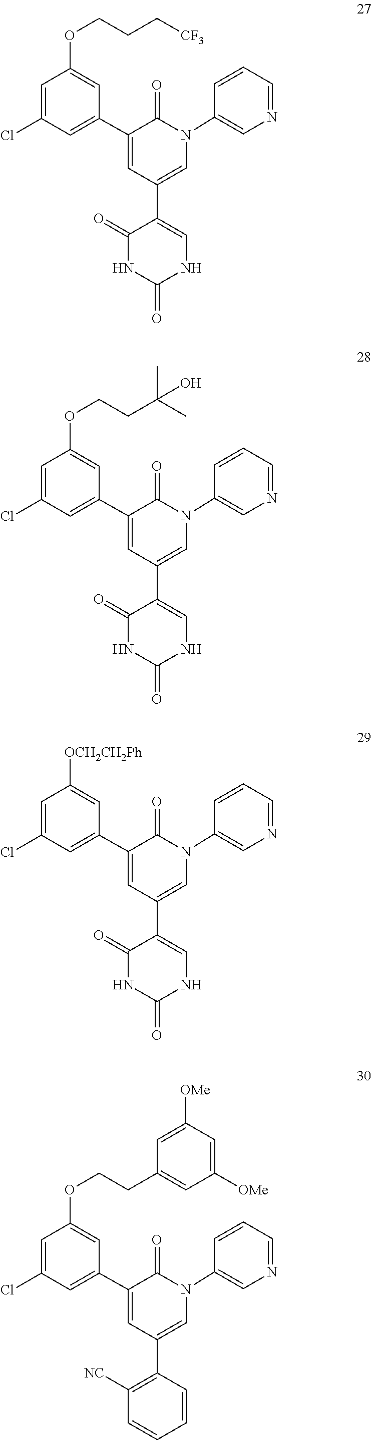

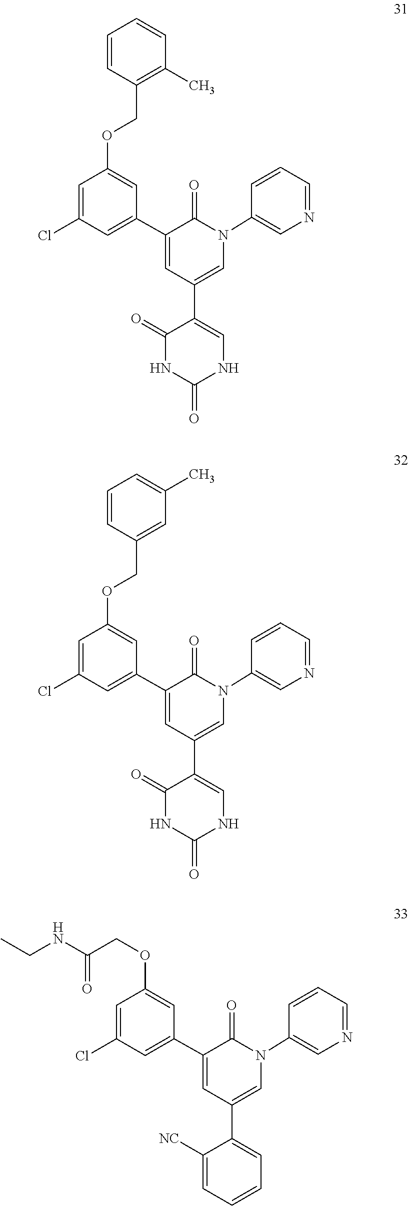 [SMILES: Cc1ccccc1COc1cc(Cl)cc(-c2cc(-c3c[nH]c(=O)[nH]c3=O)cn(-c3cccnc3)c2=O)c1 ; Cc1cccc(COc2cc(Cl)cc(-c3cc(-c4c[nH]c(=O)[nH]c4=O)cn(-c4cccnc4)c3=O)c2)c1 ; CCNC(=O)COc1cc(Cl)cc(-c2cc(-c3ccccc3C#N)cn(-c3cccnc3)c2=O)c1]

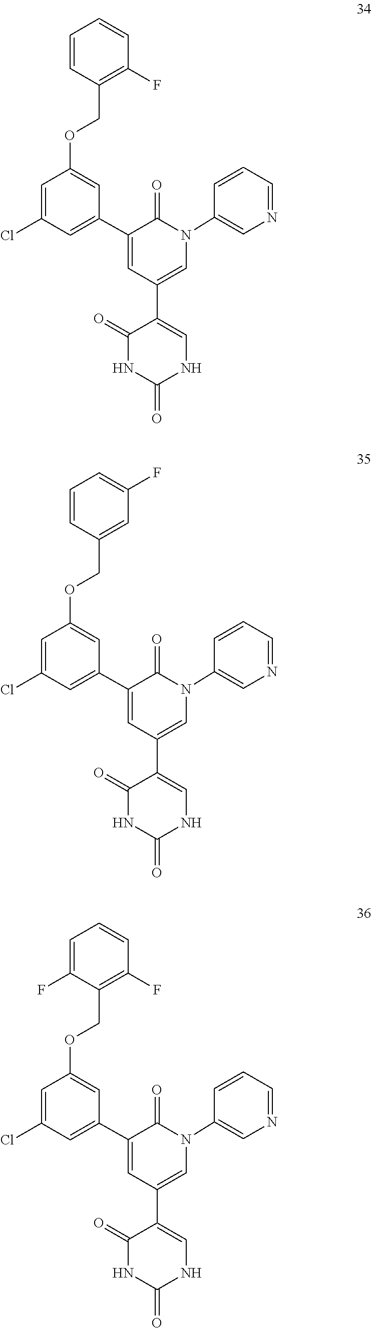 [SMILES: O=c1[nH]cc(-c2cc(-c3cc(Cl)cc(OCc4ccccc4F)c3)c(=O)n(-c3cccnc3)c2)c(=O)[nH]1 ; O=c1[nH]cc(-c2cc(-c3cc(Cl)cc(OCc4cccc(F)c4)c3)c(=O)n(-c3cccnc3)c2)c(=O)[nH]1 ; O=c1[nH]cc(-c2cc(-c3cc(Cl)cc(OCc4c(F)cccc4F)c3)c(=O)n(-c3cccnc3)c2)c(=O)[nH]1]

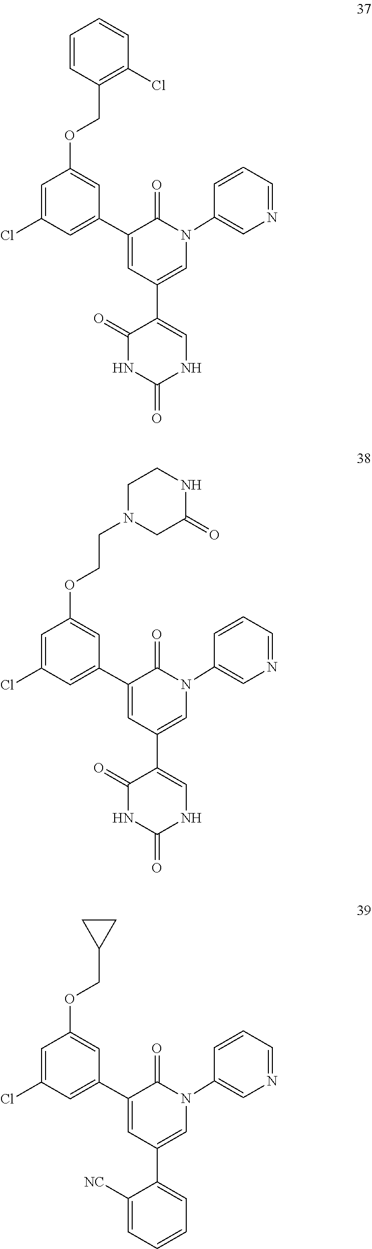 [SMILES: O=c1[nH]cc(-c2cc(-c3cc(Cl)cc(OCc4ccccc4Cl)c3)c(=O)n(-c3cccnc3)c2)c(=O)[nH]1 ; O=C1CN(CCOc2cc(Cl)cc(-c3cc(-c4c[nH]c(=O)[nH]c4=O)cn(-c4cccnc4)c3=O)c2)CCN1 ; N#Cc1ccccc1-c1cc(-c2cc(Cl)cc(OCC3CC3)c2)c(=O)n(-c2cccnc2)c1]

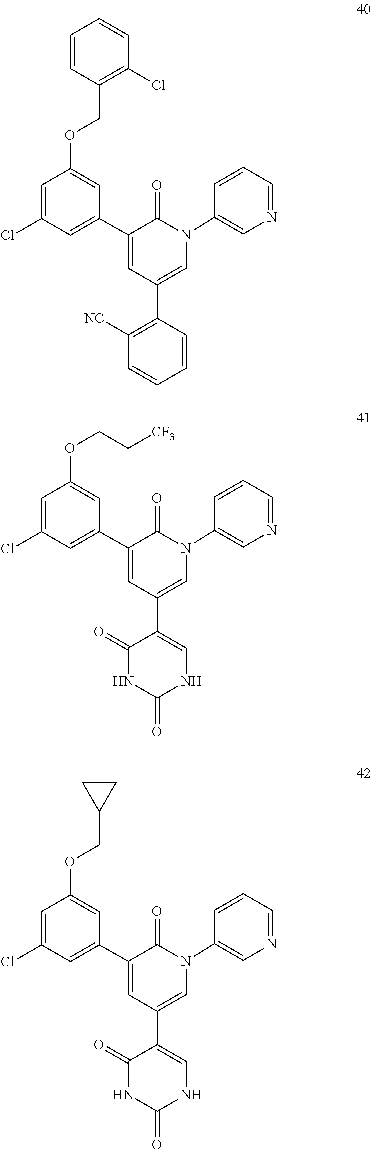 [SMILES: N#Cc1ccccc1-c1cc(-c2cc(Cl)cc(OCc3ccccc3Cl)c2)c(=O)n(-c2cccnc2)c1 ; O=c1[nH]cc(-c2cc(-c3cc(Cl)cc(OCCC(F)(F)F)c3)c(=O)n(-c3cccnc3)c2)c(=O)[nH]1 ; O=c1[nH]cc(-c2cc(-c3cc(Cl)cc(OCC4CC4)c3)c(=O)n(-c3cccnc3)c2)c(=O)[nH]1]

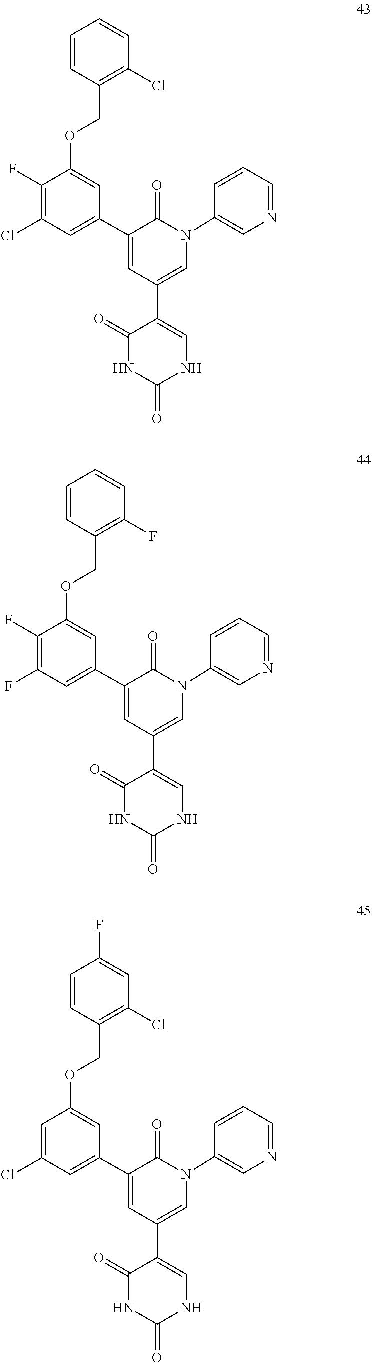 [SMILES: O=c1[nH]cc(-c2cc(-c3cc(Cl)c(F)c(OCc4ccccc4Cl)c3)c(=O)n(-c3cccnc3)c2)c(=O)[nH]1 ; O=c1[nH]cc(-c2cc(-c3cc(F)c(F)c(OCc4ccccc4F)c3)c(=O)n(-c3cccnc3)c2)c(=O)[nH]1 ; O=c1[nH]cc(-c2cc(-c3cc(Cl)cc(OCc4ccc(F)cc4Cl)c3)c(=O)n(-c3cccnc3)c2)c(=O)[nH]1]

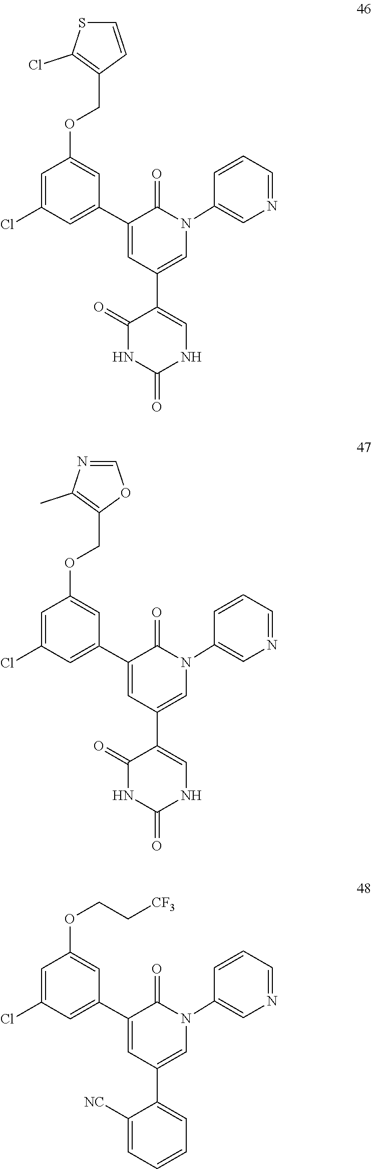 [SMILES: O=c1[nH]cc(-c2cc(-c3cc(Cl)cc(OCc4ccsc4Cl)c3)c(=O)n(-c3cccnc3)c2)c(=O)[nH]1 ; Cc1ncoc1COc1cc(Cl)cc(-c2cc(-c3c[nH]c(=O)[nH]c3=O)cn(-c3cccnc3)c2=O)c1 ; N#Cc1ccccc1-c1cc(-c2cc(Cl)cc(OCCC(F)(F)F)c2)c(=O)n(-c2cccnc2)c1]

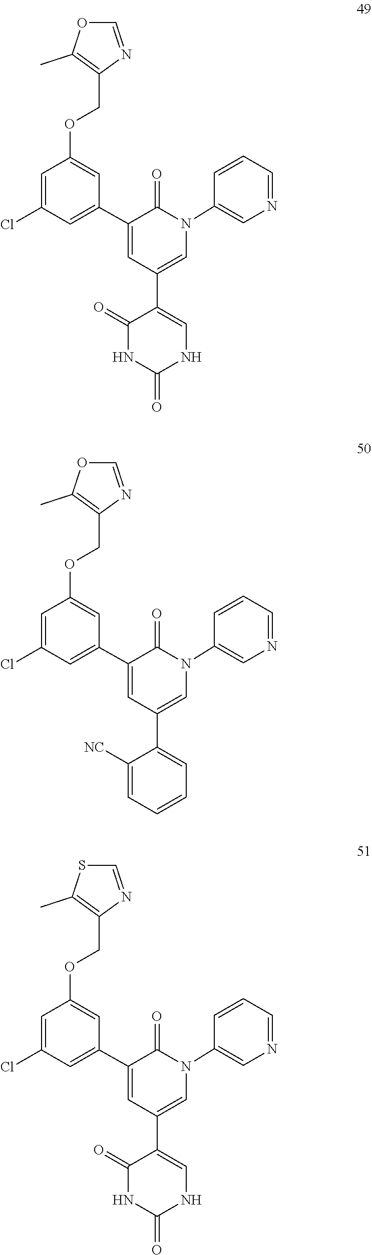 [SMILES: Cc1ocnc1COc1cc(Cl)cc(-c2cc(-c3c[nH]c(=O)[nH]c3=O)cn(-c3cccnc3)c2=O)c1 ; Cc1ocnc1COc1cc(Cl)cc(-c2cc(-c3ccccc3C#N)cn(-c3cccnc3)c2=O)c1 ; Cc1scnc1COc1cc(Cl)cc(-c2cc(-c3c[nH]c(=O)[nH]c3=O)cn(-c3cccnc3)c2=O)c1]

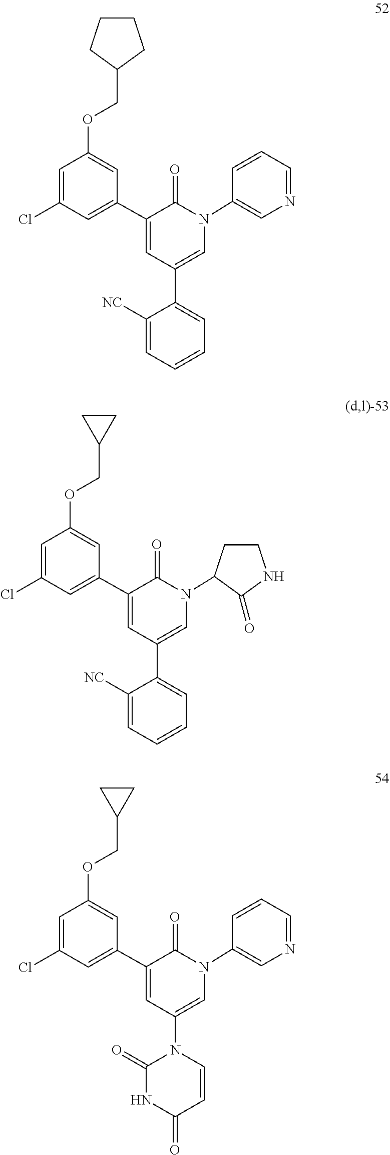 [SMILES: N#Cc1ccccc1-c1cc(-c2cc(Cl)cc(OCC3CCCC3)c2)c(=O)n(-c2cccnc2)c1 ; N#Cc1ccccc1-c1cc(-c2cc(Cl)cc(OCC3CC3)c2)c(=O)n(C2CCNC2=O)c1 ; O=c1ccn(-c2cc(-c3cc(Cl)cc(OCC4CC4)c3)c(=O)n(-c3cccnc3)c2)c(=O)[nH]1]

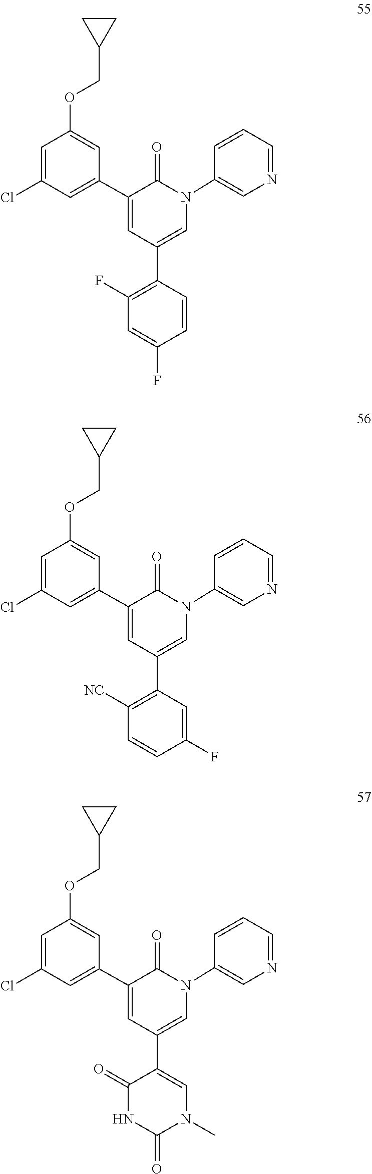 [SMILES: O=c1c(-c2cc(Cl)cc(OCC3CC3)c2)cc(-c2ccc(F)cc2F)cn1-c1cccnc1 ; N#Cc1ccc(F)cc1-c1cc(-c2cc(Cl)cc(OCC3CC3)c2)c(=O)n(-c2cccnc2)c1 ; Cn1cc(-c2cc(-c3cc(Cl)cc(OCC4CC4)c3)c(=O)n(-c3cccnc3)c2)c(=O)[nH]c1=O]

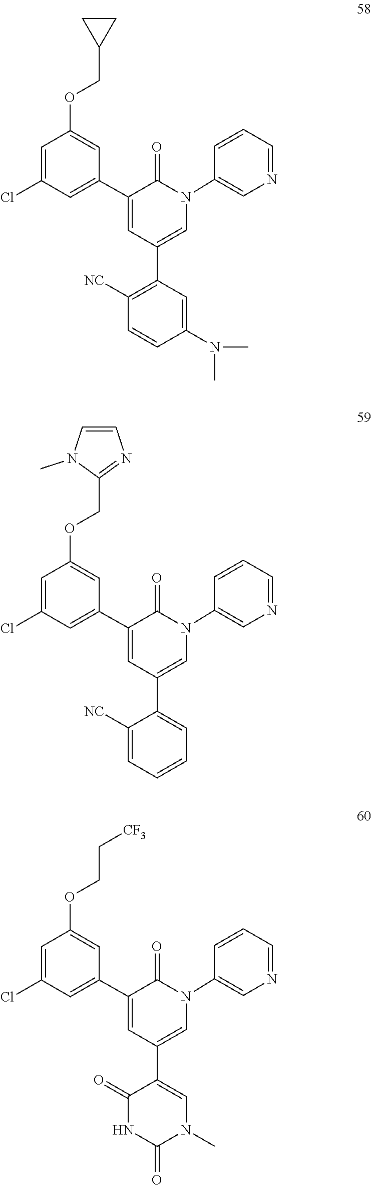 [SMILES: CN(C)c1ccc(C#N)c(-c2cc(-c3cc(Cl)cc(OCC4CC4)c3)c(=O)n(-c3cccnc3)c2)c1 ; Cn1ccnc1COc1cc(Cl)cc(-c2cc(-c3ccccc3C#N)cn(-c3cccnc3)c2=O)c1 ; Cn1cc(-c2cc(-c3cc(Cl)cc(OCCC(F)(F)F)c3)c(=O)n(-c3cccnc3)c2)c(=O)[nH]c1=O]

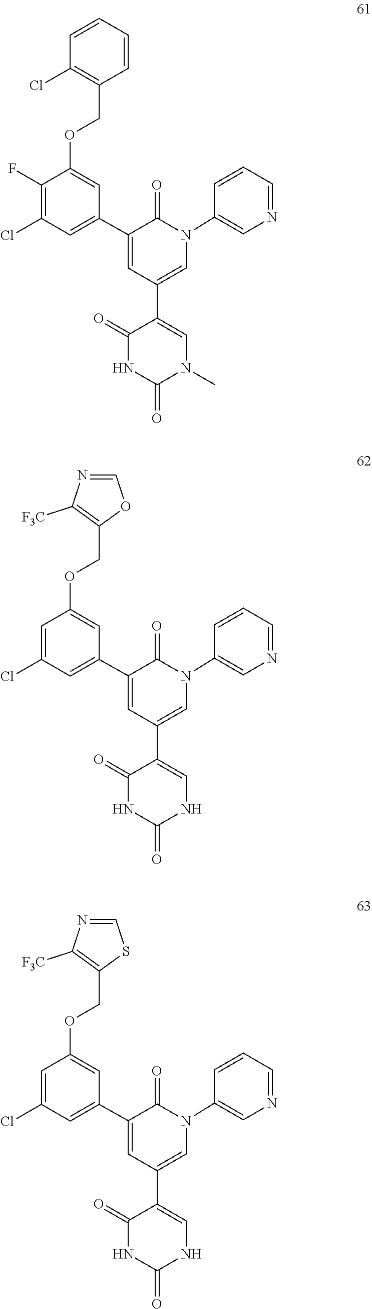 [SMILES: Cn1cc(-c2cc(-c3cc(Cl)c(F)c(OCc4ccccc4Cl)c3)c(=O)n(-c3cccnc3)c2)c(=O)[nH]c1=O ; O=c1[nH]cc(-c2cc(-c3cc(Cl)cc(OCc4ocnc4C(F)(F)F)c3)c(=O)n(-c3cccnc3)c2)c(=O)[nH]1 ; O=c1[nH]cc(-c2cc(-c3cc(Cl)cc(OCc4scnc4C(F)(F)F)c3)c(=O)n(-c3cccnc3)c2)c(=O)[nH]1]

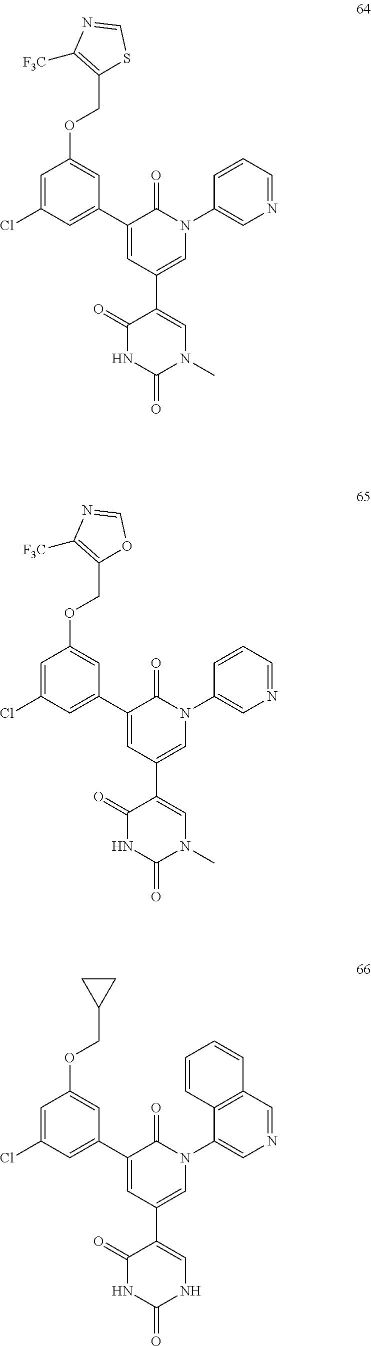 [SMILES: Cn1cc(-c2cc(-c3cc(Cl)cc(OCc4scnc4C(F)(F)F)c3)c(=O)n(-c3cccnc3)c2)c(=O)[nH]c1=O ; Cn1cc(-c2cc(-c3cc(Cl)cc(OCc4ocnc4C(F)(F)F)c3)c(=O)n(-c3cccnc3)c2)c(=O)[nH]c1=O ; O=c1[nH]cc(-c2cc(-c3cc(Cl)cc(OCC4CC4)c3)c(=O)n(-c3cncc4ccccc34)c2)c(=O)[nH]1]

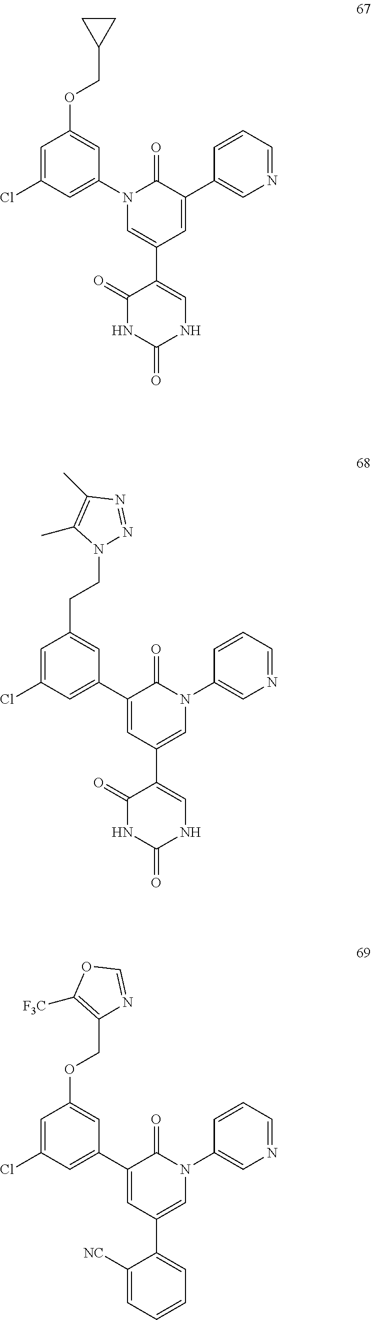 [SMILES: O=c1[nH]cc(-c2cc(-c3cccnc3)c(=O)n(-c3cc(Cl)cc(OCC4CC4)c3)c2)c(=O)[nH]1 ; Cc1nnn(CCc2cc(Cl)cc(-c3cc(-c4c[nH]c(=O)[nH]c4=O)cn(-c4cccnc4)c3=O)c2)c1C ; N#Cc1ccccc1-c1cc(-c2cc(Cl)cc(OCc3ncoc3C(F)(F)F)c2)c(=O)n(-c2cccnc2)c1]

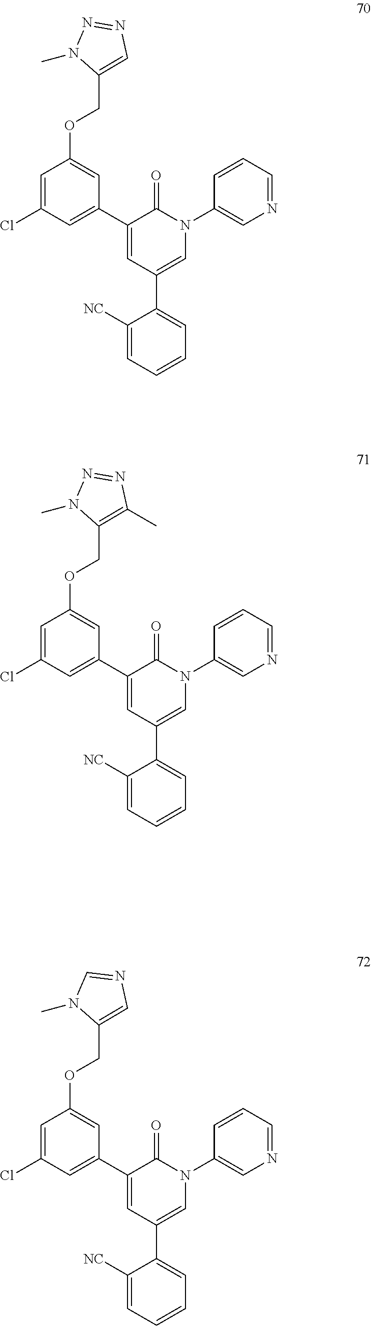 [SMILES: Cn1nncc1COc1cc(Cl)cc(-c2cc(-c3ccccc3C#N)cn(-c3cccnc3)c2=O)c1 ; Cc1nnn(C)c1COc1cc(Cl)cc(-c2cc(-c3ccccc3C#N)cn(-c3cccnc3)c2=O)c1 ; Cn1cncc1COc1cc(Cl)cc(-c2cc(-c3ccccc3C#N)cn(-c3cccnc3)c2=O)c1]

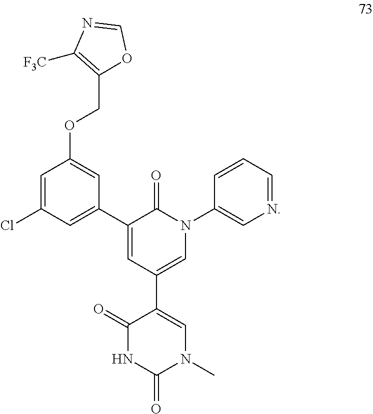 [SMILES: Cn1cc(-c2cc(-c3cc(Cl)cc(OCc4ocnc4C(F)(F)F)c3)c(=O)n(-c3cccnc3)c2)c(=O)[nH]c1=O]

18. (canceled)

19. A method of:
i) treating COVID-19 in a subject in need thereof; or
ii) treating, reducing, or ameliorating one or more symptoms associated with COVID-19 infection in a subject in need thereof, wherein the one or more symptoms is at least one of fever, cough, myalgia, fatigue, sputum production, headache, diarrhea, vomiting, dyspnea, lymphopenia, hypoalbuminemia, and combinations thereof,
the method comprising administering to the subject in need thereof a therapeutically effective amount of a compound of formula (I) or formula (I-A), or a salt, solvate, enantiomer, diastereomer, tautomer, or N-oxide thereof: 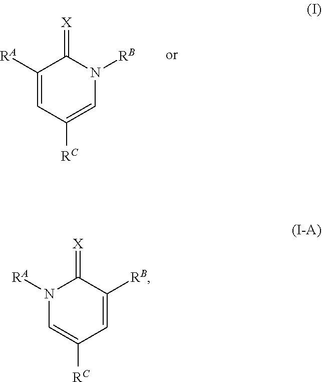
wherein:
each occurrence of R^{A} is independently a 5, 6, 7, or 8-membered heterocyclyl;
each occurrence of R^{B} is independently a 5, 6, 7, 8, 9 or 10-membered heterocyclyl;
each occurrence of R^{C} is independently a 5, 6, 7, or 8-membered heterocyclyl; and
each occurrence of X is independently O, S, or N—OR:
wherein each of R^{A}, R^{B}, and R^{C} is independently substituted by 1 to 5 substituents independently selected from the group consisting of hydrogen, C₆₋₁₄ aryl, C₆₋₁₄ heteroaryl, C₁₋₁₀ alkoxy, C₁₋₁₀ alkyl, C₃₋₁₀ cycloalkyl, C₃₋₁₀ cycloheteroalkyl, C₂₋₁₀ alkenyl, C₂₋₁₀ alkynyl, OR, OC(O)N(R)₂, OCH₂C(O)N(R)₂, O (oxo), F, Cl, Br, I, NO₂, CN, CF₃, OCF₃, N(R)₂, SR, SOR, SO₂R, SO₂N(R)₂, SO₃R, C(O)R, C(O)OR, OC(O)R, C(O)N(R)₂, and combinations thereof; and
wherein each occurrence of R is independently hydrogen, C₁₋₁₂ alkyl, C₂₋₁₂ alkenyl, C₂₋₁₂ alkynyl, C₅₋₁₂ cycloalkyl, C₆₋₁₀ aryl, C₅₋₁₀ heteroaryl, or combinations thereof.

20. (canceled)

21. The method of claim 19, wherein the administering is by a route selected from the group consisting of oral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

22. The method of claim 19, further comprising administering at least one additional therapeutic agent sequentially or concurrently with the compound of formula (I) or formula (I-A).

23-37. (canceled)

38. A compound having the structure of formula II: 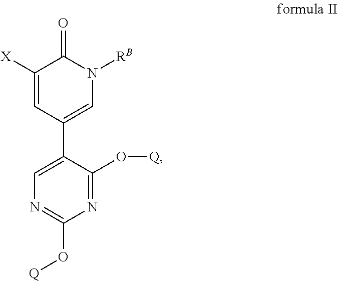
wherein:
X is selected from the group consisting of F, Cl, Br, and I;
R^{B} is 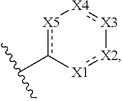
wherein
each X1-X5 is independently C—Y, N, or NR;
each Y is independently selected from the group consisting of hydrogen, O (oxo), F, Cl, C₁₋₄ alkyl, and OC₁₋₄ alkyl; [symbol]
is a single or double bond; and
provided that
1 to 3 of X1-X5 is N or NR, and
if at least one of X1-X5 is NR then an adjacent position to the NR is C═O; and
wherein Q is C₁₋₄ alkyl.

39. The compound of claim 38, having the structure: [chemical structure]


SEQUENCE LISTING (4 sequences)

SEQ ID NO: 1 - DNA, 68 bp, synthetic construct
  misc_feature 1..68: note = Recombinant
  source 1..68: mol_type = other DNA
gagttgttatttctagtgatgttcttgttaacaactaaacgaacaatggtcttcacactcgaagattt

SEQ ID NO: 2 - DNA, 30 bp, synthetic construct
  misc_feature 1..30: note = Recombinant
  source 1..30: mol_type = other DNA
gccttcataggggcgtccgaaatagtcgat

SEQ ID NO: 3 - DNA, 39 bp, synthetic construct
  misc_feature 1..39: note = Recombinant
  source 1..39: mol_type = other DNA
cgactatttcggacgcccctatgaaggcatcgccgtgtt

SEQ ID NO: 4 - DNA, 68 bp, synthetic construct
  misc_feature 1..68: note = Recombinant
  source 1..68: mol_type = other DNA
cagttccaattgtgaagattctcataaacaaatccataagttcgtttacgccagaatgcgttcgcaca